(19) United States
(12) Patent Application Publication
(10) Pub. No.: US 2026/0137697 A1
(43) Pub. Date: May 21, 2026

(54) THIAZOLO[5,4-D]PYRIMIDINE COMPOUNDS, COMPOSITIONS COMPRISING THEM AND USES THEREOF

(71) Applicant: University de Montreal, Montreal (CA)

(72) Inventors: Pierre Louis BEAULIEU, Resemère (CA); Eric BEAULIEU, Mercier (CA); Emeline BENOIT, Montréal (CA); Jonne TAN, San Mateo, CA (US); Yannick ROSE, Montréal (CA); Michael DORE, Saint-Bruno-de-Montarville (CA); Mukund GHAVRE, Saint-Laurent (CA); Doris SCHUETZ, Montréal (CA)

(21) Appl. No.: 19/120,446

(22) PCT Filed: Oct. 12, 2023
(86) PCT No.: PCT/CA2023/051356

§ 371 Date: Apr. 11, 2025

Related U.S. Application Data
(60) Provisional application No. 63/379,316, filed on Oct. 13, 2022.

(51) Int. Cl. A61K 31/5386; A61K 31/519; A61K 31/541; A61P 35/00; C07D 513/04; C07D 519/00

(52) U.S. Cl. CPC A61K 31/5386; A61K 31/519; A61K 31/541; A61P 35/00; C07D 513/04; C07D 519/00

(57) ABSTRACT

Compounds, compositions and their use in the treatment of a proliferative disease or condition such as a said proliferative disease or disorder associated with a RAF gene mutation and/or a RAS gene mutation. The compounds disclosed are of Formula (I) or a pharmaceutically acceptable salt and/or solvate thereof, wherein R¹, R², X¹, X², X³, and X⁴ are as defined herein.

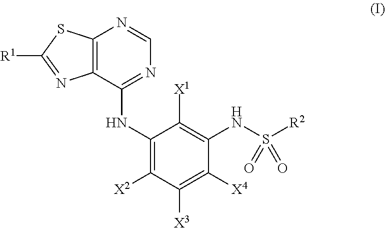

### TECHNICAL FIELD

This disclosure generally relates to thiazolo[5,4-d]pyrimidine compounds, pharmaceutical compositions comprising the same and their use in the treatment and prevention of diseases characterized by dysregulation of the RAS-ERK pathway (e.g. cancer, RASopathies).

### BACKGROUND

The RAS-RAF-MEK-ERK (RAS: rat sarcoma; RAF: rapidly accelerated fibrosarcoma; MEK: mitogen-activated protein kinase; ERK: extracellular signal-regulated kinase) signaling pathway (hereafter referred to as the RAS-ERK pathway) plays a critical role in transmitting proliferation signals generated by growth factor receptors from the plasma membrane to the nucleus. The pathway is dysregulated in a large proportion of cancers by activation of receptor tyrosine kinases (RTKs) (e.g. ERB1, ERBB2, FLT3, RET, KIT), activation or inactivation of RAS regulators (SOS1 and NF1) as well as constitutively activating mutations in RAS genes (H-, K- and NRAS; overall 30% of cancers) or in the BRAF gene (8% of cancers). The prevalence of KRAS mutations is especially high in pancreatic (>90%), colorectal (50%), and lung (30%) cancers. For their part, BRAF mutations are found with notably high frequencies in malignant melanoma (70%), thyroid cancer (40%) and colorectal cancer (10%) (mutation frequencies based on COSMIC (Catalogue Of Somatic Mutations In Cancer; Wellcome Trust Sanger Institute) release v95, November 24 2021).

RAS proteins are small GTPases that convey extracellular growth signals to intracellular effectors to control vital processes like cell differentiation, proliferation and survival (Nat. Rev. Cancer 2003, 3, 459). Physiological activation of RAS occurs at the plasma membrane after stimulation of RTKs, which leads to GTP loading of the GTPase and thus its activation. Activated RAS interacts and activates a battery of effector molecules, with the RAF kinases being the most critical RAS interactors in the context of cancer development (Nature Rev. Drug Discov. 2014, 13, 828). Oncogenic mutations at Glycine 12, Glycine 13 or Glutamine 61 in RAS isoforms lead to aberrant and constitutive signaling in human cancer (Nat. Rev. Cancer 2003, 3, 459) (COSMIC release v95, November 24 2021).

Downstream of RAS, mammalian cells express three RAF paralogs (ARAF, BRAF and CRAF) that share a conserved C-terminal kinase domain (KD) (Nat. Rev. Mol. Cell Biol. 2015, 16, 281) and an N-terminal regulatory region (NTR) comprising a RAS-binding domain (RBD). In unstimulated cells, RAF proteins are sequestered in the cytoplasm as monomers. Binding of GTP-bound activated RAS to the RBD induces membrane anchoring of RAF kinases (Nat. Rev. Mol. Cell Biol. 2015, 16, 281). Concomitantly, RAF proteins undergo kinase domain side-to-side dimerization and catalytic activation (Nature 2009, 461, 542). Activated RAF proteins convey signals through a phosphorylation cascade from RAF to MEK and then MEK to ERK, leading to phosphorylation by ERK of an array of substrates eliciting cell-specific responses (Nat. Rev. Mol. Cell Biol. 2020, Oct., 21(10), 607).

Activating mutations in RAF isoforms have so far been mostly restricted to the BRAF gene, although rare variants were observed in ARAF and CRAF, underlining the functional importance of this isoform (COSMIC release v95, November 24 2021). The most common cancer mutation in BRAF, a valine to glutamic acid substitution at position 600 (referred to as BRAFV^{600E}) enhances BRAF activity by stabilizing its active form (Cell 2004, 116, 855). Apart from the V600E allele, a diverse set of mutations occur at other residues (e.g. G466V, D594G, etc.) that lead to increased RAF signaling through a variety of mechanisms (Nat. Rev. Mol. Cell Biol. 2015, 16, 281). These have been grouped in three main classes (1 to 3) depending on their level of dependence to RAS activity and to RAF dimerization (Nature 2017 Aug. 10, 548(7666), 234-238). The key role of wild-type BRAF and CRAF in mediating RAS-driven oncogenesis by stimulating ERK signaling is extensively validated (Cancer Cell 2011, 19, 652; Cancer Discov. 2012, 2, 685; Nat. Commun. 2017, 8, 15262). Tumor cells thus rely on elevated and continued signaling of the RAS-ERK pathway through RAS and RAF activation, providing strong support for the concept of targeting RAF family kinases in cancers.

To address existing medical needs, the past decade has seen the development of a broad set of ATP-competitive RAF inhibitors (Nat. Rev. Cancer 2017, 17, 676). Efforts have focused mainly on the most common RAS-independent BRAF mutation (BRAF^{V600E}), leading to the development and FDA approval of sulfonamide derivatives such as vemurafenib and dabrafenib. Some of these RAF inhibitors have shown impressive efficacy against metastatic melanomas harboring the recurrent BRAF^{V600E} allele and have been approved for treating this patient population (N. Engl. J. Med. 2011, 364, 2507; Lancet 2012, 380, 358). The clinical responses against BRAF^{V600E}-dependent melanomas result from potent ATP-competitive inhibition of the monomeric form of this specific dimerization-independent BRAF mutant protein (Cancer Cell 2015, 28, 370). Unfortunately, acquired resistance to these agents invariably develops, which is mostly caused by re-activation of the RAS-ERK pathway in part through mechanisms that stimulate RAF dimerization. These include upregulation of RTK signaling, RAS mutations, and BRAF^{V600E} amplification or truncation (Sci. Signal. 2010, 3, ra84; Nature 2010, 468, 973; Nature 2011, 480, 387; Nature Commun. 2012, 3, 724).

Concurrently, tumors exhibiting RAS activity—owing to activating RAS mutations or elevated RTK signaling, but which are otherwise wild-type for BRAF—show primary resistance to BRAF^{V600E} inhibitors (Nature 2010, 464, 431). RAF inhibitors were oppositely found to induce ERK signaling in conditions where RAS activity is elevated and therefore enhances tumor cell proliferation (Nature 2010, 464, 431). This counterintuitive phenomenon, known as the paradoxical effect, was also observed in normal tissues relying on physiological RAS activity and is the basis for some of the adverse effects seen with RAF inhibitors in melanoma patients such as the development of new secondary tumors (e.g. squamous cell carcinomas and keratoacanthomas) (Nat. Rev. Cancer 2014, 14, 455). As a consequence, BRAF^{V600E} inhibitors are ineffective and even contraindicated against RAS-driven cancers. The underlying mechanism results from the compounds' ability to promote RAF kinase domain dimerization in the presence of active RAS (Nature 2010, 464, 431). This event is not restricted to BRAF, but also involves other RAF family members and is dictated by the compound binding mode and affinity (Nat. Chem. Biol. 2013, 9, 428).

Two strategies have recently been pursued to circumvent the limitation of first-generation RAF inhibitors in RAS-mutated cancers. The first one relies on the observation that paradoxical ERK activation is a dose-dependent phenomenon, i.e. induction occurs at sub-saturating inhibitor concentrations but the pathway is suppressed at saturating concentrations when the compound occupies both protomers of RAF dimers. The first strategy therefore focused on developing molecules with higher binding affinities to all RAF paralogs in order to saturate RAF proteins at lower drug concentration thereby reducing paradoxical pathway induction (Bioorg. Med. Chem. Lett. 2012, 22, 6237; Cancer Res. 2013, 73, 7043; J. Med. Chem. 2015, 58, 4165; Cancer Cell 2017, 31, 466; J Med Chem. 2020, 63, 2013; Clin Cancer Res. 2021, 27, 2061; Nature 2021, 594, 418). These compounds however retain strong RAF dimer induction capabilities and thus paradoxically stimulate RAS-ERK signaling, although with a lower amplitude than previous generations of RAF inhibitors. Although this class of compounds show improved properties, they were recently found to mostly spare the ARAF isoform, which leads to paradoxical pathway activation and primary resistance as well as to the emergence of acquired resistance in vitro and in clinical settings (Clin Cancer Res. 2021, 27, 2061; Nature 2021, 594, 418). The second strategy consisted in designing compounds that conformationally bias the BRAF kinase domain in the inactive state and thus do not paradoxically induce ERK signaling. This has given rise to the “Paradox Breaker” (PB) molecule PLX8394, a derivative of PLX4032/vemurafenib (Nature 2015, 526, 583). These molecules retained high potency against BRAF^{V600E} and therefore should prove useful for treating BRAF^{V600E}-dependent melanomas. However, while PLX8394 does not induce ERK signaling in RAS-mutant cell lines that have been tested, it remains ineffective and is not useful against RAS-mutant tumors.

There remains a need for inhibitors that potently and consistently block RAS-ERK signaling and cellular proliferation in human tumor cells bearing a variety of RAS and RAF genotypes. The development of such inhibitors importantly being also devoid of paradoxical pathway induction in a variety of RAS-mutant tumor cell lines is highly desirable.

### SUMMARY

According to one aspect, the present technology relates to a compound of Formula I:

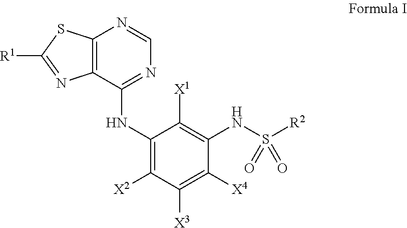

wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl;
R² is selected from substituted C₆aryl or C₅₋₁₀heteroaryl, substituted or unsubstituted C₄₋₈heterocycloalkyl, and N(R³)₂;
R³ is independently in each occurrence selected from substituted or unsubstituted C₁₋₈alkyl, C₃₋₈cycloalkyl, C₄₋₈heterocycloalkyl, C₆₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
or a pharmaceutically acceptable salt or solvate thereof.

The compounds of Formula I are also defined according to any of the embodiments, alone or in combination, and examples described throughout the present document.

According to another aspect, the present technology relates to a pharmaceutical composition for a use as defined in any one of the aforementioned embodiments, the composition comprising a compound as herein defined together with a pharmaceutically acceptable carrier, diluent or excipient.

In a further aspect, the present technology relates to the use of a compound as herein defined for the treatment of a disease or disorder selected from a proliferative disease or disorder, a developmental anomaly caused by dysregulation of the RAS-ERK signaling cascade (RASopathies), or an inflammatory disease or an immune system disorder.

The present technology also further relates to a method for the treatment of a disease or disorder selected from a proliferative disease or disorder, a developmental anomaly caused by dysregulation of the RAS-ERK signaling cascade (RASopathies), or an inflammatory disease or an immune system disorder, comprising administering a compound as herein defined to a subject in need thereof. A method for inhibiting abnormal proliferation of cells, comprising contacting the cells with a compound as defined herein is also contemplated.

In one embodiment of the above uses and methods, the disease or disorder is selected from a neoplasm and a developmental anomaly, for instance, a disease or disorder associated with a RAF gene mutation (e.g. ARAF, BRAF or CRAF), a disease or disorder associated with a RAS gene mutation (e.g. KRAS), or a disease or disorder associated with both a RAF gene mutation and a RAS gene mutation. In one embodiment, the disease or disorder is associated with a receptor tyrosine kinase mutation or amplification (e.g. EGFR, HER2) or a mutation in a regulator of RAS downstream of the receptor (e.g. SOS1 gain of function, NF1 loss of function.

For instance, the disease or disorder is a neoplasm, such as those selected from melanoma, thyroid carcinoma (e.g. papillary thyroid carcinoma), colorectal, ovarian, breast cancer, endometrial cancer, liver cancer, sarcoma, stomach cancer, pancreatic carcinoma, Barret's adenocarcinoma, glioma (e.g. ependymoma), lung cancer (e.g. non-small cell lung cancer), head and neck cancer, acute lymphoblastic leukemia, acute myelogenous leukemia, non-Hodgkin's lymphoma, and hairy-cell leukemia. For instance, the neoplasm is selected from colon or colorectal cancer, lung cancer, pancreatic cancer, thyroid cancer, breast cancer and melanoma. For instance, any of the present uses and methods comprises inhibiting the RAS-ERK signaling pathway without substantial induction of a paradoxical pathway.

Additional objects and features of the present compound, compositions, methods and uses will become more apparent upon reading of the following non-restrictive description of exemplary embodiments and examples section, which should not be interpreted as limiting the scope of the invention.

### DESCRIPTION OF THE FIGURES

FIG. 1 shows representative IC₅₀ inhibition dose response curves for compounds as described herein that do not induce paradoxical induction of pERK signaling (Y_{MIN}>−20%) in RAS-mutant HCT116 cells (Examples 44 and 122) and a compound (PLX4720; CAS No. 918505-84-7) that causes strong induction of the pathway in the same cell line (Y_{MIN}˜−600%).

### DETAILED DESCRIPTION

All technical and scientific terms and expressions used herein have the same definitions as those commonly understood by a person skilled in the art to which the present technology pertains. The definition of some terms and expressions used is nevertheless provided below. To the extent the definitions of terms in the publications, patents, and patent applications incorporated herein by reference are contrary to the definitions set forth in this specification, the definitions in this specification will prevail. The section headings used herein are for organizational purposes only and are not to be construed as limiting the subject matter disclosed.

### i. Definitions

Chemical structures described herein are drawn according to conventional standards. Also, when an atom, such as a carbon atom, as drawn seems to include an incomplete valency, then the valency is assumed to be satisfied by one or more hydrogen atoms even though these are not necessarily explicitly drawn. Hydrogen atoms should be inferred to be part of the compound.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the context clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms “compounds”, “compounds herein described”, “compounds of the present application”, “thiazolo[5,4-d]pyrimidine compounds”, “thiazolopyrimidine compounds” and equivalent expressions refer to compounds described in the present application, e.g. those encompassed by structural Formula I, optionally with reference to any of the applicable embodiments, and also includes exemplary compounds, such as the compounds of Examples 1 to 159, as well as their pharmaceutically acceptable salts, solvates, esters, and prodrugs when applicable. When a zwitterionic form is possible, the compound may be drawn as its neutral form for practical purposes, but the compound is understood to also include its zwitterionic form. Embodiments herein may also exclude one or more of the compounds. Compounds may be identified either by their chemical structure or their chemical name. In a case where the chemical structure and chemical name would conflict, the chemical structure will prevail.

Unless otherwise stated, structures depicted herein are also meant to include all isomeric (e.g., enantiomeric, diastereomeric, and geometric (or conformational)) forms of the structure when applicable; for example, the R and S configurations for each asymmetric center. Therefore, single stereochemical isomers as well as enantiomeric, diastereomeric, and geometric (or conformational) mixtures of the present compounds are within the scope of the present description. The therapeutic compound unless otherwise noted, also encompasses all possible tautomeric forms of the illustrated compound, if any. The term also includes isotopically labeled compounds where one or more atoms have an atomic mass different from the atomic mass most abundantly found in nature. Examples of isotopes that may be incorporated into the present compounds include, but are not limited to, ²H (D), ³H (T), ¹¹C, ¹³C, ¹⁴C, ¹⁵N, ¹⁸O, ¹⁷O, any one of the isotopes of sulfur, etc. The compound may also exist in unsolvated forms as well as solvated forms, including hydrated forms. The compound may exist in multiple crystalline or amorphous forms. In general, all physical forms are equivalent for the uses contemplated herein and are intended to be within the scope of the present invention.

Where a particular enantiomer is preferred, it may, in some embodiments be provided substantially free of the corresponding enantiomer and may also be enantiomerically enriched. “Enantiomerically enriched” means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including high-pressure liquid chromatography (HPLC) on chiral support and the formation and crystallization of chiral salts or be prepared by asymmetric syntheses.

The expression “pharmaceutically acceptable salt” refers to those salts of the compounds of the present description which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the present description, or separately by reacting a free base function of the compound with a suitable organic or inorganic acid (acid addition salts) or by reacting an acidic function of the compound with a suitable organic or inorganic base (base-addition salts).

The term “solvate” refers to a physical association of one of the present compound with one or more solvent molecules, including water and non-aqueous solvent molecules. This physical association may include hydrogen bonding. In certain instances, the solvate will be capable of isolation, for example when one or more solvent molecules are incorporated in the crystal lattice of a crystalline solid. The term “solvate” encompasses both solution-phase and isolable solvates. Exemplary solvates include, without limitation, hydrates, hemihydrates, ethanolates, hemiethanolates, n-propanolates, iso-propanolates, 1-butanolates, 2-butanolate, and solvates of other physiologically acceptable solvents, such as the Class 3 solvents described in the International Conference on Harmonization (ICH), Guide for Industry, Q3C Impurities: Residual Solvents (1997). Accordingly, the compound as herein described also includes each of its solvates and mixtures thereof.

As used herein, the expression “pharmaceutically acceptable ester” refers to esters of the compounds formed by the process of the present description which may hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates of hydroxyl groups, and alkyl esters of an acidic group. Other ester groups include sulfonate or sulfate esters.

The expression “pharmaceutically acceptable prodrugs” as used herein refers to those prodrugs of the compounds formed by the process of the present description which are, within the scope of sound medical judgment, suitable for use in contact with the tissues of humans and lower animals with undue toxicity, irritation, allergic response, and the like, commensurate with a reasonable benefit/risk ratio, and effective for their intended use. “Prodrug”, as used herein means a compound which is convertible in vivo by metabolic means (e.g. by hydrolysis) to afford any compound delineated by the formulae of the instant description.

Abbreviations may also be used throughout the application, unless otherwise noted, such abbreviations are intended to have the meaning generally understood by the field. Examples of such abbreviations include Me (methyl), Et (ethyl), Pr (propyl), i-Pr (isopropyl), Bu (butyl), t-Bu (tert-butyl), i-Bu (iso-butyl), s-Bu (sec-butyl), c-Bu (cyclobutyl), Ph (phenyl), Bn (benzyl), Bz (benzoyl), CBz or Cbz or Z (carbobenzyloxy), Boc or BOC (tert-butoxycarbonyl), and Su or Suc (succinimide). For more certainty, additional definitions of specific abbreviations are also included in the introduction of the Examples section.

The number of carbon atoms in a hydrocarbyl substituent can be indicated by the prefix “Cₓ-C_{y}” or “C_{x-y}” where x is the minimum and y is the maximum number of carbon atoms in the substituent. However, when the prefix “Cₓ-C_{y}” or “C_{x-y}” is associated with a group incorporating one or more heteroatom(s) by definition (e.g. heterocycloalkyl, heteroaryl, etc), then x and y define respectively the minimum and maximum number of atoms in the cycle, including carbon atoms as well as heteroatom(s).

The term “alkyl” as used herein, refers to a saturated, straight- or branched-chain hydrocarbon radical typically containing from 1 to 20 carbon atoms. For example, “C₁₋₈alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds and typically from 2 to 20 carbon atoms. For example, “C₂₋₈alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds and typically from 2 to 20 carbon atoms. For example, “C₂₋₈alkynyl” contains from two to eight carbon atoms. Representative alkynyl groups include, but are not limited to, for example, ethynyl,1-propynyl, 1-butynyl, heptynyl, octynyl and the like.

The terms “cycloalkyl”, “alicyclic”, “carbocycle”, “carbocyclic” and equivalent expressions refer to a group comprising a saturated or partially unsaturated (non aromatic) carbocyclic ring in a monocyclic or polycyclic ring system, including spiro (sharing one atom), fused (sharing at least one bond) or bridged (sharing two or more bonds) carbocyclic ring systems, having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term cycloalkyl includes both unsubstituted cycloalkyl groups and substituted cycloalkyl groups. For example, the term “C₃₋ₙcycloalkyl” refers to a cycloalkyl group having from 3 to the indicated “n” number of carbon atoms in the ring structure. Unless the number of carbons is otherwise specified, “lower cycloalkyl” groups as herein used, have at least 3 and equal or less than 8 carbon atoms in their ring structure.

As used herein, the terms “heterocycle”, “heterocycloalkyl”, “heterocyclyl”, “heterocyclic radical”, and “heterocyclic ring” are used interchangeably and refer to a chemically stable 3- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocyclic ring can be attached to its pendant group at any heteroatom or carbon atom that results in a chemically stable structure and any of the ring atoms can be optionally substituted. Examples of heterocycloalkyl groups include, but are not limited to, 1,3-dioxolanyl, pyrrolidinyl, pyrrolidonyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, tetrahydrofuranyl, tetrahydropyranyl, tetrahydrothiopyranyl, tetrahydrodithienyl, tetrahydrothienyl, thiomorpholino, thioxanyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, 2H-pyranyl, 4H-pyranyl, dioxanyl, dithianyl, dithiolanyl, dihydropyranyl, dihydrothienyl, dihydrofuranyl, 3-azabicyclo[3,1,0]hexanyl, 3-azabicyclo[4,1,0]heptanyl, quinolizinyl, quinuclidinyl, tetrahydroquinolinyl, tetrahydroisoquinolinyl, decahydroquinolinyl, and the like. Heterocyclic groups also include groups in which a heterocyclic ring is fused to one or more aryl, heteroaryl, or cycloaliphatic rings, such as indolinyl, 3H-indolyl, chromanyl, chromenyl, phenanthridinyl, 2-azabicyclo[2.2.1]heptanyl, octahydroindolyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted. For instance, the term “C₃₋ₙheterocycloalkyl” refers to a heterocycloalkyl group having from 3 to the indicated “n” number of atoms in the ring structure, including carbon atoms and heteroatoms.

As used herein, the term “partially unsaturated” refers to a ring moiety that includes at least one double or triple bond between ring atoms but is not aromatic. The term “partially unsaturated” is intended to encompass rings having multiple sites of unsaturation but is not intended to include aryl or heteroaryl moieties, as herein defined.

The term “aryl” used alone or as part of a larger moiety as in “aralkyl”, “aralkoxy”, “aryloxy”, or “aryloxyalkyl”, refers to aromatic groups having 4n+2 conjugated π(pi) electrons, wherein n is an integer from 1 to 3, in a monocyclic moiety or a bicyclic or tricyclic fused ring system having a total of six to 15 ring members, wherein at least one ring in the system is aromatic and wherein each ring in the system contains three to seven ring members. The term “aryl” may be used interchangeably with the term “aryl ring”. In certain embodiments of the present description, “aryl” refers to an aromatic ring system which includes, but not limited to, phenyl, biphenyl, naphthyl, azulenyl, anthracyl and the like, which may bear one or more substituents. The term “aralkyl” or “arylalkyl” refers to an alkyl residue attached to an aryl ring. Examples of aralkyl include, but are not limited to, benzyl, phenethyl, and the like. Also included within the scope of the term “aryl”, as it is used herein, is a group in which an aromatic ring is fused to one or more non-aromatic rings, such as indanyl, indenyl, phthalimidyl, naphthimidyl, fluorenyl, phenanthridinyl, or tetrahydronaphthyl, and the like. For example, the term “C₆₋ₙaryl” refers to an aryl group having from 6 to the indicated “n” number of atoms in the ring structure.

The term “heteroaryl”, used alone or as part of a larger moiety, e.g., “heteroaralkyl”, or “heteroaralkoxy”, refers to aromatic groups having 4n+2 conjugated π(pi) electrons, wherein n is an integer from 1 to 3 (e.g. having 5 to 18 ring atoms, preferably 5, 6, or 9 ring atoms; having 6, 10, or 14 π electrons shared in a cyclic array); and having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. The term “heteroaryl”, as used herein, also includes groups in which a heteroaromatic ring is fused to one or more aryl, cycloaliphatic, or heterocyclic rings, where the radical or point of attachment is on the heteroaromatic ring. Nonlimiting examples of heteroaryl groups include thienyl, furanyl (furyl), pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, triazinyl, indolyl, 3H-indolyl, isoindolyl, indolizinyl, benzothienyl (benzothiophenyl), benzofuranyl, dibenzofuranyl, indazolyl, benzimidazolyl, benzoxazolyl, benzothiazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), furopyridinyl, purinyl, imidazopyrazinyl (e.g. imidazo[4,5-b]pyrazinyl), quinolyl (quinolinyl), isoquinolyl (isoquinolinyl), quinolonyl, isoquinolonyl, cinnolinyl, phthalazinyl, quinazolinyl, quinoxalinyl, 4H-quinolizinyl, naphthyridinyl, and pteridinyl carbazolyl, acridinyl, phenanthridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. Heteroaryl groups include rings that are optionally substituted. The term “heteroaralkyl” refers to an alkyl group substituted by a heteroaryl, wherein the alkyl and heteroaryl portions independently are optionally substituted. Examples include, but are not limited to, pyridinylmethyl, pyrimidinylethyl and the like. For instance, the term “C₅₋ₙheteroaryl” refers to a heteroaryl group having from 5 to the indicated “n” number of atoms in the ring structure, including carbon atoms and heteroatoms.

As described herein, compounds of the present description may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

The term “halo” designates a halogen atom, i.e. a fluorine, chlorine, bromine or iodine atom, preferably fluorine or chlorine.

The term “optionally substituted” refers to groups that are substituted or unsubstituted by independent replacement of one, two, or three or more of the hydrogen atoms thereon with substituents including, but not limited to F, Cl, Br, I, OH, CO₂H, alkoxy, oxo, thiooxo, NO₂, CN, CF₃, NH₂, NHalkyl, NHalkenyl, NHalkynyl, NHcycloalkyl, NHaryl, NHheteroaryl, NHheterocyclic, dialkylamino, diarylamino, diheteroarylamino, O-alkyl, O-alkenyl, O-alkynyl, O-cycloalkyl, O-aryl, O-heteroaryl, O-haloalkyl, O-heterocyclic, C(O)alkyl, C(O)alkenyl, C(O)alkynyl, C(O)cycloalkyl, C(O)aryl, C(O)heteroaryl, C(O)heterocycloalkyl, CO₂alkyl, CO₂alkenyl, CO₂alkynyl, CO₂cycloalkyl, CO₂aryl, CO₂heteroaryl, CO₂heterocycloalkyl, OC(O)alkyl, OC(O)alkenyl, OC(O)alkynyl, OC(O)cycloalkyl, OC(O)aryl, OC(O)heteroaryl, OC(O)heterocycloalkyl, C(O)NH₂, C(O)NHalkyl, C(O)NHalkenyl, C(O)NHalkynyl, C(O)NHcycloalkyl, C(O)NHaryl, C(O)NHheteroaryl, C(O)NHheterocycloalkyl, OCO₂alkyl, OCO₂alkenyl, OCO₂alkynyl, OOO₂cycloalkyl, OCO₂aryl, OOO₂heteroaryl, OOO₂heterocycloalkyl, OC(O)NH₂, OC(O)NHalkyl, OC(O)NHalkenyl, OC(O)NHalkynyl, OC(O)NHcycloalkyl, OC(O)NHaryl, OC(O)NHheteroaryl, OC(O)NHheterocycloalkyl, NHC(O)alkyl, NHC(O)alkenyl, NHC(O)alkynyl, NHC(O)cycloalkyl, NHC(O)aryl, NHC(O)heteroaryl, NHC(O)heterocycloalkyl, NHCO₂alkyl, NHCO₂alkenyl, NHCO₂alkynyl, NHCO₂cycloalkyl, NHCO₂aryl, NHCO₂heteroaryl, NHCO₂heterocycloalkyl, NHC(O)NH₂, NHC(O)NHalkyl, NHC(O)NHalkenyl, NHC(O)NHalkenyl, NHC(O)NHcycloalkyl, NHC(O)NHaryl, NHC(O)NHheteroaryl, NHC(O)NHheterocycloalkyl, NHC(S)NH₂, NHC(S)NHalkyl, NHC(S)NHalkenyl, NHC(S)NHalkynyl, NHC(S)NHcycloalkyl, NHC(S)NHaryl, NHC(S)NHheteroaryl, NHC(S)NHheterocycloalkyl, NHC(NH)NH₂, NHC(NH)NHalkyl, NHC(NH)NHalkenyl, NHC(NH)NHalkenyl, NHC(NH)NHcycloalkyl, NHC(NH)NHaryl, NHC(NH)NHheteroaryl, NHC(NH)NHheterocycloalkyl, NHC(NH)alkyl, NHC(NH)alkenyl, NHC(NH)alkenyl, NHC(NH)cycloalkyl, NHC(NH)aryl, NHC(NH)heteroaryl, NHC(NH)heterocycloalkyl, C(NH)NHalkyl, C(NH)NHalkenyl, C(NH)NHalkynyl, C(NH)NHcycloalkyl, C(NH)NHaryl, C(NH)NHheteroaryl, C(NH)NHheterocycloalkyl, P(O)(alkyl)₂, P(O)(alkenyl)₂, P(O)(alkynyl)₂, P(O)(cycloalkyl)₂, P(O)(aryl)₂, P(O)(heteroaryl)₂, P(O)(heterocycloalkyl)₂, P(O)(Oalkyl)₂, P(O)(OH)₂, P(O)(Oalkenyl)₂, P(O)(Oalkynyl)₂, P(O)(Ocycloalkyl)₂, P(O)(Oaryl)₂, P(O)(Oheteroaryl)₂, P(O)(Oheterocycloalkyl)₂, S(O)alkyl, S(O)alkenyl, S(O)alkynyl, S(O)cycloalkyl, S(O)aryl, S(O)₂alkyl, S(O)₂alkenyl, S(O)₂alkynyl, S(O)₂cycloalkyl, S(O)₂aryl, S(O)heteroaryl, S(O)heterocycloalkyl, SO₂NH₂, SO₂NHalkyl, SO₂NHalkenyl, SO₂NHalkynyl, SO₂NHcycloalkyl, SO₂NHaryl, SO₂NHheteroaryl, SO₂NHheterocycloalkyl, NHSO₂alkyl, NHSO₂alkenyl, NHSO₂alkynyl, NHSO₂cycloalkyl, NHSO₂aryl, NHSO₂heteroaryl, NHSO₂heterocycloalkyl, CH₂NH₂, CH₂SO₂CH₃, alkyl, alkenyl, alkynyl, aryl, arylalkyl, heteroaryl, heteroarylalkyl, heterocycloalkyl, cycloalkyl, carbocyclic, heterocyclic, polyalkoxyalkyl, polyalkoxy, methoxymethoxy, methoxyethoxy, SH, S-alkyl, S-alkenyl, S-alkynyl, S-cycloalkyl, S-aryl, S-heteroaryl, S-heterocycloalkyl, or methylthiomethyl.

### ii. Compounds

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof. As such, the following embodiments are present alone or in combination if applicable.

The present compounds present a thiazolo[5,4-d]pyrimidine core structure to which is attached defined substituents to achieve the product's beneficial activity. Examples of thiazolopyrimidine compounds as defined herein are illustrated by general Formula I:

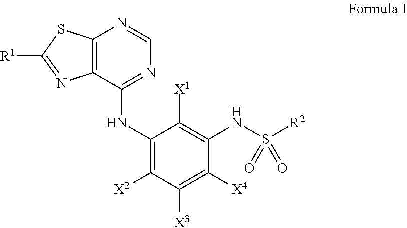

wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl, for example, selected from substituted or unsubstituted SR³, N(R³)₂, C₄₋₈heterocycloalkyl, C₆₋₁₀aryl and C₅₋₁₀heteroaryl, preferably a substituted or unsubstituted C₆₋₁₀aryl or C₅₋₁₀heteroaryl;
R² is selected from substituted C₆aryl or C₅₋₁₀heteroaryl, substituted or unsubstituted C₄₋₈heterocycloalkyl, and N(R³)₂;
R³ is independently in each occurrence selected from substituted or unsubstituted C₁₋₈alkyl, C₃₋₈cycloalkyl, C₄₋₈heterocycloalkyl, C₆₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
or a pharmaceutically acceptable salt or solvate thereof.

For example, the electron-withdrawing group is selected from perhaloalkyl (e.g. CF₃ or CCl₃), CN, NO₂, sulfonate, alkylsulfonyl (e.g. SO₂Me or SO₂CF₃), alkylcarbonyl (e.g. C(O)Me), carboxylate, alkoxycarbonyl (e.g. C(O)OMe), and aminocarbonyl (e.g. C(O)NH₂). In one embodiment, X¹ is Cl and X² is F, or X¹ is F and X² is H, or X¹ and X² are both F. In another embodiment, X³ and X⁴ are each H. In yet another embodiment, X³ is F and X⁴ is H.

For instance, the aminoarylsulfonamide moiety in Formula I may be designated L and preferably be selected from:

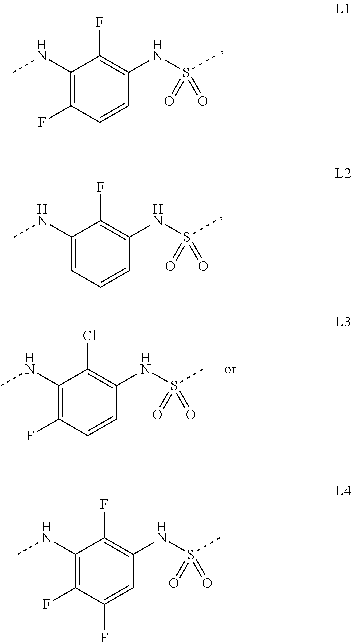

where the dashed line ( - - - ) represents a bond serving as a point of attachment between L and the rest of the molecule.

In a further embodiment, R² is a substituted C₆aryl or C₅₋₁₀heteroaryl, e.g. R² is a C₆aryl substituted with at least one group selected from F, Cl, Br, CN, NO₂, and a substituted or unsubstituted C₁₋₃ alkyl, C₃₋₄cycloalkyl or OC₁₋₃alkyl. For instance, R² is a group of the formula:

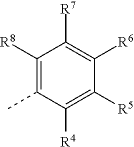

wherein:
R⁴ is selected from H, F, Cl, Br, CN, and a substituted or unsubstituted C₁₋₃alkyl, C₃₋₄cycloalkyl or OC₁₋₃alkyl, e.g. R⁴ is selected from H, F, Cl, Br, Me, Et, CN, CHF₂, and CF₃;
R⁵ is selected from H, F, Cl, CN, and a substituted or unsubstituted C₁₋₃alkyl, C₃₋₄cycloalkyl, or OC₁₋₃alkyl, e.g. R⁵ is selected from H, F, Me, CF₃, CN, and Cl;
R⁶ is selected from H, F, C, Br, NO₂, NH₂, and a substituted or unsubstituted C₁₋₃alkyl, C₃₋₄cycloalkyl or OC₁₋₃alkyl, e.g. R⁶ is selected from H, F, C, Br, and a substituted or unsubstituted C₁₋₃alkyl, C₃₋₄cycloalkyl or OC₁₋₃alkyl, or R⁶ is selected from H, F, C, Me, Et, and OMe;
R⁷ is selected from H, F, C, and a substituted or unsubstituted C₁₋₃alkyl, e.g. R⁷ is selected from H, Me, F, and Cl;
R³ is selected from H, F, and a substituted or unsubstituted C₁₋₃alkyl, e.g. R³ is selected from H, Me and F;
or R⁴ and R⁵ or R⁵ and R⁶ are taken together with their adjacent carbon atoms to form a substituted or unsubstituted carbocycle or heterocycle provided that the heterocycle (R²) is not a benzoxazolinone; and
( - - - ) represents a bond serving as a point of attachment between R² and the rest of the molecule;
wherein when R⁴ is H or F, then at least one of R⁵, R⁶, R⁷ or R³ is other than H or F; and
wherein when R⁵ is CN, then at least one of R⁴, R⁶, R⁷ or R³ is other than H.

In one embodiment, R³ is H. In another embodiment, R⁴ is selected from F, Cl, Et and Me, R⁵, R⁷, and R⁸ are each H, and R⁶ is selected from H, CI, Me and OMe. In a further embodiment, R⁴ is selected from F, Cl, and Me, R⁶, R⁷ and R³ are each H, and R⁵ is selected from F and Cl.

In a further embodiment, R⁴ is selected from Cl and a substituted or unsubstituted C₁₋₃alkyl (e.g. Me); R⁵ is selected from H, F, Cl, and a substituted or unsubstituted C₁₋₃alkyl (e.g., Me); R⁶ is selected from H and a substituted or unsubstituted OC₁₋₃alkyl (e.g. OCH₃); and R⁷ and R⁸ are each H.

In yet another embodiment, R⁴ is selected from H, Cl, Br and methyl; R⁵ is selected from H, F, and Cl; R⁶ is selected from H, F, Cl, Me and OMe; and R⁷ and R³ are each H.

In a further embodiment, R⁴ is selected from Cl and a substituted or unsubstituted C₁₋₃alkyl, preferably R⁴ is Cl or Me; R⁵ is selected from H, F, Cl, and a substituted or unsubstituted C₁₋₃alkyl (e.g., Me), preferably R⁵ is F, Cl or Me; R⁶ is selected from H, F, Cl, a substituted or unsubstituted C₁₋₃alkyl (e.g., Me), and a substituted or unsubstituted OC₁₋₃alkyl (e.g. OCH₃), preferably R⁶ is H or F, or R⁶ is Cl or a substituted or unsubstituted C₁₋₃alkyl or substituted or unsubstituted OC₁₋₃alkyl, or R⁶ is CH₃ or OCH₃; and R⁷ and R³ are each H. In yet another embodiment, R⁶ is a substituted C₁₋₃alkyl.

In another example, R² is a substituted C₅heteroaryl, such as a group of the formula:

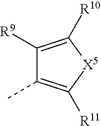

wherein:
X⁵ is selected from NH, NC₁₋₃alkyl, NC₃₋₄cycloalkyl, O and S;
R⁹, R¹⁰, R¹¹ are each independently selected from H, F, Cl, CN, and a substituted or unsubstituted C₁₋₃alkyl, C₃₋₄cycloalkyl, C(O)OC₁₋₃alkyl or OC₁₋₃alkyl, provided that one of R⁹ and R¹¹ is H and the other is not H; and
( - - - ) represents a bond serving as a point of attachment between R² and the rest of the molecule.

Alternatively, R² is a group of the formula:

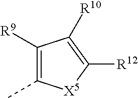

wherein:
X⁵ is selected from NH, NC₁₋₃alkyl, NC₃₋₄cycloalkyl, O and S;
R⁹ is selected from F, Cl, CN, and a substituted or unsubstituted C₁₋₃alkyl, C₃₋₄cycloalkyl, C(O)OC₁₋₃alkyl or OC₁₋₃alkyl;
R¹⁰ and R¹² are each independently selected from H, F, Cl, CN, and a substituted or unsubstituted C₁₋₃alkyl, C₃₋₄cycloalkyl, C(O)OC₁₋₃alkyl or OC₁₋₃alkyl; and
( - - - ) represents a bond serving as a point of attachment between R² and the rest of the molecule.

In a preferred embodiment, R⁹ and R¹⁰ are each independently selected from F, Cl, CN, and a substituted or unsubstituted C₁₋₃alkyl, C₃₋₄cycloalkyl, C(O)OC₁₋₃alkyl or OC₁₋₃alkyl, preferably Cl and a substituted or unsubstituted C₁₋₃alkyl, more preferably R⁹ and R¹⁰ are both Cl. In another embodiment, X⁵ is O or S, preferably S.

In another embodiment, R² is a substituted C₅₋₁₀heteroaryl, such as a group of the formula:

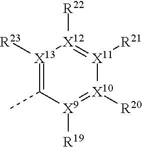

wherein:
X⁹, X¹⁰, X¹¹, X¹², and X¹³ are independently selected from N and C, wherein at least one and at most two of X⁹, X¹⁰, X¹¹, X¹², and X¹³ are N; and
R¹⁹, R²⁰, R²¹, R²² and R²³ are selected from H, F, Cl, Br, CN, NO₂, NH₂, and a substituted or unsubstituted C₁₋₃alkyl, C₃₋₄cycloalkyl or OC₁₋₃alkyl, or are absent when their attached X⁹, X¹⁰, X¹¹, x¹², or X¹³ is N;
wherein at least one of X⁹ and X¹³ is not N;
wherein when one of X⁹ and X¹³ is N, then the other is not N or CH; and
( - - - ) represents a bond serving as a point of attachment between R² and the rest of the molecule.

In another example, R² is a C₅heterocycloalkyl. For instance, R² is a group of the formula:

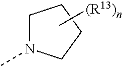

wherein:
R¹³ is independently in each occurrence selected from F, Cl, and a substituted or unsubstituted C₁₋₃alkyl, C₃₋₄cycloalkyl, or C₁₋₃alkoxy;
n is an integer selected from 0 to 8; or
n is between 2 and 8 and two R¹³ are taken together with their adjacent carbon atoms to form a C₃₋₄cycloalkyl; and
( - - - ) represents a bond serving as a point of attachment between R² and the rest of the molecule.

In one embodiment, R¹³ is in the 3-position. In another embodiment, R¹³ is selected from F, Me, OMe, and CH₂OMe, and n is 1 or 2. For instance, R¹³ is a methoxy group in the 3-position and n is 1.

In a further example, R² is N(R³)₂. For instance, R² is N(R³)₂ and R³ is selected from substituted or unsubstituted C₁₋₈alkyl or C₃₋₈cycloalkyl.

In yet another embodiment, the compound of Formula I is a compound of Formula II, or a pharmaceutically acceptable salt or solvate thereof:

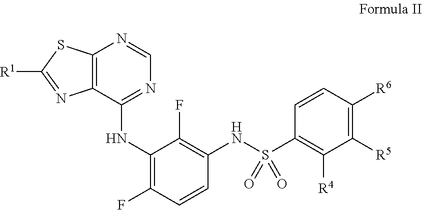

wherein R¹, R⁴, R⁵, and R⁶ are each independently as defined herein, preferably R⁴ is selected from Cl, Br and methyl; R⁵ is selected from H, F, Cl and methyl; R⁶ is selected from H, F, Cl, Me and OMe.

In a further embodiment, the compound of Formula I is a compound of Formula III, or a pharmaceutically acceptable salt or solvate thereof:

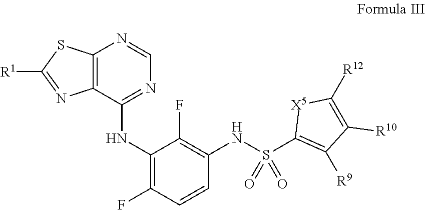

wherein R¹, R⁹, R¹⁰, R¹², and X⁵ are each independently as defined herein.

Exemplary R² groups are illustrated by groups B1 to B77 defined as follows:

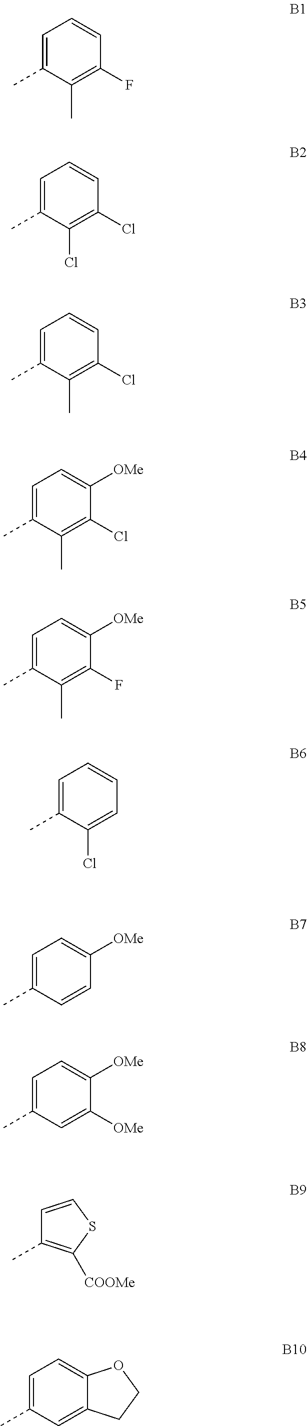

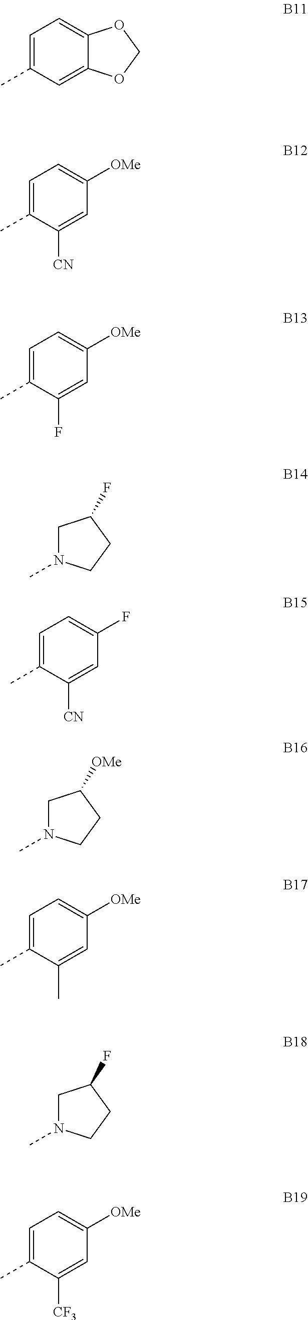

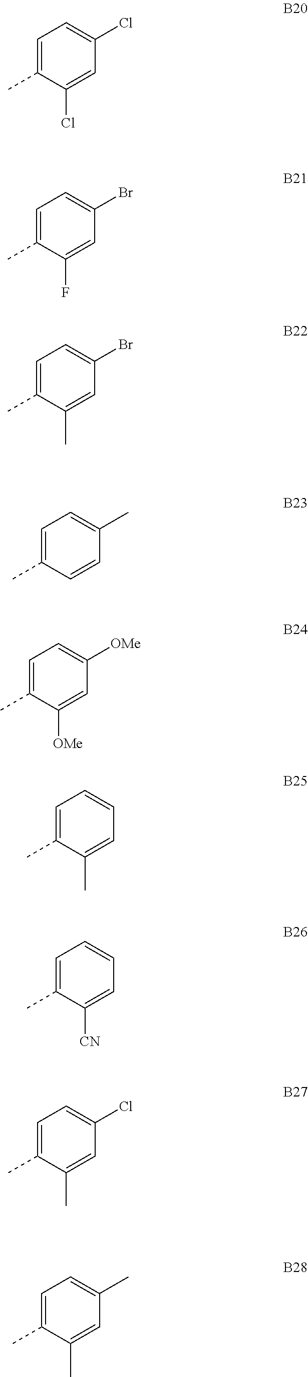

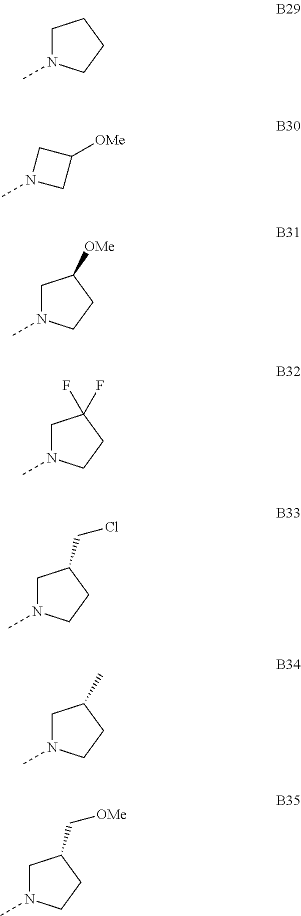

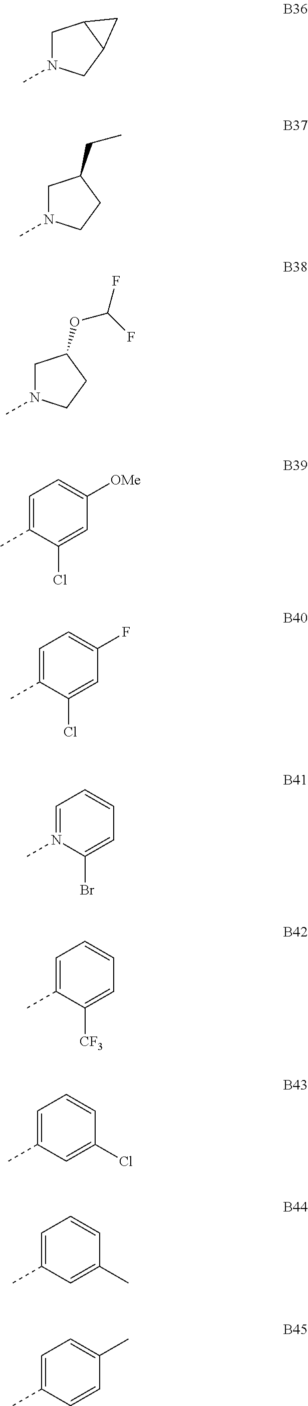

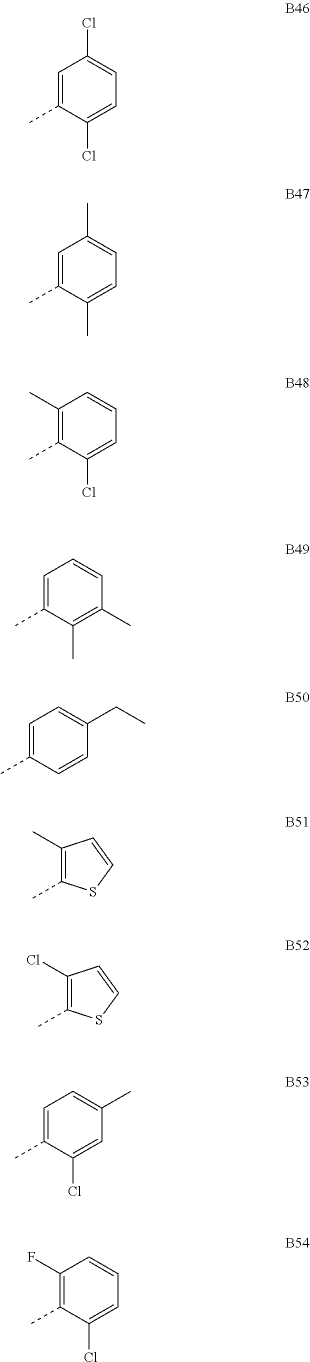

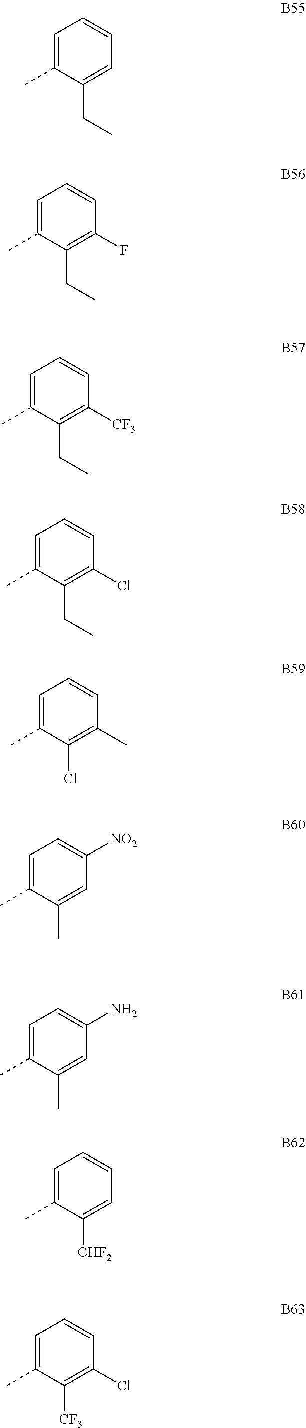

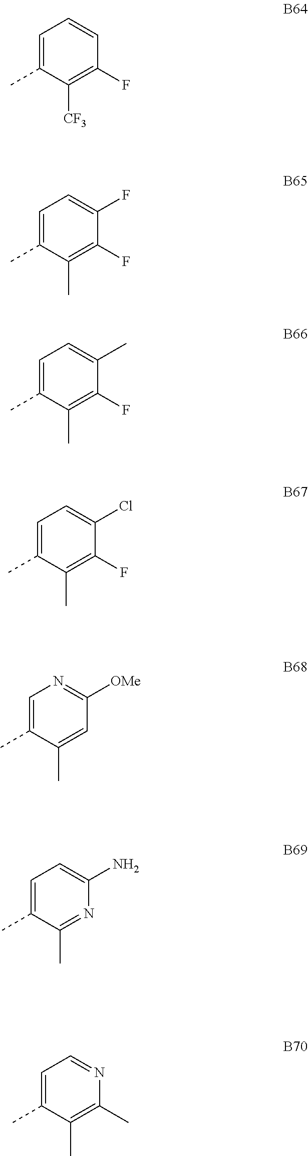

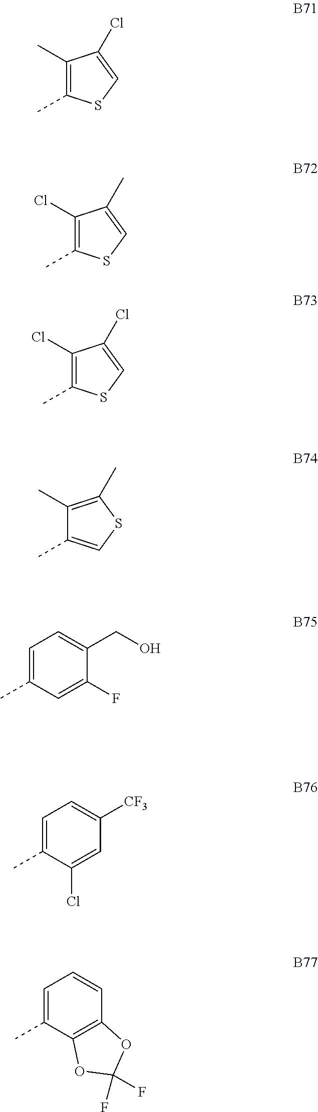

wherein ( - - - ) represents a bond serving as a point of attachment between R² and the rest of the molecule.

In one embodiment, R² is selected from groups B1 to B77, or preferably R² is selected from groups 1 to B6.

In one embodiment of the compound of Formula I, R¹ is OR³ or SR³, for instance, R¹ is SR³. In various embodiments, R³ is a substituted or unsubstituted C₁₋₈alkyl (e.g. C₁₋₃alkyl).

In another embodiment, R¹ is a substituted or unsubstituted C₆aryl group. In another embodiment, R¹ is a substituted or unsubstituted C₄₋₆heterocycloalkyl group. For instance, R¹ is a C₄₋₅heterocycloalkyl optionally substituted with one or two groups selected from halo, OH, C₁₋₆alkyl, and OC₁₋₆alkyl. For instance, R¹ is a N-pyrrolidinyl group substituted with one or two groups selected from F and OH.

In another embodiment, R¹ is a substituted or unsubstituted C₅₋₆heteroaryl group or a substituted or unsubstituted C₉heteroaryl group. In another emobodiment, R¹ is a substituted or unsubstituted group selected from thienyl, imidazolyl, pyrazolyl, triazolyl, thiazolyl, pyridyl, pyrimidinyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, imidazopyrazinyl (e.g. imidazo[4,5-b]pyrazinyl), and quinolyl (quinolinyl), preferably R¹ is a substituted or unsubstituted group selected from imidazolyl, pyrazolyl, triazolyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, and imidazopyrazinyl (e.g. imidazo[4,5-b]pyrazinyl), more preferably attached to the thiazolopyrimidine core through a nitrogen atom.

Examples of R¹ include a substituted or unsubstituted group selected from:

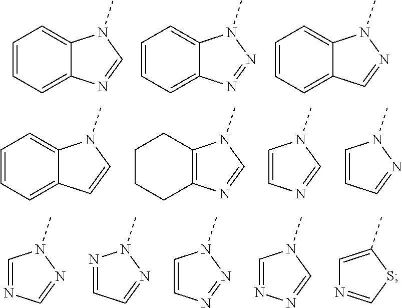

wherein ( - - - ) represents a bond serving as a point of attachment between R¹ and the rest of the molecule.

For instance, R¹ is a substituted or unsubstituted group selected from:

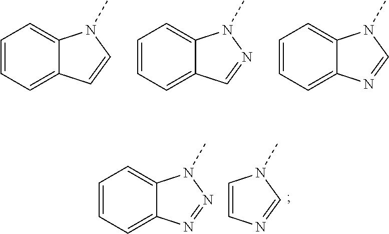

wherein ( - - - ) represents a bond serving as a point of attachment between R¹ and the rest of the molecule.

In one embodiment, R¹ is one of the above groups further substituted with at least one substituent selected from OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein:
   R¹⁴ is independently in each occurrence selected from H, C₁₋₆alkyl, C₂₋₆alkenyl, C₂₋₆alkynyl, C₃₋₁₀cycloalkyl, C₄₋₁₀heterocycloalkyl, C₆aryl, and C₅₋₁₀heteroaryl, or two R¹⁴ are taken together with their adjacent nitrogen atom to form a C₄₋₁₀heterocycloalkyl group;
   R¹⁵ is independently in each occurrence selected from C₁₋₆alkyl, C₂₋₆alkenyl, C₂₋₆alkynyl, C₃₋₁₀cycloalkyl, C₆aryl, and C₅₋₁₀heteroaryl; and
   R¹⁶ is independently in each occurrence selected from H, C₁₋₆alkyl, C₂₋₆alkenyl, C₂₋₆alkynyl, C₃₋₁₀cycloalkyl, C₆aryl, and C₅₋₁₀heteroaryl;
wherein said alkyl, alkenyl, alkynyl, cycloalkyl, heterocycloalkyl, aryl or heteroaryl group, included in R¹ (including in the definitions of R¹⁴, R¹⁵, and R¹⁶), is optionally further substituted.

In another embodiment, R¹ is a group of the formula:

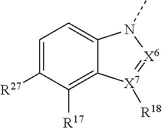

wherein:
R¹⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
R²⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, preferably H, halo (e.g. F), optionally substituted C₁₋₆alkyl, or optionally substituted OC₁₋₆alkyl;
X⁶ is N or CH; and
X⁷ is N and R¹⁸ is absent; or
X⁷ is C and R¹⁸ is selected from C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein R¹⁴, R¹⁵, and R¹⁶ are as defined above;
wherein said alkyl, alkenyl, alkynyl, cycloalkyl, heterocycloalkyl, or heteroaryl, included in R¹ (including in the definitions of R¹⁴, R¹⁵, R¹⁶, R¹⁷ and R¹⁸), is optionally further substituted; and
wherein ( - - - ) represents a bond serving as a point of attachment between R¹ and the rest of the molecule.

In another embodiment, R¹ is a group of the formula:

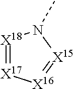

wherein:
X¹⁵, X¹⁶, X¹⁷, and X¹⁸ are independently selected from O, N, S, and CR¹⁷, wherein R¹⁷ is as previously defined;
wherein at most two of X¹⁵, X¹⁶, X¹⁷, and X¹⁸ are O, N, or S; and
wherein ( - - - ) represents a bond serving as a point of attachment between R¹ and the rest of the molecule.

In one embodiment, the compound of Formula I is a compound of Formula IV or V, or a pharmaceutically acceptable salt or solvate thereof:

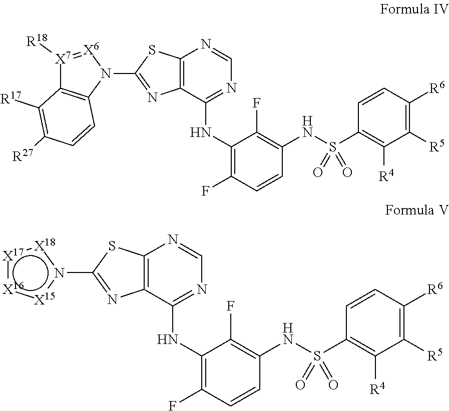

wherein R⁴, R⁵, R⁶, R¹⁷, R¹⁸, R²⁷, X⁶, X⁷, X¹⁵, X¹⁶, X¹⁷, and X¹⁸ are each independently as defined herein, preferably R⁴ is selected from Cl, Br and methyl; R⁵ is selected from H, F, Cl and methyl; R⁶ is selected from H, Cl, F, Me and OMe.

In a further embodiment, the compound of Formula I is a compound of Formula VI or VII, or a pharmaceutically acceptable salt or solvate thereof:

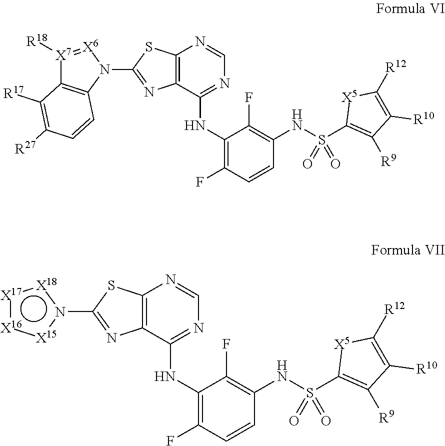

wherein R⁹, R¹⁰, R¹², R¹⁷, R¹⁸, R²⁷, X⁵, X⁶, X⁷, X¹⁵, X¹⁶, X¹⁷ and X¹⁸ are each independently as defined herein.

In one embodiment of the above formulae, X⁶ is N. In another embodiment, X⁶ is CH.

In another embodiment, X⁷ is N, R¹⁷ is selected from H, OH, halo, CN, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, and R¹⁸ is absent, wherein said alkyl, alkenyl, alkynyl, cycloalkyl, heterocycloalkyl, or heteroaryl in R¹, R¹⁵, R¹⁶, or R¹⁷, is optionally further substituted, preferably R¹⁷ is selected from C₁₋₆alkyl, C₅₋₁₀heteroaryl, C₄₋₁₀heterocycloalkyl, N(R¹⁴)₂, N(R¹⁶)C(O)R¹⁵, N(R¹⁶)SO₂R¹⁵, C(O)N(R¹⁴)₂, and SO₂N(R¹⁴)₂, wherein said alkyl, alkenyl, alkynyl, cycloalkyl, heterocycloalkyl, or heteroaryl in R¹⁴, R¹⁵, R¹⁶, or R¹⁷, is optionally further substituted. For instance, R¹⁷ is selected from H, F, NH₂, and an optionally substituted C₅₋₁₀heteroaryl or C₄₋₁₀heterocycloalkyl, preferably R¹⁷ is an optionally substituted C₅₋₁₀heteroaryl or C₄₋₁₀heterocycloalkyl.

In a further embodiment, R¹⁷ is an optionally substituted C₄₋₁₀heterocycloalkyl, wherein said heterocycloalkyl may be mono or bicyclic and include from 1 to 3 heteroatoms, preferably wherein X⁷ is N. In a preferred embodiment, the heterocycloalkyl is substituted, for instance, with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.). For instance, the heterocycloalkyl may be selected from optionally substituted piperidine, piperazine, thiomorpholine, and morpholine groups, or a bicyclic structure (bridged or spiro) containing a piperidine, piperazine, thiomorpholine, or morpholine ring.

In a further embodiment, X⁷ is C, for instance, X⁷ is C and R¹⁸ is selected from C₁₋₆alkyl, C₅₋₁₀ heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, wherein said alkyl, alkenyl, alkynyl, cycloalkyl, heterocycloalkyl, in R¹⁴, R¹⁵, R¹⁶, or R¹⁸, or heteroaryl is optionally further substituted, preferably R¹⁸ is selected from C(O)N(R¹⁴)₂, SO₂R¹⁵, and SO₂N(R¹⁴)₂. In a subclass of these embodiments, R¹⁷ is selected from H, halo, OH, C₁₋₆alkyl, N(R¹⁴)₂, and an optionally substituted C₅₋₁₀heteroaryl. For instance, R¹⁷ is selected from H, F, NH₂, and an optionally substituted C₅₋₁₀heteroaryl, preferably H, F, or NH₂.

In yet another embodiment, R¹⁴ is independently in each occurrence selected from H, optionally substituted C₁₋₆alkyl, optionally substituted C₃₋₁₀cycloalkyl, optionally substituted C₄₋₁₀heterocycloalkyl, and optionally substituted C₅₋₆heteroaryl, or two R¹⁴ are taken together with their adjacent nitrogen atom to form a C₄₋₁₀heterocycloalkyl group.

In another embodiment, R¹⁷ is N(R¹⁴)₂ wherein said R¹⁴ are taken together with their adjacent nitrogen atom to form a C₄₋₁₀heterocycloalkyl group, wherein said heterocycloalkyl may be mono or bicyclic and include from 1 to 3 heteroatoms, preferably wherein X⁷ is N. In a preferred embodiment, the heterocycloalkyl is substituted, for instance, with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.). For instance, the heterocycloalkyl may be selected from optionally substituted piperidine, piperazine, thiomorpholine, and morpholine groups, or a bicyclic structure (bridged or spiro) containing a piperidine, piperazine, thiomorpholine, or morpholine ring.

In another example, R¹ is selected from:

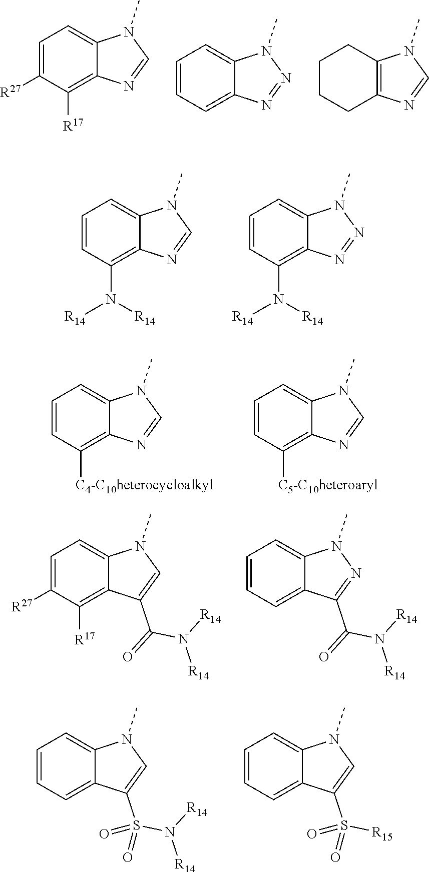

wherein R¹⁴, R¹⁷, and R²⁷ are as defined herein and ( - - - ) represents a bond serving as a point of attachment between R¹ and the rest of the molecule.

In a further example, R¹ is selected from:

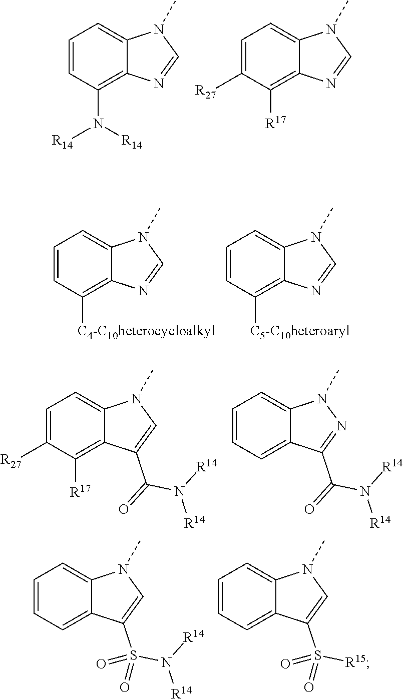

wherein R¹⁴, R¹⁷, and R²⁷ are as defined herein and ( - - - ) represents a bond serving as a point of attachment between R¹ and the rest of the molecule.

Further subgeneric embodiments are also presented in the examples section, wherein each substituent group R¹ (group), R² (B group), and L are defined. Examples of combinations are also set forth further below and in Tables 3 and 4. Representative preferred compounds of Examples 1 to 159 are also described herein.

More specifically, exemplary R¹ groups are illustrated as A1 to A550 defined as follows:

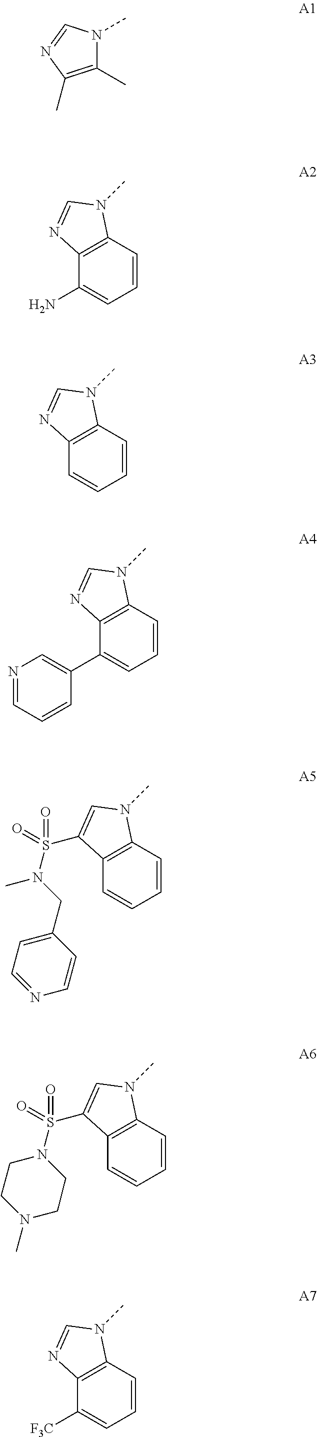

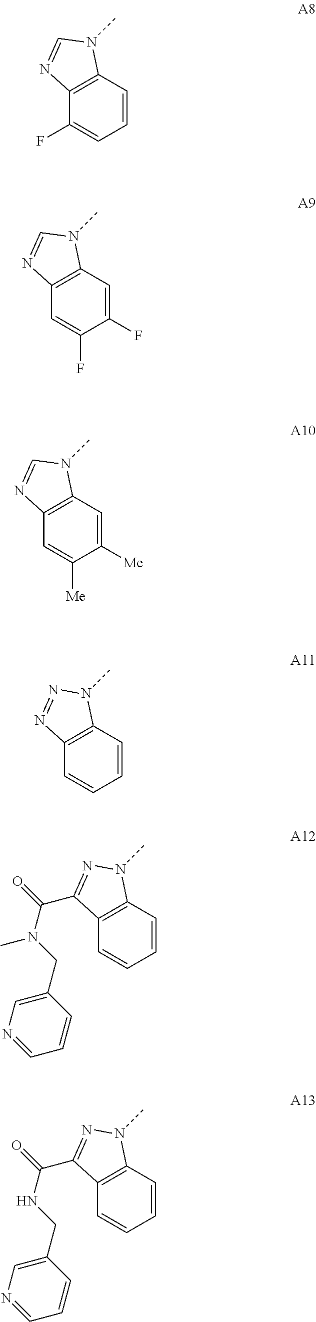

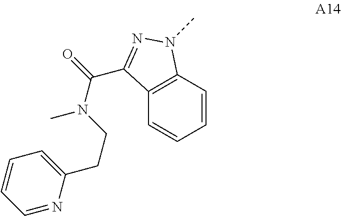

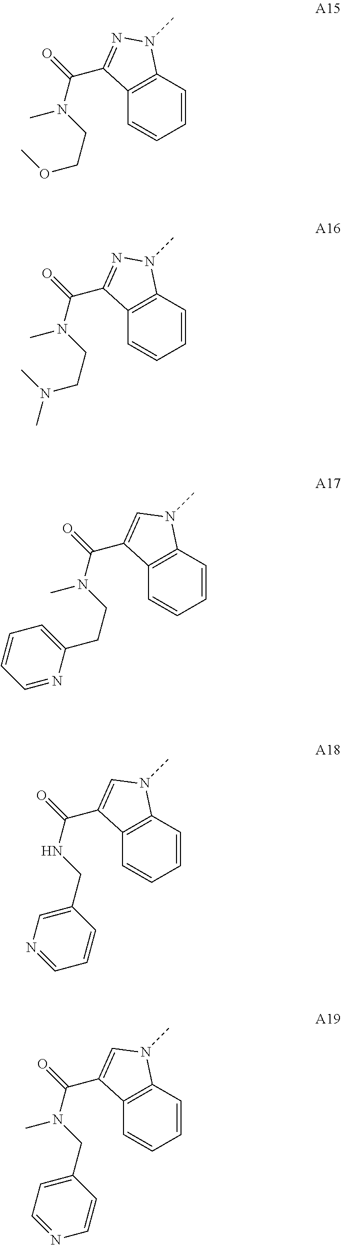

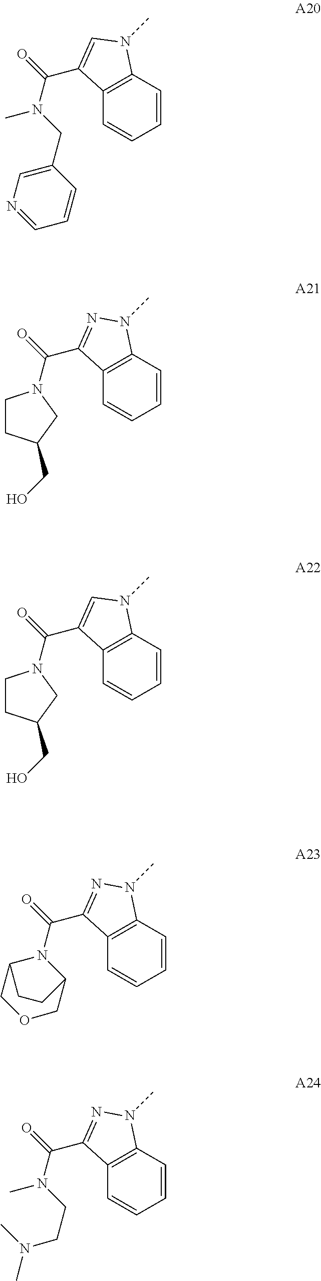

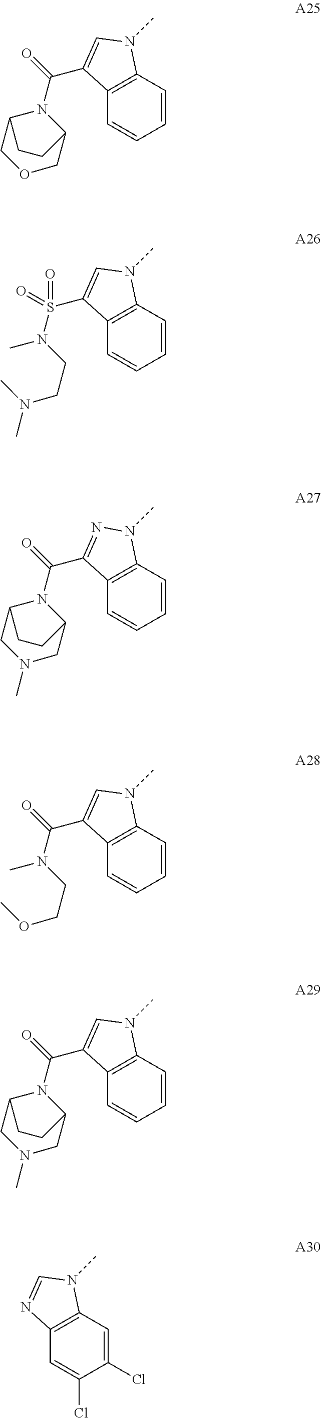

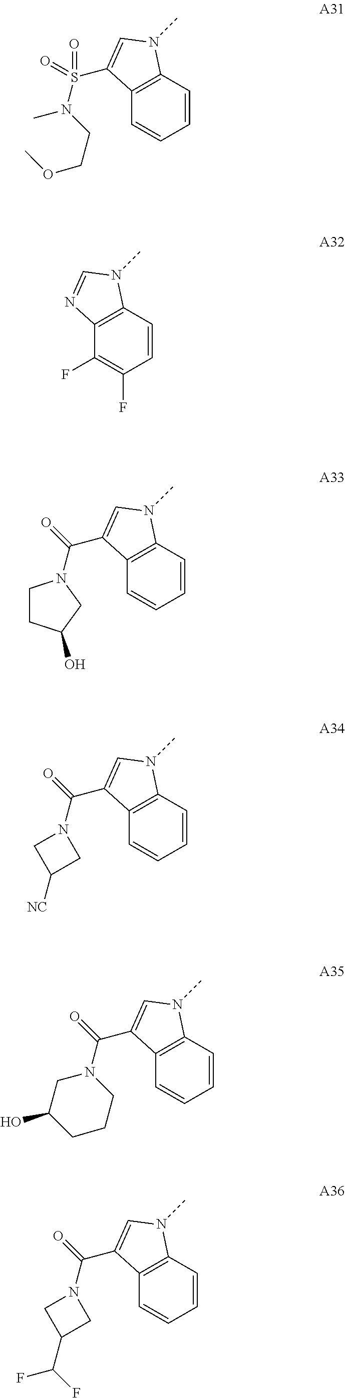

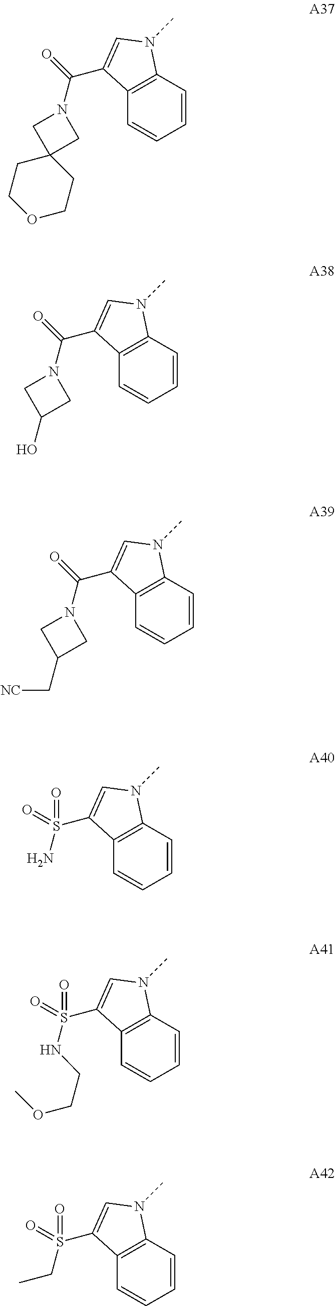

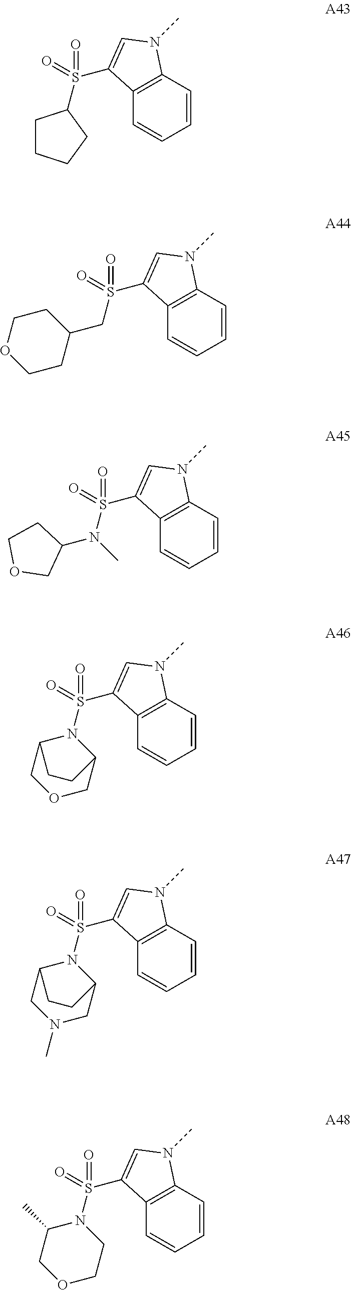

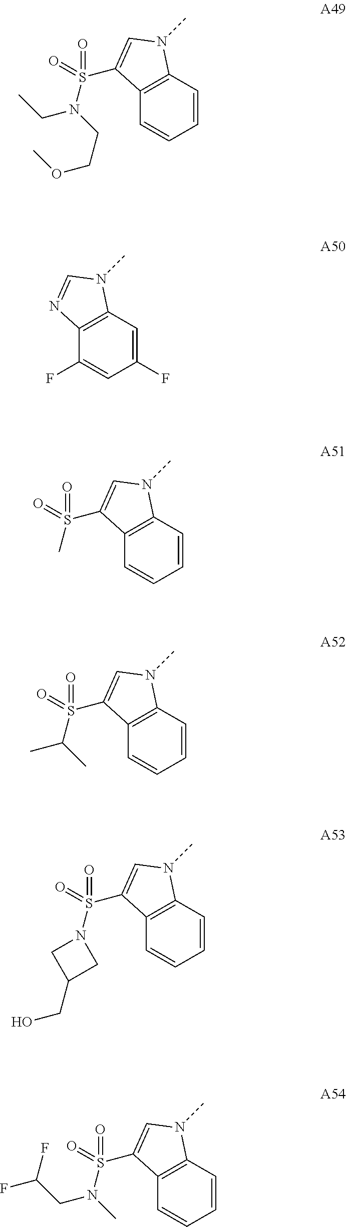

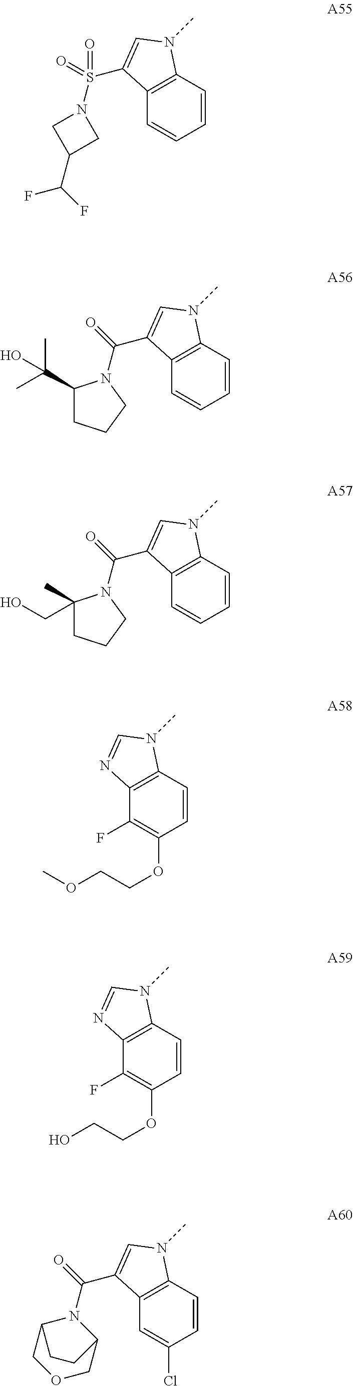

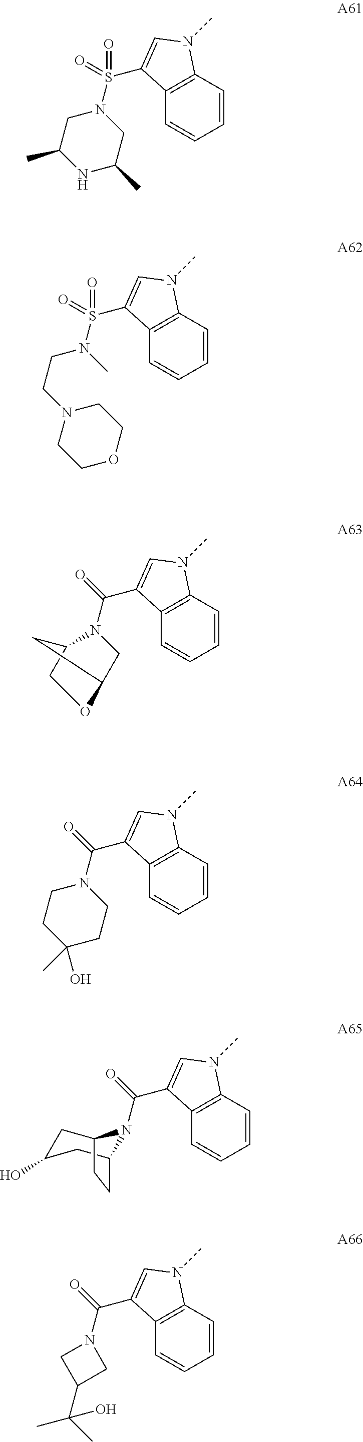

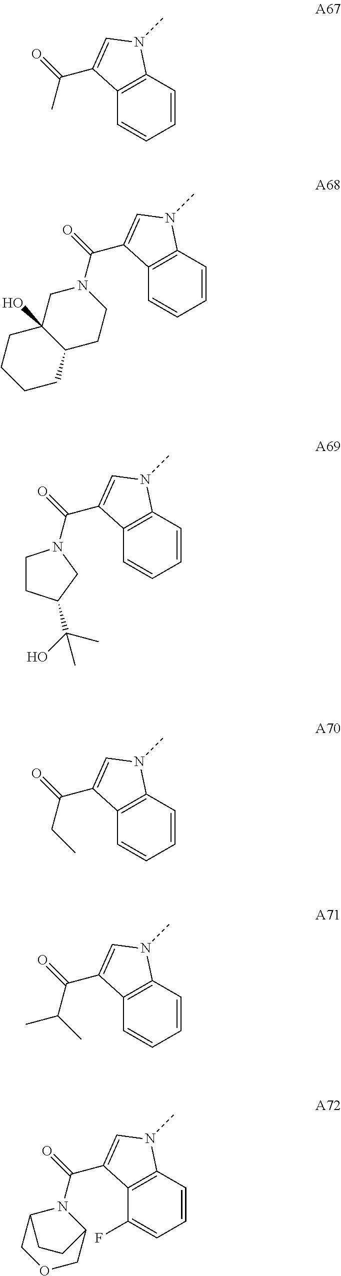

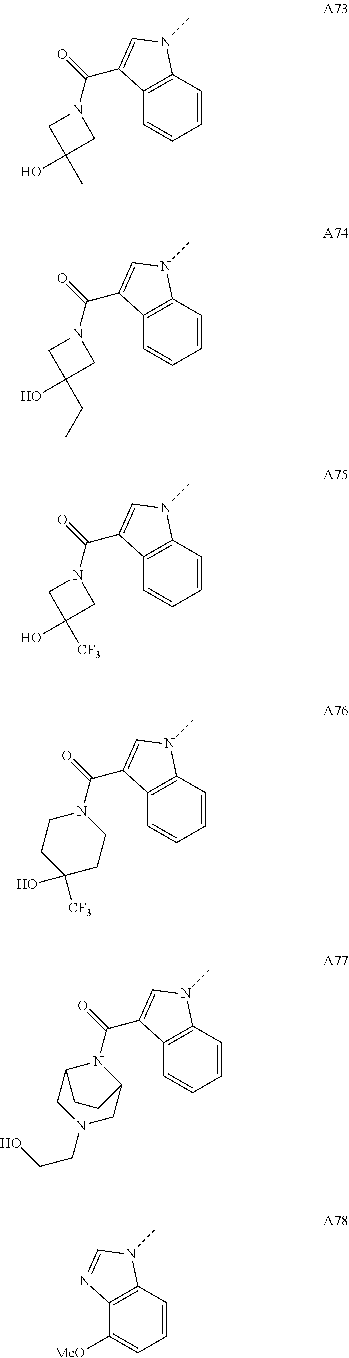

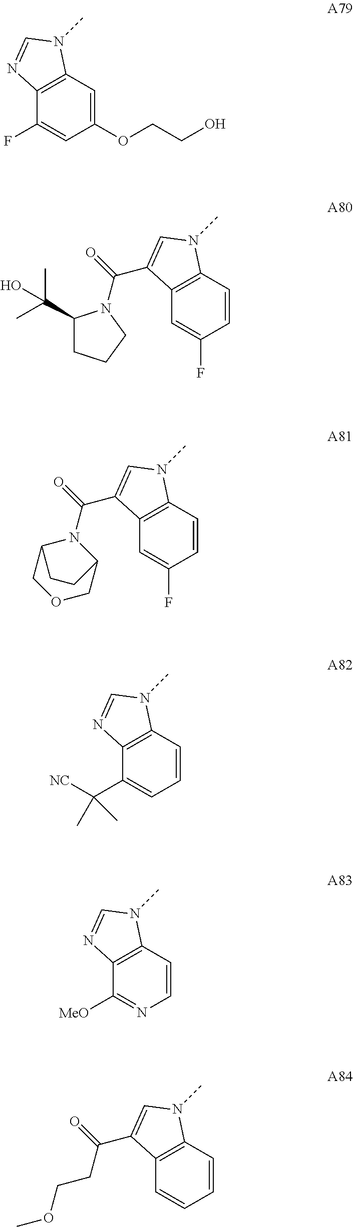

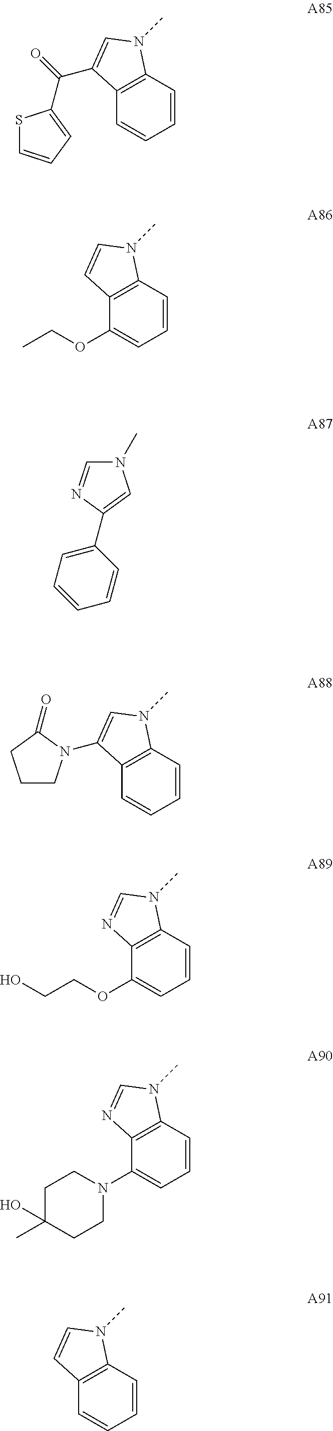

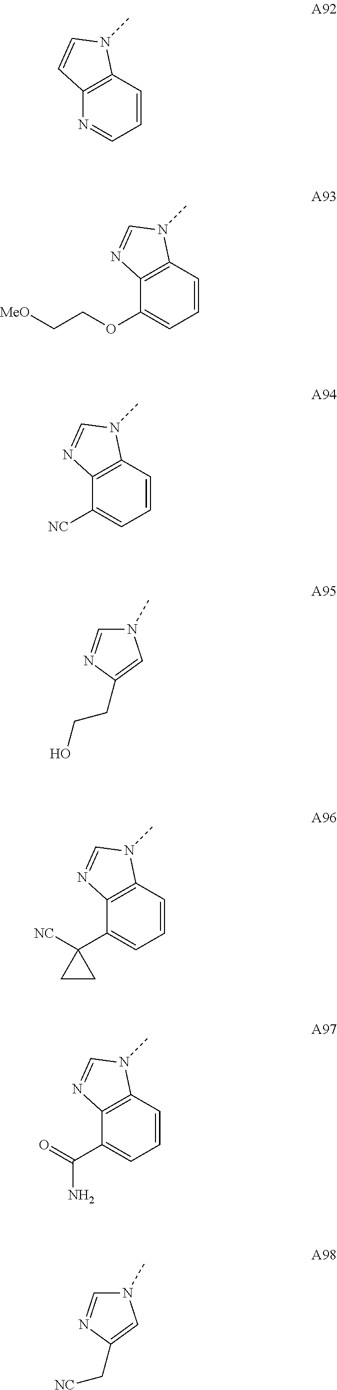

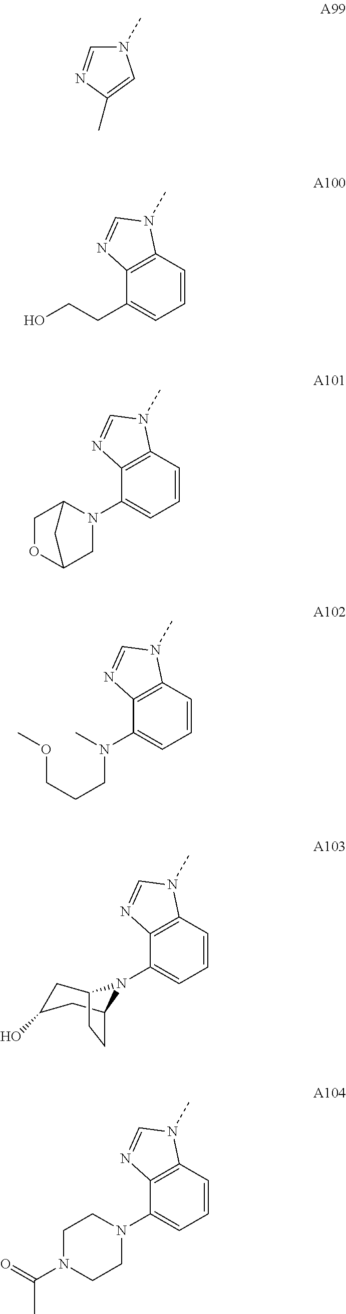

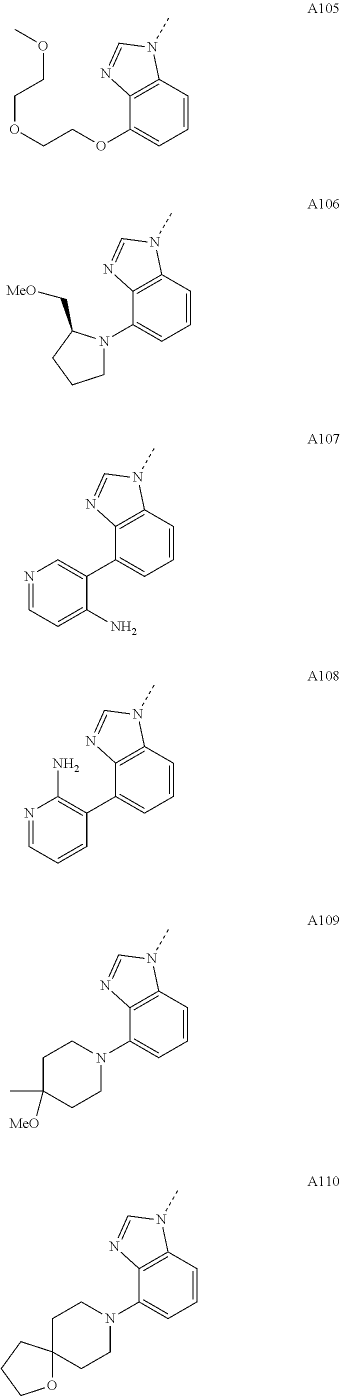

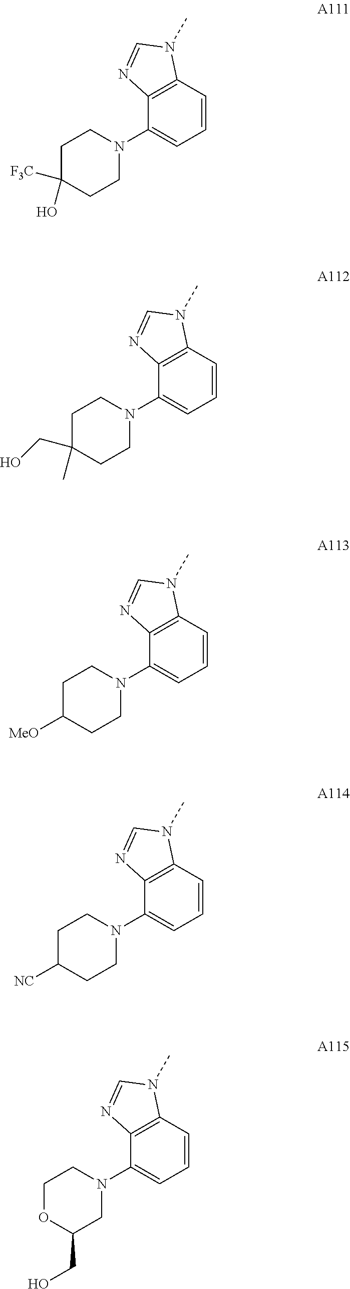

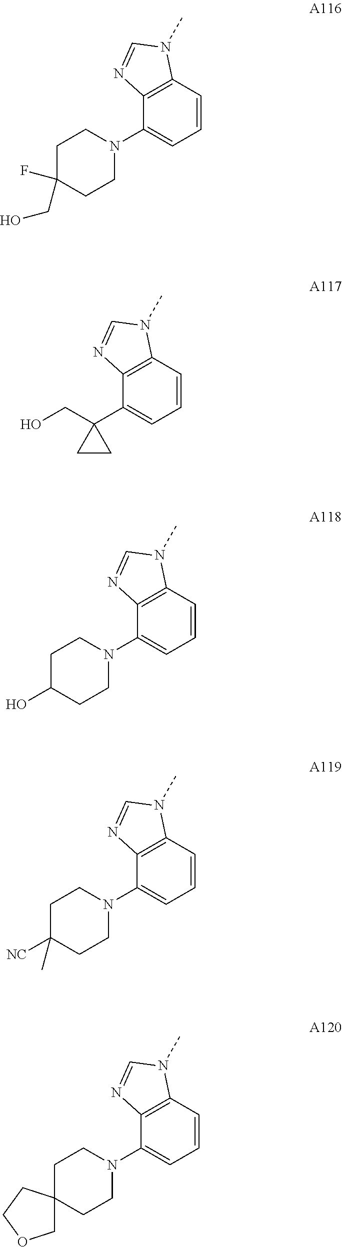

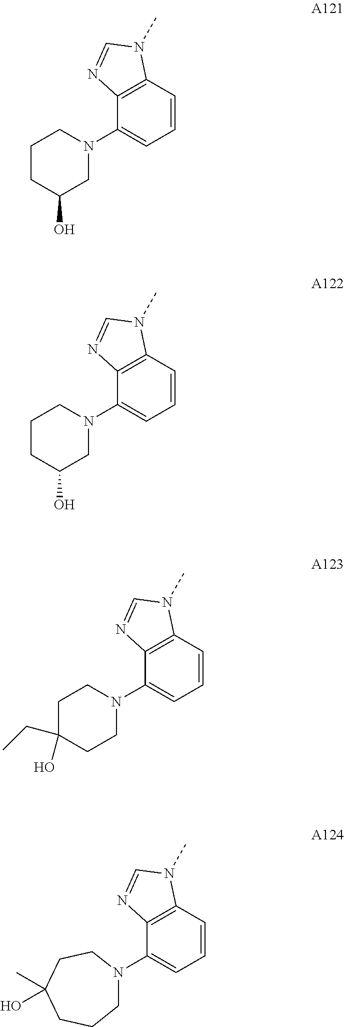

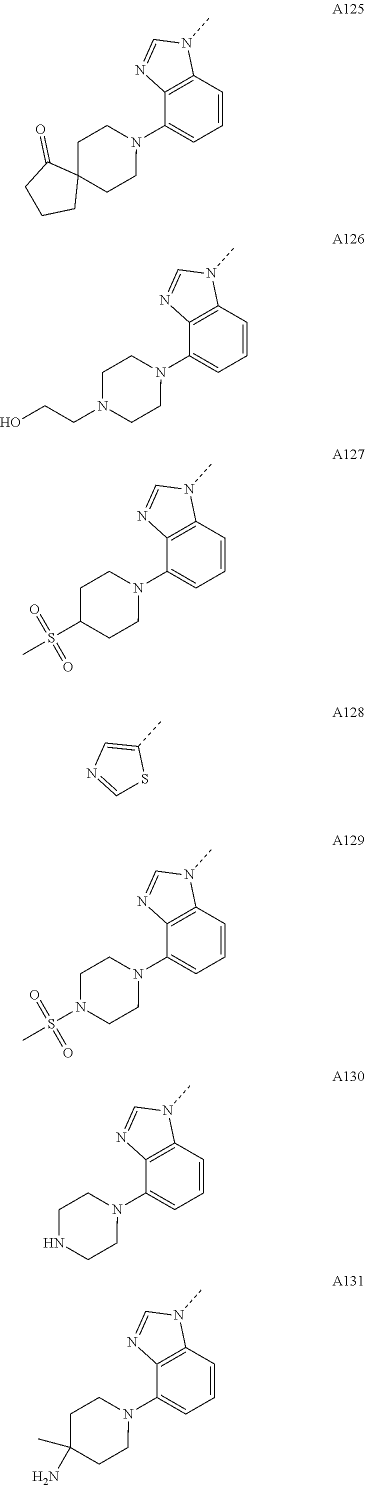

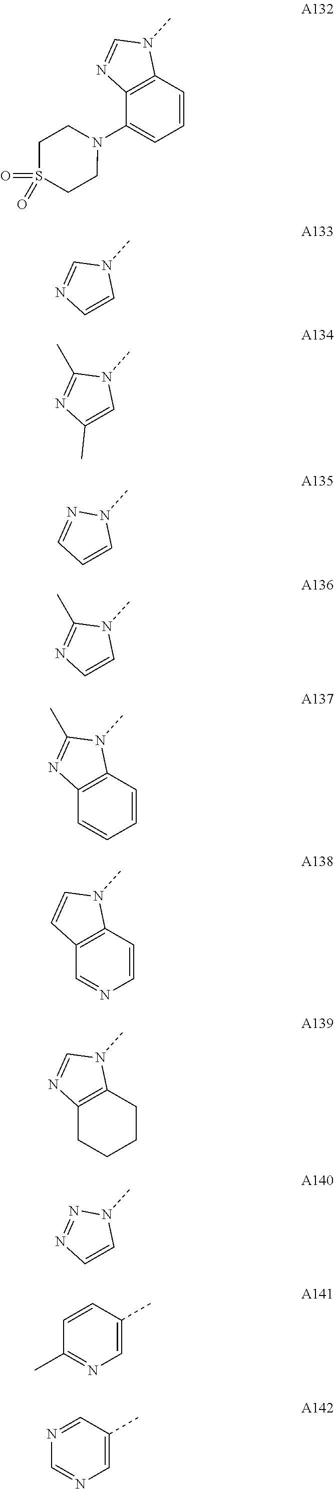

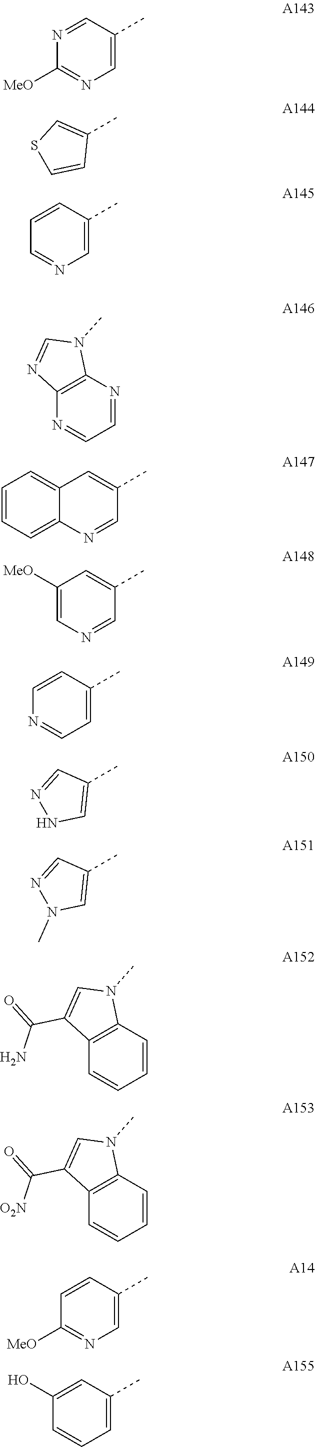

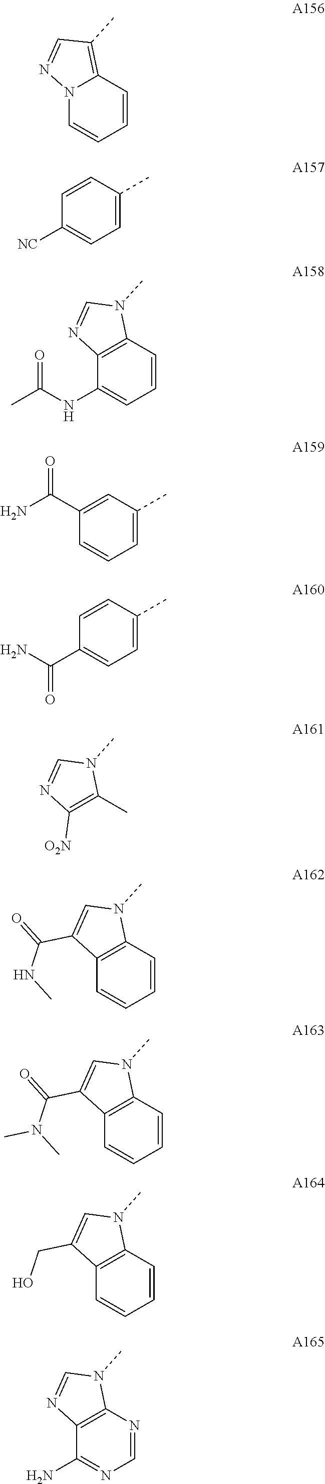

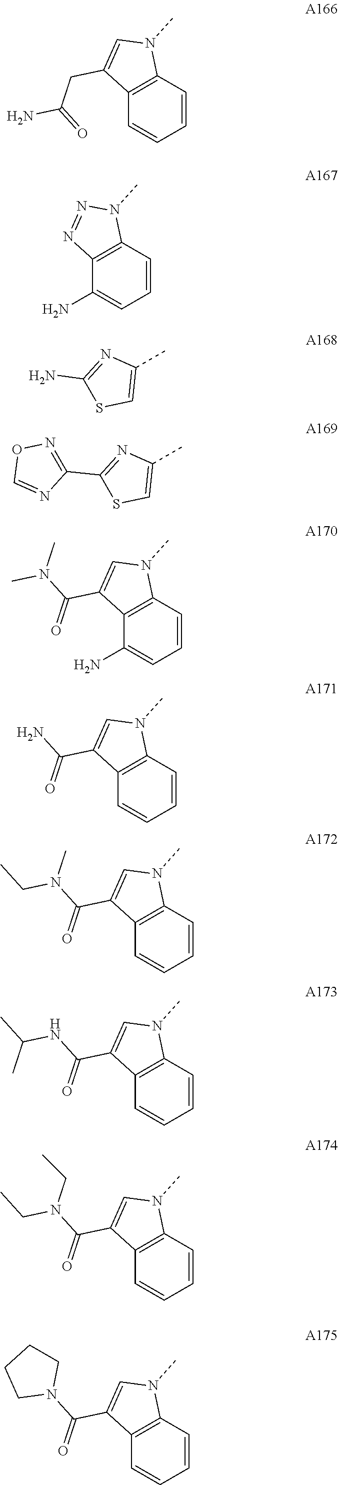

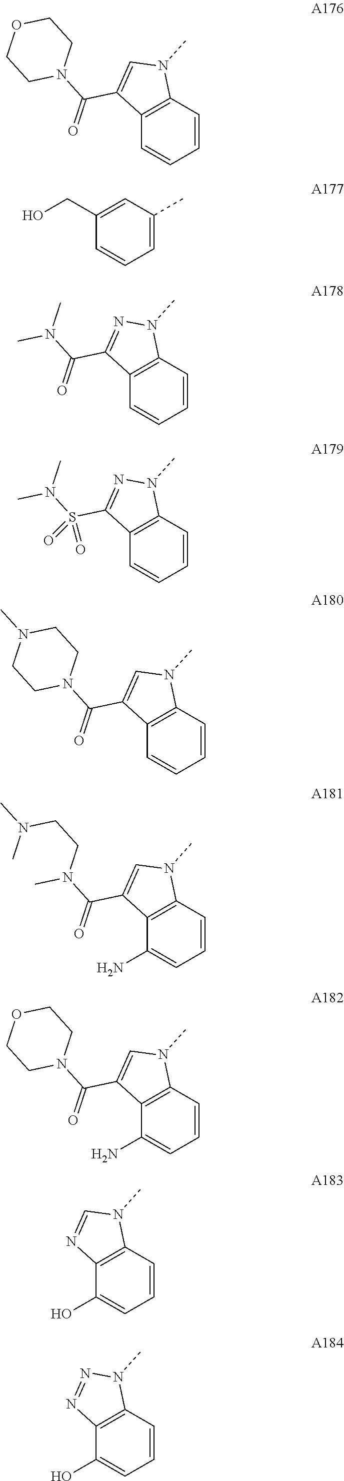

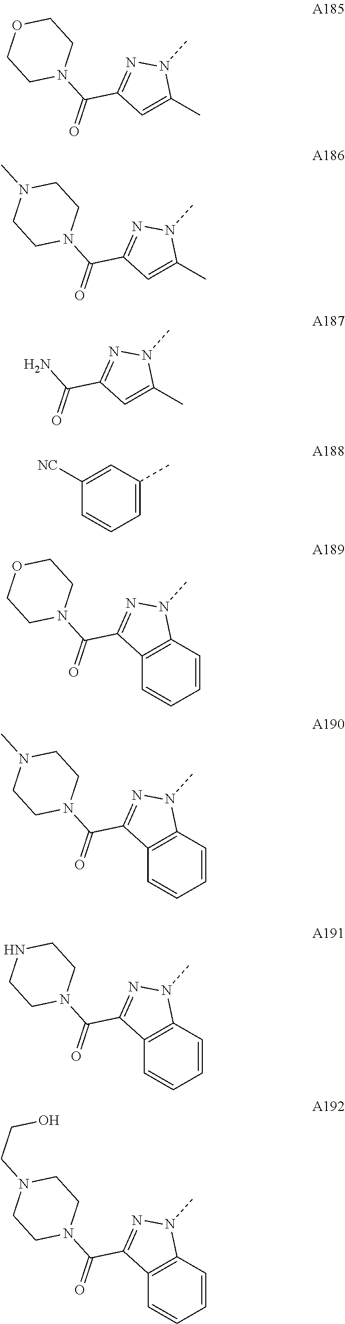

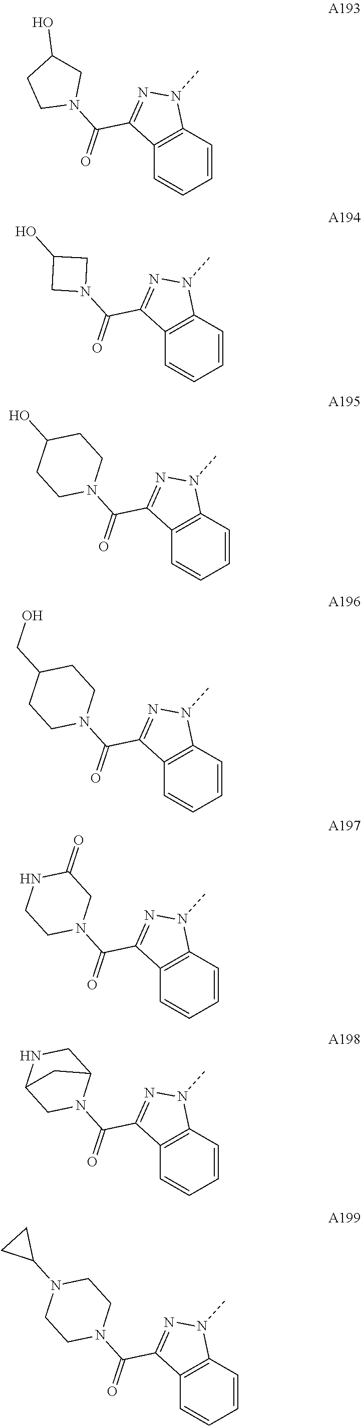

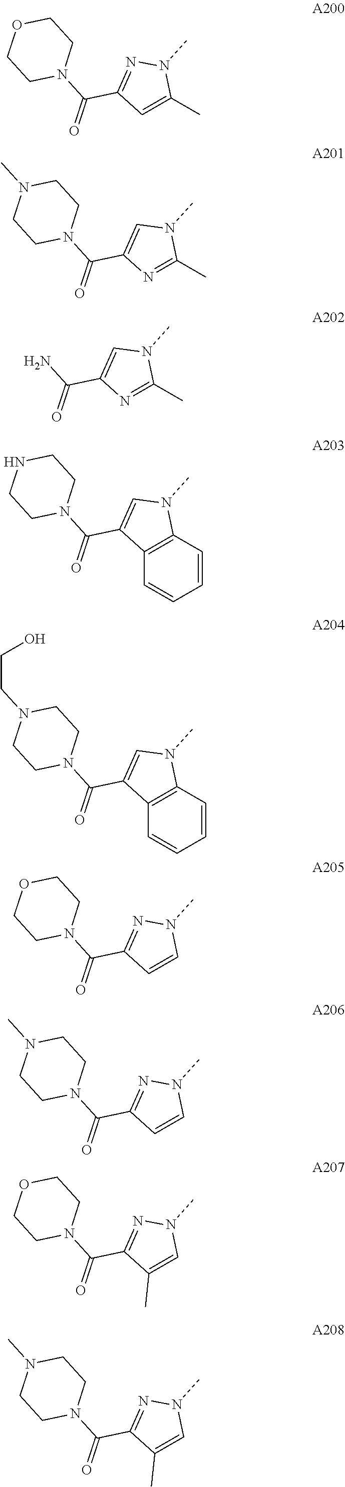

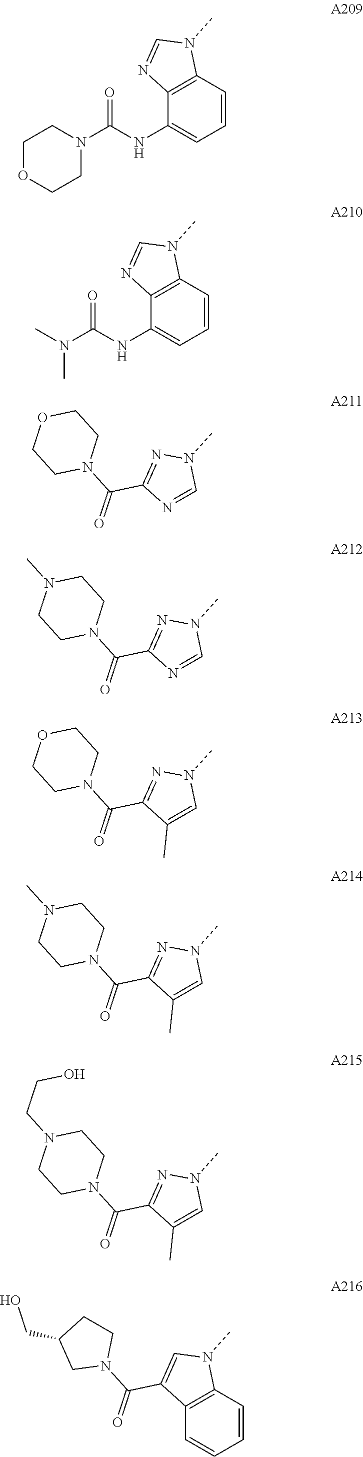

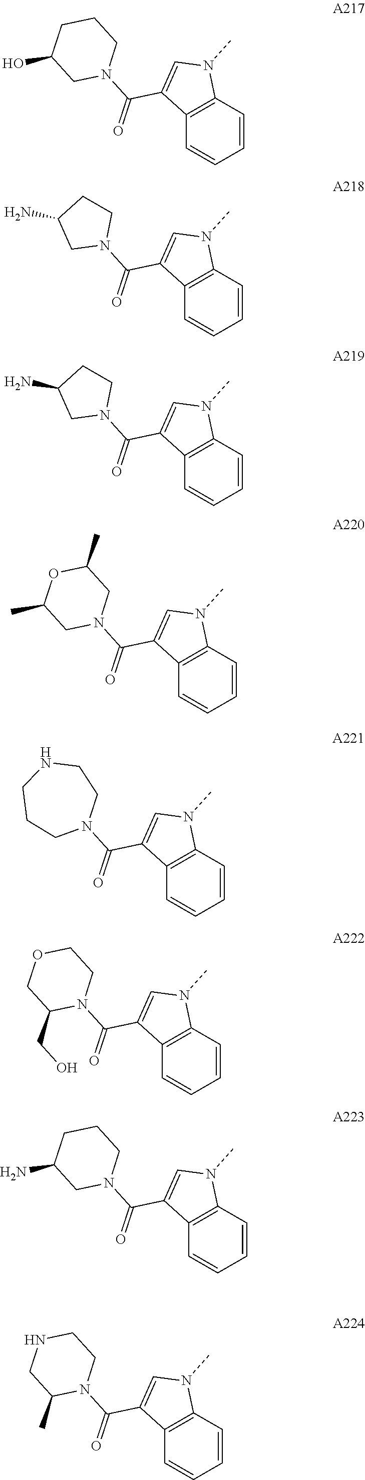

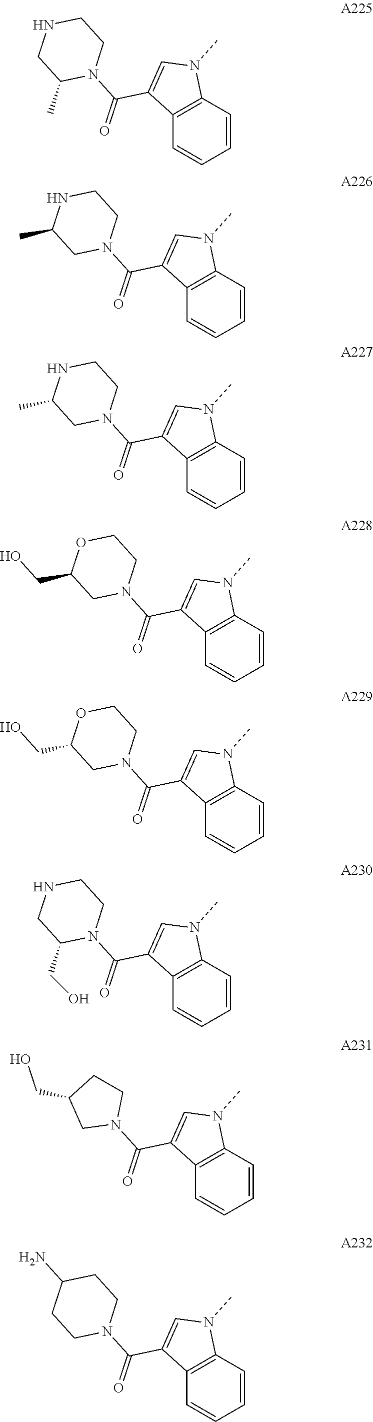

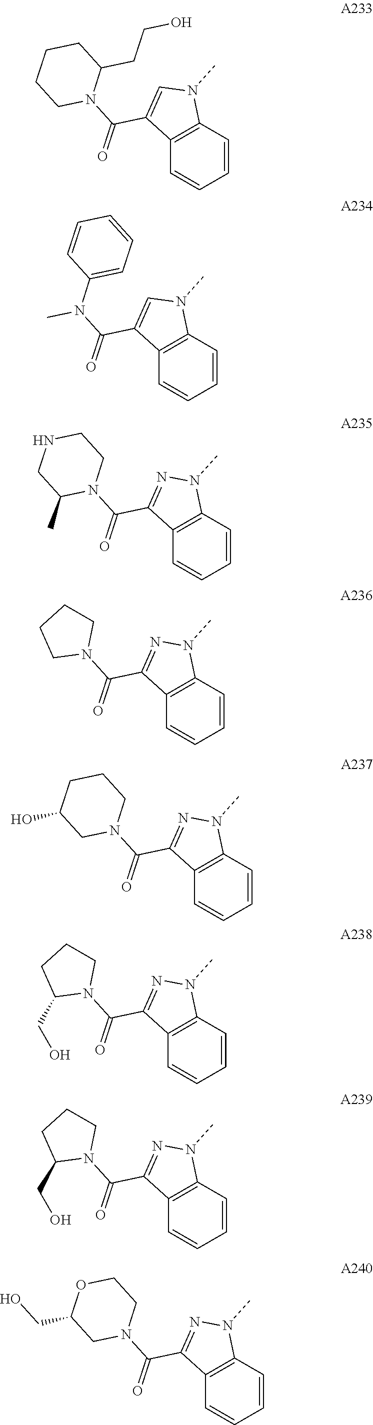

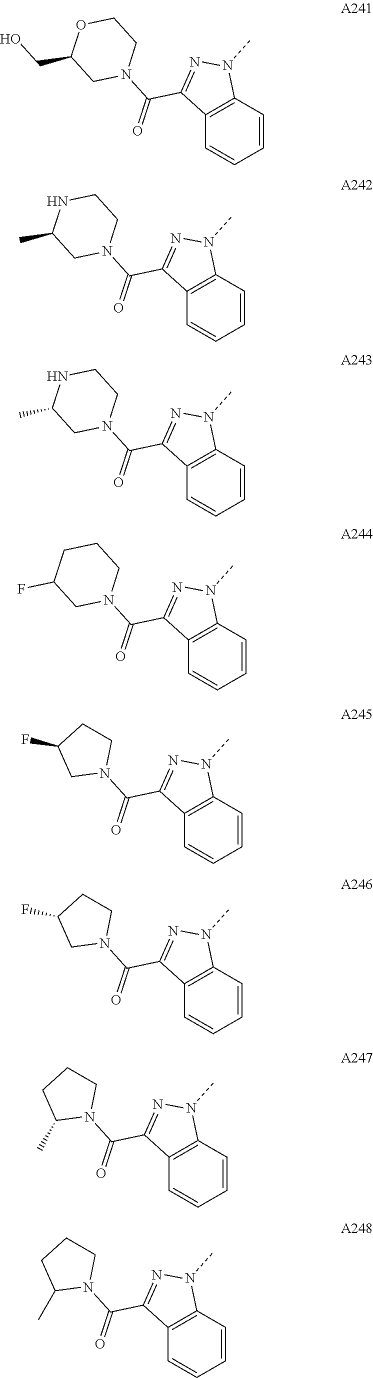

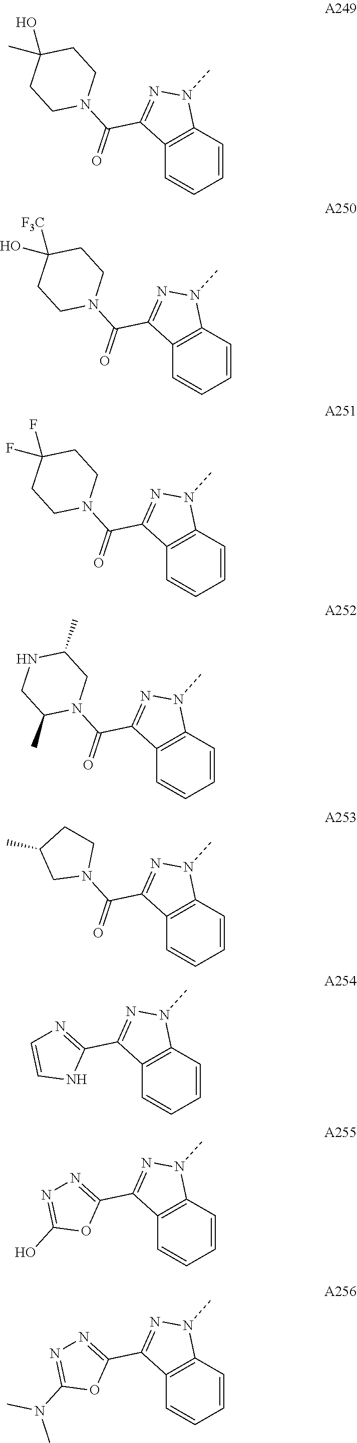

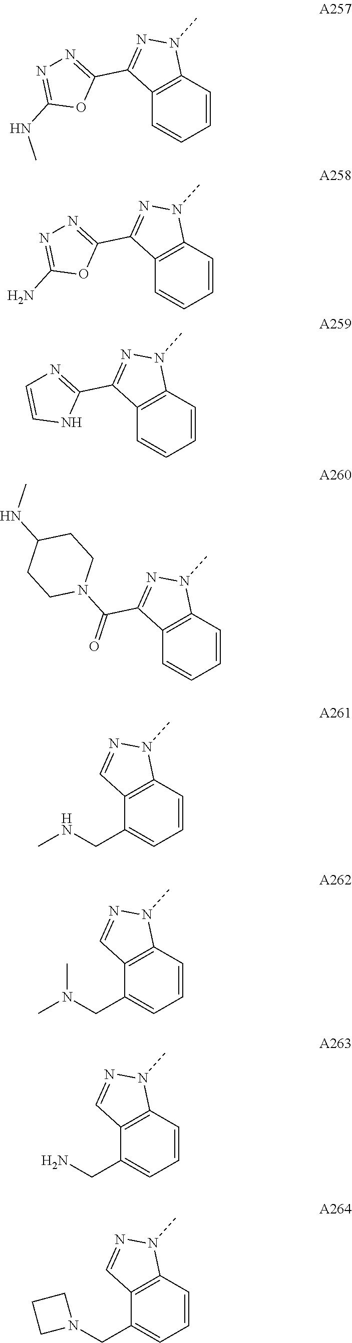

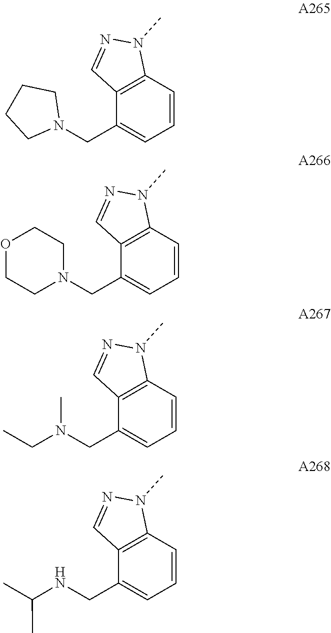

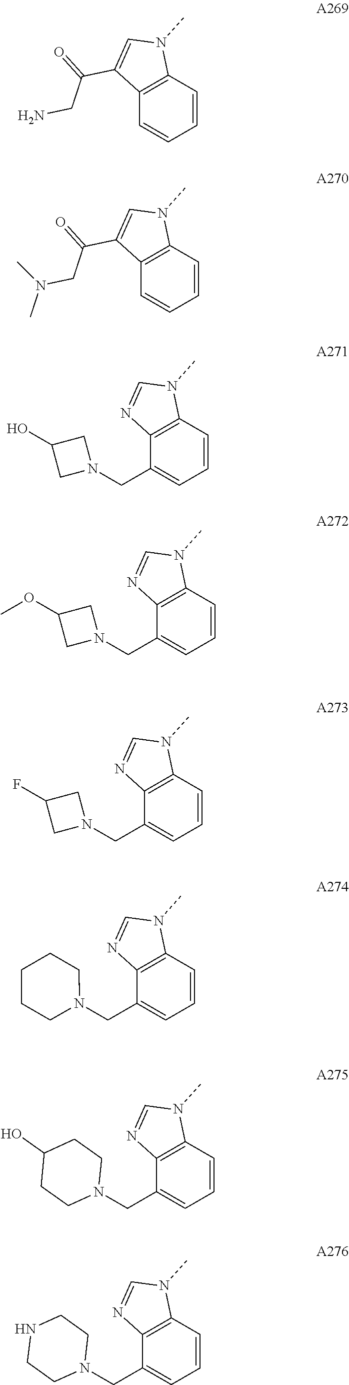

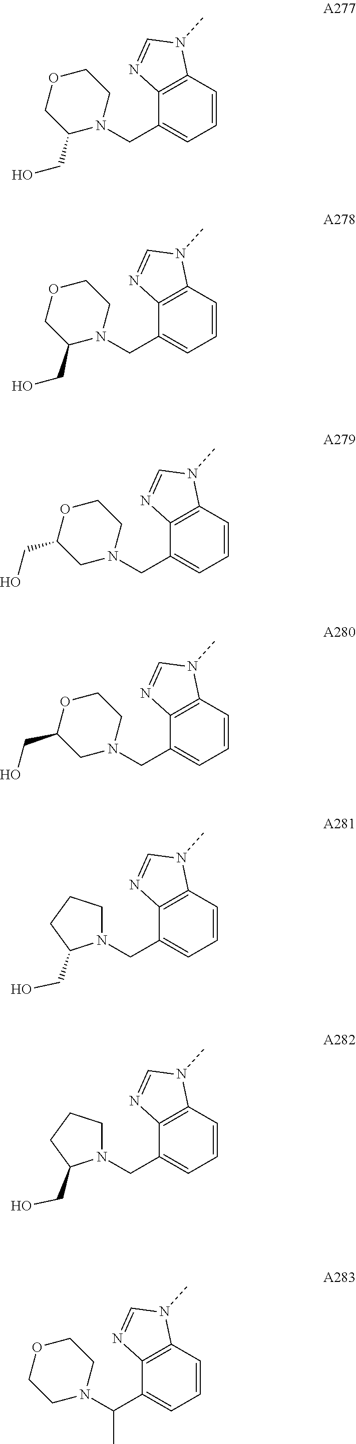

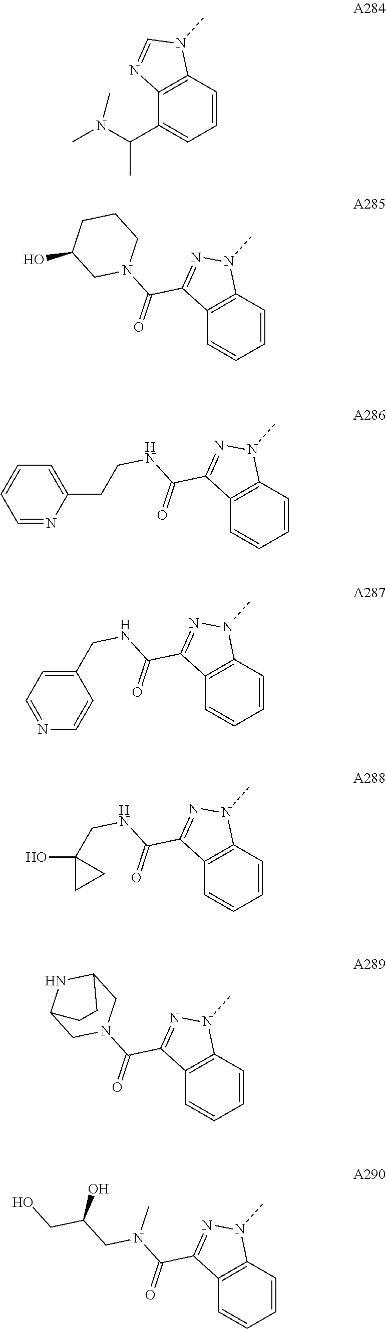

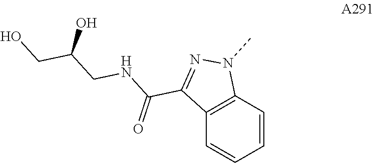

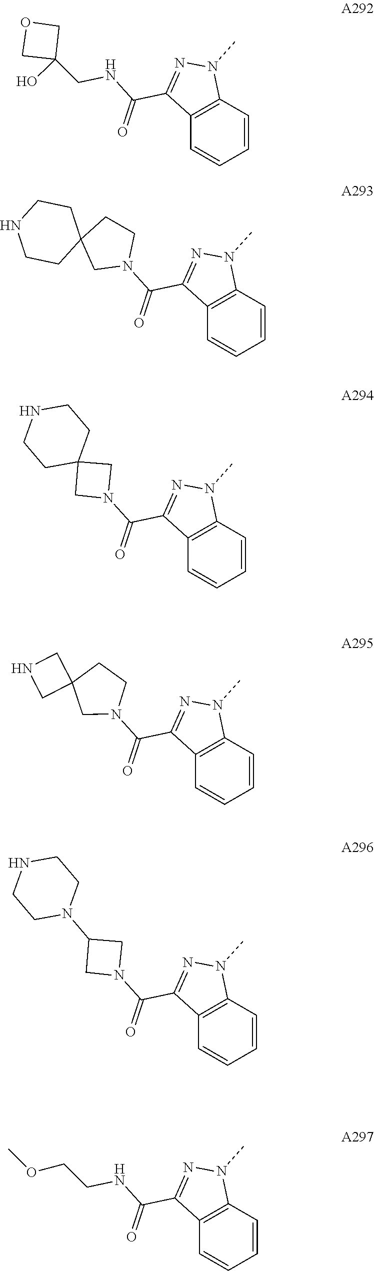

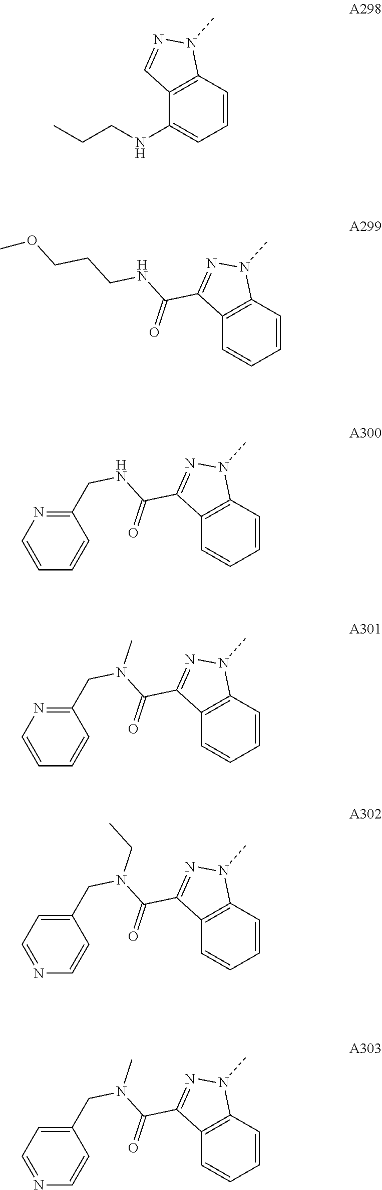

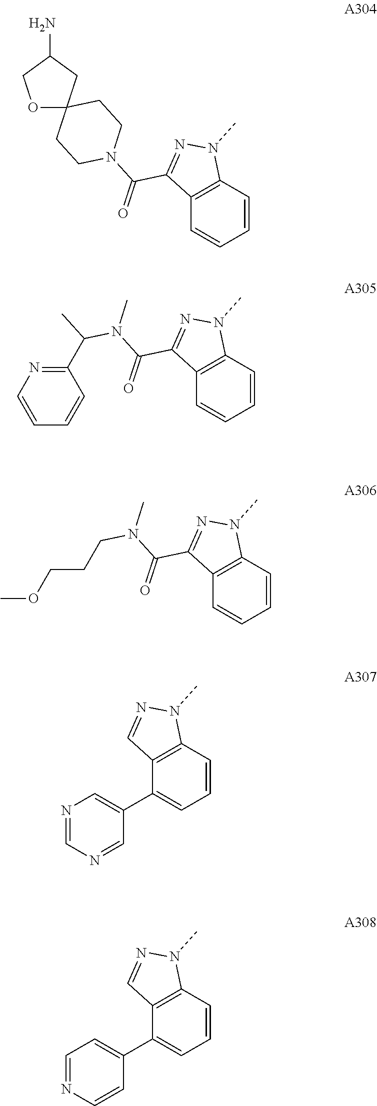

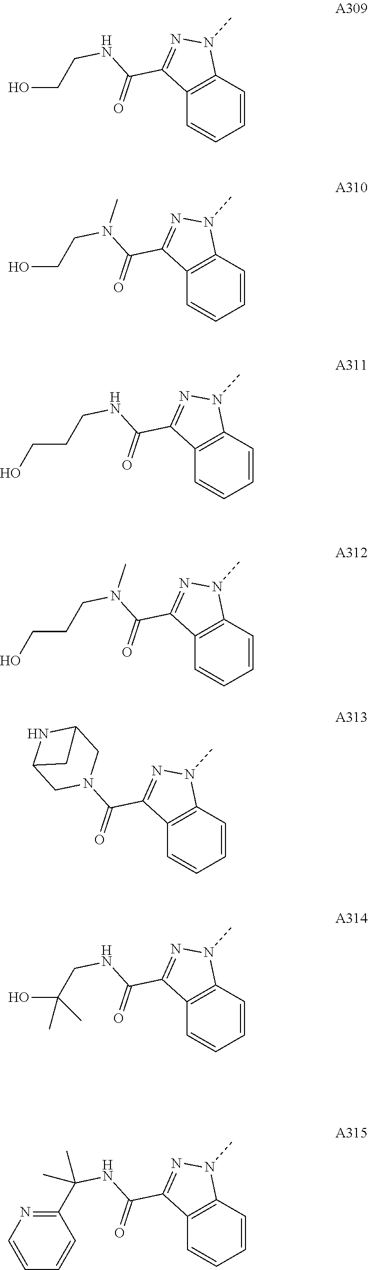

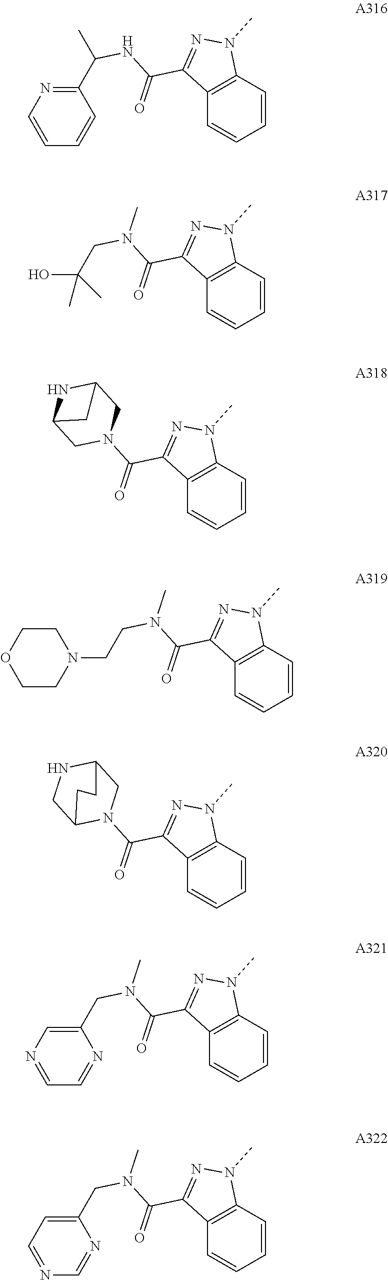

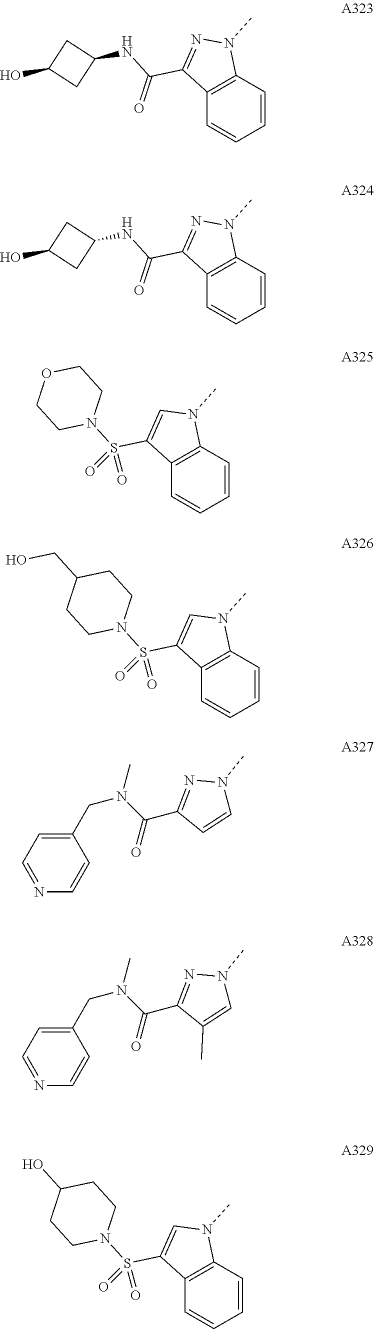

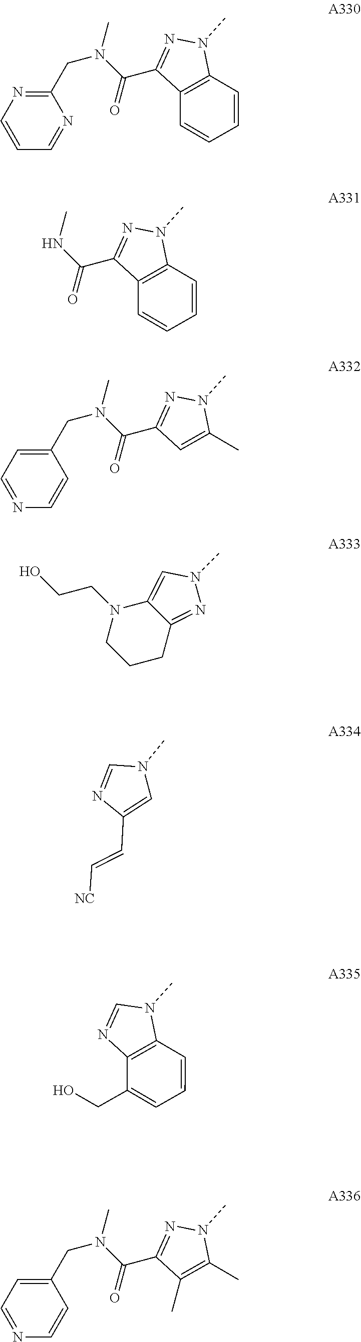

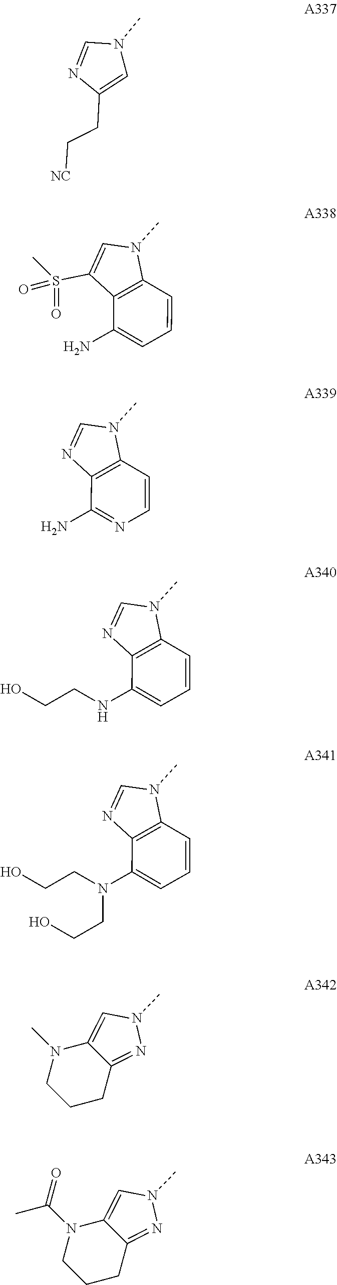

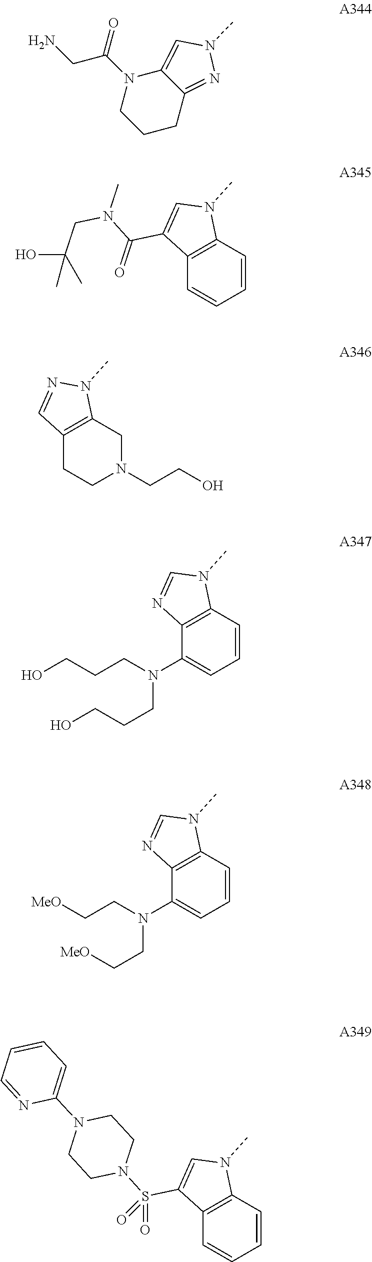

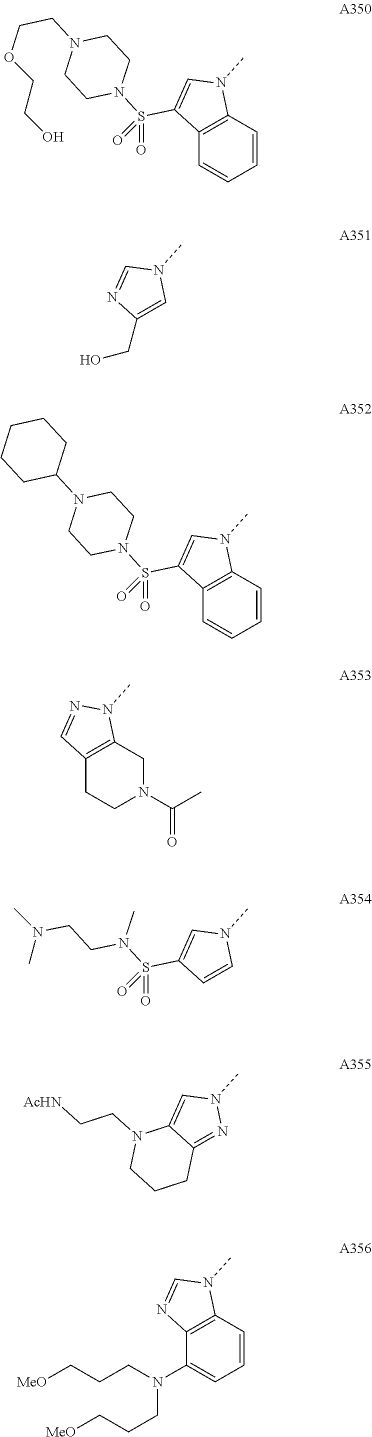

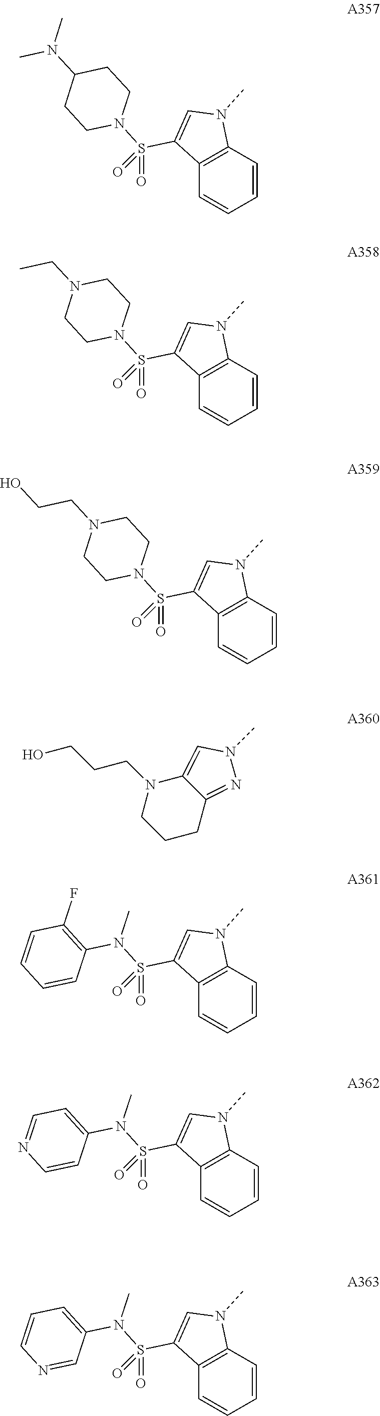

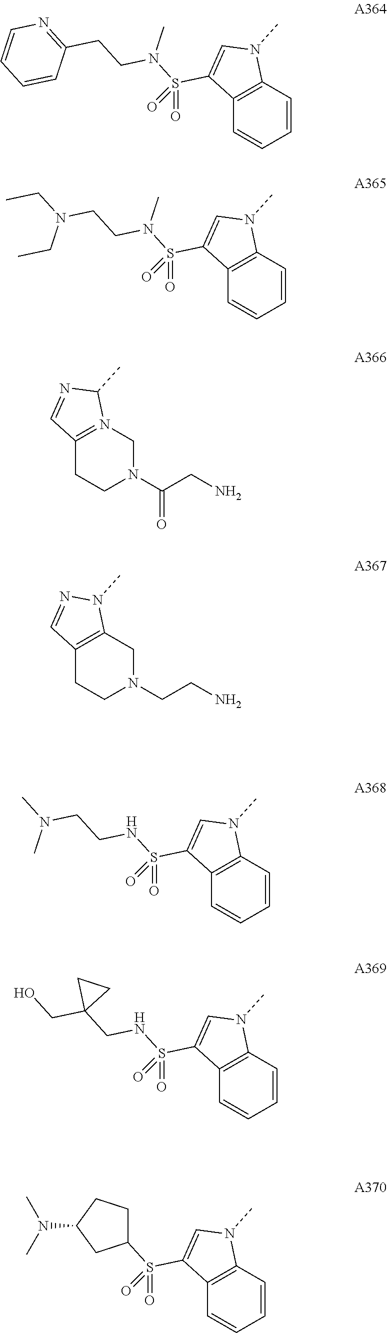

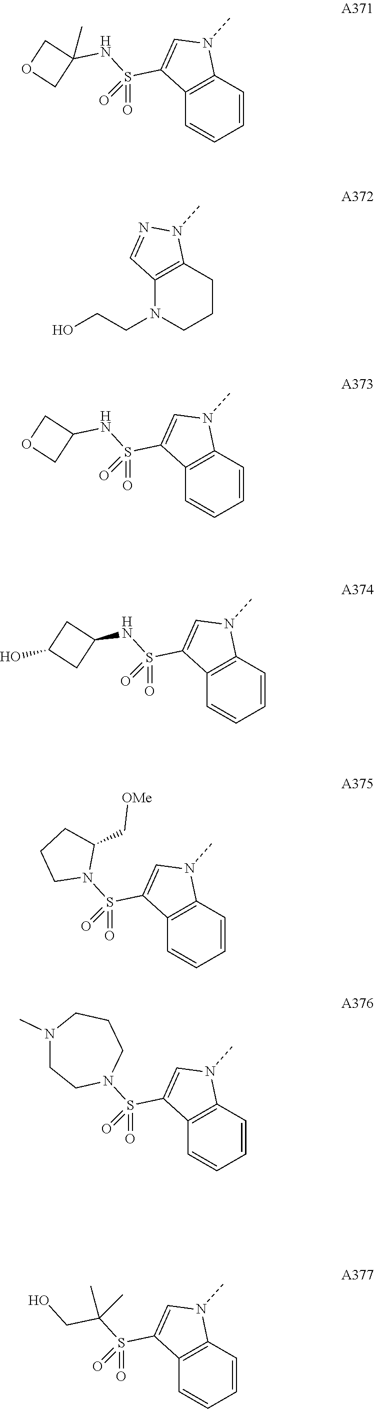

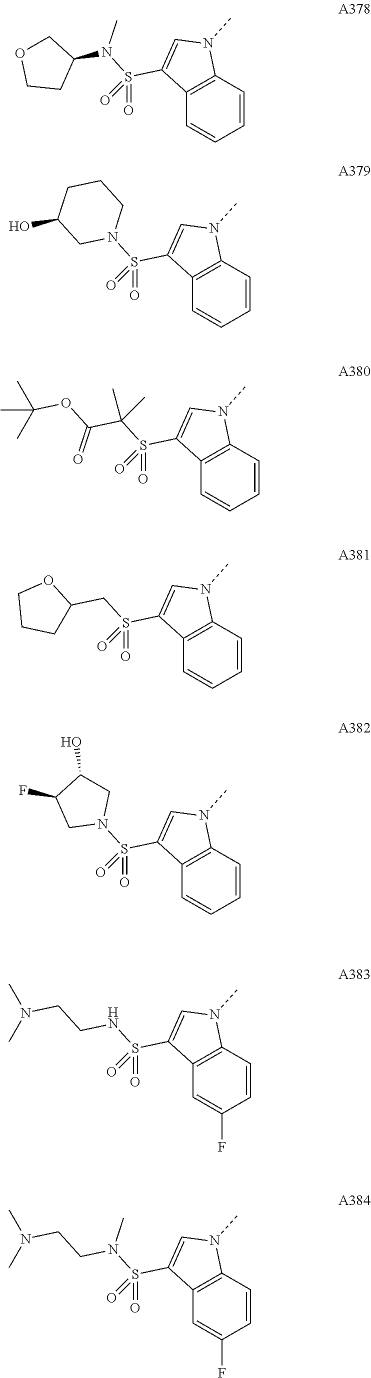

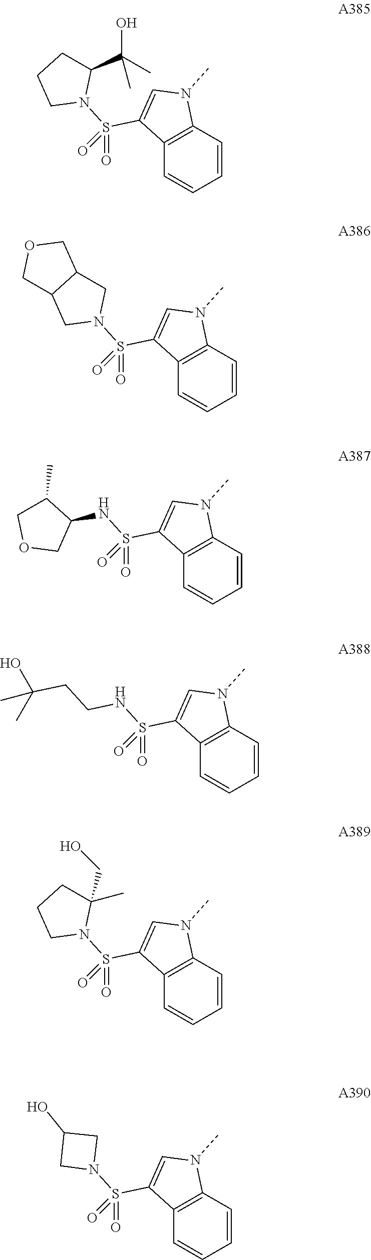

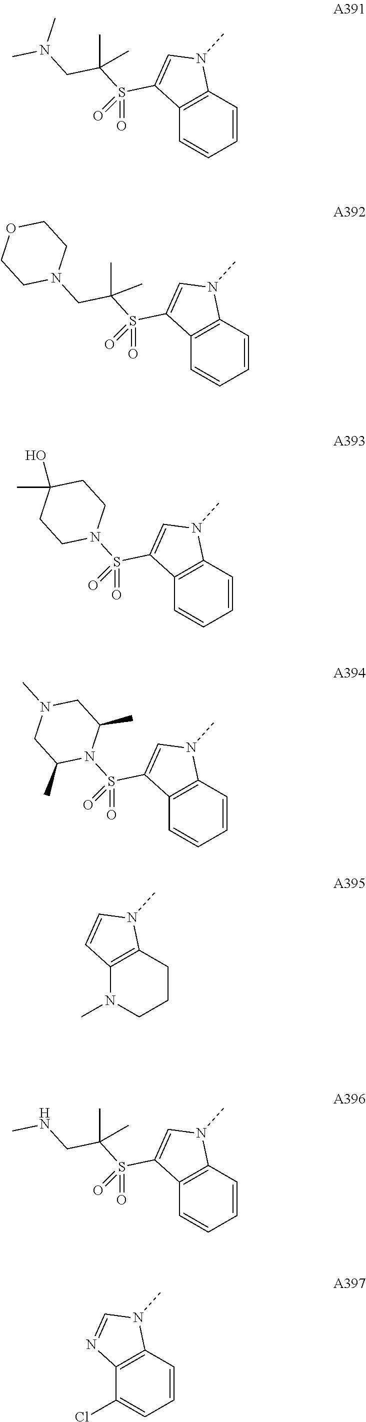

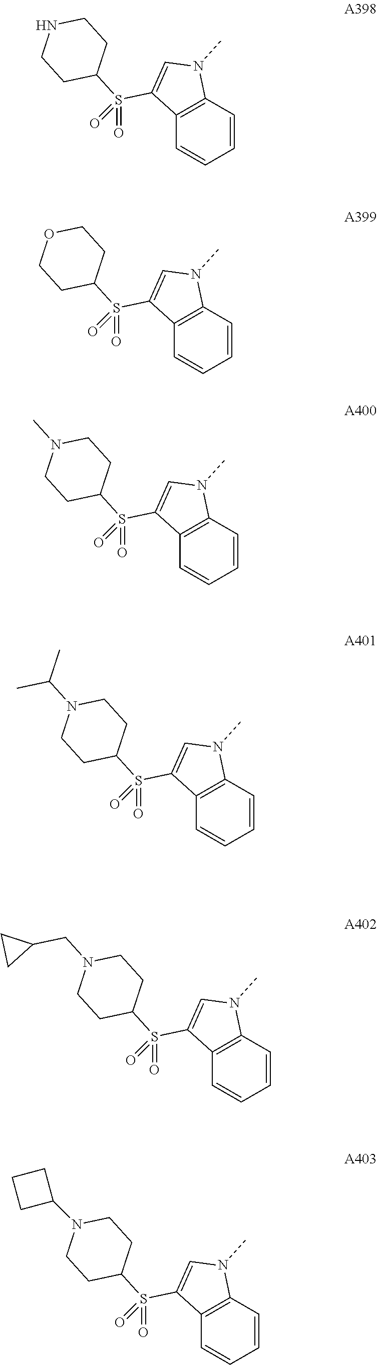

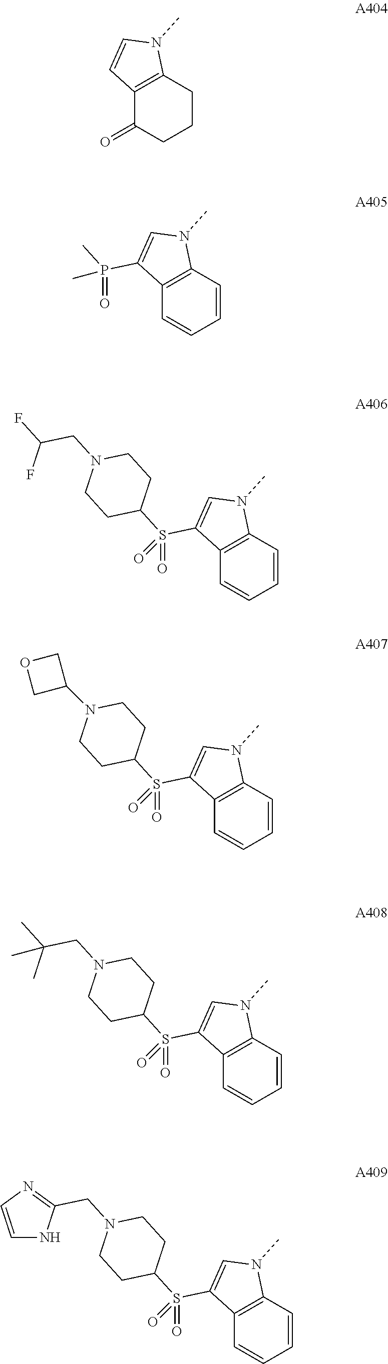

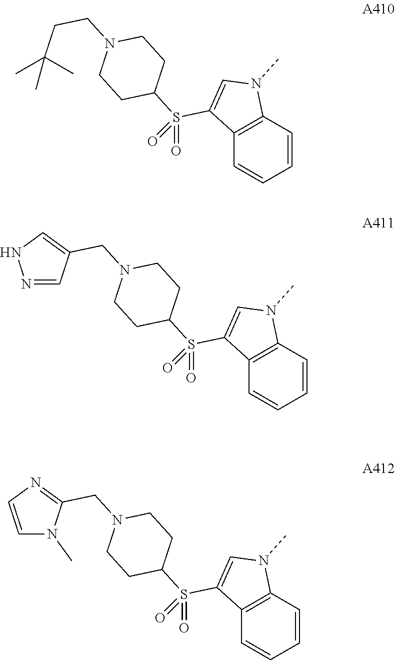

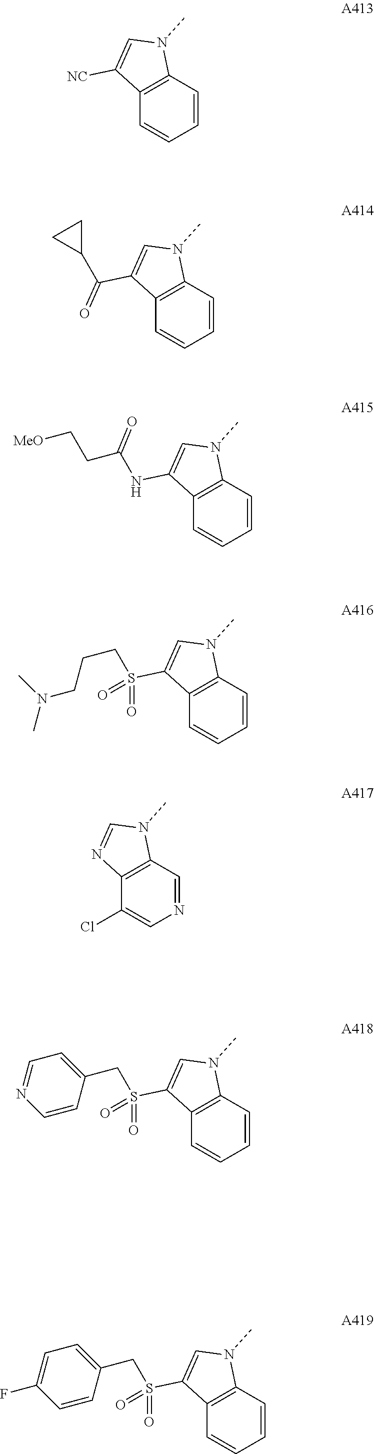

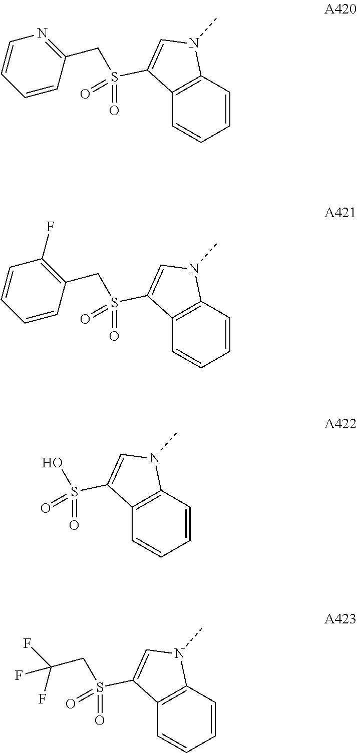

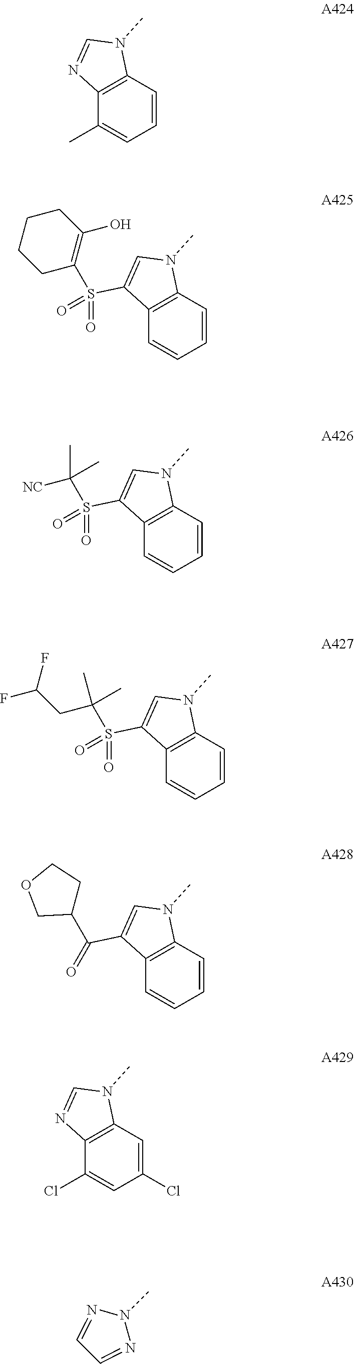

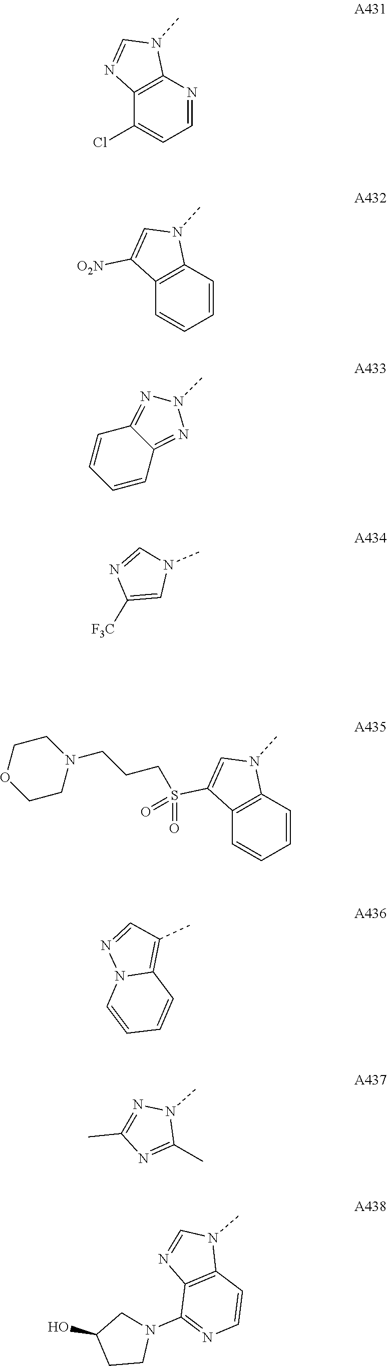

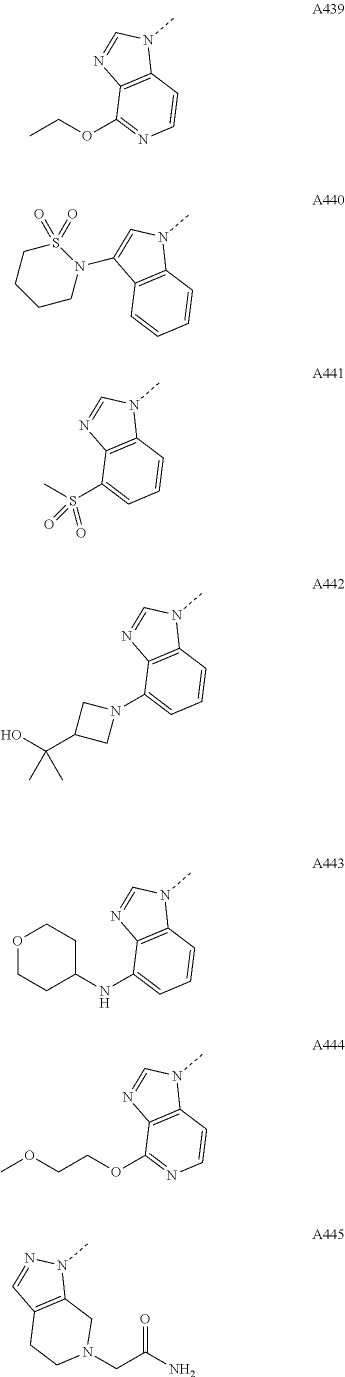

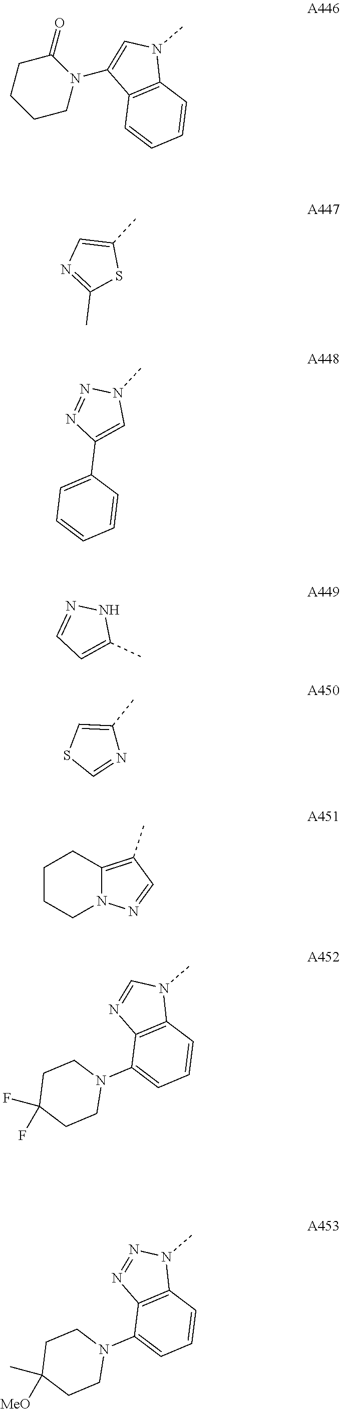

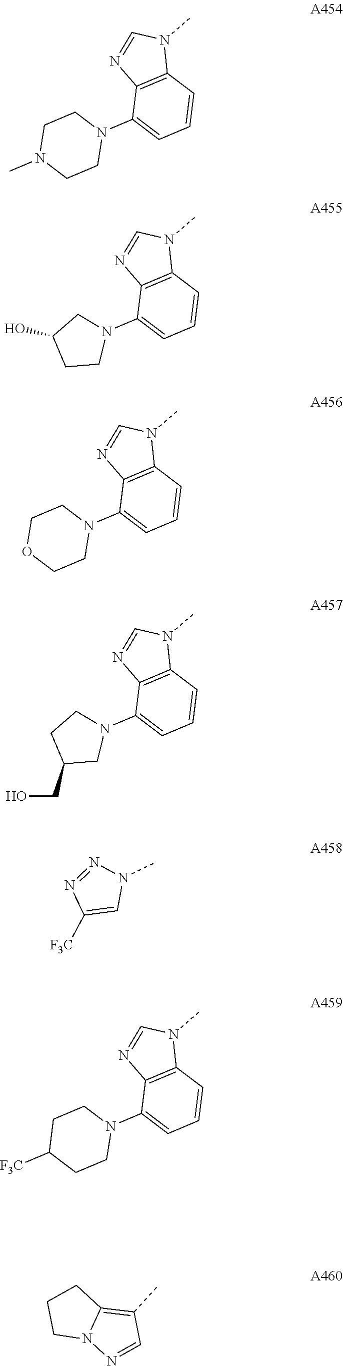

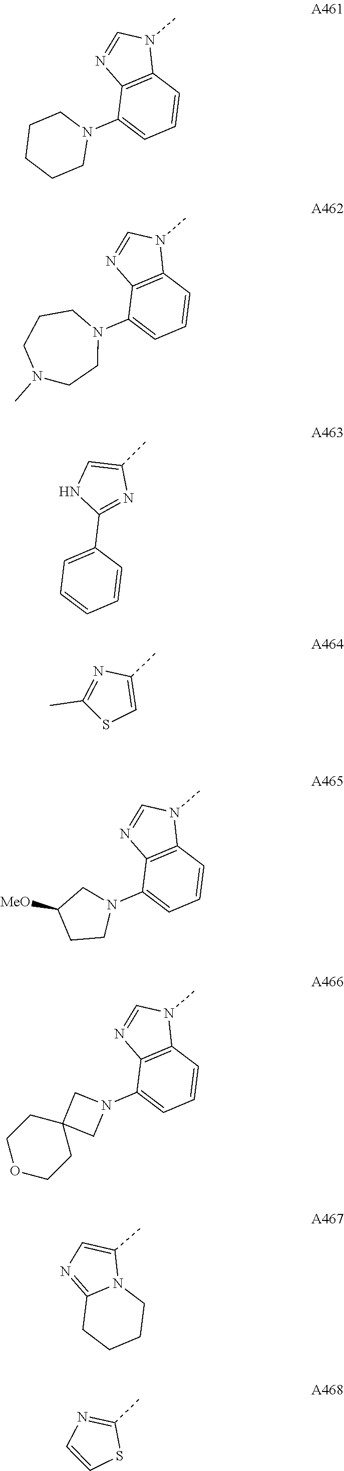

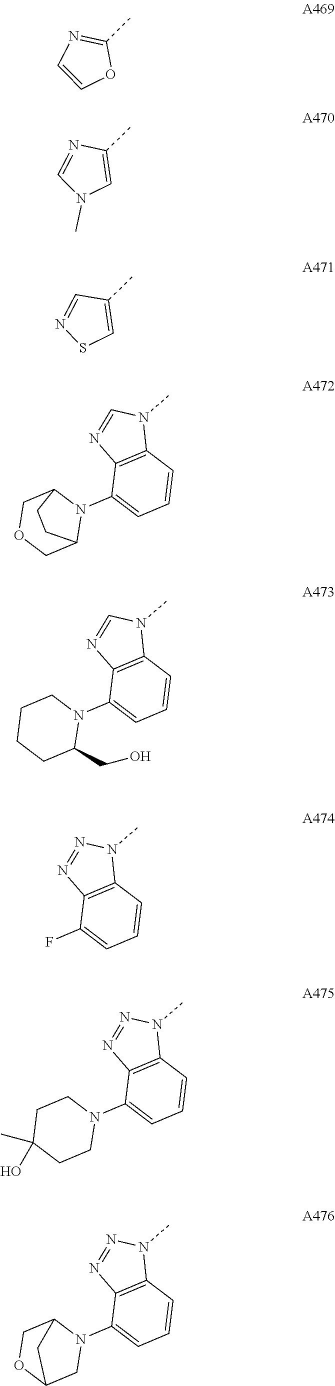

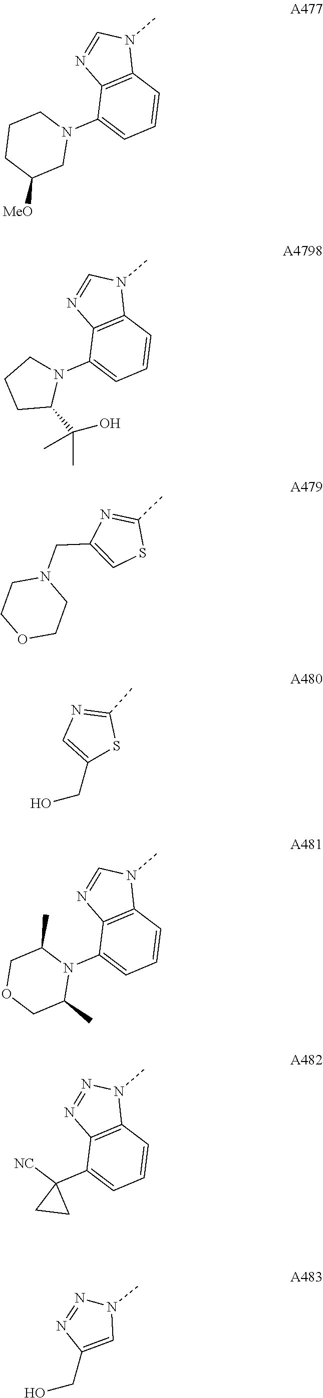

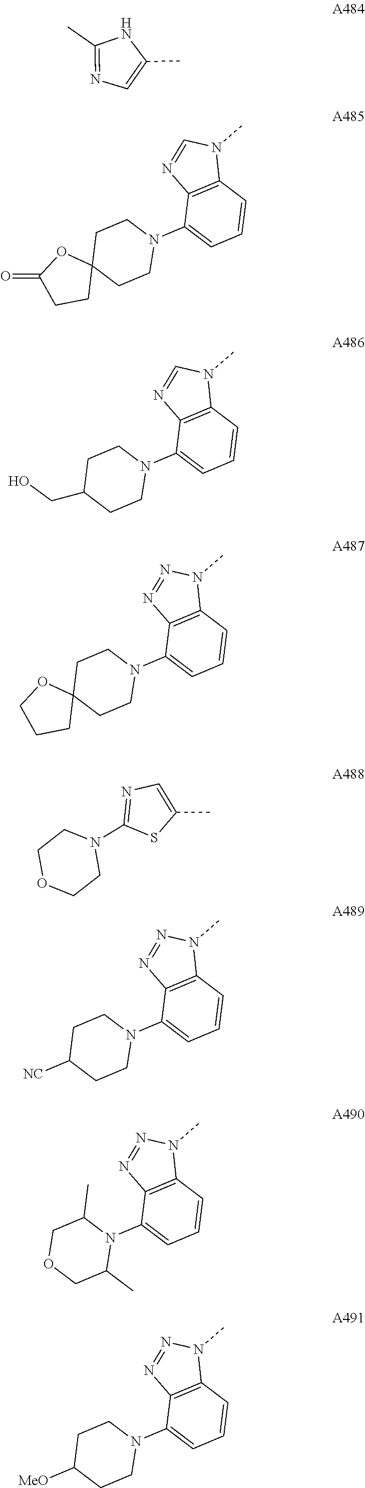

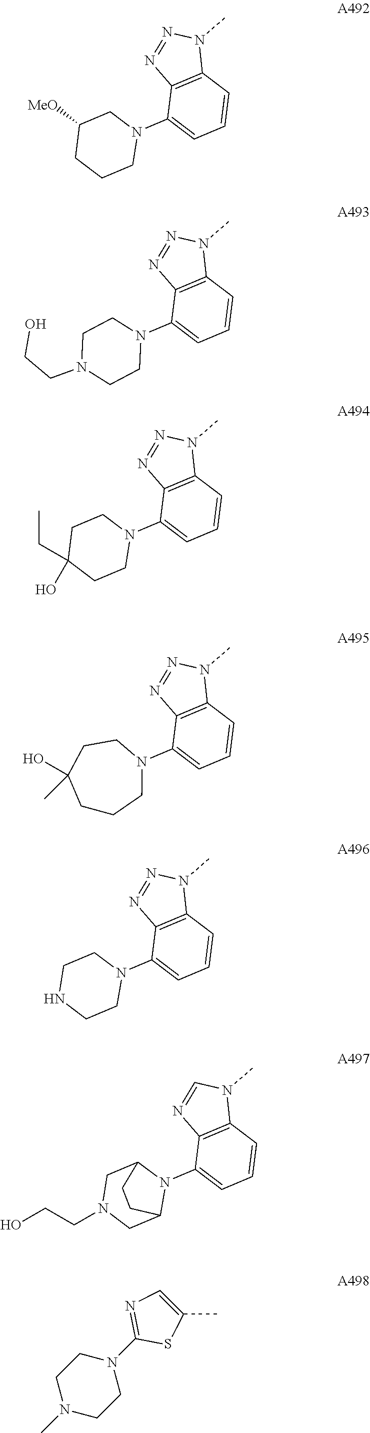

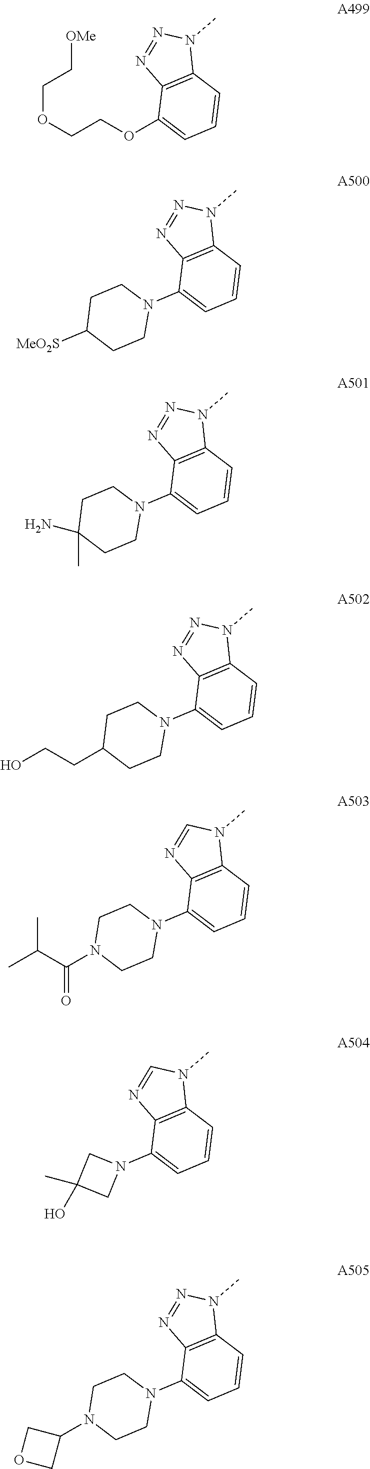

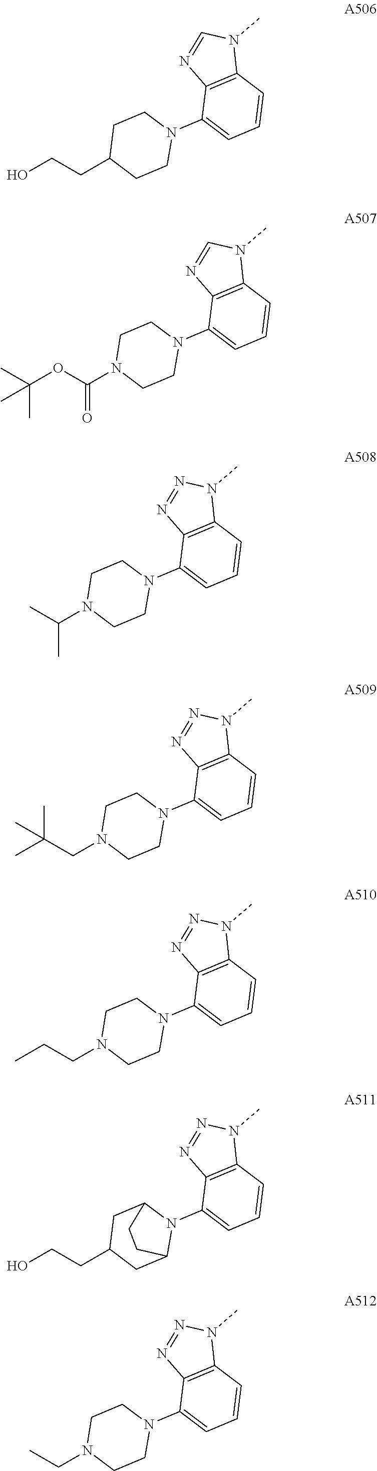

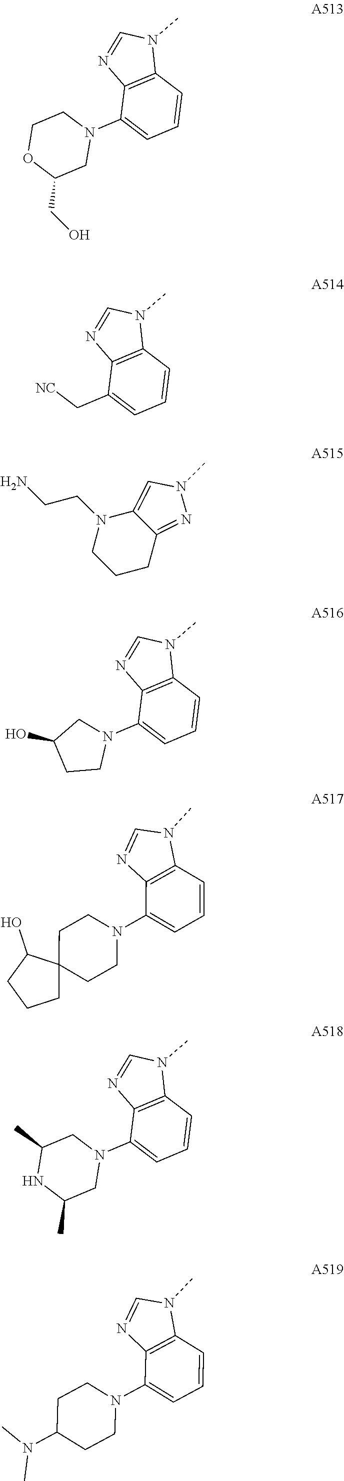

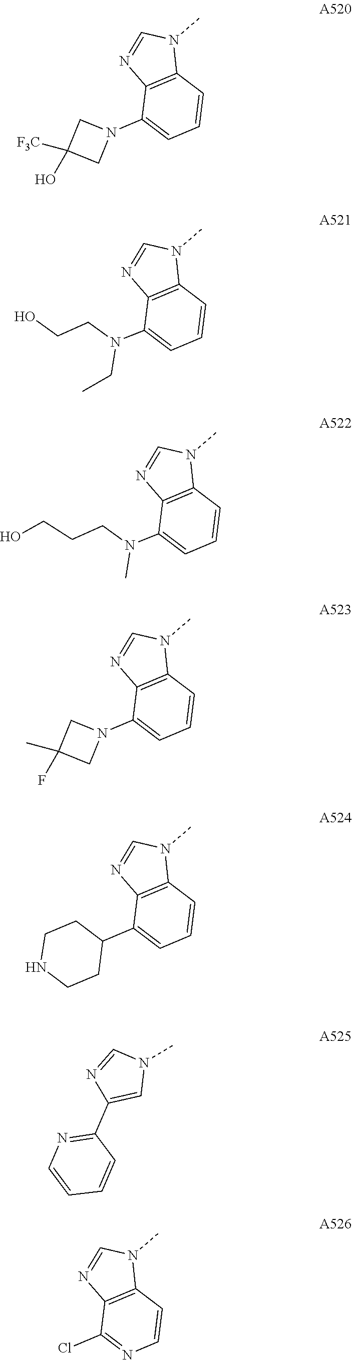

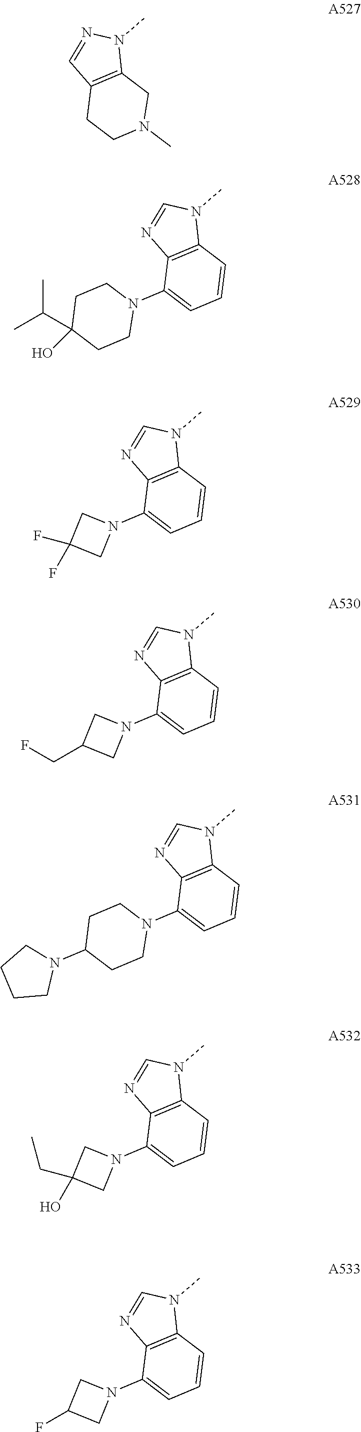

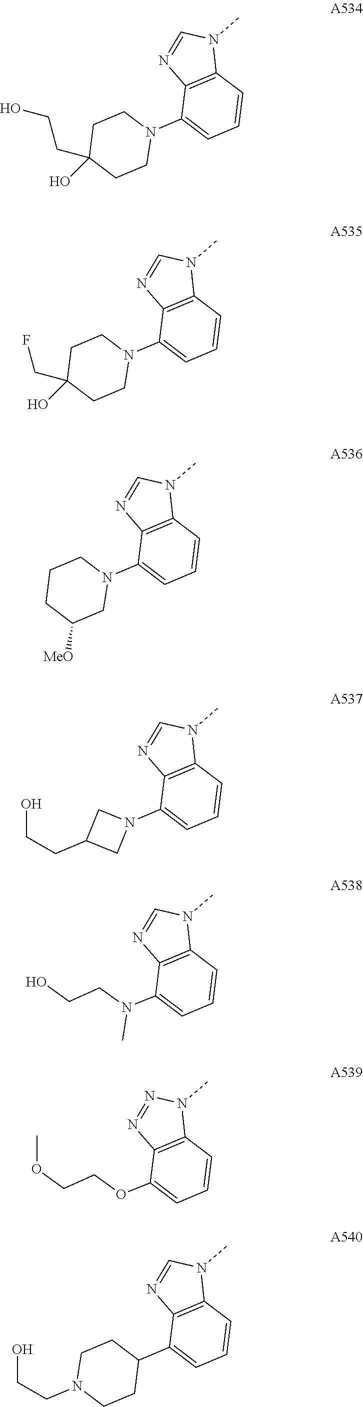

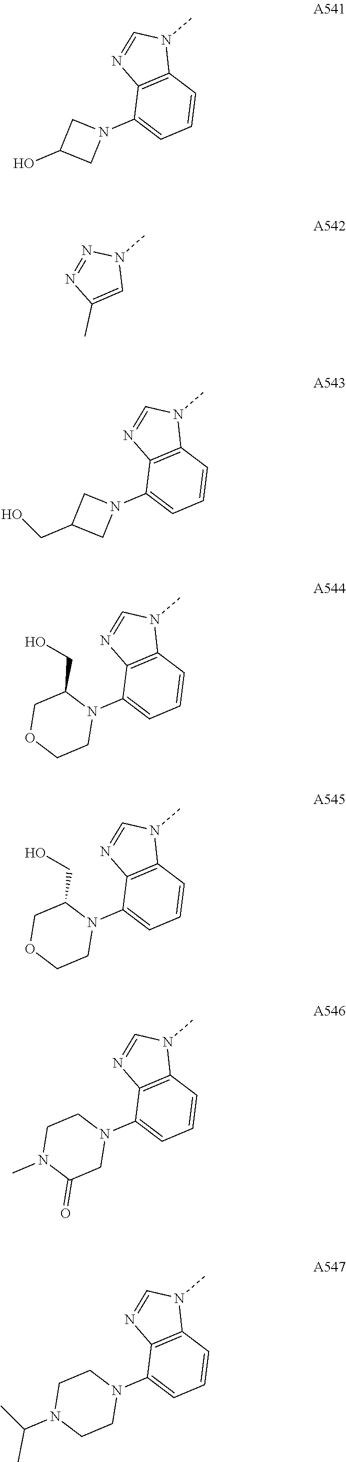

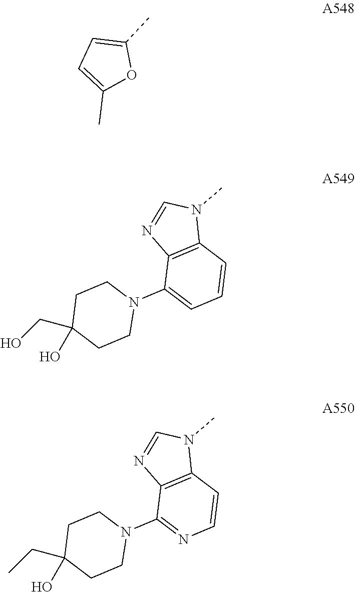

wherein ( - - - ) represents a bond serving as a point of attachment between R¹ and the rest of the molecule.

In one embodiment, R¹ is selected from groups A1 to A550 or R¹ is selected from groups A1 to A3, A8, A19, A20, A22, A23, A25, A28, A29, A32 to A39, A60, A63 to A66, A69, A72 to A78, A81 to A83, A86, A89, A96, A100, A101, A104, A105, A109 to A111, A113, A115, A118, A121 to A123, A127 and A132, for instance, R¹ is selected from groups A1 to A3, A8, A19, A22, A25, A28, A29, A32, A36, A37, A64, A67, A74, A77, A78, A82, A83, A89, A96, A109, A110 and A111.

In one embodiment, R¹ is selected from groups A1 to A3, A8, A19, A20, A22, A23, A25, A28, A29, A32 to A39, A60, A63 to A66, A69, A72 to A78, A81 to A83, A86, A89, A93, A96, A100, A101, A104, A105, A109 to A111, A113, A115, A118, A121 to A123, A127 and A132, for instance, R¹ is selected from groups A1 to A3, A8, A19, A22, A25, A28, A29, A32, A36, A37, A64, A67, A74, A77, A78, A82, A83, A89, A93, A96, A109, A110 and A111.

The following embodiments depict combinations of R¹ (A1 to A550), R² (1 to B77) and L (L1 to L4) groups that can be combined to produce compounds of Formula I.

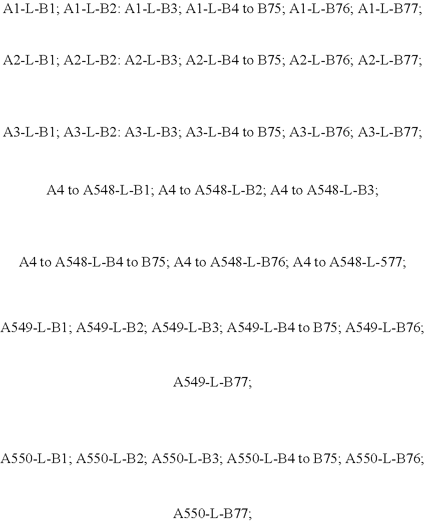

Exemplary compounds as defined herein include each single compound covered in Tables 3 and 4 under Examples 1 to 159.

Examples of preferred compounds are, namely, Examples 2, 4, 6, 7, 14, 16, 18, 30, 31, 33 to 37, 40, 43 to 46, 49, 51 to 60, 81, 84 to 88, 90, 93 to 99, 102 to 105, 108, 111, 112, 116 to 119, 122, 126, 127, 130, 131, 135 to 137, 139, 141, 144, 147, 148, 149, 153, and 158, or a salt and/or solvate thereof.

Examples of more preferred compounds include Examples 4, 6, 7, 14, 16, 18, 30, 33, 35, 36, 37, 40, 43 to 45, 49, 51, 56 to 58, 85, 88, 95, 98, 99, 103, 104, 105, 111, 112, 116, 122, 135, and 136, or a salt and/or solvate thereof.

It is understood that any of the above compounds may be in any amorphous, crystalline or polymorphic form, including any salt or solvate form, or a mixture thereof. The compounds of the present description may be further modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

These compounds may be prepared by conventional chemical synthesis, such as those exemplified in the Schemes and Examples of the present disclosure. As can be appreciated by the skilled artisan, further methods of synthesizing the compounds of the formulae herein will be evident to those of ordinary skill in the art. Additionally, the various synthetic steps may be performed in an alternate sequence or order to give the desired compounds.

### iii. Methods, Uses, Formulations and Administration

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in treatment, healing, prevention, or amelioration of a disease, disorder, or symptom thereof, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In one embodiment, the disease or condition to be treated is a proliferative disease or disorder or a kinase-mediated disease or disorder. More specifically, the disease or disorder to be treated include a proliferative disease or disorder, a developmental anomaly caused by dysregulation of the RAS-ERK signaling cascade (RASopathies), an inflammatory disease or an immune system disorder.

According to some examples, the proliferative disease or disorder to be treated is a neoplasm, an inflammatory disease or condition or a developmental anomaly, involving a constitutively activating mutation in RAS and/or RAF genes (e.g. KRAS and/or ARAF, BRAF or CRAF mutations). The disease or disorder may also be further associated with a receptor tyrosine kinase mutation or amplification (e.g. EGFR, HER2) or a mutation in a regulator of RAS downstream of the receptor (e.g. SOS1 gain of function, NF1 loss of function). For instance, the compounds as defined herein are inhibitors of signal enzymes (ex. B- and CRAF) which are involved in controlling cell proliferation not only in tumors harboring RAF mutations (e.g. BRAF^{V600E}) but importantly also in the context of mutated RAS-driven cancers. Thus, the present compounds may be used for example for the treatment of diseases connected with the activity of these signal enzymes and characterized by excessive or abnormal cell proliferation.

According to one embodiment, the disease or disorder is characterized by uncontrolled cell proliferation, i.e. a “proliferative disorder” or “proliferative disease”. More specifically, these diseases and disorders relate to cells having the capacity for autonomous growth, i.e. an abnormal state of condition characterized by rapidly proliferating cell growth which generally forms a distinct mass that show partial or total lack of structural organization and functional coordination with normal tissue.

For instance, the proliferative disorder or disease is defined as a “neoplasm”, “neoplastic disorder”, “neoplasia” “cancer,” and “tumor” which terms are collectively meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (related to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures, including external epithelia (e.g., skin and linings of the gastrointestinal tract, lungs, and cervix), and internal epithelia that line various glands (e.g., breast, pancreas, thyroid). Examples of neoplasms include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer. For instance, the disease or disorder is selected from colon cancer, lung cancer, pancreatic cancer, thyroid cancer, breast cancer and skin cancer. Examples of neoplasm include melanoma, papillary thyroid carcinoma, colorectal, ovarian, breast cancer, endometrial cancer, liver cancer, sarcoma, stomach cancer, Barret's adenocarcinoma, glioma (including ependymoma), lung cancer (including non-small cell lung cancer), head and neck cancer, acute lymphoblastic leukemia, acute myelogenous leukemia, non-Hodgkin's lymphoma, and hairy-cell leukemia.

In an embodiment, patients presenting one of the above-mentioned hematopoietic or solid neoplasms have previously received treatment with a RAS-ERK pathway-targeted inhibitor (including RTK, RAF, MEK or ERK inhibitor) but have developed resistance to the said inhibitor. The inhibitor includes standard of care treatments such as vemurafenib, dabrafenib, cobimetinib, trametinib, YERVOY, OPDIVO or any combination of these pharmaceutical agents.

In an embodiment, the disease to be treated is defined by developmental anomalies caused by dysregulation of the RAS-ERK signaling cascade (RASopathies: e.g. Noonan syndrome, Costello syndrome, LEOPARD syndrome, cardiofaciocutaneous syndrome and hypertrophic cardiomyopathy).

In an embodiment, the disease to be treated is defined as an inflammatory disease or immune system disorder. Examples of such inflammatory diseases or immune system disorders including inflammatory bowel disease, Crohn's disease, ulcerative colitis, systemic lupus erythematosis (SLE), rheumatoid arthritis, multiple sclerosis, thyroiditis, type 1 diabetes, sarcoidosis, psoriasis, allergic rhinitis, asthma, COPD (chronic obstructive pulmonary disease).

In one embodiment, the compounds as herein defined are inhibitors of RAS-ERK signaling and cellular proliferation in tumor cells bearing at least one mutated RAS or RAF genotype, without or substantially without inducing the paradoxical pathway.

The term “patient or subject” as used herein refers to an animal such as a mammal. A subject may therefore refer to, for example, mice, rats, dogs, cats, horses, cows, pigs, guinea pigs, primates including humans and the like. Preferably the subject is a human.

The present description therefore further relates to a method of treating a subject, such as a human subject, suffering from a proliferative disease or disorder, e.g. a RAF-mutated and/or mutated RAS-driven cancer. The method comprises administering a therapeutically effective amount of a compound as defined herein, to a subject in need of such treatment.

In certain embodiments, the present description provides a method of treating a disorder (as described herein) in a subject, comprising administering to the subject identified as in need thereof, a compound of the present description. The identification of those patients who are in need of treatment for the disorders described above is well within the ability and knowledge of one skilled in the art. Certain of the methods for identification of patients which are at risk of developing the above disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination, medical/family history, and genetic determination.

A method of assessing the efficacy of a treatment in a subject includes determining the pre-treatment symptoms of a disorder by methods well known in the art and then administering a therapeutically effective amount of a compound of the present description, to the subject. After an appropriate period of time following the administration of the compound (e.g., 1 week, 2 weeks, one month, six months), the symptoms of the disorder are determined again. The modulation (e.g., decrease) of symptoms and/or of a biomarker (e.g. pERK or pMEK) of the disorder indicates efficacy of the treatment. The symptoms and/or biomarker of the disorder may be determined periodically throughout treatment. For example, the symptoms and/or biomarker of the disorder may be checked every few days, weeks or months to assess the further efficacy of the treatment. A decrease in symptoms and/or biomarker of the disorder indicates that the treatment is efficacious.

In some embodiments, the therapeutically effective amount of a compound as defined herein can be administered to a patient alone or in a composition, admixed with a pharmaceutically acceptable carrier, adjuvant, or vehicle.

The expression “pharmaceutically acceptable carrier, adjuvant, or vehicle” and equivalent expressions, refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally, by inhalation spray, topically, rectally, nasally, buccally, or via an implanted reservoir. The term “parenteral” as used herein includes subcutaneous, intravenous, intramuscular, intraarticular, intrasynovial, intrasternal, intrathecal, intrahepatic, intralesional and intracranial injection or infusion techniques. Other modes of administration also include intradermal or transdermal administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, surfactants, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled.

Examples of other biodegradable polymers include poly(orthoesters) and poly(anhydrides). Depot injectable formulations are also prepared by entrapping the compound in liposomes or microemulsions that are compatible with body tissues.

Compositions for rectal administration are preferably suppositories which can be prepared by mixing the compounds of the present description with suitable non-irritating excipients or carriers such as cocoa butter, polyethylene glycol or a suppository wax which are solid at ambient temperature but liquid at body temperature and therefore melt in the rectum and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone (PVP), sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The composition can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present description include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the present description. Additionally, the description contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promotors to enhance bioavailability, fluorocarbons, and/or other conventional solubilizing or dispersing agents.

Pharmaceutically acceptable compositions provided herein may be formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compound that may be combined with carrier materials to produce a composition in a single dosage form will vary depending upon the patient to be treated and the particular mode of administration. Provided compositions may be formulated such that a dosage of between 0.01-100 mg/kg body weight/day of the inhibitor can be administered to a patient receiving these compositions.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the symptoms associated with the proliferative disease or disorder. The amount of a provided compound in the composition will also depend upon the particular compound in the composition.

Compounds or compositions described herein may be administered using any amount and any route of administration effective for treating or lessening the severity of the symptoms as contemplated herein. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment.

Pharmaceutically acceptable compositions of this disclosure can be administered to humans and other animals orally, rectally, parenterally, intracisternally, intraperitoneally, topically (as by powders, ointments, or drops), buccally, as an oral or nasal spray, or the like, depending on the severity of the infection being treated. In certain embodiments, provided compounds may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

It will be understood, that the total daily usage of the compounds and compositions of the present description will be decided by the attending physician within the scope of sound medical judgment. The total daily inhibitory dose of the compound of the present description administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In one embodiment, treatment regimens according to the present description comprise administration to a patient in need of such treatment from about 10 mg to about 1000 mg of the compound(s) of the present description per day in single or multiple doses.

Depending upon the disease or disorder to be treated, additional therapeutic agents may also be present in the compositions of this disclosure or administered separately as part of a dosage regimen, e.g. an additional chemotherapeutic agent. Non-limiting examples of additional therapeutic agents which could be used in combination with the present compounds include antiproliferative compounds such as aromatase inhibitors; anti-estrogens; anti-androgens; gonadorelin agonists; topoisomerase I inhibitors; topoisomerase II inhibitors; microtubule active agents; alkylating agents; retinoids, carotenoids, tocopherol; cyclooxygenase inhibitors; MMP inhibitors; antimetabolites; platin compounds; methionine aminopeptidase inhibitors; bisphosphonates; antiproliferative antibodies; heparanase inhibitors; inhibitor of Ras oncogenic isoforms; telomerase inhibitors; proteasome inhibitors; compounds used in the treatment of hematologic malignancies; kinesin spindle protein inhibitors; Hsp90 inhibitors; mTOR inhibitors; PI3K inhibitors; Flt-3 inhibitors; CDK4/6 inhibitors; HER2 inhbiitors (Herceptin, Trastuzumab); EGFR inhibitors (Iressa, Tarceva, Nerlynx, Tykerb, Erbitux); RAS inhibitors; MEK inhibitors (Trametinib, Binimetinib, Cobimetinib); ERK inhibitors (Ulixertinib); anti-PD-1 antibodies (Opdivo, Keytruda); anti-CTLA4 antibodies (Yervoy); antitumor antibiotics; nitrosoureas; compounds targeting/decreasing protein or lipid kinase activity, compounds targeting/decreasing protein or lipid phosphatase activity, or any further anti-angiogenic compounds.

The treatment may also be complemented with other treatments or interventions such as surgery, radiotherapy (e.g., gamma-radiation, neutron beam radiotherapy, electron beam radiotherapy, proton therapy, brachytherapy, and systemic radioactive isotopes), a biologic response modifier (e.g., an interferon, an interleukin, tumor necrosis factor (TNF), and agents used to attenuate an adverse effect.

The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

### List of Abbreviations

Ac: acetyl
AcOEt or EtOAc: ethyl acetate
AcOH: acetic acid
Ar: aryl
ATCC: American Type CpLture Collection
ATP: adenosine triphosphate
BINOL: [1,1′-binaphthalene]-2,2′-diol
Boc: tert-butyloxycarbonyl
BOP: (benzotriazol-1-yloxy)tris(dimethylamino)phosphonium hexafluorophosphate
br: broad
BSA: bovine serum albumin
CCL: cancer cell lines
CDCl₃: deuterated chloroform
DCE: 1,2-dichloroethane
DCM: dichloromethane
DIEA (or DIPEA): N,N-diisopropylethylamine (Huenig's base)
DME: 1,2-dimethoxyethane
DMF: N,N-dimethylformamide
DMSO: dimethylsulfoxide
DMSO-d₆: deuterated dimethylsulfoxide
DTT: dithiothreitol
EA: ethyl acetate
EC₅₀: half-maximal effective concentration
ECL: enhanced chemiluminescence
EDTA: ethylenediamine tetraacetic acid
Et₂O: diethyl ether
EtOH: ethanol
Eu: Europium
FBS: fetal bovine serum
GST: glutathion S-transferase
HATU: O-(7-azabenzotriazol-1-yl)-N,N,N′,N′-tetramethyluronium hexafluorophosphate
HEPES: 4-(2-hydroxyethyl)-1-piperazineethanesulfonic acid
Het: heterocycle
Hex: hexanes
HRMS: high resolution mass spectrometry
HPLC: high performance liquid chromatography
HRP: horseradish peroxidase
IC₅₀: half-maximal inhibitory concentration
IPA: isopropanol
iPrOH: isopropanol
LCMS: liquid chromatography mass spectrometry
MeCN: acetonitrile
MS: mass spectrometry
NMP: N-methylpyrrolidone
NMR: nuclear magnetic resonance
ON: overnight
PBS: phosphate buffered saline
pERK: phosphorylated extracellular signal-regulated kinase
PMB: para-methoxybenzyl
PMSF: phenylmethylsulfonyl-fluoride
Rf: retention factor
RPMI-1640: Roswell Park Memorial Institute medium
RT or rt: room temperature
SDS: sodium dodecylsulfate
SDS-PAGE: sodium dodecyl-sulfate-polyacrylamide gel electrophoresis
SEM: trimethylsilylethoxymethyl
SNAr: Nucleophilic aromatic substitution
TBST: Tris buffered saline with 0.2% Tween-20
TBTU: O-(benzotriazol-1-yl)-N,N,N′N′-tetramethyluronium tetrafluoroborate
TEV: tobacco etch virus protease
TFA: trifluoroacetic acid
THF: tetrahydrofuran
TLC: silica gel thin layer chromatography
Ts: para-Toluenesulfonate
Y_{MIN}: minimal data point of a dosage-activity curve

The following non-limiting examples are illustrative embodiments and should not be construed as further limiting the scope of the present invention. These examples will be better understood with reference to the accompanying figures.

The Examples set forth herein below provide syntheses and experimental results obtained for certain exemplary compounds. As it is well known to a person skilled in the art, reactions are performed in an inert atmosphere (nitrogen or argon) where necessary to protect reaction components from air and moisture. Temperatures are given in degrees Celsius (° C.). Solution percentages and ratios express a volume to volume relationship, unless otherwise stated. The reactants used in the examples below may be obtained either as described herein, or if not described herein, are themselves either commercially available or may be prepared from commercially available materials by methods known in the art. Flash chromatography is carried out on silica (SiO₂) using a Teledyne Isco Rf Combiflash instrument at 254 nm using commercial normal phase silica. Mass spectra analyses are recorded using electrospray mass spectrometry. NMR are recorded on a 400 MHz Varian instrument or a 600 MHz Bruker instrument.

Preparative HPLC was performed using an Agilent instrument using a Phenomenex-Kinetex C18, (21×100 mm, 5 μm) column at a flow rate of 20 mL/min (RT) and UV detection at 220 and 254 nm. The mobile phase consisted of Solvent A (5% MeOH, 95% water+0.1% formic acid) and Solvent B (95% MeOH, 5% water+0.1% formic acid) unless stated otherwise. As specified in the text, 0.05% TFA or 0.1% AcOH or other additives were occasionally used as additives instead of 0.1% formic acid in both solvents. MeCN was also used instead of MeOH in both mobile phases for more challenging separations as specified in the text. Specific gradient conditions are provided in the examples but the following is representative: T(0)→T(3 min) isocratic using between 10 to 50% solvent B depending on compound polarity, followed by a 12 minutes gradient to 100% solvent B. Last 5 minutes 100% solvent B.

LCMS analyses were performed on an Agilent instrument. Liquid chromatography was performed on a Phenomenex Kinetex C18 column (2.6 μm; 100 Å; 3×30 mm) at a flow rate of 1.5 mL/min (RT) with UV detection at 220 and 254 nm. The mobile phase consisted of solvent A (95% H₂O/5% MeOH/0.1% AcOH) and solvent B (95% MeOH/5% H₂O/0.1% AcOH) using the following gradient: T(0) 100% A→T(0.5 min) 100% B→isocratic 100% B to T(2 min). MS detection was performed in parallel using APCI detection in both positive and negative modes.

Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Synthesis, Biological Activity and Characterization of Examples

All compounds as herein defined were prepared according to methods as indicated in Tables 3 and 4. Characterization data by mass spectrometry and NMR are provided for each of the Examples. The compounds are tested in the assays described in the biological Experimental section. The convention used for reporting biological data is provided as a footnote in the respective Tables.

### Synthetic Method A

Commercially available 2,6-difluoroaniline A-1 can be converted to its acetanilide A-2 using an acetylating agent such as acetic anhydride and converted to mono-protected dianiline A-3 as described in WO 2012/101238A1. Sulfonylation to sulfonamide A-4 can be achieved using sulfonylating reagents such as sulfonyl chlorides in the presence of an organic base such as pyridine, with or without a catalyst such as 4-dimethylaminopyridine and solvents such as dichloromethane or tetrahydrofuran. Treatment of acetanilide A-4 with aqueous hydrochloric acid in the presence of a co-solvent such as an alcohol provides aniline salt A-5.

Commercially available 2-amino-2-cyanoacetamide A-6 can be transformed into 5-amino-2-(methylthio)thiazole-4-carboxamide A-8 following a 2 step procedure described in Heterocycl. Commun., 2014, 20, 175. Intermediate A-8 can then be cyclized to the corresponding pyrimidone by heating in formamide as described in Indian J. Chem., 2010, 49B, 1229. Subsequent treatment with chlorinating agents such as thionyl chloride or phosphoryl chloride in the presence of a catalytic amount of DMF provides chloro compound A-10 following a procedure which is also described in Indian J. Chem., 2010, 49B, 1229.

Intermediates A-11 can be obtained by heating chloropyrimidine A-10 with aniline salts A-5 in an organic acid such as acetic acid. Inhibitors of general formulae W-I are prepared from intermediates of general formulae A-11 by a two-step procedure involving first oxidation of the thiomethyl group generally to a mixture of the corresponding methylsulfoxide and methylsulfone which is then reacted with a nucleophile (e.g. a 1° or a 2° amine or a NH-containing heterocycle, etc.). The latter step is usually carried out in the presence of a base (e.g. an organic base such as DIEA, trimethylamine, pyridine and the like or an inorganic base such as potassium carbonate, cesium carbonate and sodium carbonate) in a solvent such as DMSO or NMP at a temperature ranging from 70 to 140° C.

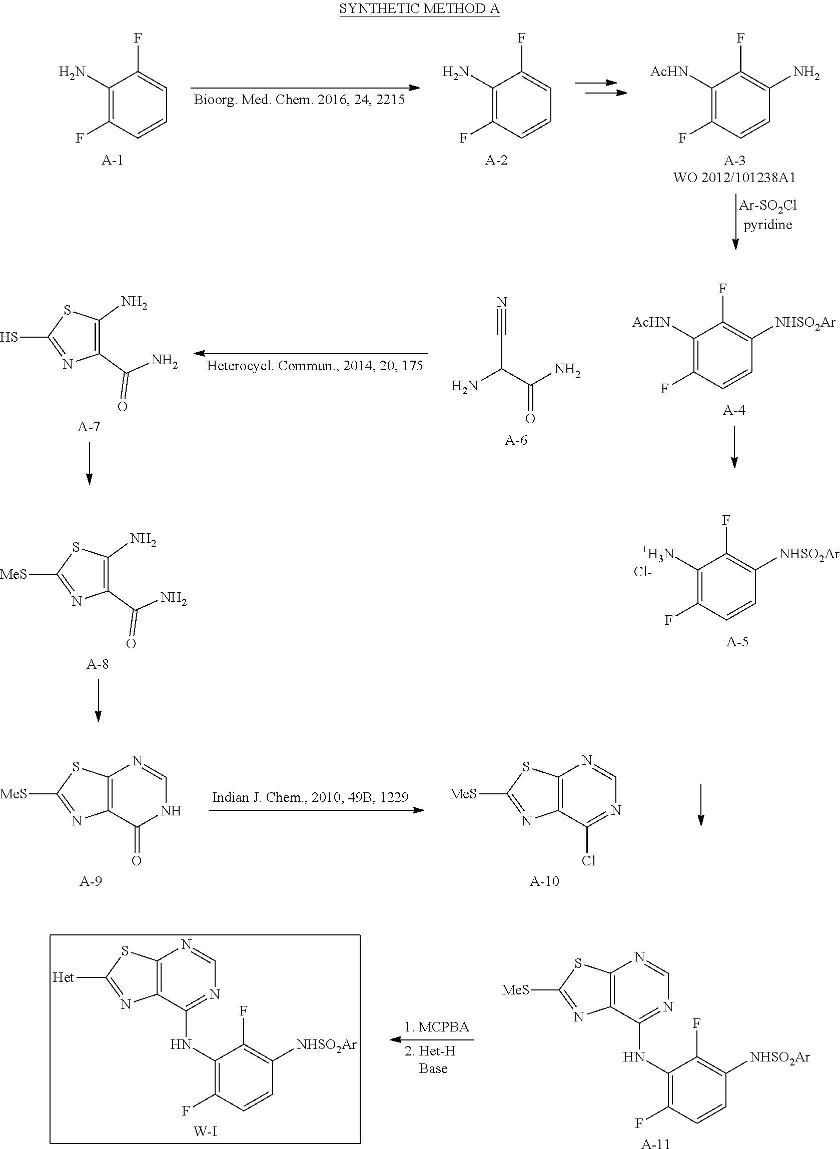

### Synthetic Method B

Inhibitors of general formulae W-II are prepared as described in Synthetic Method B. Intermediates of general formulae A-11 were first prepared following Synthetic Method A and then oxidized to a mixture of methylsulfoxide and methylsulfone, as described in Synthetic Method A. They were then coupled to a 3-indolecarboxylic acid ester (e.g. methyl ester, X═CH) or a 3-indazolecarboxylic acid ester (e.g. methyl ester, X═N) following similar protocols to those described for the introduction of a nucleophile in Synthetic Method A. The latter step is usually carried out in the presence of a base (e.g. an organic or inorganic base such as Cs₂CO₃, KOtBu, DIEA, trimethylamine, pyridine and the like) in a solvent such as THF, DMSO or NMP at temperatures ranging from 60° C. to 140° C. Deprotection of the ester protecting group using an inorganic base (e.g. NaOH or KOH) in mixtures of water and a miscible organic solvent (e.g. methanol, ethanol, THF, dioxane and the likes) at temperatures ranging from ambient to 100° C. followed by acidification with a mineral (e.g. aqueous hydrochloric or sulfuric acid), inorganic salt solution (e.g. aqueous NH₄Cl or KHSO₄) or organic acid (e.g. aqueous citric or acetic acid) provided the corresponding carboxylic acid intermediates B-1 or B-2. Coupling of intermediates B-1 or B-2 with amines using standard amide coupling reagents (e.g. TBTU, HATU, DCC, EDC and the likes) provided amide derivatives of general formulae W-II.

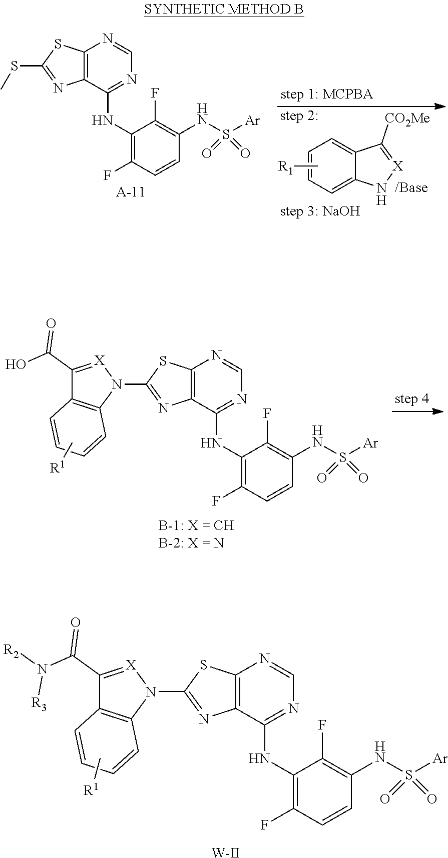

### Synthetic Method C

Cross-coupling of commercially available bromobenzimidazole C-1 to heteroaryl boronic acids or boronate esters can be performed under palladium-catalyzed Suzuki-Miyaura cross-coupling conditions in the presence of a base such as sodium or potassium carbonate in a solvent such as dioxane or dimethoxyethane and water, to provide intermediates C-2. Substituted benzimidazole derivatives C-2 can then be attached to intermediates A-11 following the oxidation of the methylsulfide moiety as described in the general protocol of Method A, to provide inhibitors of general structure W-III.

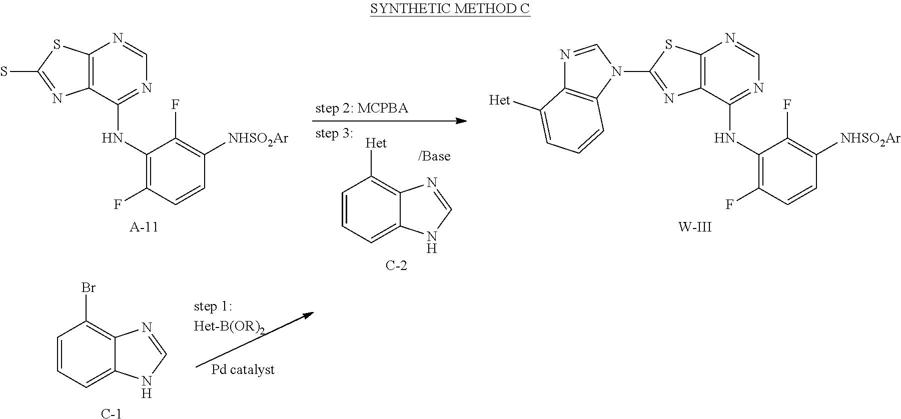

### Synthetic Method D

Unprotected 3-indolesulfonyl chloride D-2 can be prepared as described in Org. Lett. 2011, 13, 3588. This reagent can then be converted to the corresponding sulfonamide D-3 by reacting with a 1° or a 2° amine in the presence of an organic base such as DIEA or triethylamine. D-3 fragments can then be attached to intermediates A-11 following the oxidation of the methylsulfide moiety as described in the general protocol of Method A, to provide inhibitors of general structure W-IV.

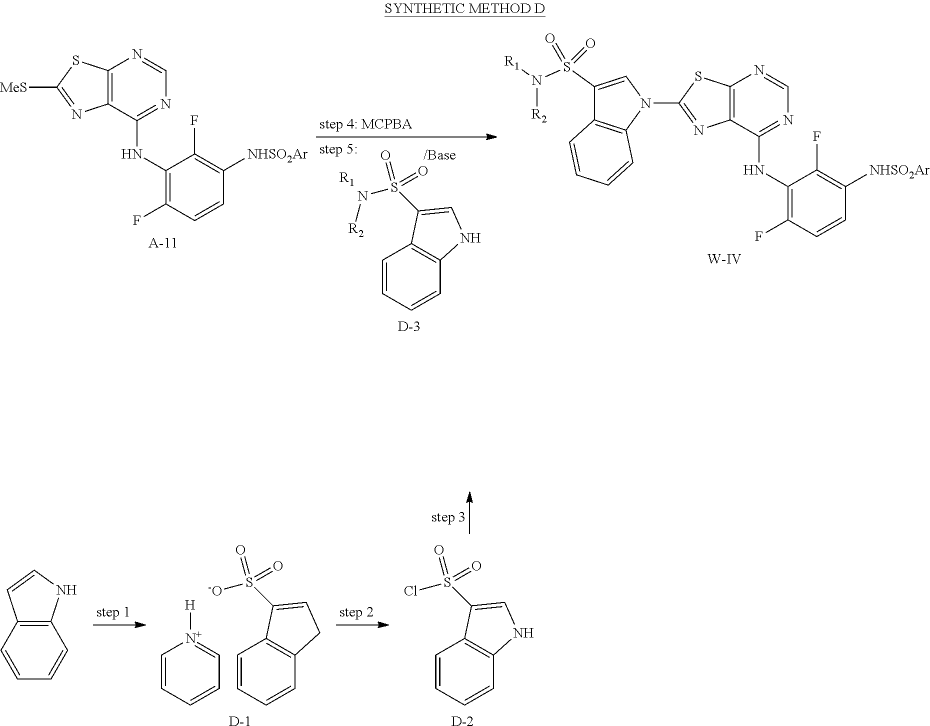

### Synthetic Method E

Alternatively, inhibitors of general structure W-IV can be accessed through N-tosyl protected indole-3-sulfonyl chloride E-1, prepared following the procedure described in Chemical and Pharmaceutical Bulletin 2009, 57, 591. E-1 can then be converted to the corresponding sulfonamide E-2 by reacting with a 1° or a 2° amine in the presence of an organic base such as DIEA or triethylamine followed by removal of the tosyl protecting group with an aqueous inorganic base such as KOH. E-2 fragments can then be attached to intermediates A-11 following the oxidation of the methylsulfide moiety as described previously.

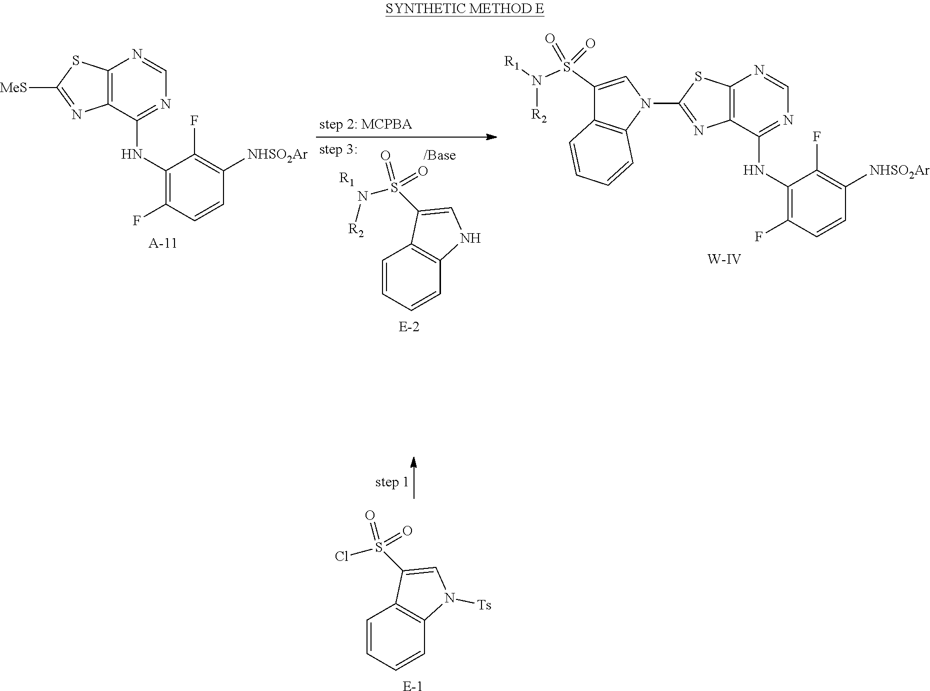

### Synthetic Method F

3-Indolethiocyanate F-1 (prepared following the procedure described in Phosphorus, Sulfur and Silicon and the Related Elements 2014, 189, 1378) is reduced to the corresponding sulfide salt using a reducing agent such as sodium sulfide nonahydrate and directly alkylated without isolation with an alkyl halide to provide sulfide intermediates F-2. Sulfide intermediates F-2 are then converted to sulfone intermediates F-3 using an oxidizing agent such as 3-chloroperoxybenzoic acid. Final inhibitors of the general structure W-V are then obtained by attaching indolesulfones F-3 with A-11 intermediates following the oxidation of the methylsulfide moiety as described previously.

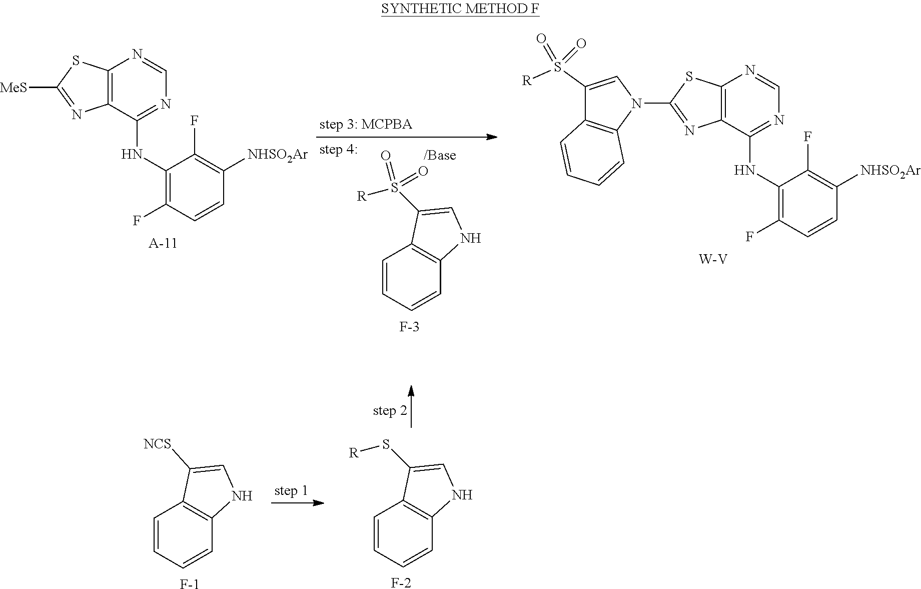

### Synthetic Method G

A bright red solution of commercially available 3-fluoro-2-nitroaniline G-1 reacts with primary or secondary amines in solvents such as MeCN, DMSO, NMP or DMF and in the presence of an inorganic base such as potassium carbonate or an organic base such as DIEA to provide intermediates G-2 upon heating at temperature ranging from 40° C. to 120° C. under thermal or microwave conditions. The reduction of the nitro group of intermediate G-2 and subsequent cyclization of the resulting 1,2-phenylenediamine intermediates to the desired benzimidazole intermediates G-3 can be performed in a single operation employing iron metal, ammonium chloride and formic acid in an alcoholic solvent such as isopropanol upon heating at temperatures ranging from 40° C. to 90° C. Final inhibitors of the general structure W-VI are then obtained by heating substituted benzimidazoles G-3 in the presence of a base, with intermediates A-11 having been oxidized to the corresponding methylsulfone/methylsulfoxide, under usual conditions as described previously.

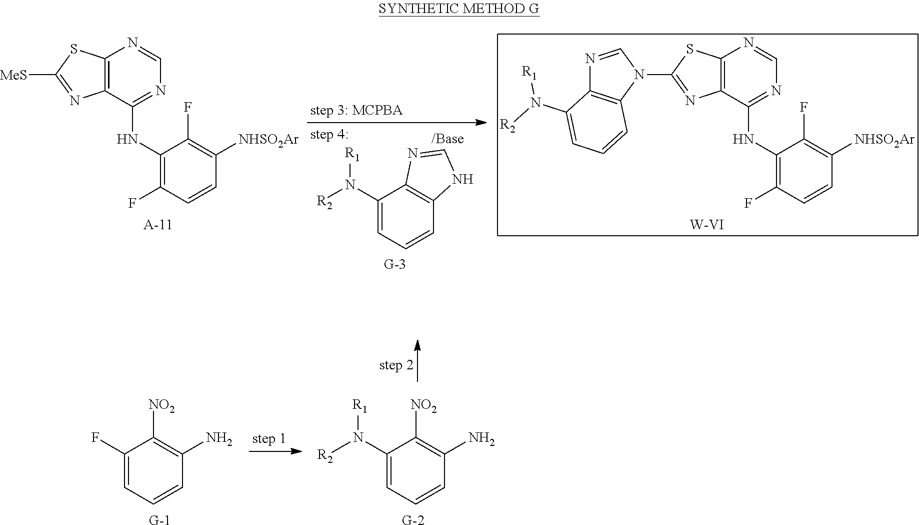

The procedures to prepare inhibitors that do not or only partially fall under the above general synthetic procedures are described specifically in the following.

### Sulfonyl Chlorides:

The following sulfonyl chlorides were obtained from commercial sources and used as received: 2-chlorobenzenesulfonyl chloride, 2,3-dichlorobenzenesulfonyl chloride, 3-chloro-2-methylbenzenesulfonyl chloride, 3-fluoro-2-methylbenzenesulfonyl chloride.

Other sulfonyl chlorides were prepared by using or adapting literature procedures as described below.

### 3-Fluoro-2-methyl-4-methoxybenzenesulfonyl chloride

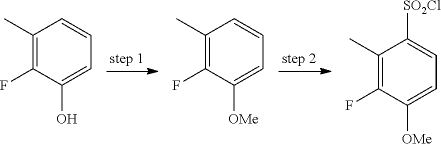

Step 1: To a solution of 2-fluoro-3-methylphenol (4.32 mL, 39.7 mmol) in acetone (50 mL) was added potassium carbonate (6.58 g, 47.6 mmol) followed by iodomethane (2.75 mL, 43.7 mmol). The reaction mixture was then refluxed overnight at 60° C. The reaction mixture was then cooled to RT, filtered (2×10 mL acetone for rinses) and concentrated under reduced pressure. The crude product was extracted from water (30 mL) and EtOAc (2×50 mL). The organic layer was then separated, dried with Na₂SO₄, filtered and concentrated under reduced pressure. The residue was purified by flash chromatography on silica gel using 0 to 5% EtOAc/hexanes to afford 2-fluoro-3-methylanisole as a clear colorless liquid (5.30 g, 95% yield): ¹H NMR (CDCl₃) δ: 6.95 (td, J=8.0, 1.4 Hz, 1H), 6.85-6.71 (m, 2H), 3.87 (s, 3H), 2.28 (d, J=2.3 Hz, 3H).

Step 2: To a solution of 2-fluoro-3-methylanisole from step 1 (1.00 g, 7.13 mmol) in DCM (5.6 mL) was added a solution of chlorosulfonic acid (1.13 mL, 16.5 mmol) in DCM (5.6 mL) over a 5 minute period. The pale brown reaction mixture containing a viscous liquid layer was stirred at RT for 10 min and then quenched by pouring into a mixture of water (10 mL) and ice (5 g). The aqueous phase was extracted with DCM (2×10 mL), dried on Na₂SO₄, filtered and concentrated under reduced pressure to give the desired sulfonyl chloride as a colorless liquid (1.70 g, 100% yield). The material was used without further purification: ¹H NMR (CDCl₃) δ: 7.87 (dd, J=9.0, 1.8 Hz, 1H), 6.97-6.86 (m, 1H), 3.97 (s, 3H), 2.66 (d, J=2.8 Hz, 3H).

### 3-Chloro-2-methyl-4-methoxybenzenesulfonyl chloride

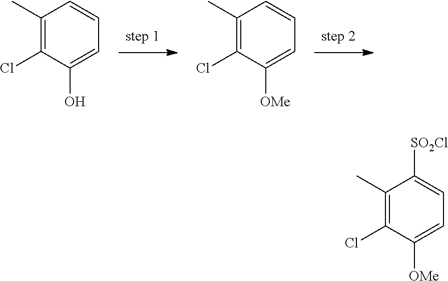

Following a similar procedure (Step 1) as for 3-fluoro-2-methyl-4-methoxybenzenesulfonyl chloride and starting from 2-chloro-3-methylphenol, 2-chloro-3-methylanisole was obtained in quantitative yield as a colorless liquid: ¹H NMR (CDCl₃) δ: 7.12 (t, J=7.9 Hz, 1H), 6.87-6.83 (m, 1H), 6.79 (d, J=8.2 Hz, 1H), 3.89 (s, 3H), 2.38 (s, 3H).

Treatment with chlorosulfonic acid as described for 3-fluoro-2-methyl-4-methoxybenzenesulfonyl chloride (Step 2) provided the desired 3-chloro-2-methyl-4-methoxybenzenesulfonyl chloride as a colorless liquid in 96% yield: ¹H NMR (CDCl₃) δ: 8.02 (d, J=9.1 Hz, 1H), 6.90 (d, J=9.1 Hz, 1H), 4.00 (s, 3H), 2.83 (s, 3H).

### General Synthetic Method A: Preparation of difluoroaniline hydrochloride intermediate A-5 (Ar=2,3-dichlorophenyl) from acetanilide A-3

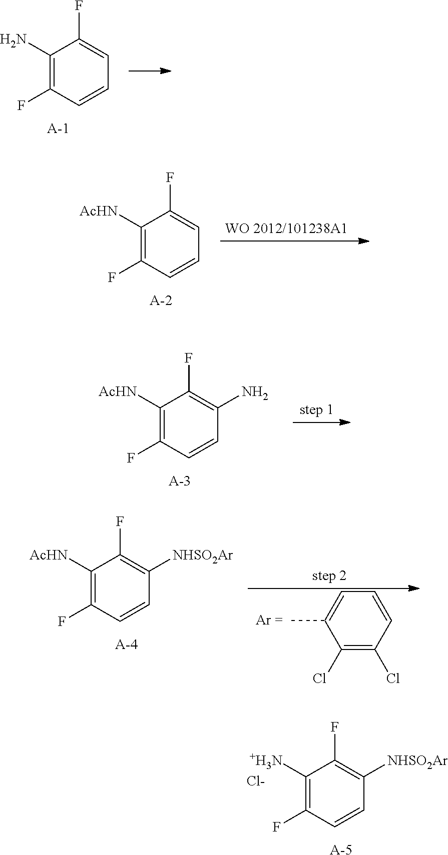

Preparation of acetanilide A-2: Acetanilide A-2 can be prepared by acetylation of 2,6-difluoroaniline A-1 with acetic anhydride following the literature procedure described in Bioorg. Med. Chem. 2016, 24, 2215. Intermediate A-2 was converted to acetanilide A-3 by sequential nitration followed by reduction of the nitro group to the aniline as described in WO 2012/101238A1.

Step 1: Preparation of sulfonamide A-4 (Ar=2,3-dichlorophenyl): Aniline A-3 (8.50 g, 45.5 mmol) was dissolved in THF (145 mL) and pyridine (4 eq., 14.7 mL) was added to the brown solution followed by 2,3-dichlorobenzenesulfonyl chloride (1.2 eq., 13.45 g). The resulting reaction mixture was stirred at 45° C. for 3.5 hours after which conversion was judged to be complete as monitored by LCMS. The reaction mixture was allowed to cool to room temperature then partitioned between EtOAc and 2-Me-THF (1:1) and water. A 1N HCl solution was added until a slightly acid pH was obtained. A significant amount of off-white solids were present in the biphasic mixture and were filtered out (first crop). The layers of the filtrate were separated, and the aqueous layer was extracted two more times with EtOAc. The combined organic extracts were washed once with water then with brine, dried over MgSO₄, filtered and concentrated down to −20 mL. The resulting suspension was sonicated, and the solids were collected by filtration and washed with EtOH (crop 2). Both crops were combined and dried under reduced pressure. A-4 (15.3 g, 85% yield) was obtained as a beige solid that was used without further purification: ¹H NMR (DMSO-d₆) δ: 10.61 (s, 1H), 9.67 (s, 1H), 7.95 (dd, J=8.0, 1.4 Hz, 1H), 7.85 (dd, J=8.0, 1.4 Hz, 1H), 7.51 (t, J=8.0 Hz, 1H), 7.05-7.18 (m, 2H), 2.00 (s, 3H). MS m/z 395.0 (MH⁺).

Step 2: Preparation of aniline hydrochloride A-5 (Ar=2,3-dichlorophenyl): In a 500 mL round-bottomed flask, acetanilide A-4 (7.00 g, 17.7 mmol) was suspended in ethanol (65 mL) and a 1:1 mixture of concentrated HCl and water (65 mL) was added. The flask was equipped with a stoppered reflux condenser and heated at 80° C. with stirring. After 24 hours, conversion was ˜70% as judged by LCMS monitoring. Additional EtOH (65 mL) and 6N HCl (65 mL) were added to the suspension and stirring at 80° C. resumed for 7 more hours after which LCMS indicated complete conversion to the desired aniline. The reaction mixture was diluted with 50 mL of water while still warm and filtered through a plug of cotton to remove a small amount of insoluble materials. It was then concentrated to dryness under reduced pressure. The residue was azeotropically dried by evaporation of toluene 3× under reduced pressure then dried under vacuum, affording 7.2 g of the desired product A-5 as its HCl salt in the form of a yellow solid: ¹H NMR (DMSO-d₆) δ: 10.30 (s, 1H), 7.93 (dd, J=8.2, 1.2 Hz, 1H), 7.83 (dd, J=8.0, 1.4 Hz, 1H), 7.49 (t, J=8.0 Hz, 1H), 6.68-6.96 (m, 1H), 6.31 (td, J=8.6, 5.5 Hz, 1H). MS m/z 350.9 (M-H).

The following A-5 intermediates (Table 1) were prepared in a similar fashion employing a relevant sulfonyl chloride:

TABLE 1

| | MS | |
|---|---|---|
| | m/z | |
| A-5 | (M − H) | ¹H NMR (400 MHz) |
| 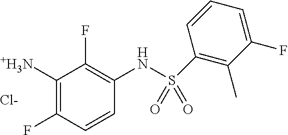 [SMILES: [Cl-] ; Cc1c(F)cccc1S(=O)(=O)Nc1ccc(F)c([NH3+])c1F] | 315.0 | ¹H NMR (DMSO-d₆) δ: 10.10 (s, 1H), 7.54 (d, J = 7.8 Hz, 1H), 7.46 (td, J = 9.0, 1.2 Hz, 1H), 7.35 (td, J = 8.2, 5.5 Hz, 1H), 6.79 (ddd, J = 10.9, 9.1, 2.0 Hz, 1H), 6.32 (td, J = 8.6, 5.5 Hz, 1H), 2.49 (d, J = 2.0 Hz, 3H) |
| 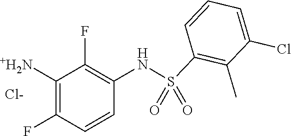 [SMILES: [Cl-] ; Cc1c(Cl)cccc1S(=O)(=O)Nc1ccc(F)c([NH3+])c1F] | 331.0 | ¹H NMR (CDCl₃) δ: 7.84 (d, J = 7.8 Hz, 1H), 7.58 (d, J = 7.8 Hz, 1H), 7.21 (t, J = 8.0 Hz, 1H), 6.68-6.74 (m, 2H), 6.63 (br. s., 1H), 2.75 (s, 3H) |
| 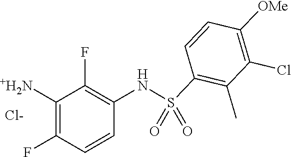 [SMILES: [Cl-] ; COc1ccc(S(=O)(=O)Nc2ccc(F)c([NH3+])c2F)c(C)c1Cl] | 363.2 | ¹H NMR (DMSO-d₆) δ: 10.00 (s, 1H), 7.67 (d, J = 8.9 Hz, 1H), 7.07 (d, J = 9.0 Hz, 1H), 6.78 (ddd, J = 9.0, 6.5, 1.9 Hz, 1H), 6.33 (td (J = 8.7, 5.5 Hz, 1H), 3.89 (s, 3H), 2.65 (s, 3H) |
| 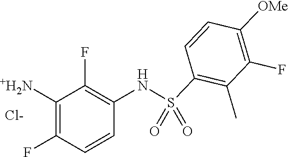 [SMILES: [Cl-] ; COc1ccc(S(=O)(=O)Nc2ccc(F)c([NH3+])c2F)c(C)c1F] | 347.1 | ¹H NMR (DMSO-d₆) δ: 9.94 (s, 1H), 7.48 (dd, J = 8.9, 1.5 Hz, 1H), 7.07 (t, J = 8.6 Hz, 1H), 6.78 (ddd, J = 10.8, 9.0, 1.9 Hz, 1H), 6.33 (td, J = 8.6, 5,5 Hz, 1H), 3.86 (s, 3H), 2.48 (d, J = 2.7 Hz, 3H) |
| 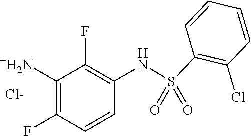 [SMILES: [Cl-] ; [NH3+]c1c(F)ccc(NS(=O)(=O)c2ccccc2Cl)c1F] | 317.0 | ¹H NMR (DMSO-d₆) δ: 10.12 (s, 1H), 7.85 (dd, J = 7.8, 1.6 Hz, 1H), 7.59-7.71 (m, 2H), 7.46 (td, J = 7.5, 1.4 Hz, 1H), 6.74-6.82 (m, 1H), 6.31 (td, J = 8.6, 5.5 Hz, 1H) |

### General Synthetic Method A—Preparation of Inhibitors W-I from Intermediates A-5

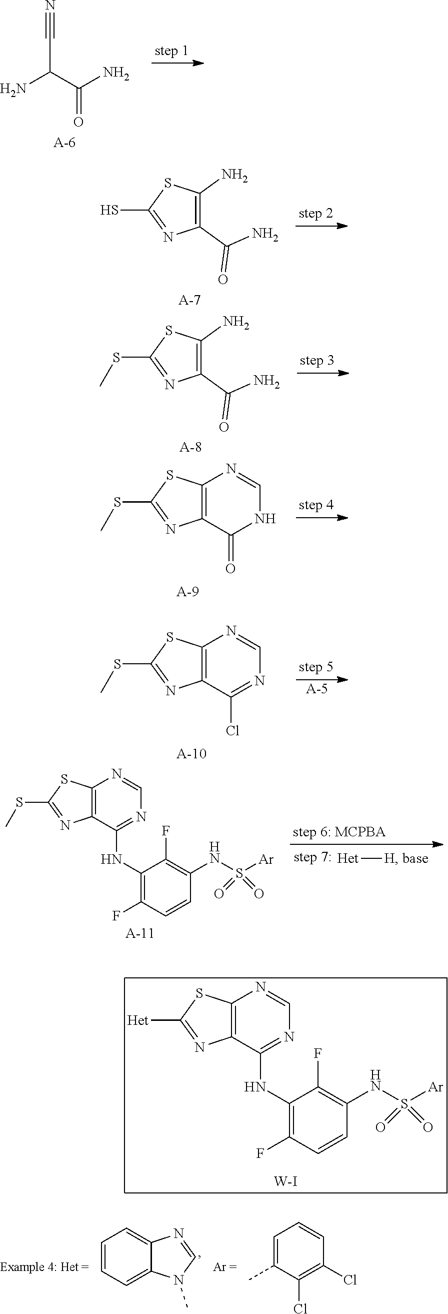

Steps 1 and 2: Preparation of intermediate A-8: These two steps were performed as described by Wang et al. in Heterocycl. Commun., 2014, 20,175.

Step 3: Preparation of intermediate A-9 (adapted from Indian J. Chem., 2010, 49B, 1229): 5-amino-2-methylsulfanyl-thiazole-4-carboxamide (3.19 g, 16.855 mmol) was divided equally into two microwaveable 20 mL vials to which was added formamide (13.4 mL, 337 mmol) in each. The vials were sealed and heated in the microwave at 185° C. each for 20 minutes then at 190° C. for another 15 minutes. The mixtures were allowed to cool to room temperature then combined and added slowly to ice cold water (60 mL) containing 1.3 mL of acetic acid. The resulting yellow solids were collected by filtration and washed with some cold water. After suction drying on the filter, the solids were transferred into a vial and dried under reduced pressure. Affords 2-methylsulfanyl-6,7a-dihydro-3aH-thiazolo[5,4-d]pyrimidin-7-one (2.34 g, 69% yield) as a yellow to beige solid. MS m/z 202.2 (MH⁺). ¹H NMR (DMSO-d₆) δ: 12.81 (br. s., 1H), 8.15 (s, 1H), 2.75 (s, 3H).

Step 4: Preparation of intermediate A-10 (adapted from Indian J. Chem., 2010, 49B, 1229): 2-methylsulfanyl-6H-thiazolo[5,4-d]pyrimidin-7-one (1.00 g, 5.02 mmol) was suspended in thionyl chloride (10 mL, 137 mmol) then 3 drops of DMF were added. The resulting mixture was brought to reflux in an oil bath set at 90° C. After 3 hours of stirring, the mixture had become a clear solution. It was allowed to cool to room temperature then diluted with some toluene and concentrated to dryness under reduced pressure. The residue was resuspended in toluene and concentrated once more to dryness. The product was then dried under reduced pressure. Affords 7-chloro-2-methylsulfanyl-thiazolo[5,4-d]pyrimidine (1.10 g, 100% yield) as a beige to brown solid that was used without further purification. MS m/z 218.0 (MH⁺). ¹H NMR (CHLOROFORM-d) δ: 8.77 (s, 1H), 2.88 (s, 3H).

Step 5: Preparation of intermediate A-11 (Ar=2,3-dichlorophenyl): N-(3-amino-2,4-difluoro-phenyl)-2,3-dichloro-benzenesulfonamide; hydrochloride (811 mg, 2.08 mmol) and 7-chloro-2-methylsulfanyl-thiazolo[5,4-d]pyrimidine (544 mg, 2.50 mmol) were charged in a 20 mL vial and suspended in 10 mL of acetic acid. The mixture was then heated at 65° C. for 23 hours. It was then allowed to cool to room temperature and slowly poured into 30 mL of water containing sodium acetate (370 mg, 4.51 mmol). The resulting suspension was sonicated for homogeneity then the solids were collected by filtration and washed with a bit of water. The solids were suction dried on the filter then further dried under reduced pressure. Affords 2,3-dichloro-N-[2,4-difluoro-3-[(2-methylsulfanylthiazolo[5,4-d]pyrimidin-7-yl)amino]phenyl]benzenesulfonamide (1.17 g, 100% yield) as a yellow solid which was used without further purification. MS m/z 534.0 (MH⁺). ¹H NMR (DMSO-d₆) δ: 10.65 (s, 1H), 9.76 (s, 1H), 8.26 (s, 1H), 7.95 (dd, J=8.0, 1.4 Hz, 1H), 7.88 (dd, J=8.0, 1.4 Hz, 1H), 7.52 (t, J=8.0 Hz, 1H), 7.24 (td, J=8.6, 5.9 Hz, 1H), 7.17 (td, J=9.4, 1.2 Hz, 1H), 2.79 (s, 3H).

The following A-11 intermediates were prepared using a similar sequence as described (Table 2):

TABLE 2

| | MS | |
|---|---|---|
| | m/z | |
| A-11 | (MH⁺) | ¹H NMR (400 MHz) |
| 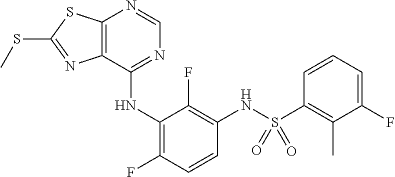 [SMILES: CSc1nc2c(Nc3c(F)ccc(NS(=O)(=O)c4cccc(F)c4C)c3F)ncnc2s1] | 498.2 | ¹H NMR (DMSO-d₆) δ: 10.44 (br. s., 1H), 9.69 (s, 1H), 8.27 (s, 1H), 7.61 (d, J = 7.4 Hz, 1H), 7.28-7.45 (m, 2H), 7.18 (td, J = 8.6, 5.5 Hz, 1H), 7.04 (br. s., 1H), 2.79 (s, 3H), 2.48 (d, J = 2.3 Hz, 3H) |
| 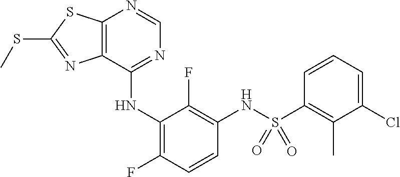 [SMILES: CSc1nc2c(Nc3c(F)ccc(NS(=O)(=O)c4cccc(Cl)c4C)c3F)ncnc2s1] | 514.2 | ¹H NMR (DMSO-d₆) δ: 10.50 (s, 1H), 9.76 (s, 1H), 8.27 (s, 1H), 7.72-7.77 (m, 2H), 7.37 (t, J = 8.2 Hz, 1H), 7.24 (td, J = 9.0, 5.7 Hz, 1H), 7.17 (td, J = 9.0, 1.2 Hz, 1H), 2.79 (s, 3H), 2.64 (s, 3H) |
| 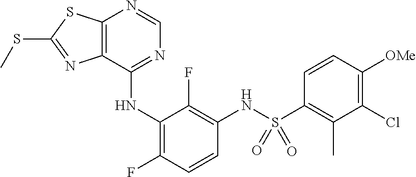 [SMILES: COc1ccc(S(=O)(=O)Nc2ccc(F)c(Nc3ncnc4sc(SC)nc34)c2F)c(C)c1Cl] | 544.2 | ¹H NMR (DMSO-d₆) δ: 10.32 (s, 1H), 9.75 (s, 1H), 8.25 (s, 1H), 7.74 (d, J = 9.0 Hz, 1H), 7.23 (td, J = 8.7, 5.7 Hz, 1H), 7.15 (td, J = 9.0, 1.2 Hz, 1H), 7.11 (d, J = 9.0 Hz, 1H), 3.90 (s, 3H), 2.79 (s, 3H), 2.64 (s, 3H) |
| 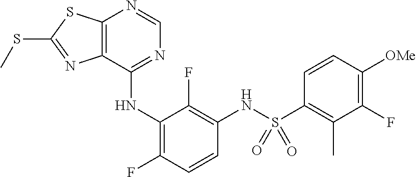 [SMILES: COc1ccc(S(=O)(=O)Nc2ccc(F)c(Nc3ncnc4sc(SC)nc34)c2F)c(C)c1F] | 528.2 | ¹H NMR (DMSO-d₆) δ: 10.26 (s, 1H), 9.75 (s, 1H), 8.26 (s, 1H), 7.55 (dd, J = 9.0, 1.6 Hz, 1H), 7.23 (td, J = 8.6, 5.9 Hz, 1H), 7.15 (td, J = 9.0, 1.2 Hz, 1H), 7.11 (t, J = 8.6 Hz, 1H), 3.87 (s, 3H), 2.79 (s, 3H), 2.46 (d, J = 2.7 Hz, 3H) |
| 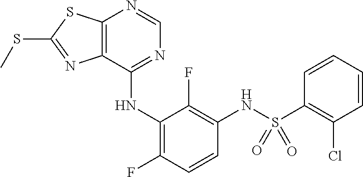 [SMILES: CSc1nc2c(Nc3c(F)ccc(NS(=O)(=O)c4ccccc4Cl)c3F)ncnc2s1] | 500.2 | ¹H NMR (DMSO-d₆) δ: 10.46 (s, 1H), 9.76 (s, 1H), 8.27 (s, 1H), 7.89 (dd, J = 7.8, 1.2 Hz, 1H), 7.68 (dd, J = 7.8, 1.6 Hz, 1H), 7.64 (td, J = 8.2, 1.6 Hz, 1H), 7.50 (ddd, J = 7.8, 7.0, 1.6 Hz, 1H), 7.21 (td, J = 9.0, 5.5 Hz, 1H), 7.15 (td, J = 9.0, 0.8 Hz, 1H), 2.78 (s, 3H) |

Step 6: Preparation of methylsulfone and methylsulfoxide mixtures from intermediates A-11 (Ar=2,3-dichlorophenyl): 2,3-dichloro-N-[2,4-difluoro-3-[(2-methylsulfanylthiazolo[5,4-d]pyrimidin-7-yl)amino]phenyl]benzenesulfonamide (1.00 g, 1.87 mmol) was suspended in DCM (25 mL). 3-chloroperoxybenzoic acid (839 mg, 3.74 mmol) was then added in one portion. The mixture was allowed to stir at room temperature for 23 hours. A saturated solution of sodium thiosulfate was added (˜1 mL) and the mixture was stirred vigorously for 10 minutes. It was then diluted with DCM and washed 3× with a 1:1 mixture of a sat. solution of NaHCO₃ and water. The aqueous layer was brought to neutrality (pH 7) with the slow addition of acetic acid. The aqueous layer was then back extracted twice with DCM and the combined organic layers were washed once with water then with brine. The organic layer was then dried over Na₂SO₄, filtered, concentrated and dried under reduced pressure. The resulting beige solid was adsorbed onto Celite® and silica gel then purified by flash chromatography through a 25 g silica gel column using a 5% to 100% EtOAc in hexanes gradient. Two main peaks were isolated separately:

Methylsulfone: rf=0.72@8:2 EtOAc/hex., 710 mg (67% yield) as a beige solid. MS m/z 566.2 (MH⁺). ¹H NMR (DMSO-d₆) δ: 10.68 (s, 1H), 10.52 (s, 1H), 8.49 (s, 1H), 7.96 (dd, J=8.2, 1.6 Hz, 1H), 7.88 (dd, J=7.8, 1.6 Hz, 1H), 7.53 (t, J=8.0 Hz, 1H), 7.29 (td, J=8.6, 5.5 Hz, 1H), 7.21 (td, J=9.0, 1.2 Hz, 1H), 3.58 (s, 3H)

Methylsulfoxide: rf=0.31@8:2 EtOAc/hex., 229.1 mg (22% yield) as a beige solid. MS m/z 550.2 (MH⁺). ¹H NMR (DMSO-d₆) δ: 10.67 (s, 1H), 10.24 (s, 1H), 8.41 (s, 1H), 7.96 (dd, J=8.2, 1.6 Hz, 1H), 7.88 (dd, J=8.0, 1.4 Hz, 1H), 7.53 (t, J=8.0 Hz, 1H), 7.27 (td, J=8.6, 5.9 Hz, 1H), 7.19 (td, J=9.0, 0.8 Hz, 1H), 3.11 (s, 3H)

Note: Separation and purification by chromatography of the two products is not necessary for subsequent reactions (i.e. step 7 of General Synthetic Method A) which can be used as crude mixtures.

Step 7: Preparation of W-I (Ar=2,3-dichlorophenyl, Het=1-benzimidazolyl) Example 4: 2,3-dichloro-N-[2,4-difluoro-3-[(2-methylsulfonylthiazolo[5,4-d]pyrimidin-7-yl)amino]phenyl]benzenesulfonamide (50 mg, 0.0883 mmol) and 1-H-benzimidazole (26 mg, 0.221 mmol) were dissolved in 0.6 mL of NMP in a 1 dram vial. N,N-diisopropylethylamine (0.077 mL, 0.441 mmol) was then added and the mixture was heated with stirring at 105° C. for 22 hours. The reaction was allowed to cool to room temperature then quenched by the addition of a formic acid (0.1 mL) solution in methanol (1 mL). The solution was then filtered and purified by reversed-phase preparative HPLC using a 40% to 90% MeOH in water gradient with 0.1% formic acid modifier. The appropriate fractions were pooled and concentrated. The residues were lyophilized from a water and MeCN mixture. Affords N-[3-[[2-(benzimidazol-1-yl)thiazolo[5,4-d]pyrimidin-7-yl]amino]-2,4-difluoro-phenyl]-2,3-dichloro-benzenesulfonamide (Example 4: 22 mg, 40% yield) as a beige solid.

Other examples of inhibitors prepared in a similar fashion are listed under method A in Table 3.

### General Method for the Synthesis of Inhibitors of Formula W-II (Method B, X═CH)—Synthesis of Example 28

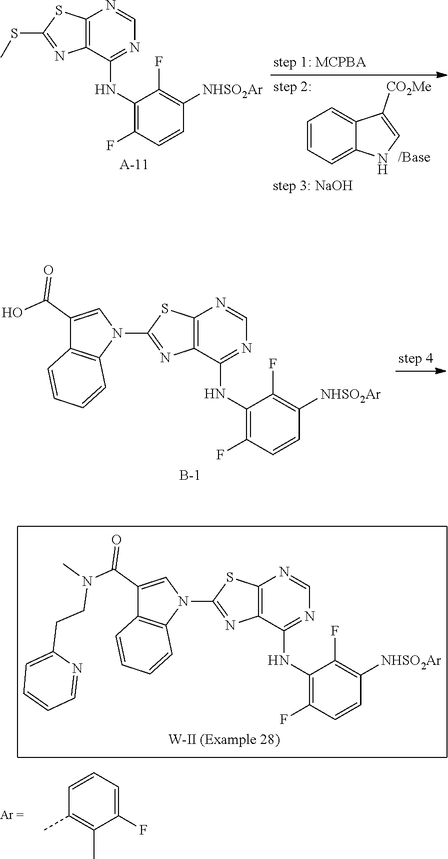

Step 1: Preparation of methylsulfone and methylsulfoxide mixtures from intermediates A-11 (Ar=2-methyl-3-fluorophenyl): N-[2,4-difluoro-3-[(2-methylsulfanylthiazolo[5,4-d]pyrimidin-7-yl)amino]phenyl]-3-fluoro-2-methyl-benzenesulfonamide (449 mg, 0.902 mmol) was suspended in DCM (10 mL). 3-chloroperoxybenzoic acid (384 mg, 1.71 mmol) was then added in one portion. The mixture was allowed to stir at room temperature for 23 hours. A saturated solution of sodium thiosulfate was added (0.2 mL) and the mixture was stirred vigorously for 10 minutes. It was then diluted with DCM and washed 3× with a 1:1 mixture of a saturated solution of NaHCO₃ and water. The combined aqueous layers were back extracted once with DCM and the combined organic layers were washed once with water then with brine. The organic layer was then dried over Na₂SO₄, filtered, concentrated and dried under reduced pressure. The resulting beige solid (445.5 mg, 93% yield as a 2:1 mixture of sulfone to sulfoxide) was used as such without purification. MS m/z 530.2 and 514.2 (MH⁺).

Steps 2 and 3: Preparation of carboxylic acid B-1: The crude mixture of sulfone and sulfoxide from above (300 mg, 0.567 mmol) and indole-3-carboxylic acid methyl ester (149 mg, 0.850 mmol) were charged in a 20 mL vial and suspended in NMP (3.6 mL). DIPEA (0.49 mL, 2.83 mmol) was then added to the orange solution and the reaction mixture was stirred at 105° C. for 24 h.

The reaction mixture was allowed to cool to room temperature and a 4N solution of sodium hydroxide (0.85 mL, 3.40 mmol) was added. The resulting mixture was stirred at 50° C. for another hour. The reaction mixture was allowed to cool to room temperature and then transferred into 100 mL of 1 N HCl. After sonication of the resulting suspension, the solids were collected by filtration and washed with water. They were then dried under reduced pressure to afford 1-[7-[2,6-difluoro-3-[(3-fluoro-2-methyl-phenyl)sulfonylamino]anilino]thiazolo[5,4-d]pyrimidin-2-yl]indole-3-carboxylic acid (292 mg, 84.3% yield) as a brown solid. The crude material was used without further purification for the next step. MS m/z 611.2 (MH⁺).

Step 4: Preparation of W-II Example 28: 1-[7-[2,6-difluoro-3-[(3-fluoro-2-methyl-phenyl)sulfonylamino]anilino]thiazolo[5,4-d]pyrimidin-2-yl]indole-3-carboxylic acid (35 mg, 0.0573 mmol) and HATU (44 mg, 0.115 mmol) were dissolved in NMP (1 mL) followed by DIPEA (0.060 mL, 0.344 mmol). The orange solution was allowed to stir at room temperature for 2-3 minutes and then N-methyl-2-(pyridin-2-yl)ethan-1-amine (0.016 mL, 0.115 mmol) was added. The reaction mixture was allowed to stir at room temperature for 2 hours. The reaction was then quenched by the addition of 0.5 mL of formic acid, diluted with a bit of DMSO and then purified by preparative reversed-phase HPLC to provide the compound of example 28 (9.8 mg, 23% yield) as a beige solid after lyophilisation from a MeCN/water mixture.

Other examples of inhibitors prepared in a similar fashion are listed under method B in Table 3.

### General Method for the Synthesis of Inhibitors of Formula W-II (Method B, X═N)—Synthesis of Example 23

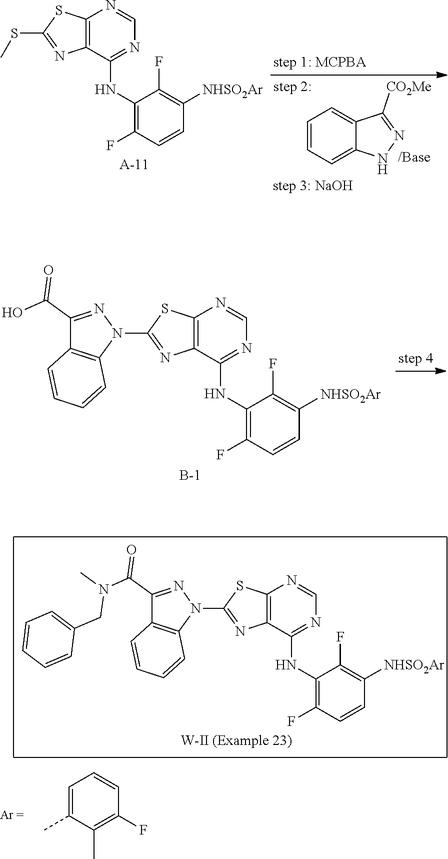

Step 1: Preparation of the methylsulfone and methylsulfoxide mixture from intermediate A-11 was performed as described above for Example 28 (step 1).
Steps 2 and 3: Preparation of carboxylic acid B-2: The crude mixture of sulfone and sulfoxide from above (300 mg, 0.567 mmol) and methyl indazolyl-3-carboxylate (150 mg, 0.850 mmol) were charged in a 20 mL vial and suspended in NMP (3.6 mL). DIPEA (0.49 mL, 2.83 mmol) was then added to the orange solution and the reaction mixture was stirred at 105° C. for 24 h. It was then was allowed to cool to room temperature and 4N sodium hydroxide (0.85 mL, 3.40 mmol) in water was added. The resulting mixture was stirred at 50° C. for another hour. The reaction mixture was allowed to cool to room temperature and then transferred into 100 mL of 1N HCl. After sonication of the resulting suspension, the solids were collected by filtration, washed with water and dried under reduced pressure to afford 1-[7-[2,6-difluoro-3-[(3-fluoro-2-methyl-phenyl)sulfonylamino]anilino]thiazolo[5,4-d]pyrimidin-2-yl]indazole-3-carboxylic acid (300 mg, 87% yield) as a pale brown solid. MS m/z 612.2 (MH⁺).

Step 4: Preparation of W-II Example 23: 1-[7-[2,6-difluoro-3-[(3-fluoro-2-methyl-phenyl)sulfonylamino]anilino]thiazolo[5,4-d]pyrimidin-2-yl]indazole-3-carboxylic acid (35 mg, 0.0572 mmol) and HATU (44 mg, 0.114 mmol) were dissolved in NMP (1 mL). DIPEA (0.060 mL, 0.343 mmol) was then added. The orange solution was allowed to stir at room temperature for 2-3 minutes and then N-methyl-1-(3-pyridinyl)methanamine (0.013 mL, 0.114 mmol) was added. The reaction mixture was allowed to stir at room temperature overnight. The reaction was then quenched by the addition of 0.5 mL of formic acid, diluted with DMSO and then purified by reversed-phase preparative HPLC using a 15% to 55% MeCN in water gradient with 0.1% formic acid modifier. The appropriate fractions were combined and lyophilized from a MeCN/water mixture. Affords the compound of example 23 (14 mg, 34% yield) as a beige solid.

Other examples of inhibitors prepared in a similar fashion are listed under method B in Table 3.

### General Method for the Synthesis of Inhibitors of Formula W-III (Method C)—Synthesis of Example 8

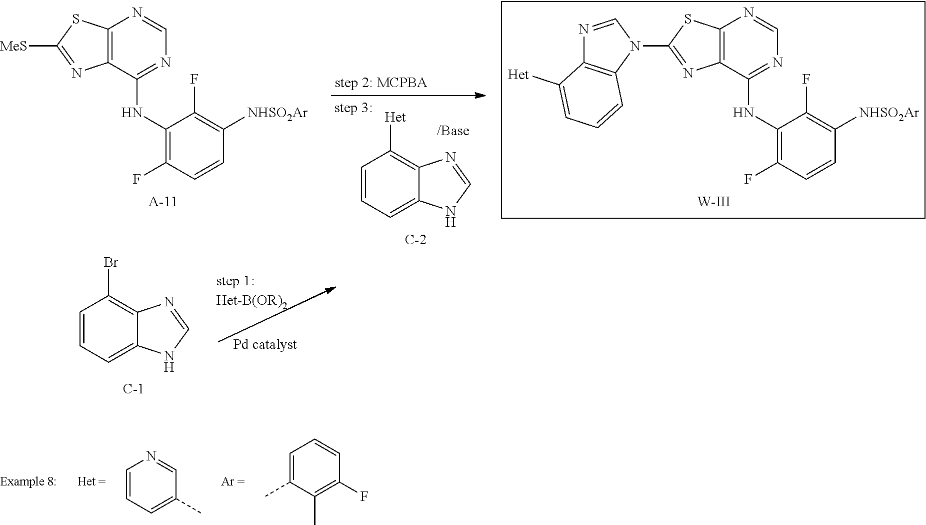

Step 1: Preparation of substituted benzimidazole intermediate C-2: Bromobenzimidazole C-1 (70 mg, 0.355 mmol), potassium carbonate (196 mg, 1.42 mmol) and 3-pyridylboronic acid (57 mg, 0.46 mmol) were charged in a 4 mL vial and dioxane (2 mL) and water (0.7 mL) were added. Argon gas was bubbled through the mixture for 1 minute and then tetrakis(triphenylphosphine)palladium (0) (16.4 mg, 0.014 mmol) was added. Argon gas was again bubbled through the solution for 3 min, the vial was sealed and heated at 100° C. for 2 hours (conversion to desired product is complete as judged by LCMS analysis). The reaction mixture was allowed to cool to RT, diluted with EtOAc and washed with brine. After drying on MgSO₄, the extract was concentrated under reduced pressure and the residue purified by flash chromatography using Et₃N pre-treated silica and a DCM−20% iPrOH/DCM gradient to provide the desired benzimidazole intermediate (58 mg, 84% yield): ¹H NMR (DMSO-d₆) δ: 12.71 (broad s, 1H), 9.24 (s. 1H), 8.57 (dd, J=5.1, 1.6 Hz, 1H), 8.43 (broad d, J=5.5 Hz, 1H), 8.31 (s, 1H), 7.61 (d, J=7.8 Hz, 1H), 7.52 (ddd, J=7.8, 4.7, 0.8 Hz, 1H), 7.47 (d, J=7.4 Hz, 1H), 7.34 (t, J=7.8 Hz, 1H). MS m/z 196.1 (MH⁺).

Step 2: Preparation of the methylsulfone and methylsulfoxide mixture from intermediate A-11 was performed as described above in Method B for Example 28 (step 1).

Step 3: Preparation of W-III (Example 8): The substituted benzimidazole from Step 1 was coupled to the crude methylsulfone/methylsulfoxide mixture from A-11 (Ar=2-methyl-3-fluorophenyl) using DIPEA in NMP as described for Example 4 (step 7) in general method A.

Other compounds (i.e. Examples 9 and 10) were prepared in a similar fashion using the appropriate intermediate A-11 (Ar=2,3-dichlorophenyl or 2-methyl-3-chlorophenyl) and are listed under method C in Table 3.

### General Method for the Synthesis of Inhibitors of Formula W-IV (Method D)—Synthesis of Example 12

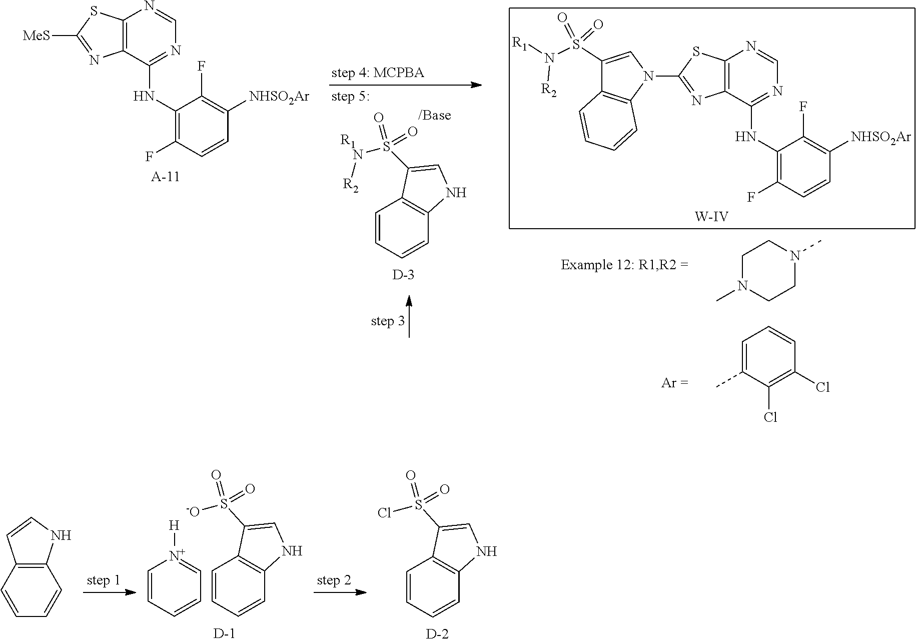

Step 1: 3-indolesulfonyl chloride D-2 was prepared as described in Org. Lett. 2011, 13, 3588. A solution of indole (3 g, 25.6 mmol) and sulfur trioxide-pyridine (4.08 g, 25.6 mmol) in pyridine (15 mL) was heated to reflux (115° C.) under stirring for 2 h. After 2 h, the reaction was cooled down to room temperature and was diluted with water (20 mL). The aqueous layer was washed with diethyl ether (20 mL) twice. The aqueous layer was evaporated to dryness to give crude pyridinium 1H-indole-3-sulfonate D-1 (5.30 g, 75% yield) as a white solid. The crude material was used as such in the next step.

Step 2: The crude pyridinium 1H-indole-3-sulfonate from step 1 (4.60 g, 16.7 mmol) was dissolved in a 1:1 mixture of sulfolane:acetonitrile (50 mL). The white suspension was cooled to 0° C., and POCl₃ (3.42 mL, 36.7 mmol) was added dropwise under stirring resulting a light brown solution. The reaction was heated to 70° C. for 1 h. After 1 h, the orange solution was cooled to 0° C. The cold orange solution was added dropwise to 250 mL of iced water. During the addition, a white precipitate was formed. The solid was filtered, washed with water and dried under vacuum to afford 1H-indole-3-sulfonyl chloride D-2 (740 mg, 21% yield) as a grey solid.

Step 3: 1H-indole-3-sulfonyl chloride (0.40 g, 1.86 mmol) was charged into a 25 mL flask to which was added 6 mL of THF. The solution was cooled to 0° C. then 1-methylpiperazine (0.41 mL, 3.71 mmol) was added dropwise. The mixture went from a red solution to a pale yellow solution with a large amount of gummy solid in the bottom of the flask. DIPEA (0.97 mL, 5.56 mmol) was then added and the mixture was allowed to warm to room temperature. After 30 minutes of stirring at room temperature, the mixture was diluted with EtOAc and water. 3-4 mL of a saturated solution of ammonium chloride was added (pH of aqueous layer ˜7) and the layers were separated. The aqueous layer was extrated twice more with EtOAc and the combined organic layers were washed once with water then once with brine. They were then dried over MgSO₄, filtered and concentrated to dryness. The resulting viscous oil was treated with a 2:1 mixture of hexanes and diethyl ether (5-6 mL), sonicated (affords a slightly gummy solid) and the liquid was decanted out. The residue was dried under reduced pressure affording 3-((4-methylpiperazin-1-yl)sulfonyl)-1H-indole D-3 (344 mg, 66% yield) as a beige foam that was used as such without further purification. MS m/z 280.1 (MH⁺). ¹H NMR (DMSO-d₆) δ: 12.20 (br. s., 1H), 7.95 (d, J=2.7 Hz, 1H), 7.78 (d, J=8.2 Hz, 1H), 7.53 (d, J=8.2 Hz, 1H), 7.26 (td, J=7.6, 1.2 Hz, 1H), 7.20 (ddd, J=8.2, 7.0, 1.2 Hz, 1H), 2.89 (br. s., 4H), 2.34 (t, J=4.9 Hz, 4H), 2.10 (s, 3H).

Step 4: Preparation of the methylsulfone and methylsulfoxide mixture from intermediate A-11 was performed as described above in Method B for Example 28 (step 1).

Step 5: Preparation of W-IV (Example 12): Sulfonamide intermediate D-3 (3-((4-methylpiperazin-1-yl)sulfonyl)-1H-indole) was coupled to the crude methylsulfone/methylsulfoxide mixture from A-11 (Ar=2,3-dichlorophenyl) using DIPEA in NMP as described for Example 4 (step 7) in general method A.

Other examples of inhibitors prepared in a similar fashion are listed under method D in Table 3.

### General Method for the Synthesis of Inhibitors of Formula W-IV (Method E)—Synthesis of Example 70

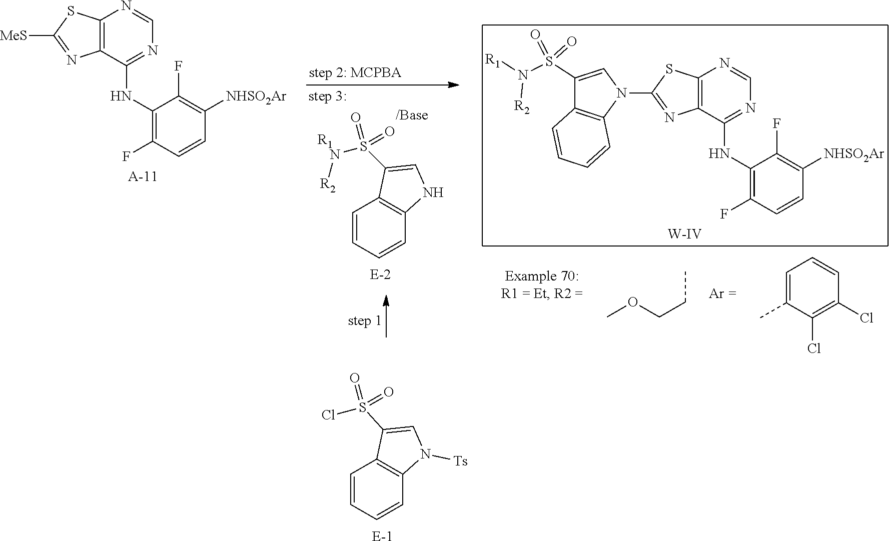

Step 1: Preparation of 3-indolesulfonamide fragment E-2: To a vial with a stirring bar was added 1-(p-tolylsulfonyl)indole-3-sulfonyl chloride (117 mg, 0.316 mmol) (Chemical and Pharmaceutical Bulletin 2009, 57, 591) in anhydrous THF (1.5 mL). The solution was cooled to 0° C. and DIEA (0.11 mL, 0.633 mmol) was added dropwise. N-(2-methoxyethyl)ethylamine (0.039 mL, 0.316 mmol) was then added dropwise and the reaction mixture was gently warmed to room temperature. The reaction was stirred at room temperature for 1 h. After completion, 10% aqueous KOH was added dropwise (same volume as solvent). The reaction was heated at 60° C. overnight. After completion, the reaction mixture was diluted with EtOAc and aq.NH4Cl was added. The layers were separated. The organic layer was washed with brine then dried over MgSO₄, filtered and concentrated to dryness to afford the expected sulfonamide derivative (86 mg, 96%) as a light orange oil. MS m/z 283.2 (MH⁺).

Step 2: Preparation of the methylsulfone and methylsulfoxide mixture from intermediate A-11 was performed as described above in Method B for Example 28 (step 1).

Step 3: Preparation of W-IV (Example 70): Sulfonamide intermediate E-2 (N-ethyl-N-(2-methoxyethyl)-1H-indole-3-sulfonamide) was coupled to the crude methylsulfone/methylsulfoxide mixture from A-11 (Ar=2,3-dichlorophenyl) using DIPEA in DMSO as described for Example 4 (step 7) in general method A.

Other examples of inhibitors prepared in a similar fashion are listed under method E in Table 3.

### General Method for the Synthesis of Inhibitors of Formula W-V (Method F)—Synthesis of Example 63

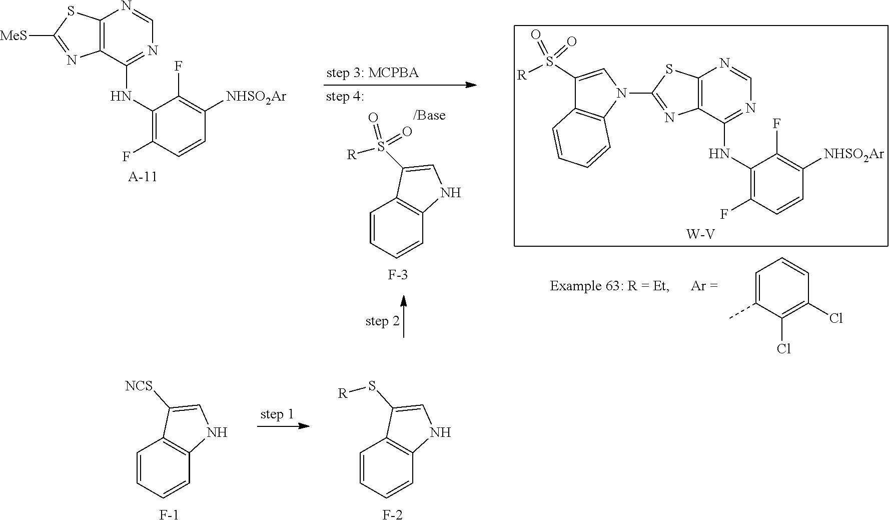

Step 1: Preparation of indole-3-thioether (R=Et) fragment F-2: To a solution of 1H-indol-3-yl-thiocyanate (Phosphorus, Sulfur and Silicon and the Related Elements 2014, 189, 1378) (175 mg, 1.0 mmol) in IPA (3.0 mL) was added sodium sulfide nonahydrate (724 mg, 3.0 mmol) dissolved in water (0.5 mL) and the mixture was then stirred at 50° C. for 2 hrs. Then to the resulting mixture was added iodoethane (0.12 mL, 1.5 mmol) and the mixture stirred at 50° C. overnight. The mixture was then diluted with DCM. The organic layer was washed with a saturated aqueous solution of ammonium chloride followed by brine then dried over MgSO₄, filtered and concentrated in vacuo to give the crude sulfide F-2 (170 mg, 95%), which was used in the next step directly without further purification. MS m/z 178.2 (MH⁺).

Step 2: Preparation of indole-3-ethylsulfone (R=Et) intermediate F-3: To a solution of 3-ethylsulfanyl-1H-indole (170 mg, 0.96 mmol) in DCM (4.8 mL) was added 3-chloroperoxybenzoic acid (537 mg, 2.40 mmol) at room temperature. After 4 hours of stirring at this temperature, the mixture was diluted with EtOAc and the resulting solution was washed with a saturated aqueous solution of NaHCO₃ twice followed by brine. The organic layer was then dried over MgSO₄, filtered and then concentrated in vacuo. The residue 3-ethylsulfonyl-1H-indole F-3 (90 mg, 45% yield) was then dissolved in DMSO and used directly in the next step (step 4) without further purification. MS m/z 210.2 (MH⁺).

Step 3: Preparation of the methylsulfone and methylsulfoxide mixture from intermediate A-11 was performed as described above in Method B for Example 28 (step 1).

Step 4: Preparation of W-V (Example 63): 3-ethylsulfonyl-1H-indole intermediate F-3 was coupled to the crude methylsulfone/methylsulfoxide mixture from A-11 (Ar=2,3-dichlorophenyl) using DIPEA in DMSO as described for Example 4 (step 7) in general method A.

Other examples of inhibitors prepared in a similar fashion are listed under method F in Table 3.

### General Method for the Synthesis of Inhibitors of Formula W-VI (Method G)—Synthesis of Example 113

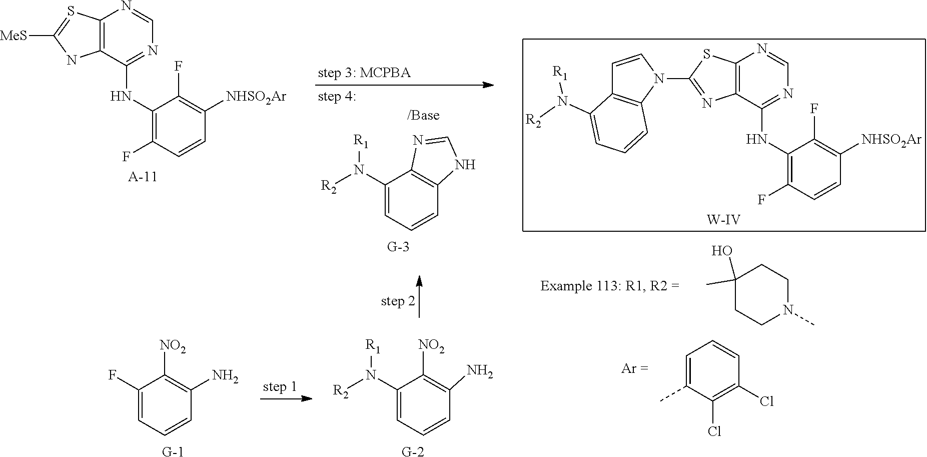

Step 1: 4-Methylpiperidin-4-ol (0.24 g, 1.84 mmol) and potassium carbonate (0.49 g, 3.52 mmol) were added to a solution of 3-fluoro-2-nitro-aniline (0.25 g, 1.60 mmol) in MeCN (2.6 mL). The resulting mixture was stirred at 85° C. for 10 h. MeCN was removed under reduce pressure and EtOAc was added. The suspension was centrifuged and the supernatant poured into a flask. The solution was concentrated and the crude 1-(3-amino-2-nitro-phenyl)-4-methyl-piperidin-4-ol (0.40 g, 94% yield) was used without further purification for the next step. MS m/z 252.2 (MH⁺).

Step 2: Iron (0.37 g, 6.70 mmol) and ammonium chloride (0.36 g, 6.70 mmol) were added to a mixture of 1-(3-amino-2-nitro-phenyl)-4-methyl-piperidin-4-ol 0.34 g, 1.34 mmol) in iPrOH (6.5 mL) and formic aicd (1.9 mL, 49.6 mmol). The resulting mixture was heated to 90° C. and stirred for 10 h. The reaction mixture was cooled down to room temperature and filtered through Celite®. The solution was concentrated and the crude was purified by column chromatography (silica gel, 0-15% MeOH in DCM) to afford 1-(1H-benzimidazol-4-yl)-4-methyl-piperidin-4-ol (0.17 g, 55% yield) as a reddish foamy solid. MS m/z 232.2 (MH⁺). ¹H NMR (400 MHz, DMSO-d₆) δ: 12.21 (br. s, 1H), 8.02 (s, 1H), 6.85-7.20 (m, 2H), 6.33-6.67 (m, 1H), 4.24 (s, 1H), 3.15-3.26 (m, 2H), 2.48 (td, J=1.66, 3.72 Hz, 2H), 1.41-1.74 (m, 4H), 1.16 (s, 3H).

Step 3: Preparation of the methylsulfone and methylsulfoxide mixture from intermediate A-11 was performed as described above in Method B for Example 28 (step 1) (Ar=2,3-dichlorophenyl).

Step 4: Preparation of W-VI (Example 113): 1-(1H-benzimidazol-4-yl)-4-methyl-piperidin-4-ol intermediate G-3 was coupled to the crude methylsulfone/methylsulfoxide mixture from A-11 (Ar=2,3-dichlorophenyl) using cesium carbonate in DMSO in a fashion analogous to that employed for Example 4 (step 7) in general method A.

Other examples of inhibitors prepared in a similar fashion are listed under method G in Table 3.

### Preparation of Examples 17, 47 and 99

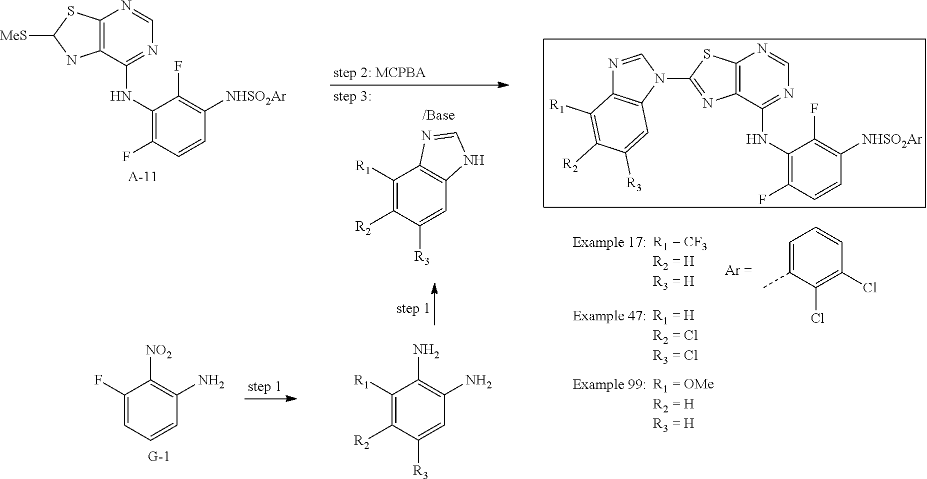

Step 1: Preparation of 4-(trifluoromethyl)-1H-benzo[d]imidazole (for Example 17): In a 10 mL vial was added 3-(trifluoromethyl)benzene-1,2-diamine (280 mg, 1.59 mmol) in formic acid (2.0 mL). The reaction was heated to 100° C. and left to stir for 3 hours. The reaction was cooled to room temperature and the formic acid was removed under reduced pressure. The residue was taken up in a saturated solution of NaHCO₃ affording a precipitate which was homogenized by sonication. The solids were collected by filtration, washed with water, briefly dried on the filter and collected. The product was dried under reduced pressure overnight to afford 4-(trifluoromethyl)-1H-benzimidazole (269 mg, 91% yield) as a light brown solid. MS m/z 187.2 (MH⁺). ¹H NMR (DMSO-d₆) δ: 12.93 (br. s., 1H), 8.39 (s, 1H), 7.88 (d, J=8.2 Hz, 1H), 7.54 (d, J=7.8 Hz, 1H), 7.36 (t, J=7.8 Hz, 1H).

5,6-dichloro-1H-benzimidazole (for Example 47) was prepared in a similar fashion starting from commercial 4,5-dichloro-1,2-phenylenediamine. The product was obtained as a pale brown solid 5 (100% yield). MS m/z 187.0 (MH⁺). ¹H NMR (CHLOROFORM-d) δ: 9.66 (br. s., 1H), 8.09 (s, 1H), 7.78 (br. s., 2H).

4-methoxy-1H-benzimidazole (for Example 99) was prepared in a similar fashion starting from commercial 3-methoxybenzene-1,2-diamine. The product was obtained as an off-white solid after silica gel chromatography (EtOAc/heptanes) (71% yield). MS m/z 149.2 (MH⁺).

Step 2: Preparation of the methylsulfone and methylsulfoxide mixture from intermediate A-11 was performed as described above in Method B for Example 28 (step 1) (Ar=2,3-dichlorophenyl).

Step 3: Preparation of Examples 17, 47 and 99 was performed by coupling the appropriate substituted benzimidazole described above to the crude methylsulfone/methylsulfoxide mixture from A-11 (Ar=2,3-dichlorophenyl) using DIPEA in NMP as described for Example 4 (step 7) in general method A.

### Preparation of Examples 49 and 71

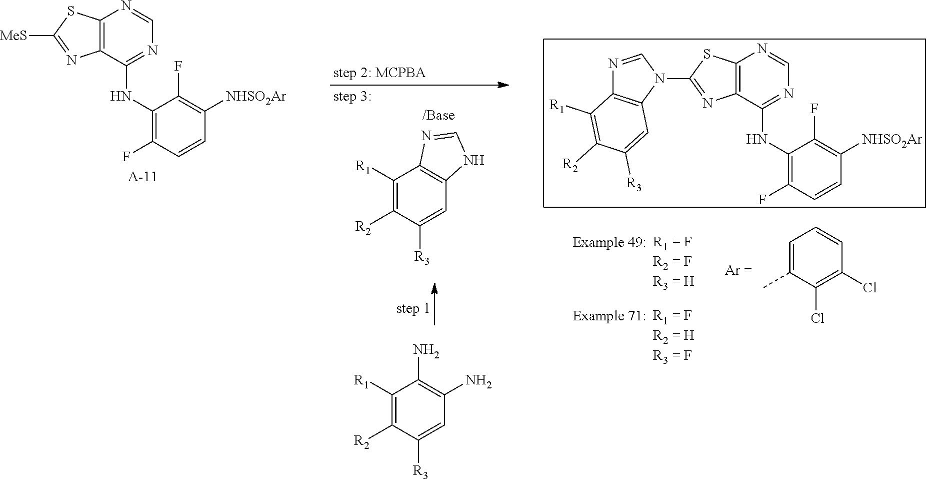

Step 1: Preparation of 4,5-difluoro-1H-benzimidazole (for Example 49): 2,3-difluoro-6-nitro-aniline (800 mg, 4.60 mmol) was charged in a flask along with ammonium chloride (2.46 g, 46.0 mmol) and iron powder (2.57 g, 46.0 mmol). IPA (11.5 mL) was then added followed by formic acid (12 mL, 305 mmol). The flask was equipped with a reflux condenser and the reaction flask was immersed in an oil bath set to 80° C. and stirred for 5 hours. After cooling, the mixture was filtered through a small pad of Celite® rinsing with IPA then the filtrate was concentrated to dryness. The residue was then neutralized with a saturated solution of NaHCO₃ and extracted with EtOAc three times. The combined organic layers were washed with brine, dried over MgSO₄, filtered and concentrated. The resulting residue was purified by silica gel chromatography using a 50% to 100% EtOAc in DCM gradient. The appropriate fractions were pooled, concentrated and dried under reduced pressure. Affords 4,5-difluoro-1H-benzimidazole (706 mg, 99% yield). MS m/z 155.2 (MH⁺). ¹H NMR (DMSO-d₆) δ: 13.26 (br. s., 0.2H), 12.83 (br. s., 0.8H), 8.32 (s, 1H), 7.43-7.54 (m, 0.2H), 7.34 (dd, J=9.0, 3.9 Hz, 0.8H), 7.17-7.31 (m, 1H).

4,6-difluoro-1H-benzimidazole (for Example 71) was prepared in a similar fashion starting from commercial 2,4-difluoro-6-nitro-aniline in 93% yield. MS m/z 155.2 (MH⁺). ¹H NMR (DMSO-d₆) δ: 12.87 (br. s., 1H), 8.28 (s, 1H), 7.25 (d, J=7.8 Hz, 1H), 7.05 (t, J=10.4 Hz, 1H).

Step 2: Preparation of the methylsulfone and methylsulfoxide mixture from intermediate A-11 was performed as described above in Method B for Example 28 (step 1) (Ar=2,3-dichlorophenyl).

Step 3: Preparation of Examples 49 and 71 was performed by coupling the appropriate substituted benzimidazole described above to the crude methylsulfone/methylsulfoxide mixture from A-11 (Ar=2,3-dichlorophenyl) using DIPEA in NMP as described for Example 4 (step 7) in general method A.

### Preparation of Example 72

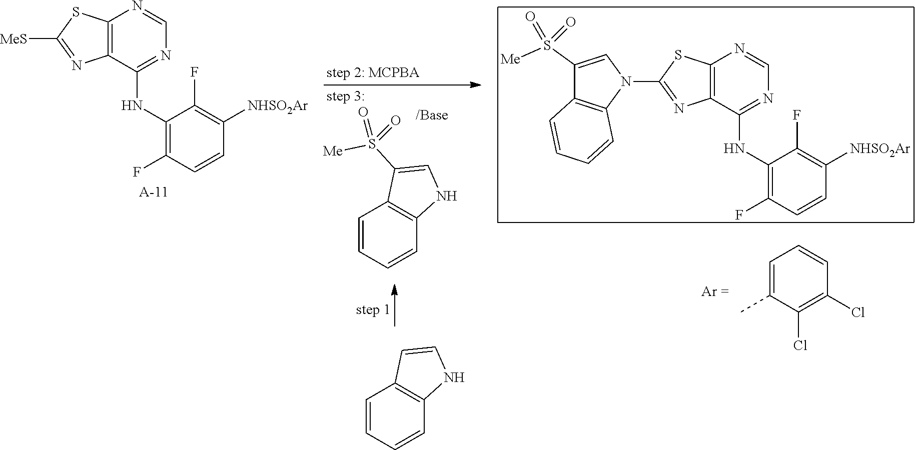

Step 1: Preparation of 3-methylsulfonyl-1H-indole: Indole (182 mg, 1.55 mmol) was dissolved in THF (17 mL) under nitrogen at room temperature. Potassium tert-butoxide (192 mg, 1.71 mmol) was added and the mixture stirred 30 min. Triethylborane (0.22 mL, 1.55 mmol) was added dropwise at RT and stirred for 30 min. Methanesulfonyl chloride (0.13 mL, 1.71 mmol) was added and the mixture was stirred at-15° C. 5 days. A saturated aqueous solution of NaHCO₃ was added then the mixture was extracted with EtOAc. The organic layer was washed with brine, dried over MgSO₄, filtered and concentrated under vacuum. The residue was purified by chromatography on silica gel using an EtOAc in hexanes gradient (20-60%) to afford 3-methylsulfonyl-1H-indole (30 mg, 10% yield).

Step 2: Preparation of the methylsulfone and methylsulfoxide mixture from intermediate A-11 was performed as described above in Method B for Example 28 (step 1) (Ar=2,3-dichlorophenyl).

Step 3: Preparation of Example 72 was performed by coupling 3-methylsulfonyl-1H-indole described above to the crude methylsulfone/methylsulfoxide mixture from A-11 (Ar=2,3-dichlorophenyl) using DIPEA in DMSO as described for Example 4 (step 7) in general method A. Preparation of Examples 79 and 80:

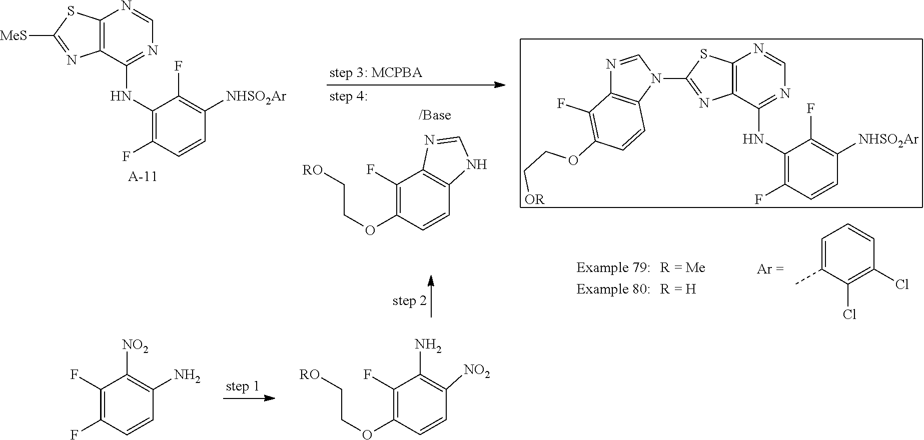

Step 1: Preparation of 2-fluoro-3-(2-methoxyethoxy)-6-nitro-aniline (for Example 79): 2,3-difluoro-6-nitro-aniline (800 mg, 4.60 mmol) was charged in a 25 mL flask and dissolved in 10 mL of DMF at room temperature affording a bright yellow solution. 2-methoxyethanol (2.2 mL, 27.6 mmol) was then added followed by potassium carbonate (2.54 g, 18.4 mmol). The color of the solution changed to bright red. The mixture was heated in an oil bath at 80° C. for 22 hours. After cooling, the mixture was diluted with EtOAc and washed with a saturated aqueous ammonium chloride solution. The aqueous layer was extracted twice with EtOAc and the combined organic layers were washed with brine, dried over MgSO₄, filtered and concentrated. The resulting residue was purified by silica gel chromatography using an EtOAc in hexanes gradient. Affords 2-fluoro-3-(2-methoxyethoxy)-6-nitro-aniline (1.01 g, 96% yield). ¹H NMR (DMSO-d₆) δ: 7.87 (dd, J=9.8, 2.0 Hz, 1H), 7.16 (br. s, 2H), 6.60 (dd, J=10.0, 8.0 Hz, 1H), 4.24-4.32 (m, 2H), 3.63-3.71 (m, 2H), 3.31 (s, 3H).

2-(3-amino-2-fluoro-4-nitro-phenoxy)ethanol (for Example 80) was prepared in a similar fashion using ethylene glycol. The product was obtained in 98% yield. ¹H NMR (DMSO-d₆) δ: 7.86 (dd, J=9.8, 2.0 Hz, 1H), 7.15 (s, 2H), 6.60 (dd, J=9.8, 7.8 Hz, 1H), 4.94 (t, J=5.5 Hz, 1H), 4.17 (t, J=4.9 Hz, 2H), 3.73 (q, J=4.8 Hz, 2H).

Step 2: Preparation of the substituted benzimidazoles from the 2-nitroanilines described in step 1 was performed using the “one-pot” procedure described in Method G for Example 113 (step 2).

4-fluoro-5-(2-methoxyethoxy)-1H-benzimidazole (for Example 79): 82% yield. MS m/z 211.2 (MH⁺). ¹H NMR (DMSO-d₆) δ: 12.90 (br. s., 0.3H), 12.57 (br. s., 0.7H), 8.20 (s, 0.7H), 8.18 (s, 0.3H), 7.39 (d, J=8.6 Hz, 0.3H), 7.25 (d, J=8.6 Hz, 0.7H), 7.09 (t, J=8.2 Hz, 0.7H), 7.06 (t, J=8.2 Hz, 0.3H), 4.19 (t, J=4.3 Hz, 0.6H), 4.16 (t, J=4.3 Hz, 1.4H), 3.62-3.70 (m, 2H), 3.32 (s, 3H).

2-[(4-fluoro-1H-benzimidazol-5-yl)oxy]ethanol (for Example 80): 53% yield. MS m/z 197.2 (MH⁺). ¹H NMR (DMSO-d₆) δ: 12.88 (br. s., 0.3H), 12.56 (br. s., 0.7H), 8.19 (s, 0.7H), 8.17 (s, 0.3H), 7.39 (d, J=9.0 Hz, 0.3H), 7.24 (d, J=8.6 Hz, 0.7H), 7.10 (t, J=8.0 Hz, 0.7H), 7.06 (t, J=8.2 Hz, 0.3H), 4.87 (t, J=5.5 Hz, 1H), 4.09 (t, J=4.7 Hz, 0.6H), 4.06 (t, J=5.1 Hz, 1.4H), 3.71 (q, J=5.2 Hz, 2H).

Step 3: Preparation of the methylsulfone and methylsulfoxide mixture from intermediate A-11 was performed as described above in Method B for Example 28 (step 1) (Ar=2,3-dichlorophenyl).

Step 4: Preparation of Examples 79 and 80 was performed by coupling the appropriate substituted benzimidazole described above to the crude methylsulfone/methylsulfoxide mixture from A-11 (Ar=2,3-dichlorophenyl) using DIPEA in NMP as described for Example 4 (step 7) in general method A.

### Preparation of Example 81

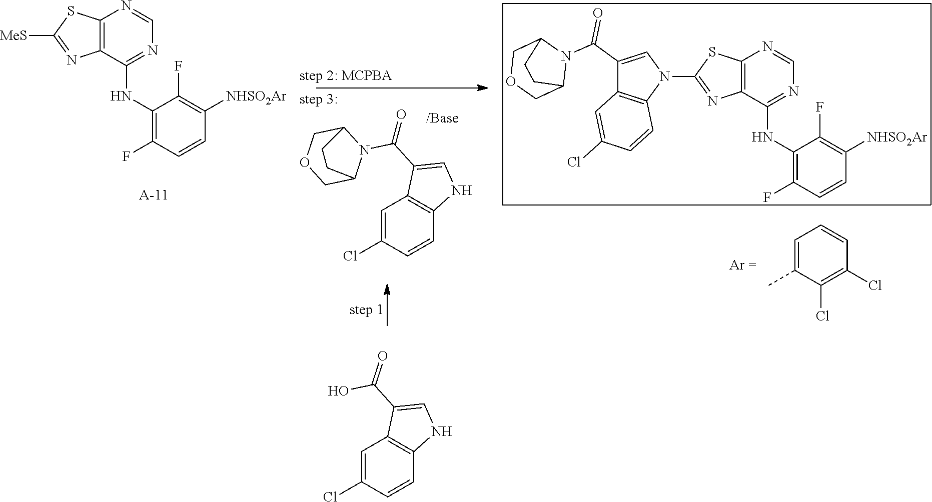

Step 1: Preparation of (5-chloro-1H-indol-3-yl)-(3-oxa-8-azabicyclo[3.2.1]octan-8-yl)methanone: 5-chloro-1H-indole-3-carboxylic acid (50 mg, 0.256 mmol) was dissolved in NMP (1.5 mL). 2-(1H-Benzotriazole-1-yl)-1,1,3,3-tetramethyluronium tetrafluoroborate (98 mg, 0.31 mmol) was added at room temperature followed by 3-Oxa-8-aza-bicyclo[3.2.1]octane HCl (42 mg, 0.28 mmol). N,N-Diisopropylethylamine (0.18 mL, 1.02 mmol) was finally added and the mixture was allowed to stir for 1 hour. It was then partitioned between EtOAc and a saturated aqueous solution of ammonium chloride. The layers were separated and the aqueous layer was further extracted twice with EtOAc. The combined organic layers were washed with brine then dried over MgSO₄, filtered and concentrated. The resulting residue was purified by silica gel chromatography using a 1:1 EtOAc in DCM to 100% EtOAc gradient followed by 2% IPA in EtOAc. Affords (5-chloro-1H-indol-3-yl)-(3-oxa-8-azabicyclo[3.2.1]octan-8-yl)methanone (65 mg, 88% yield). MS m/z 291.1 (MH⁺). ¹H NMR (DMSO-d₆) δ: 11.83 (br. s., 1H), 7.89 (s, 1H), 7.83 (d, J=2.0 Hz, 1H), 7.46 (d, J=8.6 Hz, 1H), 7.17 (dd, J=8.6, 2.3 Hz, 1H), 4.47 (br. s., 2H), 3.68 (d, J=11.0 Hz, 2H), 3.61 (dd, J=10.6, 1.2 Hz, 2H), 1.80-1.94 (m, 4H).

Step 2: Preparation of the methylsulfone and methylsulfoxide mixture from intermediate A-11 was performed as described above in Method B for Example 28 (step 1) (Ar=2,3-dichlorophenyl).

Step 3: Preparation of Example 81 was performed by coupling (5-chloro-1H-indol-3-yl)-(3-oxa-8-azabicyclo[3.2.1]octan-8-yl)methanone described above to the crude methylsulfone/methylsulfoxide mixture from A-11 (Ar=2,3-dichlorophenyl) using DIPEA in DMSO as described for Example 4 (step 7) in general method A.

### Preparation of Examples 91, 92, 106 and 107

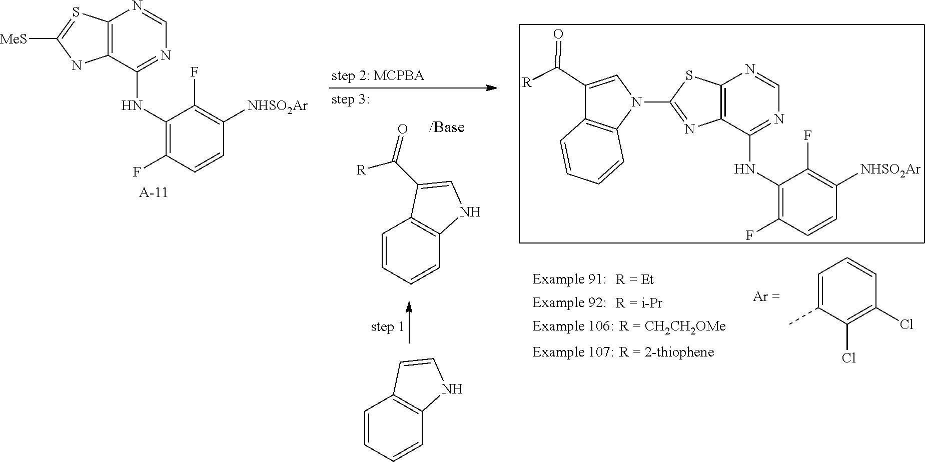

Step 1: Preparation of 1-(1H-indol-3-yl)propan-1-one (for Example 91): To a 100 mL round-bottom flask, was added indole (200 mg, 1.71 mmol) in 7 mL of DCM. The solution was cooled to 0° C. then a 1.8M (25% wt) solution of Et₂AlCl in toluene (1.42 mL, 2.56 mmol) was added dropwise. The reaction was left to stir for 30 min at 0° C. Propionyl chloride in 6 mL of DCM was added dropwise. The reaction was warmed to room temperature and left to stir for 2.5 hours. The reaction was quenched with 3 eq. of NaOAc in 5 mL of water. The reaction was diluted with DCM and filtered through Celite® to remove the aluminum salts. The filtrate was transferred to a separatory funnel and then water was added. 1M NaOH was added to help solubilize any remaining aluminum salts. The layers were separated, and the organic layer was washed with water then brine. The organic layer was then dried over MgSO₄, filtered and then concentrated under reduced pressure. The crude material was purified by silica gel chromatography using a 100% hexanes to 75% EtOAc in hexanes gradient. Affords 1-(1H-indol-3-yl)propan-1-one (228.9 mg, 77% yield) as a light yellow solid. MS m/z 174.2 (MH⁺). ¹H NMR (400 MHz, DMSO-d₆) δ: 11.88 (br. s., 1H), 8.30 (s, 1H), 8.15-8.23 (m, 1H), 7.41-7.49 (m, 1H), 7.18 (quint, J=7.2, 7.2, 7.2, 7.2, 1.4 Hz, 2H), 2.87 (q, J=7.4 Hz, 2H), 1.11 (t, J=7.4 Hz, 3H). 1-(1H-indol-3-yl)-2-methyl-propan-1-one (for Example 92) was prepared in a similar fashion using isobutyric acid chloride. The product was obtained in 94% yield. MS m/z 188.2 (MH⁺). ¹H NMR (400 MHz, DMSO-d₆) δ: 11.90 (br. s., 1H), 8.35 (s, 1H), 8.20 (dd, J=8.0, 1.4 Hz, 1H), 7.46 (dd, J=7.4, 1.2 Hz, 1H), 7.18 (quint, J=7.3, 7.3, 7.3, 7.3, 1.2 Hz, 2H), 3.45 (spt, J=6.7 Hz, 1H), 1.12 (d, J=6.7 Hz, 6H).

1-(1H-indol-3-yl)-3-methoxy-propan-1-one (for Example 106) was prepared in a similar fashion using 3-methoxypropanoyl chloride. The product was obtained in 86% yield. MS m/z 204.2 (MH⁺). 1H-indol-3-yl(2-thienyl)methanone (for Example 107) was prepared in a similar fashion using 2-thiophenecarbonyl chloride. The product was obtained in 80% yield. MS m/z 228.0 (MH⁺).

Step 2: Preparation of the methylsulfone and methylsulfoxide mixture from intermediate A-11 was performed as described above in Method B for Example 28 (step 1) (Ar=2,3-dichlorophenyl).

Step 3: Preparation of Examples 91, 92, 106 and 107 was performed by coupling the appropriate substituted indole described above to the crude methylsulfone/methylsulfoxide mixture from A-11 (Ar=2,3-dichlorophenyl) using DIPEA in DMSO as described for Example 4 (step 7) in general method A.

### Preparation of Example 98

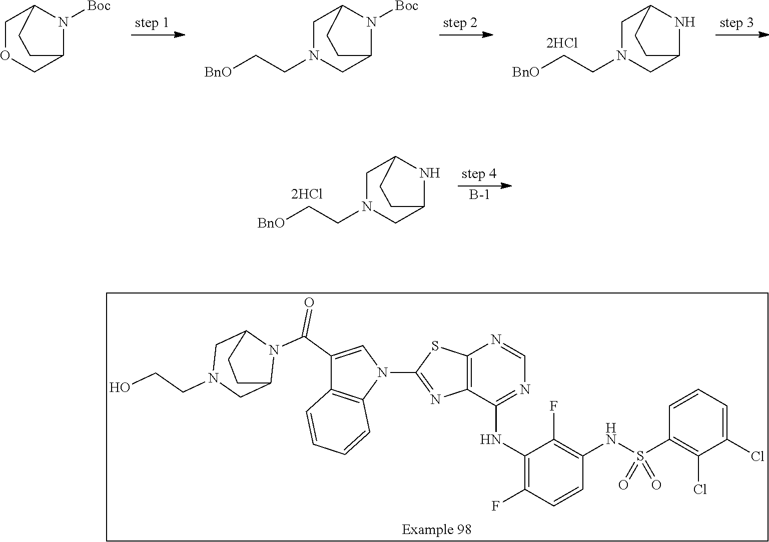

Step 1: tert-Butyl 3,8-diazabicyclo[3.2.1]octane-8-carboxylate (0.70 g, 3.30 mmol) and potassium carbonate (1.14 g, 8.24 mmol) were charged in a 25 mL flask and suspended in 10 mL of MeCN. benzyl 2-bromoethyl ether (0.57 mL, 3.63 mmol) was then added dropwise at room temperature. The resulting mixture was allowed to stir at room temperature for 22 hours then at 40° C. for 4 hours. The reaction mixture was diluted with EtOAc. The solids were removed by filtration and the filtrate was concentrated to dryness. The resulting residue was purified by flash chromatography on silica gel using a 0% to 40% EtOAc in hexanes gradient. The appropriate fractions were concentrated then dried under reduced pressure to afford tert-butyl 3-(2-benzyloxyethyl)-3,8-diazabicyclo[3.2.1]octane-8-carboxylate (678 mg, 59% yield) as a colorless oil. MS m/z 347.4 (MH⁺). ¹H NMR (DMSO-d6) δ: 7.24-7.39 (m, 5H), 4.47 (s, 2H), 3.99 (br. s., 2H), 3.51 (t, J=5.7 Hz, 2H), 2.64 (d, J=10.2 Hz, 2H), 2.50 (t, J=5.7 Hz, 2H), 2.19 (d, J=10.6 Hz, 2H), 1.60-1.82 (m, 4H), 1.39 (s, 9H).

Step 2: tert-butyl 3-(2-benzyloxyethyl)-3,8-diazabicyclo[3.2.1]octane-8-carboxylate (673 mg, 1.94 mmol) was charged in a 100 mL flask and dissolved in 5 mL of dioxane. A 4N solution of HCl in dioxane (1.94 mL, 7.76 mmol) was then added dropwise. After 1 hour of stirring, another portion of the HCl solution was added (2.91 mL, 11.64 mmol) and the mixture was warmed to 40° C. After another 2 hours of stirring at that temperature, a gum had separated at the bottom of the flask. The mixture was cooled to room temperature, diluted with methanol and concentrated to dryness. The residue was dissolved in methanol and concentrated to dryness once more then dried under reduced pressure. Affords 3-(2-benzyloxyethyl)-3,8-diazabicyclo[3.2.1]octane; dihydrochloride (620 mg, 100% yield) as a white gummy solid which was used as such. MS m/z 247.3 (MH⁺). ¹H NMR (DMSO-d6) δ: 11.36 (br. s., 1H), 10.10 (br. s., 1H), 9.64 (br. s., 1H), 7.33-7.40 (m, 4H), 7.27-7.33 (m, 1H), 4.51 (s, 2H), 4.18 (br. s., 2H), 3.88 (br. s., 2H), 3.58-3.75 (m, 2H), 3.21 (br. s., 2H), 2.34 (br. s., 2H), 2.02 (br. s., 2H) (2H hidden underwater peak).

Step 3: To a solution of 3-(2-benzyloxyethyl)-3,8-diazabicyclo[3.2.1]octane; dihydrochloride (615 mg, 1.93 mmol) in a 100 mL flask in 15 mL of methanol was added 10% palladium on charcoal (205 mg, 0.193 mmol). The flask was placed under reduced pressure then filled with hydrogen. This operation was repeated 3 more times then the mixture was stirred vigorously under a balloon atmosphere of hydrogen over weekend. The flask was flushed with nitrogen and the reaction mixture was filtered through a short pad of Celite®. The filtrate was concentrated and dried under reduced pressure. Affords 2-(3,8-diazabicyclo[3.2.1]octan-3-yl)ethanol; dihydrochloride (406 mg, 92% yield). MS m/z 157.2 (MH⁺). ¹H NMR (DMSO-d6) δ: 11.14 (br. s., 1H), 10.07 (br. s., 1H), 9.68 (br. s., 1H), 5.08 (br. s., 1H), 4.16 (br. s., 2H), 3.81 (br. s., 2H), 3.03 (br. s., 2H), 2.33 (br. s., 2H), 2.03 (br. s., 2H) (4H hidden underwater peak).

Step 4: Preparation of Example 98 was performed by coupling the amine described above to intermediate B-1 (Ar=2,3-dichlorophenyl) using TBTU instead of HATU, in a similar fashion as described in general method B for Example 28 (step 4).

### Preparation of Example 100

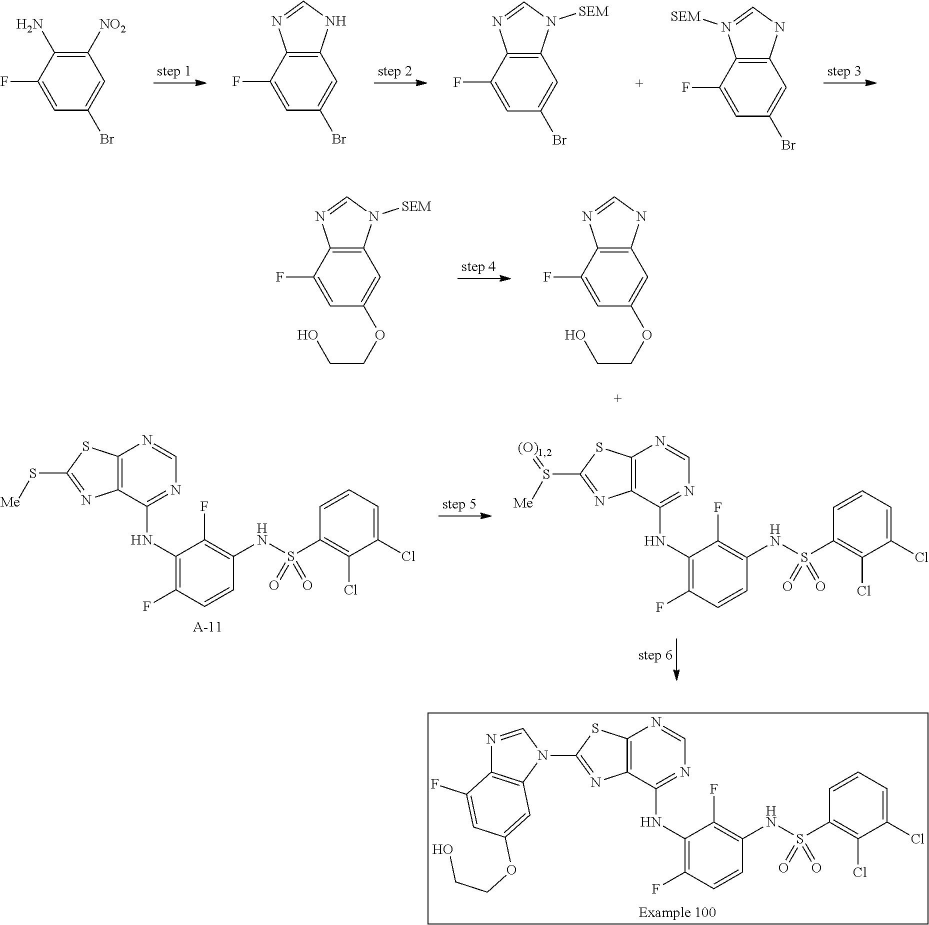

Step 1: Preparation of 6-bromo-4-fluoro-1H-benzimidazole: This compound was prepared starting with commercial 4-bromo-2-fluoro-6-nitro-aniline using iron, ammonium chloride and formic acid in a similar fashion as the benzimidazoles described for Examples 49/71 (step 1). After silica gel chromatography (EtOAc/DCM gradient), 6-bromo-4-fluoro-1H-benzimidazole was obtained as an off-white solid (96% yield). MS m/z 215.0 (MH⁺). ¹H NMR (DMSO-d6) δ: 12.94 (br. s., 1H), 8.31 (s, 1H), 7.63 (s, 1H), 7.28 (d, J=10.6 Hz, 1H).

Step 2: Preparation of 2-[(6-bromo-4-fluoro-benzimidazol-1-yl)methoxy]ethyl-trimethyl-silane and 2-[(5-bromo-7-fluoro-benzimidazol-1-yl)methoxy]ethyl-trimethyl-silane: 6-bromo-4-fluoro-1H-benzimidazole (500 mg, 2.05 mmol) was dissolved in 10 mL of DMF then potassium carbonate (848 mg, 6.14 mmol) was added followed by SEM-CI (0.44 mL, 2.46 mmol) at room temperature. The mixture was allowed to stir at the same temperature for 18 hours. Another portion of SEM-CI (0.18 mL, 1.03 mmol) was added and the mixture was allowed to stir for another hour. The mixture was poured into a saturated solution of ammonium chloride then extracted 3× with EtOAc. The combined organic layers were washed with water then brine then dried over MgSO₄, filtered and concentrated. The resulting residue was purified by silica gel chromatography using a 100% hexanes to 40% EtOAc in hexanes gradient. The appropriate fractions were pooled, concentrated and dried under reduced pressure. Affords a roughly 3:1 mixture of 2-[(6-bromo-4-fluoro-benzimidazol-1-yl)methoxy]ethyl-trimethyl-silane and 2-[(5-bromo-7-fluoro-benzimidazol-1-yl)methoxy]ethyl-trimethyl-silane (689 mg, 97% yield) as a waxy beige solid. MS m/z 385.2 (MH⁺). ¹H NMR (DMSO-d₆) δ: 8.50 (s, 0.25H), 8.46 (s, 0.75H), 7.80 (d, J=1.6 Hz, 0.75H), 7.77 (d, J=1.6 Hz, 0.25H), 7.43 (dd, J=10.6, 1.6 Hz, 0.25H), 7.36 (dd, J=10.6, 1.6 Hz, 0.75H), 5.66 (s, 1.5H), 5.64 (s, 0.5H), 3.45-3.54 (m, 2H), 0.78-0.86 (m, 2H), −0.09 (s, 6.75H), −0.11 (s, 2.25H).

Step 3: Preparation of 2-[7-fluoro-3-(2-trimethylsilylethoxymethyl)benzimidazol-5-yl]oxyethanol: The 3:1 mixture of isomers 2-[(6-bromo-4-fluoro-benzimidazol-1-yl)methoxy]ethyl-trimethyl-silane and 2-[(5-bromo-7-fluoro-benzimidazol-1-yl)methoxy]ethyl-trimethyl-silane (630 mg, 1.82 mmol) described above was charged in a 20 mL vial followed by 1,10-phenanthroline (33 mg, 0.18 mmol), copper (1) iodide (35 mg, 0.18 mmol) and cesium carbonate (1.19 g, 3.65 mmol). Ethylene glycol (8.2 mL, 147 mmol). The resulting mixture was stirred at 120° C. for 21 hours. The mixture was allowed to cool to room temperature and it was then diluted with a saturated solution of ammonium chloride, water and EtOAc. The layers were separated and the aqueous layer was further extracted twice more with EtOAc. The combined organic layers were diluted with a bit of hexanes and washed once with a saturated aqueous solution of ammonium chloride, twice with water and once with brine. The organic layer was then dried over MgSO₄, filtered and concentrated. The residue was purified by silica gel chromatography using a 30% to 90% EtOAc in hexanes gradient. After pooling, concentrating and drying the appropriate fractions, 2-[7-fluoro-3-(2-trimethylsilylethoxymethyl)benzimidazol-5-yl]oxyethanol (307 mg, 52% yield) was obtained as a single isomer, as a thick colorless oil that slowly crystallizes. MS m/z 327.3 (MH⁺). ¹H NMR (DMSO-d₆) δ: 8.26 (s, 1H), 7.06 (d, J=2.0 Hz, 1H), 6.73 (dd, J=12.3, 2.2 Hz, 1H), 5.60 (s, 2H), 4.89 (t, J=5.5 Hz, 1H), 4.03 (t, J=5.1 Hz, 2H), 3.74 (q, J=5.5 Hz, 2H), 3.49 (t, J=7.8 Hz, 2H), 0.83 (t, J=7.8 Hz, 2H), −0.09 (s, 9H).

Step 4: Preparation of 2-[(7-fluoro-3H-benzimidazol-5-yl)oxy]ethanol: 2-[7-fluoro-3-(2-trimethylsilylethoxymethyl)benzimidazol-5-yl]oxyethanol (308 mg, 0.94 mmol) was dissolved in 5 mL of THF in a 20 mL vial then a 1M THF solution of tetra-N-butylammonium fluoride (4.72 mL, 4.72 mmol) was added at room temperature. The vial was then sealed and the mixture was heated at 65° C. for 23 hours. The mixture was diluted with EtOAc and water with a bit of NaHCO₃. The layers were separated and the aqueous layer was further extracted with EtOAc another 4×. The combined organic layers were washed with brine then dried over MgSO₄, filtered and concentrated. The gummy solid residue was purified by silica gel chromatography using a 2% to 20% IPA in EtOAc gradient. The fractions containing the product were pooled, concentrated and dried under reduced pressure. Affords 2-[(7-fluoro-3H-benzimidazol-5-yl)oxy]ethanol (139 mg, 75% yield) as a white solid. ¹H NMR reveals a 85:15 mixture of tautomers. MS m/z 197.2 (MH⁺). ¹H NMR (DMSO-d₆) δ: 12.89 (br. s., 0.15H), 12.51 (br. s., 0.85H), 8.18 (br. s., 0.15H), 8.10 (s, 0.85H), 7.03 (br. s., 0.15H), 6.86 (br. s., 0.85H), 6.73 (br. s., 0.15H), 6.66 (d, J=12.1 Hz, 0.85H), 4.88 (t, J=5.5 Hz, 1H), 4.01 (t, J=4.9 Hz, 2H), 3.72 (q, J=5.3 Hz, 2H).

Step 5: Preparation of the methylsulfone and methylsulfoxide mixture from intermediate A-11 was performed as described above in Method B for Example 28 (step 1) (Ar=2,3-dichlorophenyl).

Step 6: Preparation of Example 100 was performed by coupling 2-[(7-fluoro-3H-benzimidazol-5-yl)oxy]ethanol described above to the crude methylsulfone/methylsulfoxide mixture from A-11 (Ar=2,3-dichlorophenyl) using DIPEA in DMSO as described for Example 4 (step 7) in general method A.

### Preparation of Examples 104,112,116-118

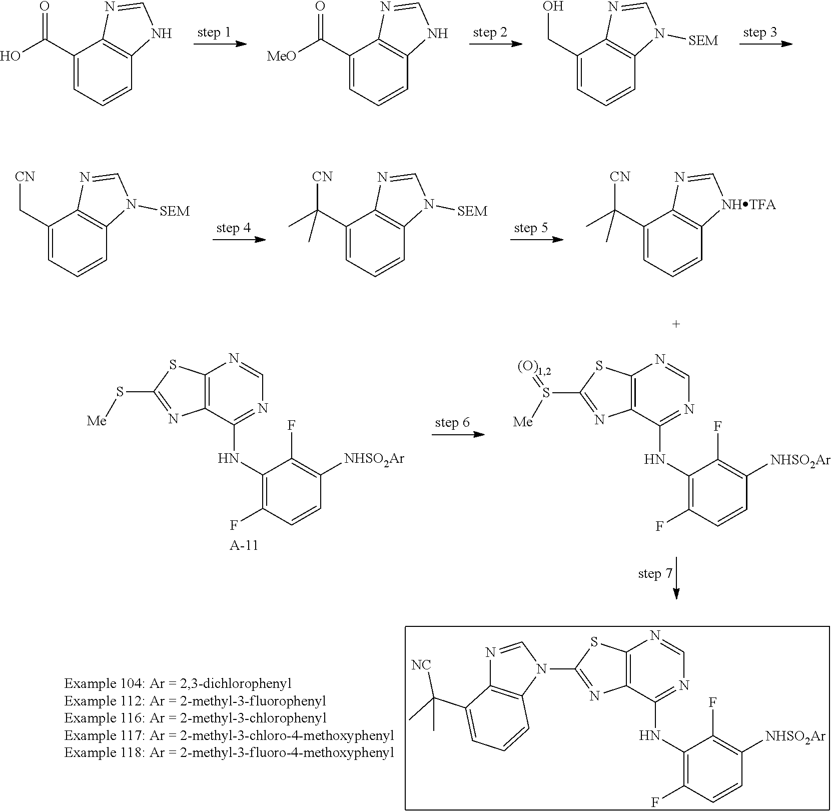

Step 1: To a 20 mL vial was added 1H-benzimidazole-4-carboxylic acid (1.00 g, 6.17 mmol) in MeOH (6.2 mL). Sulfuric acid (723 μL, 13.6 mmol) was then added. The reaction was heated in an oil bath at 70° C. for 24 hours. It was allowed to cool to room temperature. The solvent was removed under reduced pressure. The crude material was taken up in a saturated aqueous solution of NaHCO₃ and extracted three times with EtOAc. The combined organic layers were washed with water then brine, dried over MgSO₄, filtered and then concentrated under reduced pressure to afford methyl 1H-benzimidazole-4-carboxylate (895 mg, 5.08 mmol, 82% yield) as a brown solid which was used without further purification. MS m/z 177.2 (MH⁺).

Step 2: To a 250 mL round-bottom flask was added methyl 1H-benzimidazole-4-carboxylate (750 mg, 4.26 mmol) from the previous step and 2-(trimethylsilyl)ethoxymethyl chloride, stabilized, tech. (982 μL, 5.53 mmol) in anhydrous THF (21 mL) under a N₂ atmosphere. The mixture was cooled to 0° C. then a 1M THF solution of lithium bis(trimethylsily)amide (5.5 mL, 5.5 mmol) was slowly added to the reaction over the course of 20 min. The reaction was warmed to room temperature and left to stir overnight. The reaction was cooled to 0° C. and then a 1M THF solution of lithium aluminum hydride (5.1 mL, 5.1 mmol) was added. The reaction was warmed to room temperature and left to stir overnight. The reaction was quenched with MeOH at 0° C. It was then adsorbed onto Celite® and purified by normal-phase silica gel chromatography using a 0% to 10% IPA in EtOAc gradient. The desired fractions were collected and concentrated under reduced pressure to afford [1-(2-trimethylsilylethoxymethyl)benzimidazol-4-yl]methanol (461 mg, 39% yield) as a light brown solid. MS m/z 279.2 (MH⁺). ¹H NMR (400 MHz, DMSO-d₆) δ: 8.31 (s, 1H), 7.50 (d, J=9.0 Hz, 1H), 7.24-7.32 (m, 2H), 5.63 (s, 2H), 5.14 (t, J=5.5 Hz, 1H), 4.92 (d, J=5.5 Hz, 2H), 3.48 (t, J=8.0 Hz, 2H), 0.83 (t, J=8.0 Hz, 2H), −0.09 (s, 9H).

Step 3: In a 20 mL vial was added [1-(2-trimethylsilylethoxymethyl)benzimidazol-4-yl]methanol (200 mg, 0.72 mmol) from the previous step and DIPEA (500 μL, 2.87 mmol) in MeCN (8 mL). methanesulfonyl chloride (167 μL, 2.16 mmol) was then added. The reaction was allowed to stir at room temperature overnight. The reaction was quenched with the addition of a saturated aqueous solution of NaHCO₃ and then extracted with EtOAc (×3). The combined organic layers were dried over MgSO₄, filtered and then concentrated under reduced pressure.

The crude material was dissolved in DMF (8 mL) and then potassium cyanide (187 mg, 2.87 mmol) was added. The reaction was allowed to stir at room temperature overnight again. The reaction was quenched with the addition of a 10% aqueous solution of LiCl and then extracted with EtOAc (×3). The combined organic layers were dried over MgSO₄, filtered and then concentrated under reduced pressure. The crude material was adsorbed onto Celite® and then purified by normal-phase silica gel chromatography using a 0% to 100% EtOAc in hexanes gradient. The desired fractions were collected and concentrated under reduced pressure to afford 2-[1-(2-trimethylsilylethoxymethyl)benzimidazol-4-yl]acetonitrile (149 mg, 72% yield) as a clear film. MS m/z 288.2 (MH⁺).

Step 4: Sodium hydride (60 mg, 1.50 mmol) was added to 4 mL of DMF in a 20 mL vial. The mixture was cooled to 0° C. and then a solution of 2-[1-(2-trimethylsilylethoxymethyl)benzimidazol-4-yl]acetonitrile (177 mg, 0.616 mmol) from the previous step in 4 mL of DMF was slowly added. Upon addition, the reaction turned an orange colour. The reaction was warmed to room temperature. After −30 min, the reaction turned a darker orange colour. The reaction was cooled to 0° C. and then iodomethane (115 μL, 1.85 mmol) was added. The reaction turned a lighter orange colour upon addition. The reaction was warmed to room temperature and left to stir overnight. The reaction was quenched with a 1M aqueous solution of HCl and then extracted with EtOAc (×3). The combined organic layers were washed with water then brine, dried over MgSO₄, filtered and then concentrated under reduced pressure. The crude material was adsorbed onto Celite® and purified by normal-phase silica gel chromatography using a 0% to 100% EtOAc in hexanes gradient. The desired fractions were collected and concentrated under reduced pressure to afford 2-methyl-2-[1-(2-trimethylsilylethoxymethyl)benzimidazol-4-yl]propanenitrile (134 mg, 69% yield) as an orange-brown oil. MS m/z 316.2 (MH⁺).

Step 5: To a 20 mL scintillation vial was added 2-methyl-2-[1-(2-trimethylsilylethoxymethyl)benzimidazol-4-yl]propanenitrile (134 mg, 0.426 mmol) from the previous step followed by DCM (2 mL) and TFA (2 mL). The reaction was left to stir at room temperature overnight. The solvent was removed under reduced pressure. The material was dissolved in MeOH and then Amberlite IRA-67 resin was added. The resin was filtered out through a cotton plug. The organics were collected and concentrated under reduced pressure to afford 2-(1H-benzimidazol-4-yl)-2-methyl-propanenitrile; 2,2,2-trifluoroacetic acid (123 mg, 96% yield) as a clear film. MS m/z 186.0 (MH⁺).

Step 6: Preparation of the methylsulfone and methylsulfoxide mixture from an appropriate intermediate A-11 was performed as described above in Method B for Example 28 (step 1).

Step 7: Preparation of Examples 104,112,116-118 was performed by coupling the benzimidazole described in step 5 to the crude methylsulfone/methylsulfoxide mixture from A-11 (possessing an appropriate Ar group) using DIPEA in DMSO as described for Example 4 (step 7) in general method A.

### Preparation of Example 105

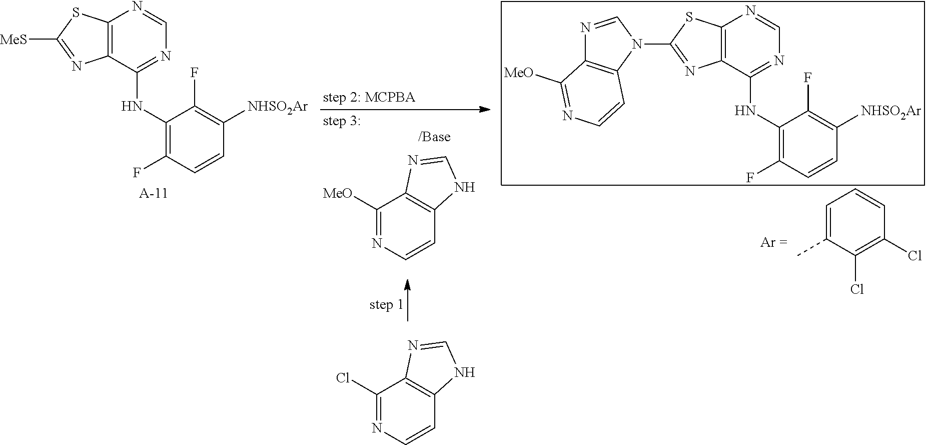

Step 1: Preparation of 4-methoxy-1H-imidazo[4,5-c]pyridine: In a pressure resistant tube, sodium hydride (104 mg, 2.60 mmol) was slowly added at 0° C. to 3.3 mL of MeOH. The resulting mixture was stirred for 5 min and commercial 4-chloro-1H-imidazo[4,5-c]pyridine (0.10 g, 0.65 mmol) was added. The tube was sealed and the reaction mixture was heated at 120° C. and stirred for 12 hours. After cooling to room temperature, the resulting mixture was concentrated under reduced pressure. The crude product was purified by silica gel chromatography using a 0% to 10% MeOH in DCM gradient. Affords 4-methoxy-1H-imidazo[4,5-c]pyridine (51 mg, 53% yield) as a tan solid. MS m/z 150.2 (MH⁺).

Step 2: Preparation of the methylsulfone and methylsulfoxide mixture from intermediate A-11 (Ar=2,3-dichlorophenyl) was performed as described above in Method B for Example 28 (step 1).

Step 3: Preparation of Example 105 was performed by coupling 4-methoxy-1H-imidazo[4,5-c]pyridine described in step 1 to the crude methylsulfone/methylsulfoxide mixture from A-11 (Ar=2,3-dichlorophenyl) using cesium carbonate in DMSO in a fashion analogous to that employed for Example 4 (step 7) in general method A.

### Preparation of Example 108

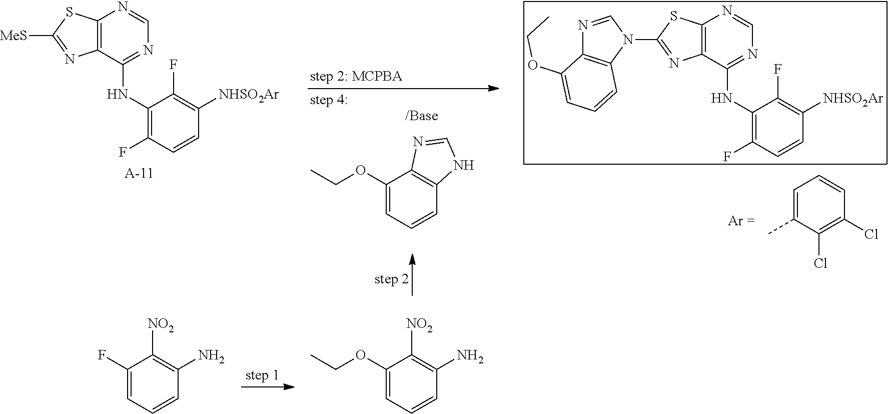

Step 1: Preparation of 3-ethoxy-2-nitro-aniline: A solution of sodium ethoxide (21% in EtOH, 1.63 mL, 4.36 mmol) was added to a solution of 3-fluoro-2-nitro-aniline (0.23 g, 1.45 mmol) in EtOH (8 mL). The resulting mixture was stirred at 80° C. for 10 h. The reaction mixture was concentrated and water was added. The aqueous mixture was extracted with EtOAc. The organic layers were combined, washed with brine, dried with Na₂SO₄, filtered and concentrated. The crude product was purified by column chromatography (silica gel, 0-100% EtOAc in hexanes) to afford 3-ethoxy-2-nitro-aniline (0.25 g, 94% yield). MS m/z 183.0 (MH⁺). ¹H NMR (400 MHz, DMSO-d₆) δ: 7.10 (t, J=8.4 Hz, 1H), 6.42 (dd, J=8.4, 1.0 Hz, 1H), 6.28 (dd, J=8.2, 1.2 Hz, 1H), 5.94 (br. s, 2H), 4.03 (q, J=6.9 Hz, 2H), 1.25 (t, J=7.0 Hz, 3H).

Step 2: Preparation of 4-ethoxy-1H-benzimidazole: Iron (0.37 g, 6.70 mmol) and ammonium chloride (0.36 g, 6.70 mmol) were added to a mixture of 3-ethoxy-2-nitro-aniline (0.24 g, 1.34 mmol) from the previous step in iPrOH (4.0 mL) and formic acid (1.9 mL, 49.6 mmol). The resulting mixture was heated at 90° C. and stirred for 10 h. The reaction mixture was cooled down to rt and filtered through Celite®. The solution was concentrated and the crude product was purified by column chromatography (silica gel, 0-10% MeOH in DCM) to afford 4-ethoxy-1H-benzimidazole (133 mg, 61% yield) as an off-white solid. MS m/z 163.0 (MH⁺). ¹H NMR (400 MHz, DMSO-d₆) δ: 7.98-8.10 (m, 1H), 7.97-8.21 (m, 2H), 7.01-7.11 (m, 1H), 6.70 (d, J=7.4 Hz, 1H), 4.23 (q, J=7.0 Hz, 2H), 1.27-1.50 (m, 3H).

Step 3: Preparation of the methylsulfone and methylsulfoxide mixture from intermediate A-11 (Ar=2,3-dichlorophenyl) was performed as described above in Method B for Example 28 (step 1).

Step 4: Preparation of Example 108 was performed by coupling the benzimidazole described in step 2 to the crude methylsulfone/methylsulfoxide mixture from A-11 (Ar=2,3-dichlorophenyl) using cesium carbonate in DMSO in a fashion analogous to that employed for Example 4 (step 7) in general method A.

### Preparation of Example 110

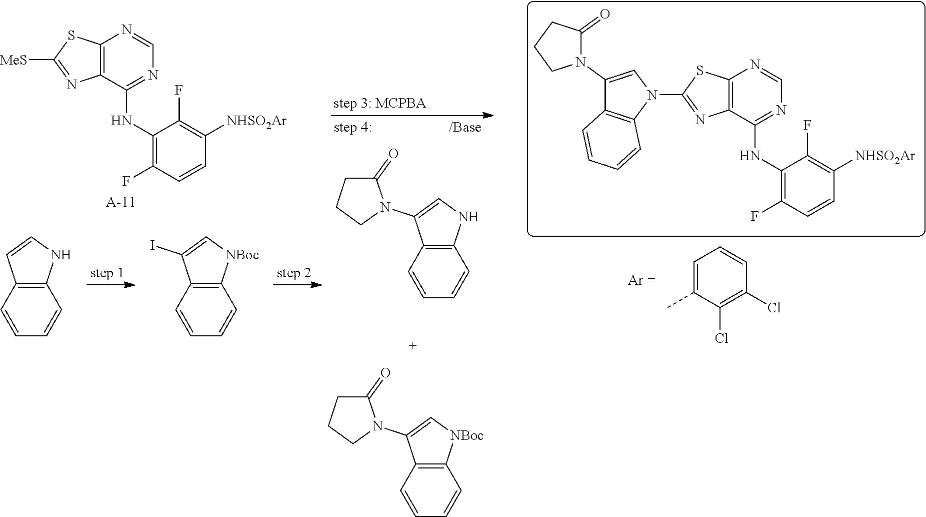

Step 1: Preparation of tert-butyl 3-iodoindole-1-carboxylate: Indole (1.00 g, 8.54 mmol) was dissolved in 26 mL of DMF in a 200 mL flask. Iodine (2.38 g, 9.39 mmol) and potassium hydroxide (1.20 g, 21.3 mmol) were then added. The reaction was stirred at room for 5 hours. It was then added to 100 mL of a saturated aqueous solution of Na₂SO₃. The mixture was then extracted with EtOAc three times. The combined organic layers were washed with water then brine, dried over MgSO₄, filtered and then concentrated under reduced pressure. To this crude material was added 43 mL of THF and 4-dimethylaminopyridine (10 mg, 0.09 mmol) followed by di-tert-butyl dicarbonate (2.05 g, 9.39 mmol). The resulting mixture was stirred at room temperature for 18 hours then purified by silica gel chromatography using a 0% to 75% EtOAc in hexanes gradient. After pooling and concentrating the appropriate fractions, tert-butyl 3-iodoindole-1-carboxylate (2.79 g, 95% yield) was obtained as a brown oil. ¹H NMR (400 MHz, DMSO-d₆) δ: 8.07 (d, J=8.2 Hz, 1H), 7.89 (s, 1H), 7.39-7.45 (m, 1H), 7.32-7.39 (m, 2H), 1.62 (s, 9H).

Step 2: Preparation of 1-(1H-indol-3-yl)pyrrolidin-2-one and tert-butyl 3-(2-oxopyrrolidin-1-yl)indole-1-carboxylate: A flask was loaded with tert-butyl 3-iodoindole-1-carboxylate (100 mg, 0.29 mmol), 2-pyrrolidinone (74 mg, 0.87 mmol), cesium carbonate (0.285 g, 0.87 mmol, Cul (28 mg, 0.15 mmol) and N,N″-dimethylethylenenediamine (31 μL, 0.29 mmol) in dioxane (1.5 mL).

The resulting mixture was heated at 80° C. and stirred for 22 h. The reaction mixture was filtered through Celite® and the crude product was purified by column chromatography (silica gel, 0-100% EtOAc in hexanes followed by a 0-10% methanol in EtOAc gradient) to afford 1-(1H-indol-3-yl)pyrrolidin-2-one (11.9 mg, 20% yield) as a white solid (MS m/z 201.2) and tert-butyl 3-(2-oxopyrrolidin-1-yl)indole-1-carboxylate (59 mg, 68% yield) as a white solid. MS m/z 301.2 (MH⁺).

Step 3: Preparation of the methylsulfone and methylsulfoxide mixture from intermediate A-11 (Ar=2,3-dichlorophenyl) was performed as described above in Method B for Example 28 (step 1).

Step 4: Preparation of Example 110: 1-(1H-indol-3-yl)pyrrolidin-2-one (10.9 mg, 0.05 mmol) described in step 2 was charged in a 4 mL vial along with the crude methylsulfone/methylsulfoxide mixture from A-11 (Ar=2,3-dichlorophenyl) (51 mg, 0.09 mmol) and DMSO (1 mL). DIPEA (47 μL, 0.27 mmol) was added and the mixture was stirred at 105° C. overnight. The mixture was cooled to room temperature then sodium bis(trimethylsilyl)amide (68.131 uL, 0.07 mmol) was added as a 1M solution in THF. The resulting mixture was stirred at 105° C. for a total of 3 days. The mixture was cooled to room temperature then quenched with 0.2 mL of glacial acetic acid. The product was isolated by prep HPLC using a 55% to 85% methanol in water gradient (with 0.1% formic acid modifier) over 15 minutes. After lyophilization from a water and acetonitrile mixture, 2,3-dichloro-N-[2,4-difluoro-3-[[2-[3-(2-oxopyrrolidin-1-yl)indol-1-yl]thiazolo[5,4-d]pyrimidin-7-yl]amino]phenyl]benzenesulfonamide (Example 110; 10.7 mg, 33% yield) was obtained as a beige solid.

### Preparation of Examples 111, 119 and 131

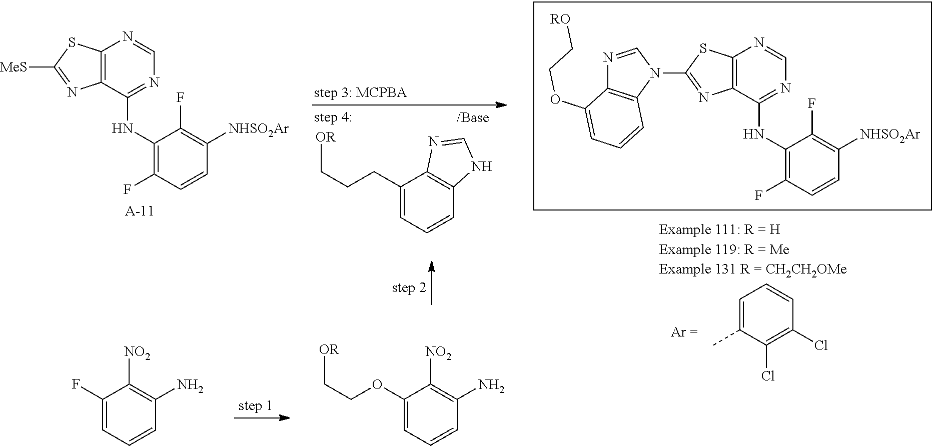

Step 1 (R=Me for Example 119): Potassium carbonate (0.35 g, 2.56 mmol) and 2-methoxyethanol (0.40 mL, 5.12 mmol) were added to a solution of 3-fluoro-2-nitro-aniline (0.10 g, 0.641 mmol) in DMF (3.2 mL). The resulting mixture was stirred at 80° C. for 10 h. Water was added, and the aqueous mixture was extracted with EtOAc. The organic layers were combined, washed with brine, dried with Na₂SO₄, filtered and concentrated. The crude product was purified by column chromatography (silica gel, 0-100% EtOAc in hexanes) to afford 3-(2-methoxyethoxy)-2-nitro-aniline (52 mg, 38% yield). MS m/z 213.1 (MH⁺).

Step 2: Preparation of the substituted benzimidazoles from the 2-nitroanilines described in step 1 was performed using the “one-pot” procedure described in Method G for Example 113 (step 2).

Step 3: Preparation of the methylsulfone and methylsulfoxide mixture from intermediate A-11 (Ar=2,3-dichlorophenyl) was performed as described above in Method B for Example 28 (step 1).

Step 4: Preparation of Examples 111, 119 and 131 was performed by coupling the appropriate substituted benzimidazole from step 2 to the crude methylsulfone/methylsulfoxide mixture from A-11 (Ar=2,3-dichlorophenyl) using either DIPEA (Example 111) or cesium carbonate (Examples 119 and 131) in DMSO in a fashion analogous to that employed for Example 4 (step 7) in general method A.

### Preparation of Example 120

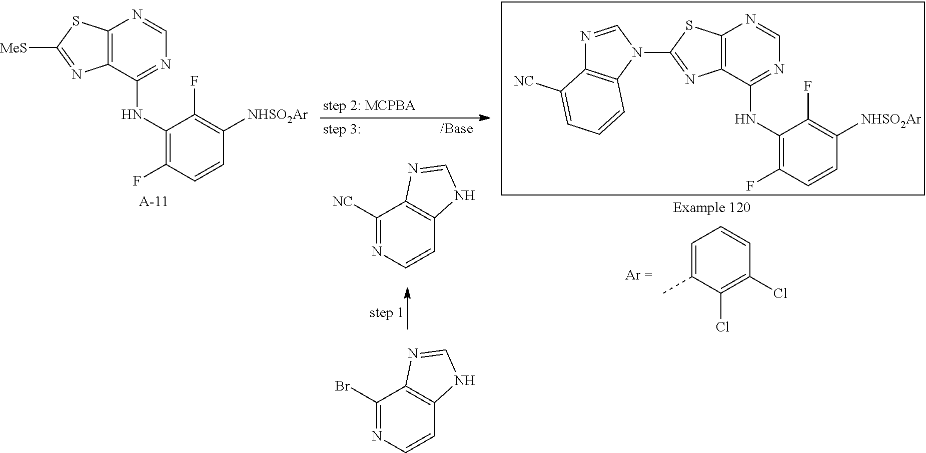

Step 1: Preparation of 1H-benzo[d]imidazole-4-carbonitrile: To a nitrogen-purged (×3) 10 mL microwave vial equipped with a magnetic stir bar, was added 4-bromo-1H-benzo[d]imidazole (99 mg, 0.502 mmol), dicyanozinc (70.8 mg, 0.603 mmol), palladium tetrakistriphenylphosphine (116 mg, 0.100 mmol) in DMF (4 mL). The reaction was heated to 90° C. and allowed to stir for 16 h. The reaction was cooled to room temperature, diluted with water and then extracted with EtOAc. The organic layers were washed with NaHCO₃, brine, dried over Na₂SO₄ and then loaded on Celite®. The crude material was purified by silica gel chromatography using a MeOH in DCM gradient to afford 1H-benzo[d]imidazole-4-carbonitrile (31 mg, 43% yield) as a light pink solid. ¹H NMR (400 MHz, DMSO-d₆) δ: 13.04 (br. s., 1H), 8.45 (s, 1H), 7.90 (d, J=7.83 Hz, 1H), 7.68 (d, J=7.83 Hz, 1H), 7.20-7.44 (m, 1H). MS m/z 142.2 (MH⁺).

Step 2: Preparation of the methylsulfone and methylsulfoxide mixture from intermediate A-11 (Ar=2,3-dichlorophenyl) was performed as described above in Method B for Example 28 (step 1).

Step 3: Preparation of Examples 111, 119 and 131 was performed by coupling 1H-benzo[d]imidazole-4-carbonitrile from step 1 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Example 122

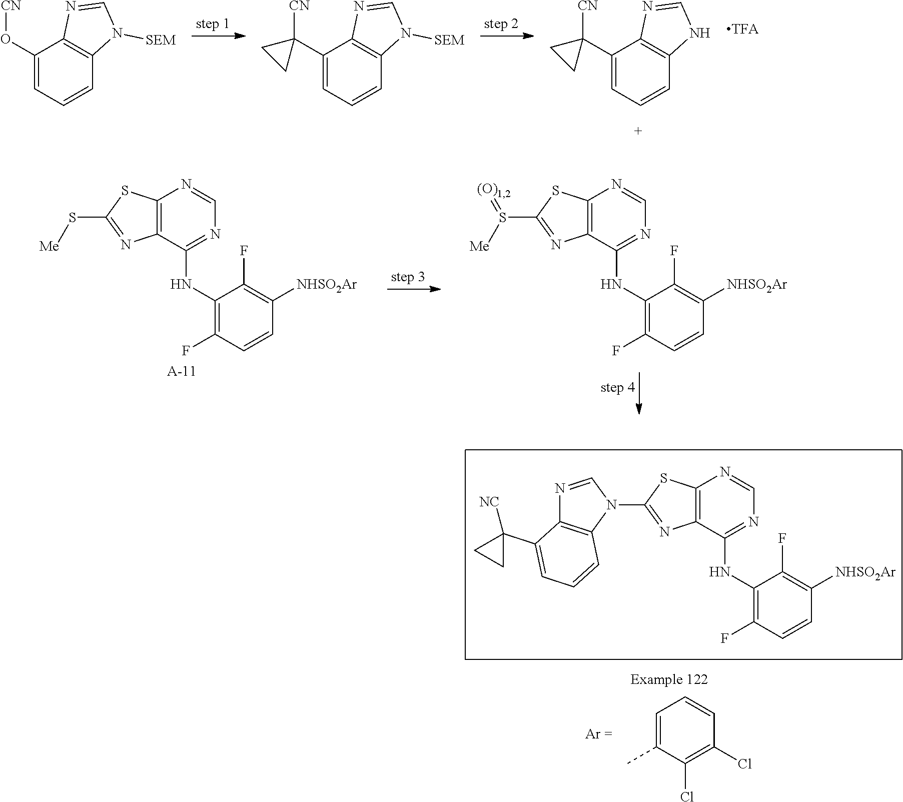

Step 1: The synthesis of 2-[1-(2-trimethylsilylethoxymethyl)benzimidazol-4-yl]acetonitrile was described for the synthesis of Examples 104, 112, 116-118. Sodium hydride (100 mg, 2.51 mmol) was added to anhydrous DMF (5 mL) in a 20 mL vial. The mixture was cooled to 0° C. 2-[1-(2-trimethylsilylethoxymethyl)benzimidazol-4-yl]acetonitrile (240 mg, 0.84 mmol) in 5 mL of DMF was added slowly. The reaction was warmed to room temperature then was cooled back down to 0° C. 1,2-dibromoethane (76 μL, 0.878 mmol) and sodium iodide (138 mg, 0.92 mmol) were added. The reaction was warmed to room temperature and then heated to 105° C. The reaction mixture was left to stir at that temperature for 24 hours. The reaction was quenched with 1M HCl at room temperature and then extracted with EtOAc three times. The combined organic layers were washed with water then brine, dried over MgSO₄, filtered and then concentrated under reduced pressure. The crude material was adsorbed onto Celite® and then purified by normal-phase silica gel chromatography using a 0% to 100% EtOAc in hexanes gradient. The desired fractions were collected and concentrated under reduced pressure to afford 1-[1-(2-trimethylsilylethoxymethyl)benzimidazol-4-yl]cyclopropanecarbonitrile (111 mg, 42% yield) as a light brown oil. MS m/z 314.2 (MH⁺).

Step 2: 1-[1-(2-trimethylsilylethoxymethyl)benzimidazol-4-yl]cyclopropanecarbonitrile (118 mg, 0.376 mmol) was dissolved in a mixture of DCM (2 mL) and TFA (2 mL) in a 20 mL vial. The reaction was left to stir at room temperature overnight. The solvent was removed under reduced pressure. The material was dissolved in MeOH and then Amberlite IRA-67 resin was added. The resin was filtered out and then the filtrate was concentrated under reduced pressure to afford 1-(1H-benzimidazol-4-yl)cyclopropanecarbonitrile; 2,2,2-trifluoroacetic acid (114 mg, 102% yield) as a light brown, sticky solid. MS m/z 184.0 (MH⁺).

Step 3: Preparation of the methylsulfone and methylsulfoxide mixture from intermediate A-11 (Ar=2,3-dichlorophenyl) was performed as described above in Method B for Example 28 (step 1).

Step 4: Preparation of Example 122 was performed by coupling 1-(1H-benzimidazol-4-yl)cyclopropanecarbonitrile; 2,2,2-trifluoroacetic acid from step 2 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Example 123

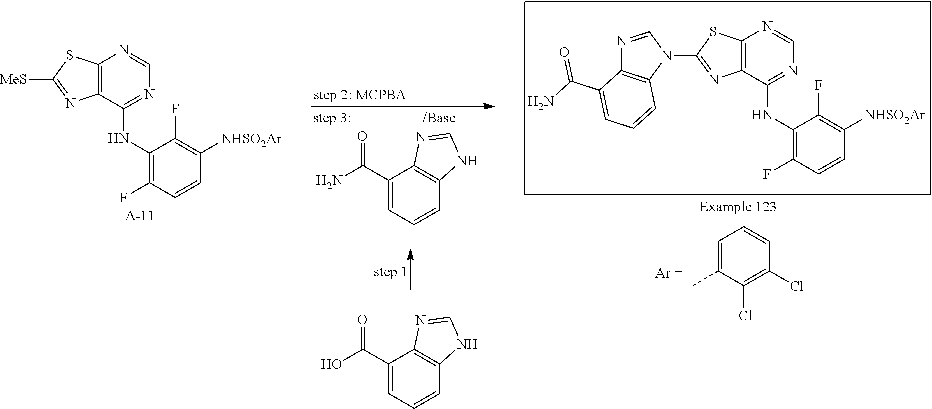

Step 1: To a 100 mL round-bottom flask equipped with a magnetic stir bar was added DMF (25 mL) followed by 1H-benzo[d]imidazole-7-carboxylic acid (1.018 g, 6.28 mmol), EDCI (2.407 g, 12.56 mmol), HOBT-H₂O (1.923 g, 12.56 mmol) and triethylamine (1.750 ml, 12.56 mmol). The reaction was cooled to 0° C. and allowed to stir for 2 h. Next, concentrated ammonium hydroxide (2 ml, 29.6 mmol) was added and the reaction was allowed to warm to room temperature and stirred for 24 hours. The reaction was diluted with water and extracted with EtOAc. The organic layer was washed with brine then dried over Na₂SO₄, filtered and then concentrated under reduced pressure. The crude material was adsorbed onto Celite® and purified by silica gel chromatography using a 0% to 15% ethanol in ethyl acetate gradient to afford 1H-benzo[d]imidazole-7-carboxamide (281.8 mg, 1.749 mmol, 27.9% yield) as an off-white solid. MS m/z 160.1 (M-H⁻). ¹H NMR (400 MHz, DMSO-d₆) δ: 12.93 (br. s., 0.7H), 12.41 (br. s., 0.3H), 9.28 (br. s., 1H), 8.44 (br. s., 0.7H), 8.16 (br. s., 0.3H), 7.85 (d, J=5.1 Hz, 1H), 7.76 (d, J=7.4 Hz, 1H), 7.69 (br. s., 1H), 7.33 (br. s., 1H).

Step 2: Preparation of the methylsulfone and methylsulfoxide mixture from intermediate A-11 (Ar=2,3-dichlorophenyl) was performed as described above in Method B for Example 28 (step 1).

Step 3: Preparation of Example 123 was performed by coupling 1H-benzo[d]imidazole-7-carboxamide from step 1 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Example 126

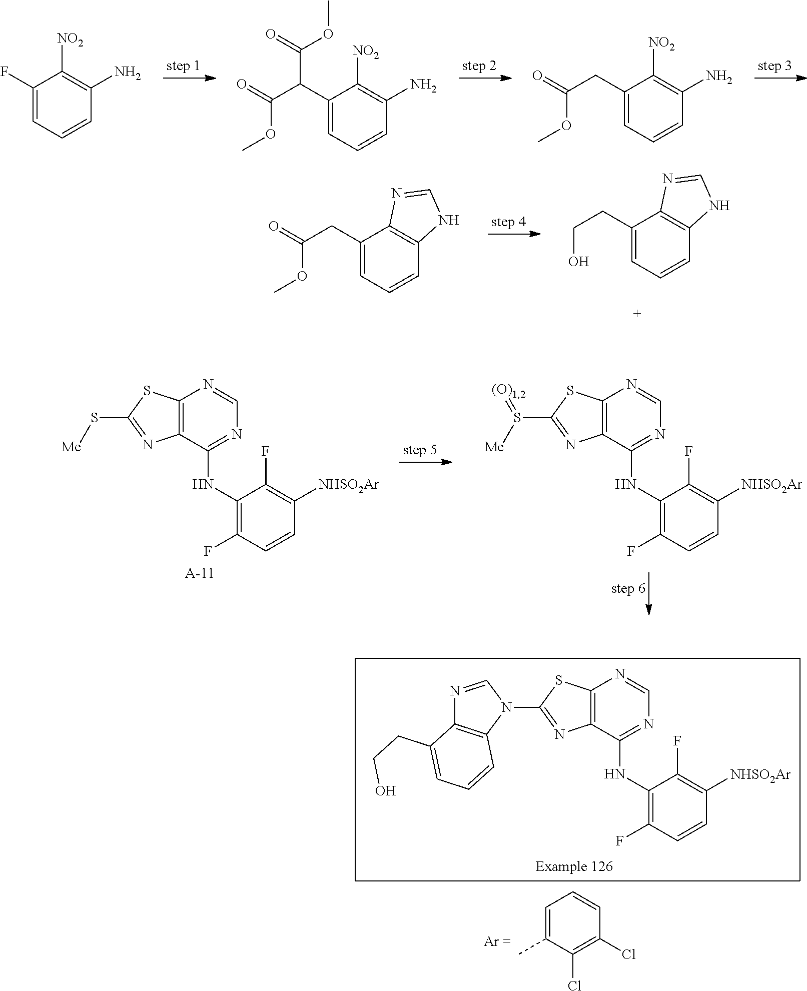

Step 1: Cesium carbonate (8.35 g, 25.6 mmol) was charged in a 50 mL flask and suspended in 6 mL of DMSO. Dimethyl malonate (2.7 mL, 30.7 mmol) was then added at room temperature. After 10 minutes of stirring the thick slurry, 3-fluoro-2-nitro-aniline (800 mg, 5.12 mmol) was added affording a bright orange to red mixture which was stirred at 70° C. for 1 hour. The mixture was allowed to cool to room temperature then diluted with EtOAc and washed with a saturated solution of ammonium chloride. The aqueous layer was extracted 3 times with EtOAc and the combined organic layers were washed with brine then dried over MgSO₄, filtered and concentrated. The residue was purified by flash chromatography on silica gel using a 5% to 50% ethyl acetate in hexanes gradient. Affords dimethyl 2-(3-amino-2-nitrophenyl)malonate (1.225 g, 89% yield): MS m/z 269.1 (MH⁺). ¹H NMR (DMSO-d₆) δ: 7.29 (dd, J=8.2, 7.4 Hz, 1H), 7.00 (dd, J=8.6, 1.2 Hz, 1H), 6.85 (s, 2H), 6.47 (dd, J=7.4, 1.2 Hz, 1H), 5.09 (s, 1H), 3.67 (s, 6H).

Step 2: Dimethyl 2-(3-amino-2-nitrophenyl)malonate (1.37 g, 5.11 mmol) and lithium chloride (260 mg, 6.13 mmol) were charged in a 40 mL vial and dissolved in 6 mL of DMSO and 0.6 mL of water. The bright red mixture was heated at 150° C. for 2.5 hours after which time the color had become a bit darker. The mixture was allowed to cool to room temperature then diluted with water and EtOAc. The biphasic mixture was filtered through a short pad of Celite® to remove insolubles creating emulsions and the layers were separated. The aqueous layer was further extracted 3 more times with EtOAc. The combined organic layers were then washed with water twice and once with brine then dried over MgSO₄, filtered and concentrated. The bright red residue was purified by flash chromatography on silica gel using a 15% to 50% EtOAc in hexanes gradient. The appropriate fractions were pooled, concentrated and dried under reduced pressure. Affords methyl 2-(3-amino-2-nitro-phenyl)acetate (749 mg, 70% yield) as a bright orange solid: ¹H NMR (DMSO-d₆) δ: 7.24 (dd, J=8.4, 7.2 Hz, 1H), 6.93 (dd, J=8.4, 1.4 Hz, 1H), 6.79 (s, 2H), 6.54 (d, J=7.0 Hz, 1H), 3.85 (s, 2H), 3.59 (s, 3H).

Step 3: Methyl 2-(3-amino-2-nitro-phenyl)acetate (545 mg, 2.59 mmol) was charged in a 100 mL flask containing a magnetic stir bar. 5% Palladium on charcoal (109 mg, 0.051 mmol) was then added and the mixture was suspended in 14 mL of methanol. Triethyl orthoformate (0.91 mL, 5.45 mmol) was added followed by 2 drops of acetic acid. The reaction flask was placed under reduced pressure then hydrogen was introduced. This operation was repeated twice more then the mixture was stirred vigorously at room temperature for 24 hours under a balloon atmosphere of hydrogen. 0.2 mL of Et₃N was added to the mixture to neutralize the AcOH then it was filtered through a short pad of Celite®, rinsing with methanol. The filtrate was concentrated to dryness then the residue was purified by flash chromatography on silica gel column using a 1:1 EtOAc/DCM to 100% EtOAc to 10% IPA in EtOAc gradient. The appropriate fractions were pooled, concentrated then dried under reduced pressure to afford methyl 2-(1H-benzimidazol-4-yl)acetate (299 mg, 61% yield). MS m/z 191.2 (MH⁺). ¹H NMR shows a 0.6 to 0.4 mixture of tautomers: ¹H NMR (DMSO-d₆) δ: 12.50 (br. s., 0.4H), 12.44 (br. s., 0.6H), 8.21 (s, 0.4H), 8.17 (s, 0.6H), 7.55 (d, J=7.4 Hz, 0.4H), 7.43 (d, J=7.8 Hz, 0.6H), 7.15 (t, J=7.4 Hz, 0.6H), 7.12 (t, J=7.8 Hz, 0.4H), 7.03-7.09 (m, 1H), 4.00 (s, 1.2H), 3.97 (s, 0.8H), 3.62 (s, 1.2H), 3.60 (s, 1.8H).

Step 4: A solution of methyl 2-(1H-benzimidazol-4-yl)acetate (40 mg, 0.21 mmol) in 2 mL of anhydrous THF was cooled in an ice/water bath and allowed to stir for 5 minutes. A 1M THF solution of lithium aluminum hydride (0.25 mL, 0.25 mmol) was then added dropwise. Some gas evolution was noticed and the clear yellow solution became a milky beige suspension. The mixture was allowed to warm to room temperature and stirred overnight. 3-4 Drops of a saturated ammonium chloride solution were added to the mixture. After 5 minutes of stirring, 100 mg of sodium sulfate decahydrate was added. After another 10 minutes of stirring, the mixture was diluted with 2-3 mL of EtOAc and filtered through a plug of Celite®, removing insoluble material. The filtrate was concentrated to a residue which was passed through a silica gel plug using a 30% IPA in EtOAc solution as eluent. The filtrate was concentrated to dryness and the resulting tan oil crystalized upon drying under reduced pressure to afford 2-(1H-benzimidazol-4-yl)ethanol (34.5 mg, 100% yield) as a tan solid which was used without further purification. MS m/z 163.0 (MH⁺).

Step 5: Preparation of the methylsulfone and methylsulfoxide mixture from intermediate A-11 (Ar=2,3-dichlorophenyl) was performed as described above in Method B for Example 28 (step 1).

Step 6: Preparation of Example 126 was performed by coupling 2-(1H-benzimidazol-4-yl)ethanol from step 4 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Example 133

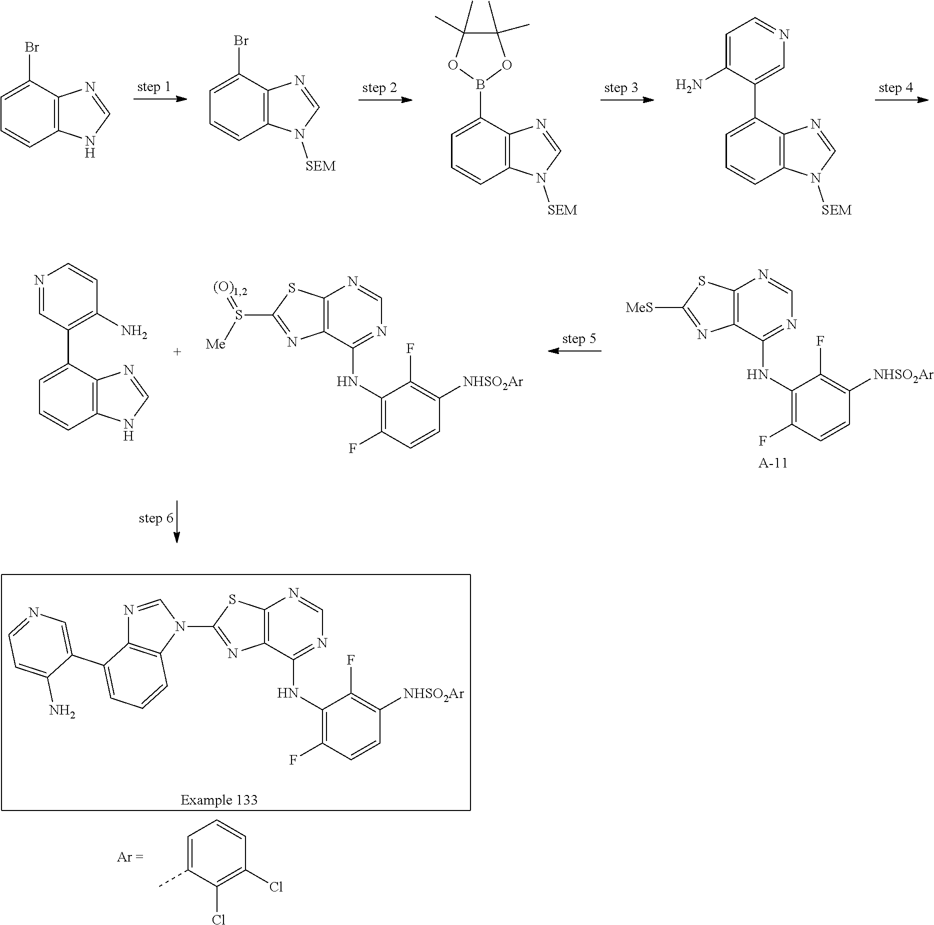

Step 1: To a 100 mL round-bottom flask, was added 4-bromo-1H-benzimidazole (1.00 g, 5.08 mmol), 2-(trimethylsilyl)ethoxymethyl chloride (1.1 mL, 6.09 mmol) and 60% NaH (244 mg, 6.09 mmol) in anhydrous DMF (10 mL). The reaction was allowed to stir at room temperature for 1 h. Upon completion, the reaction was quenched with sat. NH₄Cl and then extracted EtOAc (×3). The organics were collected washed with brine, separated then dried by MgSO₄, filtered and then concentrated under reduced pressure. The crude material was loaded onto Celite® and then purified by silica gel chromatography with EtOAc in hexanes to afford 2-[(4-bromobenzimidazol-1-yl)methoxy]ethyl-trimethyl-silane (1.18 g, 71% yield) as a brown oil. MS m/z 327.2 (MH⁺).

Step 2: To a flame-dried 5 mL microwave vial, was added 2-[(4-bromobenzimidazol-1-yl)methoxy]ethyl-trimethyl-silane (500 mg, 1.53 mmol) and bis(pinacolato)diboron (776 mg, 3.06 mmol) in DMF (0.30 mL) under a N₂ atmosphere. Next, 1,1′-bis(diphenylphosphino)ferrocene-palladium(II)dichloride dichloromethane complex (279 mg, 0.382 mmol) and potassium acetate (450 mg, 4.58 mmol) were added to the solution. The reaction was sparged with a balloon of argon for a few minutes, sealed and then heated to 100° C. and left to stir for 16 h. The reaction was cooled to room temperature. The crude reaction mixture was used as such in the next step.

Step 3: To the crude reaction mixture from step 2, were added 4-amino-3-bromopyridine (34 mg, 0.20 mmol), tetrakis(triphenylphosphine)palladium(0) (35 mg, 0.0305 mmol) and sodium carbonate (97 mg, 0.914 mmol). The reaction was sparged with a balloon of argon for ˜5-10 min, sealed and then heated to 100° C. for 24 hours. Upon completion, the reaction was cooled to room temperature. The reaction was diluted with brine and extracted with EtOAc (×3). The organic layers were collected, dried over MgSO₄, filtered and then concentrated under reduced pressure. The crude material was loaded onto Celite® and then purified on SiO₂ with MeOH in DCM to afford 3-[1-(2-trimethylsilylethoxymethyl)benzimidazol-4-yl]pyridin-4-amine (72 mg, 69% yield) as a brown film. MS m/z 341.2 (MH⁺).

Step 4: To a 20 mL scintillation vial, was added 3-[1-(2-trimethylsilylethoxymethyl)-benzimidazol-4-yl]pyridin-4-amine (72 mg, 0.210 mmol) in DCM (2 mL) and TFA (2 mL). The reaction was left to stir at room temperature for 16 h. Upon completion, the solvent was removed under reduced pressure and then dried under vacuum to afford 3-(1H-benzimidazol-4-yl)pyridin-4-amine TFA salt (68 mg, 100% yield) as a brown oil. MS m/z 211.2 (MH⁺).

Step 5: Preparation of the methylsulfone and methylsulfoxide mixture from intermediate A-11 (Ar=2,3-dichlorophenyl) was performed as described above in Method B for Example 28 (step 1).

Step 6: Preparation of Example 133 was performed by coupling 3-(1H-benzimidazol-4-yl)pyridin-4-amine TFA salt from step 4 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Example 134

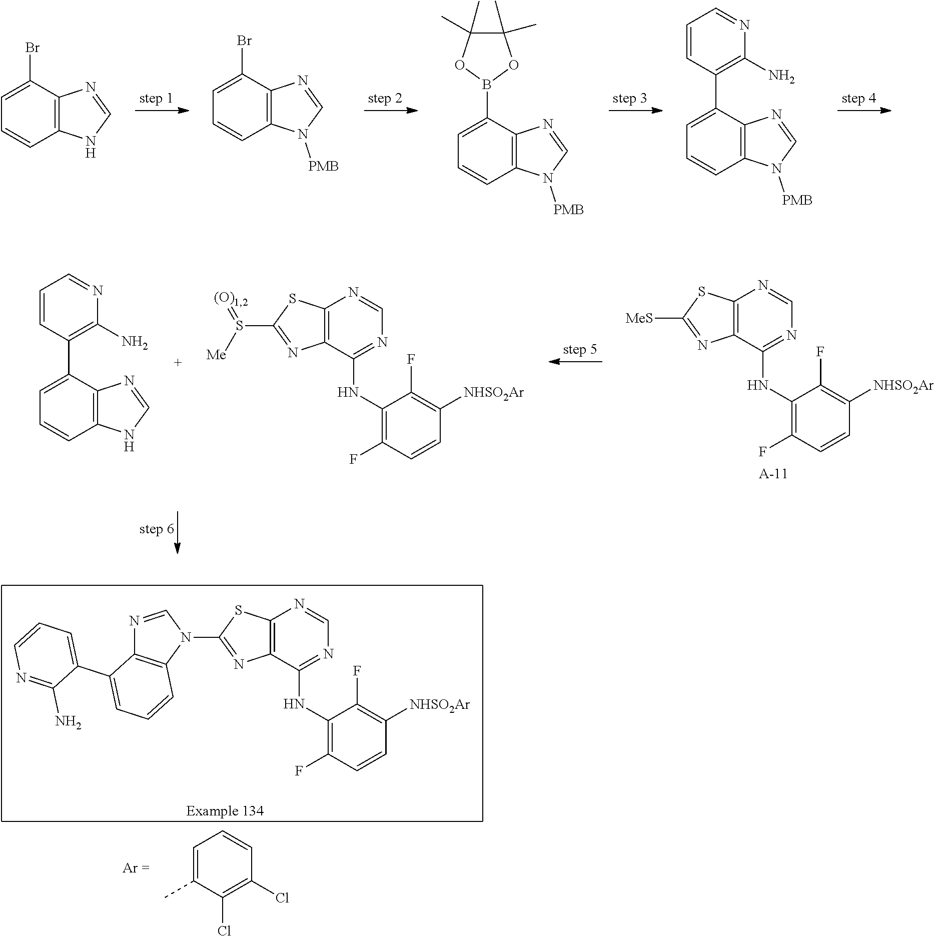

Step 1: To a 20 mL scintillation vial, was added 4-bromo-1H-benzimidazole (500 mg, 2.54 mmol), 60% NaH (122 mg, 3.05 mmol) and 4-methoxybenzyl chloride (413 μL, 3.05 mmol) in DMF (5 mL). The reaction was allowed to stir at room temperature for 2 h. The reaction was quenched with sat. NH₄Cl and then extracted with EtOAc (×3). The organics were collected, dried over MgSO₄, filtered and then concentrated under reduced pressure. The crude material was loaded onto Celite® and then purified by silica gel chromatography with EtOAc in hexanes to afford 4-bromo-1-[(4-methoxyphenyl)methyl]benzimidazole (735 mg, 91% yield) as a brown oil. ¹H NMR (400 MHz, DMSO-d₆) δ: 8.50 (s, 1H), 7.57 (dd, J=8.2, 0.8 Hz, 1H), 7.29 (ddd, J=8.6, 3.1, 2.0 Hz, 2H), 7.14 (t, J=7.8 Hz, 2H), 6.86-6.89 (m, 2H), 5.43 (s, 2H), 3.70 (s, 3H). Minor isomer: 8.46 (s, 1H), 7.70 (dd, J=8.0, 1.0 Hz, 1H), 7.42 (d, J=7.8 Hz, 2H), 7.04 (d, J=9.0 Hz, 2H), 6.90 (m, J=3.5 Hz, 2H), 5.72 (s, 2H), 3.70 (s, 3H). MS m/z 317.2 (MH⁺).

Step 2: To a flame-dried 5 mL microwave vial was added 4-bromo-1-[(4-methoxyphenyl)methyl]benzimidazole from step 1 (200 mg, 0.63 mmol) and 5 bis(pinacolato)diboron (320 mg, 1.26 mmol) in DMF (0.30 mL) under a N₂ atmosphere. Next, 1,1′-bis(diphenylphosphino)ferrocene-palladium(ii)dichloride dichloromethane complex (231 mg, 0.315 mmol) and then potassium acetate (186 mg, 1.89 mmol) was added to the solution. The reaction was sparged with a balloon of argon for a few minutes, sealed and then heated to 100° C. and left to stir for 24 h. The reaction was cooled to room temperature. The reaction was extracted diluted with brine and extracted with EtOAc (×3). The organics were collected, dried by MgSO₄, filtered through a plug of Celite® and then concentrated under reduced pressure. The crude material was loaded onto Celite® and then purified by silica gel chromatography with EtOAc in hexanes. The desired fractions were collected and concentrated under reduced pressure to afford 1-[(4-methoxyphenyl)methyl]-4-(4,4,5,5-tetramethyl-1,3,2-dioxaborolan-2-yl)benzimidazole (82 mg, 36% yield) as a brown oil. MS m/z 365.4 (MH⁺).

Step 3: To a flame-dried 5 mL microwave vial, was added 1-[(4-methoxyphenyl)methyl]-4-(4,4,5,5-tetramethyl-1,3,2-dioxaborolan-2-yl)benzimidazole (56 mg, 0.154 mmol) and 2-amino-3-bromopyridine (35 mg, 0.200 mmol) under a N₂ atmosphere. Next, sodium carbonate (49 mg, 0.461 mmol) and then tetrakis(triphenylphosphine)palladium(0) (18 mg, 0.0154 mmol) were added to the solution. The reaction was sparged with a balloon of argon for ˜5-10 min, sealed and then heated to 80° C. and left to stir 16 h. The reaction was cooled to room temperature. The reaction was diluted with brine and extracted with EtOAc (×3). The organics were collected, dried by MgSO4, filtered and then concentrated under reduced pressure. The crude material was loaded onto Celite® and then purified by silica gel chromatography with MeOH in DCM. The desired fractions were collected and concentrated under reduced pressure to afford 3-[1-[(4-methoxyphenyl)methyl]benzimidazol-4-yl]pyridin-2-amine (15 mg, 0.0445 mmol, 29% yield) as a white solid. MS m/z 331.2 (MH⁺).

Step 4: To a 20 mL scintillation vial, was added 3-[1-[(4-methoxyphenyl)methyl]-benzimidazol-4-yl]pyridin-2-amine (15 mg, 0.0445 mmol) in TFA (2 mL). The reaction was heated to 80° C. and stirred for 96 hours. The solvent was removed under reduced pressure and then the product was co-evaporated with Toluene (×3) to afford 3-(1H-benzimidazol-4-yl)pyridin-2-amine TFA salt (14 mg, 100%). MS m/z 211.2 (MH⁺).

Step 5: Preparation of the methylsulfone and methylsulfoxide mixture from intermediate A-11 (Ar=2,3-dichlorophenyl) was performed as described above in Method B for Example 28 (step 1).

Step 6: Preparation of Example 134 was performed by coupling 3-(1H-benzimidazol-4-yl)pyridin-2-amine TFA salt from step 4 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Examples 143

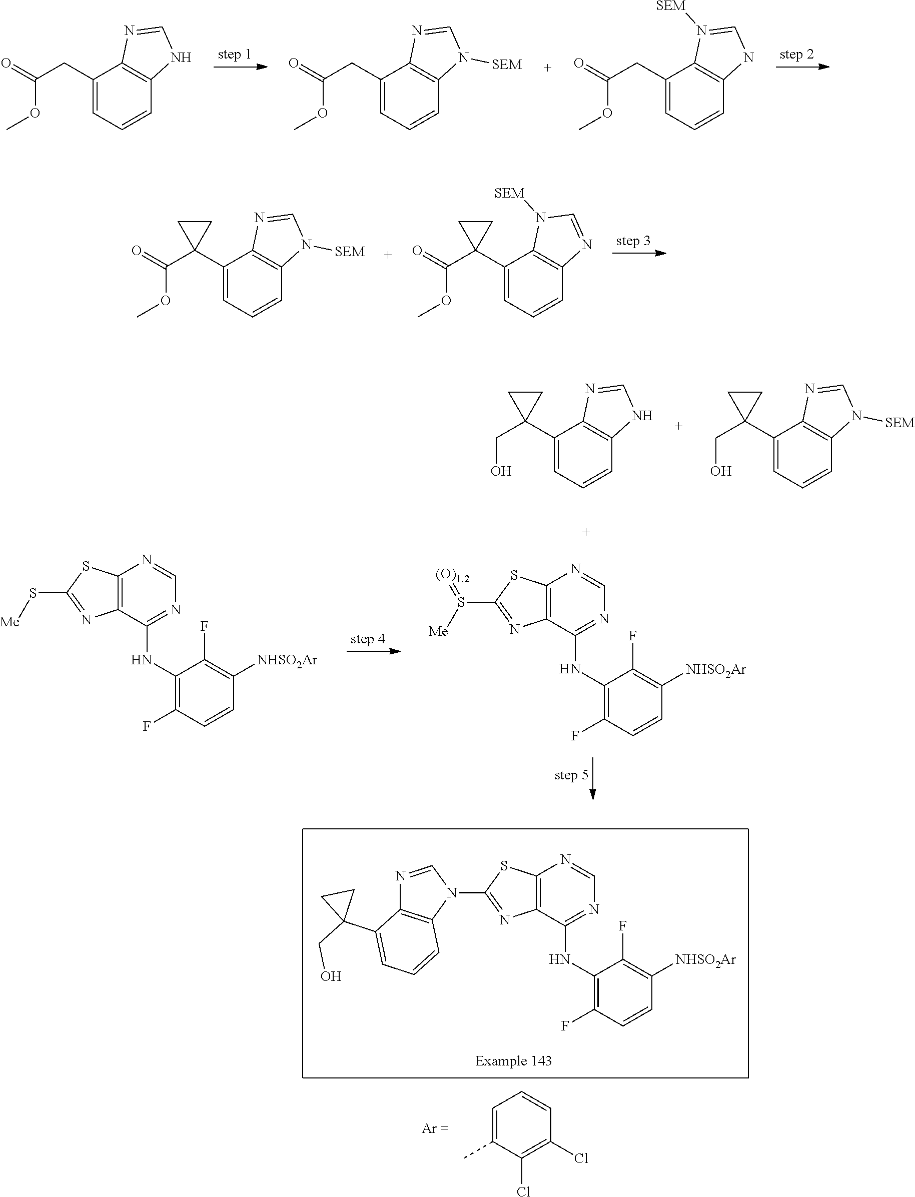

Step 1: Methyl 2-(1H-benzimidazol-4-yl)acetate prepared as described in step 3 of Example 126 (303 mg, 1.59 mmol) was dissolved in 10 mL of DMF then potassium carbonate (661 mg, 4.78 mmol) was added followed by SEM-CI (0.42 mL, 2.39 mmol) dropwise over 10 minutes at room temperature. The mixture was allowed to stir at the same temperature for 16 hours. The mixture was poured into a saturated solution of ammonium chloride then extracted 3× with EtOAc. The combined organic layers were washed with water then brine then dried over MgSO₄, filtered and concentrated. The resulting residue was purified by flash chromatography on silica gel using a 20% ethyl acetate in hexanes to 100% EtOAc gradient. The appropriate fractions were pooled, concentrated and dried under reduced pressure. Affords a mixture of methyl 2-(1-((2-(trimethylsilyl)ethoxy)methyl)-1H-benzo[d]imidazol-4-yl)acetate and methyl 2-(1-((2-(trimethylsilyl)ethoxy)methyl)-1H-benzo[d]imidazol-7-yl)acetate (260 mg, 51% yield) as a pale yellow oil.

Step 2: A mixture of methyl 2-(1-((2-(trimethylsilyl)ethoxy)methyl)-1H-benzo[d]imidazol-4-yl)acetate and methyl 2-(1-((2-(trimethylsilyl)ethoxy)methyl)-1H-benzo[d]imidazol-7-yl)acetate (265 mg, 0.83 mmol) was charged in a 25 mL flask. A solution of diphenyl(vinyl)sulfonium trifluoromethanesulfonate (479 mg, 1.32 mmol) in DMSO (4 mL) was added followed by 1,8-diazabicyclo[5.4.0]undec-7-ene (0.37 mL, 2.48 mmol) at room temperature. The mixture was stirred at that temperature for 17 hours. The reaction mixture was then diluted with water containing a bit of NH₄Cl and EtOAc. The layers were separated and the aqueous layer was extracted twice more with EtOAc. The combined organic layers were washed twice with water and once with brine. The organic layer was then dried over MgSO₄, filtered and concentrated. The resulting residue was purified by flash chromatography on silica gel using a 20% to 100% EtOAc in hexanes gradient followed by 5% IPA in EtOAc. The appropriate fractions were pooled, concentrated and dried under reduced pressure. Affords methyl 1-(1-((2-(trimethylsilyl)ethoxy)methyl)-1H-benzo[d]imidazol-4-yl)cyclopropane-1-carboxylate and methyl 1-(1-((2-(trimethylsilyl)ethoxy)methyl)-1H-benzo[d]imidazol-7-yl)cyclopropane-1-carboxylate as an inseparable mixture of isomers (250 mg, 87% yield), pale yellow oil. MS m/z 347.2 (MH⁺).

Step 3: A solution of the methyl esters from step 2 (275 mg, 0.79 mmol) in 4 mL of anhydrous THF was cooled in an ice/water bath and allowed to stir for 5 minutes. A 1M THF solution of lithium aluminum hydride (0.95 mL, 0.95 mmol) was then added dropwise. Some gas evolution was noticed. The mixture was allowed to warm to room temperature and stirred for 23 hours. Sodium sulfate decahydrate (0.7 g) was added to the mixture which was allowed to stir for another hour. The mixture was then diluted with EtOAc and filtered through a pad of Celite®, rinsing with EtOAc. The filtrate was then concentrated to dryness and the residue was purified by flash chromatography on silica gel using a 100% EtOAc to 30% IPA in EtOAc gradient.

The first product to elute is [1-[1-(2-trimethylsilylethoxymethyl)benzimidazol-4-yl]cyclopropyl]methanol (132 mg, 52% yield). MS m/z 319.2 (MH⁺). ¹H NMR (DMSO-d6) δ: 8.33 (s, 1H), 7.48 (d, J=8.2 Hz, 1H), 7.18 (t, J=7.8 Hz, 1H), 7.10 (d, J=7.4 Hz, 1H), 5.61 (s, 2H), 4.98 (t, J=5.7 Hz, 1H), 3.70 (d, J=5.5 Hz, 2H), 3.50 (t, J=8.0 Hz, 2H), 0.91 (d, J=2.7 Hz, 4H), 0.83 (t, J=8.0 Hz, 2H), −0.08 (s, 9H).

The second product to elute is [1-(1H-benzimidazol-4-yl)cyclopropyl]methanol (47 mg, 31% yield). MS m/z 186.9 (M-H⁻).

Step 4: Preparation of the methylsulfone and methylsulfoxide mixture from intermediate A-11 (Ar=2,3-dichlorophenyl) was performed as described above in Method B for Example 28 (step 1).

Step 5: Preparation of Example 143 was performed by coupling [1-(1H-benzimidazol-4-yl)cyclopropyl]methanol from step 3 to the crude methylsulfone/methylsulfoxide mixture from A-11 (Ar=2,3-dichlorophenyl) using DIPEA in DMSO in a fashion analogous to that employed for Example 4 (step 7) in general method A.

### Preparation of Examples 154

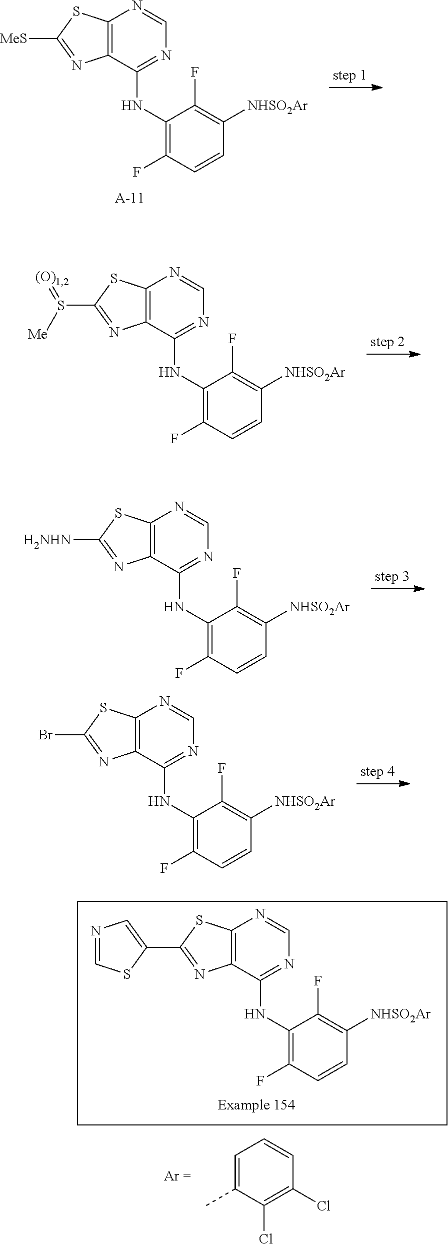

Step 1: Preparation of the methylsulfone and methylsulfoxide mixture from intermediate A-11 (Ar=2-methyl-3-fluorophenyl) was performed as described above in Method B for Example 28 (step 1).

Step 2: The methylsulfone/methylsulfoxide mixture from step 1 (82 mg, 0.16 mmol) was suspended in 1.3 mL of 95% EtOH and 0.1 mL of water. A 1M solution of hydrazine (0.77 mL, 0.77 mmol) in THF was then added. The resulting mixture was stirred at 45° C. for 2 hours. The mixture (which had become a pale beige suspension) was allowed to cool to room temperature then diluted with 10 mL of water. The solids from the resulting suspension were collected on a hardened paper filter in a Buchner funnel (very slow filtration). The solids were washed with a bit of water then dried under reduced pressure. Affords N-[2,4-difluoro-3-[(2-hydrazinothiazolo[5,4-d]pyrimidin-7-yl)amino]phenyl]-3-fluoro-2-methyl-benzenesulfonamide (71 mg, 95% yield) as an off-white solid. MS m/z 482.2 (MH⁺). ¹H NMR (400 MHz, DMSO-d6) δ:10.38 (br. s., 1H), 9.21 (s, 1H), 8.79 (s, 1H), 7.98 (s, 1H), 7.59 (d, J=7.8 Hz, 1H), 7.47 (t, J=8.6 Hz, 1H), 7.38 (td, J=7.9, 5.7 Hz, 1H), 7.17 (td, J=8.9, 5.9 Hz, 1H), 7.10 (t, J=9.0 Hz, 1H), 5.20 (s, 2H), 2.47 (d, J=2.1 Hz, 3H).

Step 3: Isopentyl nitrite (0.038 mL, 0.28 mmol) was charged in a 25 mL flask then diluted with 4 mL of acetonitrile. Copper dibromide (50.7 mg, 0.23 mmol) was then added and the dark green suspension was degassed by bubbling argon through it for 5 minutes then for another minute with sonication. N-[2,4-difluoro-3-[(2-hydrazinothiazolo[5,4-d]pyrimidin-7-yl)amino]phenyl]-3-fluoro-2-methyl-benzenesulfonamide (91 mg, 0.19 mmol) (from step 2) was then added in 4 small portions (some gas evolution was noticed) at room temperature. Once the addition was complete, the mixture was allowed to stir for 20 minutes. It was then diluted with EtOAc and washed with water containing 3-4 mL of a saturated aqueous EDTA solution. After separation of the layers, the wash was repeated twice more (until no more blue color was present in the aqueous layer). The organic layer was then washed with water and then brine. It was dried over MgSO₄, filtered and concentrated. The residue was purified by silica gel chromatography using an 80% DCM, 20% hexanes to 100% DCM gradient followed by a gradient going to 45% EtOAc in DCM. After pooling, concentrating and drying the appropriate fractions, N-[3-[(2-bromothiazolo[5,4-d]pyrimidin-7-yl)amino]-2,4-difluoro-phenyl]-3-fluoro-2-methyl-benzenesulfonamide (80 mg, 80% yield) was obtained as a white solid.

Step 4: Preparation of Example 154: N-[3-[(2-bromothiazolo[5,4-d]pyrimidin-7-yl)amino]-2,4-difluoro-phenyl]-3-fluoro-2-methyl-benzenesulfonamide (36 mg, 0.07 mmol) from step 3 was charged in 10 mL flask along with tributyl(thiazol-5-yl)stannane (38 mg, 0.10 mmol) and a magnetic stir bar. DMF (2 mL) was added and argon was bubbled through the mixture for 3-4 minutes. Tetrakis(triphenylphosphine)palladium(0) (7.8 mg, 0.007 mmol) was added. Argon was bubbled through the mixture for another 3-4 minutes with sonication. The yellow solution was immersed in an oil bath preheated to 110° C. After 6 hours of stirring at that temperature, the reaction was cooled to room temperature and acidified with a few drops of formic acid then concentrated to an oil which was diluted to 2 mL with 1:1 DMSO and methanol. The solution was filtered through a syringe filter and purified by reverse phase preparative HPLC (2 injections; 50% to 100% MeOH in water gradient, 0.1% formic acid). The appropriate fractions were pooled and concentrated. The product was lyophilized from a mixture of acetonitrile and water. Affords N-[2,4-difluoro-3-[(2-thiazol-5-ylthiazolo[5,4-d]pyrimidin-7-yl)amino]phenyl]-3-fluoro-2-methyl-benzenesulfonamide (12.7 mg, 35% yield) as an off-white solid.

### Preparation of Examples 156

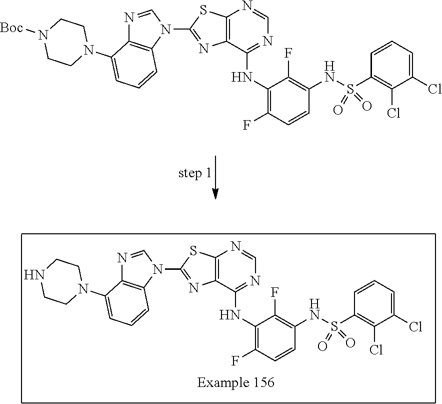 [SMILES: CC(C)(C)OC(=O)N1CCN(c2cccc3c2ncn3-c2nc3c(Nc4c(F)ccc(NS(=O)(=O)c5cccc(Cl)c5Cl)c4F)ncnc3s2)CC1 ; O=S(=O)(Nc1ccc(F)c(Nc2ncnc3sc(-n4cnc5c(N6CCNCC6)cccc54)nc23)c1F)c1cccc(Cl)c1Cl]

Step 1: Preparation of Example 156: The precursor tert-butyl 4-(1-(7-((3-((2,3-dichlorophenyl)sulfonamido)-2,6-difluorophenyl)amino)thiazolo[5,4-d]pyrimidin-2-yl)-1H-benzo[d]imidazol-4-yl)piperazine-1-carboxylate was prepared as described in general method G for Example 113 using Boc-protected piperazine for the first step. This precursor (63 mg, 0.08 mmol) was dissolved in 0.8 mL of DCM and TFA (0.061 mL, 0.80 mmol) was added at room temperature. The mixture was stirred for 20 hours then 100 mg of solid cesium carbonate was added. After stirring for a few minutes, the solids were filtered out and the filtrate was concentrated to a residue under reduced pressure and purified by reverse-phase preparative HPLC (2 injections, 50% MeOH to 100% MeOH in water gradient, 0.1% formic acid buffer) to afford 2,3-dichloro-N-[2,4-difluoro-3-[[2-(4-piperazin-1-ylbenzimidazol-1-yl)thiazolo[5,4-d]pyrimidin-7-yl]amino]phenyl]benzenesulfonamide (47 mg, 85% yield) (Example 156) as an off-white solid after lyophilisation from an acetonitrile/water mixture.

### Preparation of Example 158

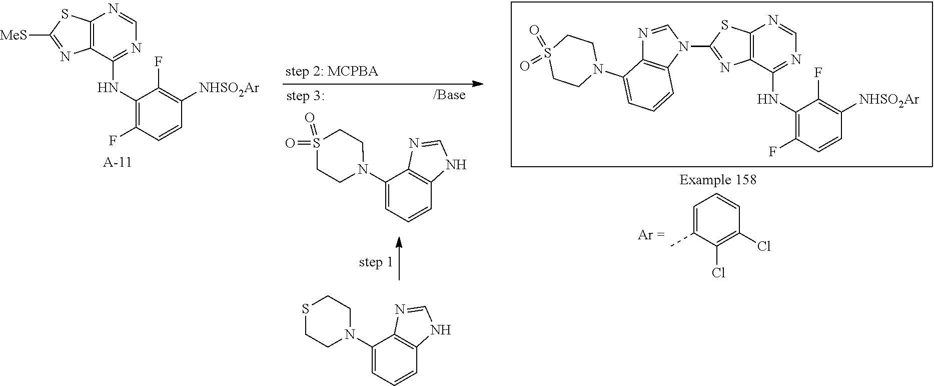

Step 1: the starting 4-thiomorpholinebenzimidazole fragment was prepared following method G. A solution of Oxone® (1.42 g, 2.32 mmol) in water (10 mL) was added to a solution of 4-(1H-benzimidazol-4-yl)thiomorpholine (0.127 g, 0.579 mmol) in methanol (20 mL). The resulting mixture was stirred at rt for 17 h. The mixture was concentrated in vacuo and the crude product was purified by column chromatography (silica gel, 0-25% MeOH in DCM) to afford 4-(1H-benzimidazol-4-yl)-1,4-thiazinane 1,1-dioxide (0.126 g, 87% yield) as a solid. MS m/z 252.2 (MH⁺).

Step 2: Preparation of the methylsulfone and methylsulfoxide mixture from intermediate A-11 (Ar=2,3-dichlorophenyl) was performed as described above in Method B for Example 28 (step 1).

Step 3: Preparation of Example 158 was performed by coupling 4-(1H-benzimidazol-4-yl)-1,4-thiazinane 1,1-dioxide from step 1 to the crude methylsulfone/methylsulfoxide mixture from A-11 (Ar=2,3-dichlorophenyl) using cesium carbonate in DMSO in a fashion analogous to that employed for Example 4 (step 7) in general method A.

### Preparation of Example 159

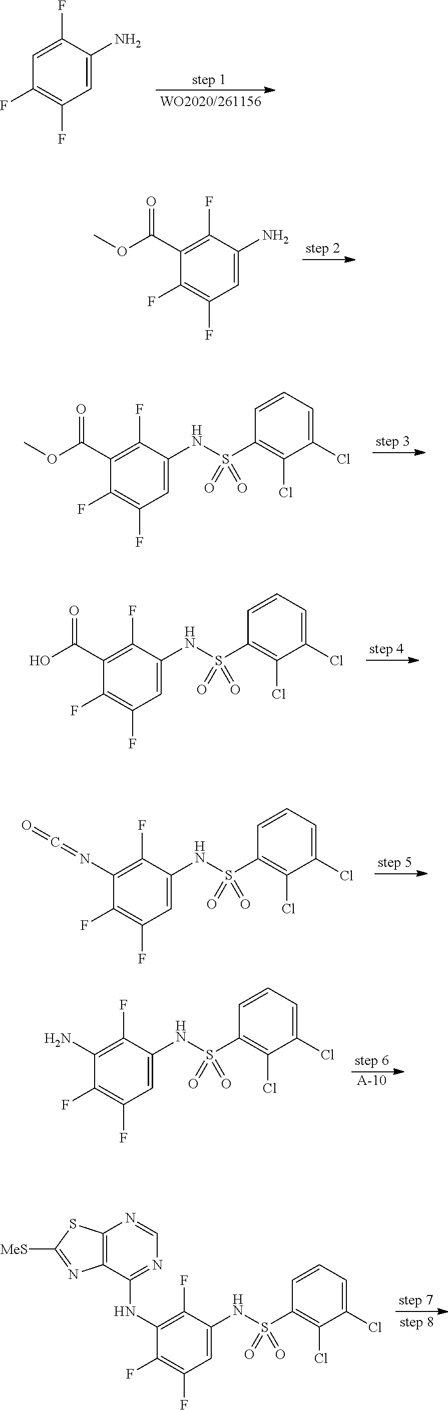

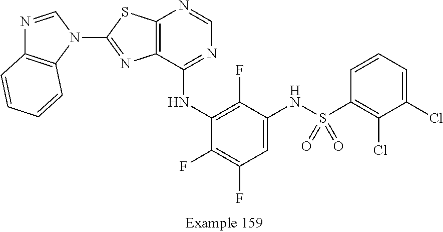 [SMILES: O=S(=O)(Nc1cc(F)c(F)c(Nc2ncnc3sc(-n4cnc5ccccc54)nc23)c1F)c1cccc(Cl)c1Cl]

Step 1: Carbomethoxylation of 2,4,5-trifluoroaniline was performed as described in patent WO2020/261156.

Step 2: To a solution of methyl 3-amino-2,5,6-trifluorobenzoate (2.72 g, 13.26 mmol) in DCE/pyridine (1:1, 16 mL) was added 2,3-dichlorobenzenesulfonyl chloride (3.91 g, 15.91 mmol) portion wise at rt. The reaction was heated to 70° C. for 16 h. The reaction was monitored by LCMS. Upon completion, it was quenched with HCl 1 M. The aqueous layer was extracted with EtOAc (15 mL) three times. The combined organic layers were washed with brine, dried over MgSO₄, filtered and evaporated to dryness. The residue was purified by chromatography on a silica gel column using 0-30% EtOAc in hexanes. The pure fractions were collected and evaporated to give methyl 3-((2,3-dichlorophenyl)sulfonamido)-2,5,6-trifluorobenzoate (5.14 g, 91% yield) as a light brown solid: MS m/z 412.0 (MH⁺).

Step 3: To a solution of methyl 3-((2,3-dichlorophenyl)sulfonamido)-2,5,6-trifluorobenzoate from step 2 (5.14 g, 12.41 mmol) in 30 mL of THF:MeOH (5:1) was added 2M KOH (37 mL, 74.5 mmol) at rt. The reaction was stirred at rt overnight. Upon completion, it was evaporated to dryness and to the residue was added water (30 mL) and diethyl ether (30 mL). The aqueous layer was washed with ether (20 mL) twice. The aqueous layer was acidified with HCl 1M to pH=2. The aqueous layer was extracted with EtOAc (30 mL) thrice. The combined organic layers were washed brine, dried over MgSO₄, filtered and evaporated to give 3-((2,3-dichlorophenyl)sulfonamido)-2,5,6-trifluorobenzoic acid (4.50 g, 91% yield) as a light orange oil. The compound was used as such in the next step. MS m/z 398.0 (MH⁺).

Step 4: To a solution of 3-((2,3-dichlorophenyl)sulfonamido)-2,5,6-trifluorobenzoic acid from step 3 (4.50 g, 11.24 mmol) in acetonitrile (30 mL) was added triethylamine (1.71 mL, 12.37 mmol) and diphenyl phosphoryl azide (2.91 mL, 13.50 mmol) at rt. The reaction was heated to 80° C. overnight. The reaction was cooled down to rt and water (30 mL) was added. The aqueous layer was extracted with EtOAc (30 mL) three times. The combined organic layers were washed with brine, dried over MgSO₄, filtered and evaporated into a dark yellow residue. The crude material was purified by chromatography on a silica gel column using 0-50% EtOAc in hexanes. The pure fractions were collected and evaporated to give 2,3-dichloro-N-(2,4,5-trifluoro-3-isocyanatophenyl)benzenesulfonamide (2.29 g, 51% yield) as a brown solid. MS m/z 391.0 (MH⁺).

Step 5: To a solution of 2,3-dichloro-N-(2,4,5-trifluoro-3-isocyanatophenyl)benzenesulfonamide from step 4 (1.35 g, 3.39 mmol) in THF (17 mL) was added aqueous LiOH 4M (17 mL). The pressure vessel was sealed and heated to 100° C. for 1 h in an oil bath. When the reaction was completed, it was quenched with a saturated NH₄Cl solution. EtOAc was added and the layers were allowed to separate. The aqueous layer was extracted with EtOAc (20 mL) two more times. The combined organic layers were washed with brine, dried over MgSO₄, filtered and evaporated to give N-(3-amino-2,4,5-trifluorophenyl)-2,3-dichlorobenzenesulfonamide (1.09 g, 86% yield) as a brown solid. The compound was carried to the next step without further purification: ¹H NMR (400 MHz, DMSO-d₆) δ: 10.59 (br. s., 1H), 7.94 (dd, J=8.2, 1.6 Hz, 1H), 7.89 (dd, J=8.2, 1.6 Hz, 1H), 7.51 (dd, J=8.0 Hz, 1H), 6.34-6.43 (m, 1H), 5.72 (s, 2H). MS m/z=369.0.

Step 6: N-(3-amino-2,4,5-trifluoro-phenyl)-2,3-dichloro-benzenesulfonamide (300 mg, 0.81 mmol) from step 5 and 7-chloro-2-methylsulfanyl-thiazolo[5,4-d]pyrimidine (A-10, 194 mg, 0.89 mmol) were added to glacial AcOH (3.2 mL). The reaction was heated at 65° C. overnight and at 85° C. for 2 hours. Another portion of 7-chloro-2-methylsulfanyl-thiazolo[5,4-d]pyrimidine (A-10, 100 mg, 0.46 mmol) was added and the mixture was stirred at 100° C. for 24 hours. It was then cooled to room temperature and concentrated to a residue under reduced pressure. Water and DCM were added and the layers separated. The aqueous layer was extracted some more with DCM. The combined organic layers were washed with brine, dried over MgSO₄, filtered and concentrated under reduced pressure to a brown solid which was carried to the next reaction without further purification. MS m/z 552.0 (MH⁺).

Step 7: To Crude 2,3-dichloro-N-[2,4,5-trifluoro-3-[(2-methylsulfanylthiazolo[5,4-d]pyrimidin-7-yl)amino]phenyl]benzenesulfonamide (238 mg, 0.43 mmol) from step 6 in DCM (5 mL) was added 3-chloroperoxybenzoic acid (149 mg, 0.862 mmol). The resulting reaction mixture was allowed to stir at room temperature overnight then diluted with EtOAc and washed with a saturated aqueous solution of NaHCO₃ followed by brine. The organic layer was dried over MgSO₄, filtered and concentrated. The resulting crude product (mostly methylsulfone, MS m/z 584.2 (MH⁺)) was carried through to the next step without further purification (66 mg, 26% yield).

Step 8: Crude 2,3-dichloro-N-[2,4,5-trifluoro-3-[(2-methylsulfonylthiazolo[5,4-d]pyrimidin-7-yl)amino]phenyl]benzenesulfonamide (66 mg, 0.11 mmol) was dissolved in 1.1 mL of DMSO.

Benzimidazole (13 mg, 0.11 mmol) and cesium carbonate (74 mg, 0.23 mmol) were added and the reaction mixture was stirred at 100° C. overnight. After cooling, the reaction mixture was filtered through a short pad of silica gel and Celite®. The filtrate was purified by reverse-phase chromatography using a methanol in water gradient with 0.1% formic acid as modifier. The appropriate fractions were pooled and concentrated. The residue was lyophilized from an acetonitrile/water mixture. Affords N-[3-[[2-(benzimidazol-1-yl)thiazolo[5,4-d]pyrimidin-7-yl]amino]-2,4,5-trifluoro-phenyl]-2,3-dichloro-benzenesulfonamide (Example 159, 12 mg, 17% yield) as a white fluffy powder.

### Biological Activity

### (a) Kinase Activity Assays for BRAF, CRAF and ARAF

Compound preparation: solid samples of each substance in 1dram vials were suspended in DMSO (Fisher Scientific) at a stock concentration of 20 mM. Stocks were kept at −20° C. and protected from light. If solubility of the compound at 20 mM appeared to be an issue, the initial concentration of the DMSO stock was changed to 10 mM or 5 mM.

In vitro enzymatic reactions were used for evaluating compounds intrinsic activity against BRAF, CRAF and ARAF. For BRAF and CRAF, 0.375 nM of purified GST-tagged kinases (cat. No. B4062-10UG and cat. No. R1656-10UG respectively from Millipore Sigma) were incubated with 75 nM of kinase-dead MEK1 substrate (cat. No. 40075; BPS Bioscience) in the presence of 10 μM of Ultrapure ATP (cat. No. V9102; Promega; part V915A) with and without the test compound in a buffer containing 50 mM HEPES pH 7.5, 10 mM MgCl2, 1 mM EDTA, 0.01% Brij-35 and 2 mM DTT. Separate reactions were performed with the MEK1 substrate and ATP as a blank control. ARAF kinase reactions were rigorously the same with the exception that kinase concentration was raised to 3.75 nM (cat. No. 1768-0000-1; Reaction Biology).

For compound treatment, 5 μL/well of test substance solution are placed in a 384-well proxyplate (Perkin Elmer) and mixed with 2× concentrated kinase reactions. The dilution series is selected so that nine concentrations cover a range from 100 nM to 0.01 nM. If necessary (if the compound exhibits low intrinsic potency) the initial concentration of 100 nM is changed to 1 μM, or 0.5 μM and further dilutions are carried out accordingly. The final concentration of DMSO in the assay is set at 0.05%.

BRAF and CRAF kinase reactions were carried out for a total of 2 hours at 30° C. and then stopped by 1/2 dilution in ADP-Glo Reagent (cat. No. V9102; Promega; part V912C). Reactions were then incubated for 1 h at room temperature before addition of one volume of Kinase Detection Reagent (cat. No. V9102; Promega; part V917A). Plates were then equilibrated at room temperature for 30 minutes before detection of luminescence on a Synergy Neo2 plate reader (Biotek). The effect of each compound dilution on BRAF and CRAF kinase activity was expressed as % inhibition and calculated as follows. First, the internal 100% inhibition control (average of luminescence in kinase reactions comprising kinase dead MEK1 substrate alone) was subtracted from each data point. The average of DMSO (vehicle) controls (set as 0% inhibition) was established and used to calculate % inhibition:

$$\% inhibition = 100 * ( 1 - ( ( Luminescence {signal}_{compound} ) / ( Luminescence {signal}_{DMSO} ) ) )$$

ARAF kinase reactions were carried out for a total of 2 hours at 30° C. and then stopped by addition of EDTA at a final concentration of 40 mM. Reactions were then detected using the AlphaLISA® SureFire® Ultra™ p-MEK 1/2 (Ser218/222) (PerkinElmer) kit. Reactions were performed with 5 μL of kinase reaction according to the manufacturer's specifications in 384-well proxyplates (Perkin Elmer) followed by overnight incubation of the AlphaLISA® reactions at room temperature in a humidified chamber. After completion of the detection reactions, the signals were recorded on a Synergy Neo2 plate reader (Biotek) equipped with AlphaLISA® filters. The effect of each compound dilution on the pMEK signal generated by ARAF reactions was expressed in % inhibition and calculated as follows. An internal 100% inhibition control (average of luminescence in kinase reactions comprising kinase dead MEK1 substrate alone) was included in each plate to measure of pMEK background and was subtracted from each data point. The average of DMSO (vehicle) controls (set as 0% inhibition) was also established and used to calculate % inhibition:

$$\% inhibition = 100 * ( 1 - ( ( pMEK {signal}_{compound} ) / ( pMEK {signal}_{DMSO} ) ) )$$

IC₅₀ values were obtained by plotting the kinase inhibition values and fitting the dose-activity curves using a log(agonist) versus response—variable slope (four parameters) function using either GraphPadPrism (V7.0) or Dotmatics Screening Ultra platform. Standards included in the ARAF kinase assay were Belvarafenib (MedChem Express cat. No. HY-109080; CAS No. 1446113-23-0), LXH254 (MedChem Express cat. No. HY-112089; CAS No. 1800398-38-2) and BGB283 (cat. No. HY-18957; CAS No. 1446090-79-4).

All substances reported here are thus BRAF, CRAF and ARAF ATP-competitive kinase inhibitors as demonstrated by direct inhibition of enzymatic activity in vitro. BRAF and CRAF inhibition potencies of compounds are listed in Tables 3 and 4 while ARAF kinase inhibition potencies of representative analogs are listed in Table A.

TABLE A
ARAF kinase inhibition results

| | Kinase IC₅₀ |
|---|---|
| | (nM) |
| Example | ARAF |
| 44 | ** |
| 122 | ** |
| Belvarafenib | * |
| BGB283 | * |
| LXH254 | * |

For the ARAF biochemical kinase assay,
* denotes an IC50 >10 nM,
** denotes a 1-10 nM IC50 range and
*** denotes an IC50 <1 nM.

### (b) General Cell Culture Methods

All cancer cell lines (A375, A101D, A2058, RKO, HT29 SK-MEL 30, IPC298, HepG2, HCT-116, Lovo, SW620, SW480, NCI-H358, NCI-H2122, Calu-6, NCIH2087, NCIH1755, NCIH1666 and Mewo) were obtained from ATCC and cultured in RPM1-1640 medium (Gibco) supplemented with 5% heat inactivated fetal bovine serum (FBS, Wisent) at 37° C. under 5% CO₂. Cells were maintained in T175 flasks (Greiner). They were passaged by removing the culture medium, washing once in 10 mL of room temperature Phosphate Buffered Saline (PBS; Wisent) and incubating at 37° C. with 2 mL of 0.05% Trypsin (Thermo-Fisher). Trypsin was then inactivated by adding complete growth medium and the cells were then replated in a T175 culture dish at the appropriate dilution. All cell lines were routinely tested for mycoplasma contamination. Tissue type and mutational status of each cell line can be found in Table B.

TABLE B
Tumor type and RAS-ERK pathway mutational status of cancer
cell lines (CCLs) used for pERK and antiproliferative
profiling of substances described in this application.

| Cell line | Tissue type | Mutational status |
|---|---|---|
| A375 | Skin | BRAF V600E |
| A101D | Skin | BRAF V600E |
| A2058 | Skin | BRAF V600E |
| RKO | Colon | BRAF V600E |
| HT29 | Colon | BRAF V600E |
| NCIH2087 | Lung | BRAF L597V; KRAS Q61K |
| NCIH1755 | Lung | BRAF G469A |
| NCIH1666 | Lung | BRAF G466V |
| SK-MEL30 | Skin | NRAS-Q61K |
| IPC298 | Skin | NRAS-Q61L |
| HepG2 | Liver | NRAS-Q61L |
| HCT-116 | Colon | KRAS G13D |
| Lovo | Colon | KRAS-G13D |
| SW620 | Colon | KRAS-G12V |
| SW480 | Colon | KRAS G12D |
| NCI-H358 | Lung | KRAS-G12C |
| NCI-H2122 | Lung | KRAS-G12C |
| Calu-6 | Lung | KRAS Q61K |
| Mewo | Skin | NF1 LOF |

(c) Measurement of Phospho-ERK Inhibition in Cultivated Human Cancer Cell Lines by the AlphaLISA® SureFire® Ultra™ p-ERK 1/2 (Thr2O2/Tyr204)

AlphaLISA® SureFire® Ultra™ p-ERK 1/2 (Thr202/Tyr204) analysis was conducted on cells plated in 100 μL of complete RPMI-1640 growth medium in 96-well flat-bottomed transparent dishes (Costar) at a density indicated in Table C. Cells were maintained overnight at 37° C. under 5% CO₂ before treatment with compounds' dilution series for one hour. The cell density in cells/cm² corresponds to cell number divided by the area of one well of a 96-well plate (0.143 cm²).

TABLE C
For each cancer cell line, number of cells plated per well.

| Cell line | Number of cells/well |
|---|---|
| A375 | 15,000 |
| NCIH2087 | 30,000 |
| NCIH1755 | 30,000 |
| NCIH1666 | 30,000 |
| SK-MEL30 | 24,000 |
| IPC298 | 20,000 |
| HepG2 | 20,000 |
| HCT-116 | 22,000 |
| Lovo | 20,000 |
| SW620 | 30,000 |
| SW480 | 15,000 |
| NCI-H358 | 20,000 |
| NCI-H2122 | 24,000 |
| Calu-6 | 15,000 |
| Mewo | 20,000 |

In a dilution series 100 μL/well of test substance dilution prepared in complete RPMI-1640 growth media was added to the cells. The dilution series is selected so that ten concentrations cover a range from 10 μM to 0.33 nM. If necessary, the initial concentration of 10 μM is increased to 100 μM or lowered to 1 μM (as in the case of A375 and H1666 cells, which are generally more sensitive to the compounds) and further dilution is carried out accordingly. The final concentration of DMSO in the assay is set at 0.5%.

After treatment, media was removed, and cells were lysed in 50 μL of 1× AlphaScreen Ultra Lysis Buffer (Perkin Elmer). The AlphaLISA® SureFire® Ultra™ p-ERK 1/2 (Thr202/Tyr204) (PerkinElmer) reactions were performed with 5 μL of cell lysate according to the manufacturer's specifications in 384-well proxyplates (Perkin Elmer) followed by overnight incubation of the reactions at room temperature in a humidified chamber. After completion of the reactions, the signals were recorded on an EnVision plate reader (Perkin Elmer) using built in AlphaLISA® settings.

The effect of each compound dilution on the pERK signal was expressed in % inhibition and calculated as follows. An internal 100% inhibition control (1 μM trametinib, cat. No. HY-10999; MedChem Express; CAS No. 871700-17-3) was included in each plate and used as a measure of pERK background. First, the value obtained for trametinib was subtracted from each data point. The average of DMSO (vehicle) controls (set as 0% inhibition) was established and used to calculate % inhibition:

$$\% inhibition = 100 * ( 1 - ( ( pERK {signal}_{compound} ) / ( pERK {signal}_{DMSO} ) ) )$$

The ability of each compound to inhibit pERK signal was expressed as an IC₅₀ value obtained by plotting the inhibition values for each data point of a dilution series and fitting the obtained curves using a log(agonist) versus response—variable slope (four parameters) function using GraphPadPrism (V7.0) or Dotmatics Screening Ultra platform.

When present, paradoxical pERK induction is inferred from the negative % inhibition values observed in the pERK IC₅₀ curves of a compound. To classify a compound as a pERK paradoxical inducer, the % inhibition of the minimal data point of the dosage-activity curve (% Y_{MIN}) was set to be lower than −20% which is considered to be within expected assay variation (e.g. a compound with % Y_{MIN}=−30% or −50% or −150% is considered to produce a paradoxical induction of the pathway while a compound that displays an IC₅₀ curve with a Y_{MIN}=−10% is considered not to produce a paradoxical activation of the pathway). Therefore, a compound was said to inhibit the pathway without paradoxical induction in a given cell line when the following criteria were met:
1. % inhibition at the highest tested dose (30 μM, 10 μM or 1 μM) exceeded 50%.
2. % Y_{MIN} of IC₅₀ curves was greater than −20%; where Y_{MIN} corresponded to the data point having the lowest value in the IC₅₀ curve of the said compound.

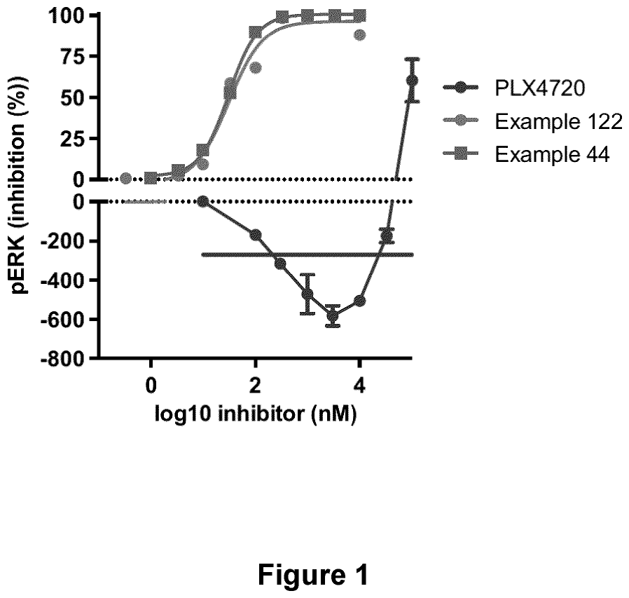

It is well known to someone skilled in the art that some variation of inhibition values is expected in such experiments. Y_{MIN} values of ±20% are considered within experimental error and are not significant. Therefore, only compounds with negative values in excess of assay variation (ca. >20%) are considered to induce a paradoxical activation of the signaling cascade and are not included within the scope of the present disclosure. FIG. 1 provides a visualization of the IC₅₀ curves for a compound that induces paradoxical pathway activation (PLX4720, commercially available from Selleck Chemicals; CAS No. 918505-84-7) and representative compounds as described herein exhibiting the unexpected and distinct induction-free profile.

FIG. 1 shows representative IC₅₀ inhibition dose response curves for compounds as described herein that do not induce paradoxical induction of pERK signaling (Y_{MIN}>−20%) in RAS-mutant HCT116 cells (Examples 44 and 122) and a compound (PLX4720) that causes strong induction of the pathway in the same cell line (Y_{MIN}˜−600%).

Significantly, the present compounds do not induce paradoxical activation of the pathway according to the criteria described above.

Example compounds 1 to 159 show pERK inhibition activity in the colon G13D Ras-mutated HCT-116 cell line as shown in Tables 3 and 4. In addition, some Examples were also shown to display paradoxical induction-free inhibition of pERK signaling in the SW480 colon cell line harboring the G12D allele of KRAS (Tables 3 and 4). Furthermore, some Examples from Tables 3 and 4 were also tested for inhibition of pERK in A375 cells that comprise the BRAF^{V600E} driver mutation and were found to be active as well (Table D-1 and D-2). All compound Examples 1-159 displayed pERK IC₅₀ values in the HCT116 cell line that were <30 μM.

Representative compounds as herein defined were also tested on additional tumor cells for their pERK inhibition activity and showed good to very good pERK inhibition activity in cancer cell lines bearing various NRAS, KRAS and NF1 alleles and representative of a large diversity of tissue types (i.e., SK-MEL 30, IPC298, HepG2, HCT-116, Lovo, SW620, SW480, NCI-H358, NCI-H2122, Calu-6 and Mewo; see Table D-1 and D-2 and refer to Table B for genotypes). The pERK inhibition activity of compounds is stronger in cancer cell lines bearing various BRAF alleles (A375, A101D, A2058, RKO, HT29, NCIH2087, NCIH1755 and NCIH1666) (Table D-1 and D-2).

TABLE D (D-1 and D-2)
Induction-free pERK IC₅₀ values and anti-proliferative EC₅₀ values for select compounds in a
panel of RAS-mutant cancer cell lines (see Table B for genotypes) and BRAF^{V600E} mutant A375.
D-1.

| | A375 | A2058 | HT-29 | RKO | A101D | SK-MEL30 | | IPC298 | | HepG2 | | HCT116 | |
|---|---|---|---|---|---|---|---|---|---|---|---|---|---|
| | pERK | pERK | pERK | pERK | pERK | pERK | | pERK | | pERK | | pERK | |
| Ex. | Prolif. | Prolif. | Prolif. | Prolif. | Prolif. | Prolif. | | Prolif. | | Prolif. | | Prolif. | |
| 44 | | | | | | +++ | (0) | +++ | (12) | +++ | (12) | ++ | (7) |
| 44 | *** | *** | *** | *** | *** | *** | | *** | | *** | | *** | |
| 122 | | | | | | +++ | (9) | ++ | (14) | ++ | (5) | ++ | (10) |
| 122 | *** | *** | *** | *** | *** | *** | | *** | | *** | | *** | |
| Belv. | | | | | | +++ | (−45) | ++ | (−63) | ++ | (−36) | ++ | (−46) |

| | | | | | | | | | |
|---|---|---|---|---|---|---|---|---|---|
| Belv. | *** | ND | *** | *** | ND | *** | *** | *** | ** |

D-2.

| | Lovo | | NCI-H358 | | SW620 | | Calu6 | | NCI-H2122 | | SW480 | | Mewo | | NCIH2087 | NCIH1755 | |
|---|---|---|---|---|---|---|---|---|---|---|---|---|---|---|---|---|---|
| | pERK | | pERK | | pERK | | pERK | | pERK | | pERK | | pERK | | pERK | pERK | |
| Ex. | Prolif. | | Prolif. | | Prolif. | | Prolif. | | Prolif. | | Prolif. | | Prolif. | | Prolif. | Prolif. | |
| 44 | ++ | (−10) | ++ | (1) | ++ | (3) | +++ | (−12) | ++ | (−11) | ++ | (−4) | +++ | (3) | +++ (4) | +++ | (−9) |
| 44 | *** | | *** | | *** | | *** | | *** | | *** | | *** | | *** | *** | |
| 122 | ++ | (13) | ++ | (−3) | ++ | (−6) | ++ | (−8) | ++ | (27) | ++ | (5) | ++ | (2) | +++ (4) | +++ | (11) |
| 122 | *** | | *** | | *** | | *** | | ** | | *** | | *** | | *** | *** | |
| Belv. | +++ | (−12) | ++ | (−122) | ++ | (−151) | ++ | (−39) | ++ | (−9) | ++ | (−100) | ++ | (−116) | +++ (6) | ++ | (−79) |

| | | | | | | | | | |
|---|---|---|---|---|---|---|---|---|---|
| Belv. | *** | ** | ** | *** | ** | ** | ** | *** | * |

In pERK: + denotes a IC₅₀ > 0.3 μM, ++ denotes a 0.03-0.3 μM IC₅₀ range, +++ denotes a IC₅₀ < 0.03 μM.
For proliferation: * denotes a proliferation EC₅₀ > 3 μM, ** denotes a 0.3-3 μM proliferation EC₅₀ range, *** denotes a proliferation EC₅₀ < 0.3 μM.
N/A: not available.
Belv.: Belvarafenib.
Values in parentheses are % inhibition.
Blank means the value was not determined.

For RAS-mutant cancer cell lines, the % Yₘᵢₙ values for pERK IC₅₀ curves were all above −20% and considered to display minimal or no induction and thus compounds do not cause detectable paradoxical activation of the pathway in this panel of cancer cell lines. In contrast, the comparative results for the molecule Belvarafenib (obtained from MedChem Express cat. No. HY-109080; CAS No. 1446113-23-0) causes mild to strong induction of the pathway in the same cell lines (Y_{MIN}<−30% in 10 of the 13 RAS-mutant cell lines tested).

### (d) Measurement of Proliferation Inhibition of Cultivated Human Cancer Cell Lines (CCLs) Using CellTiter-Glo® Reagent

CellTiter-Glo® viability analysis was conducted on cells plated in 100 μL of complete RPMI-1640 growth medium in 96-well flat-bottomed white opaque plates (Greiner or Croning) at a density indicated in Table E (for each CCL, number of cells plated per well of a 96-well plate to perform the CellTiter-Glo® cell viability assay). The cell density in cells/cm² would correspond to cell number divided by the area of one well of a 96-well plate (0.32 cm²). Cells were maintained overnight at 37° C. under 5% CO₂ before treatment with compounds' dilution series for 3 days.

TABLE E
Number of cells plated per well of a 96-well plate
to perform the CellTiter-GloR cell viability assay

| Cell Line | Number of cells/well |
|---|---|
| A375 | 2000 |
| A101D | 2000 |
| A2058 | 2000 |
| RKO | 2000 |
| HT29 | 2000 |
| NCIH2087 | 3600 |
| NCIH1755 | 3600 |
| NCIH1666 | 3600 |
| SK-MEL30 | 2400 |
| IPC298 | 2000 |
| HepG2 | 7200 |
| HCT-116 | 1500 |
| Lovo | 4800 |
| SW620 | 3600 |
| SW480 | 4800 |
| NCI-H358 | 2400 |
| NCI-H2122 | 4800 |
| Calu-6 | 2400 |
| Mewo | 2400 |

In a dilution series 100 μL/well of test substance dilution prepared in complete RPMI-1640 growth media was added to the cells plated initially in 100 μL of growth media. The dilution series is selected so that ten concentrations cover a range from 10 μM to 0.33 nM. If necessary (as in the case of A375 cells, which were more sensitive to the compounds), the initial concentration of 10 μM is lowered to 1 μM and further dilution is carried out accordingly. The final concentration of DMSO in the assay is set to 0.5%.

After 3 days of incubation, the growth media were removed by aspiration and 60 μL of diluted CellTiter-Glo® reagent (10 μL CellTiter-Glo® reagent+50 μL of PBS) was added to each well. Cells were allowed to lyse and to equilibrate in CellTiter-Glo® reagent by 5 min incubation on a plate shaker followed by 10 min incubation at room temperature. Luminescence signals were then acquired on a Synergy Neo2 plate reader (Biotek).

The effect of each compound dilution on the proliferation of cancer cell lines was expressed in % inhibition and calculated as follows. An internal 100% inhibition control (1 μM of trametinib; cat. No. HY-10999; MedChem Express; CAS No. 871700-17-3) was included in each plate and used as a measure of CellTiter-Glo® signal background. The value obtained for trametinib was subtracted from each data point. The average of DMSO (vehicle) controls (set as 0% inhibition) was established and used to calculate % inhibition:

$$\% inhibition = 100 * ( 1 - ( ( CellTiter - Glo ® {signal}_{compound} ) / ( CellTiter - Glo ® {signal}_{DMSO} ) ) )$$

The ability of each compound to inhibit proliferation was expressed as a EC₅₀ value obtained by plotting the effect values for each data point of a dilution series and fitting the obtained curves using a log(agonist) versus response—variable slope (four parameters) function using GraphPadPrism (V7.0) or Dotmatics Screening Ultra platform.

As shown in Tables D-1 and D-2, the active substances show antiproliferative activity in various NRAS-, KRAS- and NF1-mutant cancer cell lines that are representative of a large diversity of tissue types (i.e., SK-MEL 30, IPC298, HepG2, HCT-116, Lovo, SW620, SW480, NCI-H358, NCI-H2122, Calu-6 and Mewo; Tables D-1 and D-2 and refer to Table B for genotypes). Antiproliferative activity is often even stronger in cell lines carrying BRAF driver mutations (A375, A101D, A2058, RKO, HT29, NCIH2087, NCIH1755 and NCIH1666) (Tables D-1 and D-2). Of note, the IC₅₀ values of pERK reduction and the EC₅o values of the antiproliferative activity of the substances in KRAS- and BRAF-mutated cell lines correlate reasonably well with each other (Tables D-1 and D-2). The present compounds are thus effective against several tumor types and may be used in these and other indications. This demonstrates the usefulness of the compounds as described herein for the treatment of different types of tumors.

### (e) Results

The following Tables 3 and 4 summarize exemplary compounds structures and biological results. Each of these tables are followed by their respective table summarizing chemical characterization of the compounds and methods of synthesis.

TABLE 3

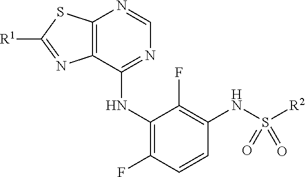

| | | | HCT116 | Kinase IC₅₀ | Kinase IC₅₀ |
|---|---|---|---|---|---|
| | | | pERK IC₅₀ | BRAF | CRAF |
| Examples | R¹ | R² | (μM) (Yₘᵢₙ %) | (nM) | (nM) |
| 1 | A1 | B1 | +++ (−11) | ** | §§§ |
| 2 | A2 | B1 | +++ (−1) | ** | §§ |
| 3 | A2 | B2 | +++ (−9) | ** | §§§ |
| 4 | A3 | B2 | ++ (0) | ** | §§§ |
| 5 | A2 | B3 | +++ (−6) | ** | §§§ |
| 6 | A3 | B3 | ++ (−4) | ** | §§§ |
| 7 | A3 | B1 | +++ (−5) | ** | §§§ |
| 8 | A4 | B1 | ++ (4) | ** | §§ |
| 9 | A4 | B2 | ++ (−9) | ** | §§§ |
| 10 | A4 | B3 | + (−2) | ** | §§ |
| 11 | A5 | B2 | + (4) | ** | §§§ |
| 12 | A6 | B2 | + (−6) | ** | §§ |
| 13 | A2 | B4 | + (−14) | ** | §§ |
| 14 | A3 | B4 | + (−14) | ** | §§ |
| 15 | A2 | B5 | +++ (−3) | ** | §§ |
| 16 | A3 | B5 | ++ (−8) | ** | §§ |
| 17 | A7 | B2 | + (−12) | ** | §§ |
| 18 | A8 | B2 | +++ (0) | *** | §§§ |
| 19 | A9 | B2 | ++ (−14) | ** | §§§ |
| 20 | A10 | B2 | +++ (−12) | ** | §§ |
| 21 | A11 | B2 | + (−4) | * | §§ |
| 22 | A11 | B1 | + (−8) | * | § |
| 23 | A12 | B1 | + (−11) | * | § |
| 24 | A13 | B1 | ++ (2) | * | §§ |
| 25 | A14 | B1 | + (2) | * | § |
| 26 | A15 | B1 | + (−13) | * | § |
| 27 | A16 | B1 | ++ (−3) | ** | §§ |
| 28 | A17 | B1 | +++ (−10) | ** | §§ |
| 29 | A18 | B1 | ++ (5) | * | § |
| 30 | A19 | B1 | +++ (−3) | ** | §§ |
| 31 | A20 | B1 | ++ (7) | ** | §§§ |
| 32 | A21 | B1 | + (−13) | ** | §§ |
| 33 | A22 | B1 | +++ (−7) | *** | §§§ |
| 34 | A23 | B1 | + (−13) | * | §§ |
| 35 | A8 | B1 | +++ (−8) | ** | §§§ |
| 36 | A8 | B3 | + (−13) | ** | §§ |
| 37 | A3 | B6 | +++ (−11) | ** | §§§ |
| 38 | A8 | B6 | ++ (−6) | ** | §§§ |
| 39 | A24 | B1 | +++ (−12) | *** | §§§ |
| 40 | A25 | B1 | +++ (−7) | *** | §§§ |
| 41 | A26 | B2 | +++ (0) | *** | §§§ |
| 42 | A27 | B1 | ++ (2) | * | §§ |
| 43 | A28 | B1 | +++ (9) | *** | §§§ |
| 44 | A29 | B1 | +++ (3) | *** | §§§ |
| 45 | A19 | B2 | +++ (−4) | *** | §§§ |
| 46 | A20 | B2 | +++ (1) | ** | §§§ |
| 47 | A30 | B2 | + (−1) | ** | §§ |
| 48 | A31 | B2 | ++ (0) | ** | §§ |
| 49 | A32 | B2 | ++ (−3) | ** | §§§ |
| 50 | A17 | B2 | +++ (1) | *** | §§§ |
| 51 | A25 | B2 | +++ (−1) | *** | §§§ |
| 52 | A22 | B2 | ++ (−9) | *** | §§§ |
| 53 | A33 | B2 | ++ (−9) | *** | §§§ |
| 54 | A34 | B2 | +++ (−6) | ** | §§§ |
| 55 | A35 | B2 | ++ (−3) | *** | §§§ |
| 56 | A29 | B2 | +++ (0) | *** | §§§ |
| 57 | A36 | B2 | +++ (4) | ** | §§ |
| 58 | A37 | B2 | +++ (−12) | ** | §§§ |
| 59 | A38 | B2 | ++ (4) | *** | §§§ |
| 60 | A39 | B2 | +++ (−2) | *** | §§§ |
| 61 | A40 | B2 | +++ (−13) | ** | §§§ |
| 62 | A41 | B2 | +++ (2) | ** | §§§ |
| 63 | A42 | B2 | +++ (−5) | ** | §§ |
| 64 | A43 | B2 | +++ (−4) | ** | § |
| 65 | A44 | B2 | ++ (−3) | ** | §§ |
| 66 | A45 | B2 | +++ (−13) | ** | §§ |
| 67 | A46 | B2 | +++ (0) | ** | § |
| 68 | A47 | B2 | +++ (−6) | ** | §§ |
| 69 | A48 | B2 | ++ (−7) | * | §§ |
| 70 | A49 | B2 | ++ (−8) | * | §§ |
| 71 | A50 | B2 | +++ (−4) | ** | §§§ |
| 72 | A51 | B2 | ++ (−8) | ** | §§§ |
| 73 | A52 | B2 | +++ (−12) | ** | § |
| 74 | A53 | B2 | + (−9) | ** | §§§ |
| 75 | A54 | B2 | + (−5) | * | §§ |
| 76 | A55 | B2 | + (−10) | * | §§ |
| 77 | A56 | B2 | ++ (−5) | ** | §§ |
| 78 | A57 | B2 | ++ (−14) | ** | §§ |
| 79 | A58 | B2 | ++ (−17) | ** | §§§ |
| 80 | A59 | B2 | + (−3) | ** | §§§ |
| 81 | A60 | B2 | ++ (−8) | ** | §§ |
| 82 | A61 | B2 | ++ (−13) | ** | §§ |
| 83 | A62 | B2 | ++ (−10) | ** | §§ |
| 84 | A63 | B2 | +++ (18) | ** | §§§ |
| 85 | A64 | B2 | +++ (8) | ** | §§§ |
| 86 | A65 | B2 | +++ (4) | ** | §§§ |
| 87 | A66 | B2 | ++ (−8) | ** | §§§ |
| 88 | A67 | B2 | + (−8) | ** | § |
| 89 | A68 | B2 | +++ (−16) | ** | § |
| 90 | A69 | B2 | +++ (−6) | ** | §§§ |
| 91 | A70 | B2 | + (−8) | * | § |
| 92 | A71 | B2 | + (−10) | * | § |
| 93 | A72 | B2 | ++ (3) | *** | §§§ |
| 94 | A73 | B2 | +++ (−10) | ** | §§§ |
| 95 | A74 | B2 | +++ (−6) | ** | §§§ |
| 96 | A75 | B2 | ++ (−1) | * | §§ |
| 97 | A76 | B2 | +++ (−10) | * | §§ |
| 98 | A77 | B2 | +++ (−6) | ** | §§§ |
| 99 | A78 | B2 | +++ (−7) | ** | §§§ |
| 100 | A79 | B2 | +++ (−10) | ** | §§§ |
| 101 | A80 | B2 | +++ (0) | * | §§ |
| 102 | A81 | B2 | +++ (0) | ** | §§§ |
| 103 | A1 | B2 | ++ (−7) | ** | §§§ |
| 104 | A82 | B2 | +++ (−10) | ** | §§ |
| 105 | A83 | B2 | +++ (4) | ** | §§§ |
| 106 | A84 | B2 | ++ (−11) | * | §§ |
| 107 | A85 | B2 | + (4) | * | § |
| 108 | A86 | B2 | +++ (−10) | ** | §§ |
| 109 | A87 | B2 | ++ (−13) | * | § |
| 110 | A88 | B2 | +++ (−6) | ** | §§ |
| 111 | A89 | B2 | +++ (−10) | *** | §§§ |
| 112 | A82 | B1 | +++ (7) | ** | §§§ |
| 113 | A90 | B2 | +++ (2) | ** | §§ |
| 114 | A91 | B2 | + (−14) | * | § |
| 115 | A92 | B2 | +++ (−11) | ** | §§§ |
| 116 | A82 | B3 | +++ (−3) | ** | §§ |
| 117 | A82 | B4 | ++ (−15) | * | §§ |
| 118 | A82 | B5 | +++ (−14) | * | § |
| 119 | A93 | B2 | +++ (−1) | *** | §§§ |
| 120 | A94 | B2 | +++ (−8) | ** | §§§ |
| 121 | A95 | B2 | ++ (−11) | ** | §§ |
| 122 | A96 | B2 | +++ (2) | ** | §§§ |
| 123 | A97 | B2 | +++ (−11) | ** | §§§ |
| 124 | A98 | B2 | +++ (12) | ** | §§§ |
| 125 | A99 | B2 | ++ (2) | ** | §§§ |
| 126 | A100 | B2 | +++ (−9) | *** | §§§ |
| 127 | A101 | B2 | +++ (1) | ** | §§ |
| 128 | A102 | B2 | +++ (1) | ** | §§ |
| 129 | A103 | B2 | +++ (0) | ** | § |
| 130 | A104 | B2 | +++ (−2) | *** | §§§ |
| 131 | A105 | B2 | +++ (0) | ** | §§§ |
| 132 | A106 | B2 | + (−11) | * | § |
| 133 | A107 | B2 | + (−12) | ** | §§ |
| 134 | A108 | B2 | +++ (−5) | ** | §§ |
| 135 | A109 | B2 | +++ (−5) | ** | § |
| 136 | A110 | B2 | +++ (−17) | * | §§ |
| 137 | A111 | B2 | +++ (−14) | * | § |
| 138 | A112 | B2 | +++ (−14) | * | § |
| 139 | A113 | B2 | +++ (−13) | ** | §§ |
| 140 | A114 | B2 | +++ (−10) | ** | §§ |
| 141 | A115 | B2 | +++ (2) | *** | §§§ |
| 142 | A116 | B2 | +++ (0) | ** | §§ |
| 143 | A117 | B2 | +++ (−8) | ** | §§§ |
| 144 | A118 | B2 | +++ (−4) | ** | §§§ |
| 145 | A119 | B2 | +++ (−13) | ** | § |
| 146 | A120 | B2 | +++ (−12) | * | § |
| 147 | A121 | B2 | +++ (−4) | *** | §§ |
| 148 | A122 | B2 | +++ (−2) | ** | §§ |
| 149 | A123 | B2 | +++ (16) | ** | §§ |
| 150 | A124 | B2 | + (−5) | * | §§ |
| 151 | A125 | B2 | +++ (−2) | ** | §§ |
| 152 | A126 | B2 | +++ (0) | *** | §§§ |
| 153 | A127 | B2 | +++ (−5) | ** | §§§ |
| 154 | A128 | B1 | + (−4) | * | § |
| 155 | A129 | B2 | +++ (−12) | ** | §§ |
| 156 | A130 | B2 | +++ (−7) | *** | §§§ |
| 157 | A131 | B2 | +++ (−14) | *** | §§§ |
| 158 | A132 | B2 | +++ (4) | *** | §§§ |

For pERK assays, + denotes a 1-10 μM IC₅₀ range, ++ denotes a 0.5-1 μM IC₅₀ range, and +++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range, and *** denotes an IC₅₀ < 1 nM. For the CRAF biochemical
kinase assay, § denotes an IC₅₀ >50 nM, §§ denotes a 10-50 nM IC₅₀ range, and §§§ denotes an IC₅₀ < 10 nM.

| | | MS | |
|---|---|---|---|
| | Synthesis | m/z | |
| Example | Method | (MH⁺) | ¹H NMR (400 MHz) |
| 1 | A | 546.1 | ¹H NMR (DMSO-d₆) δ: 10.47 (br. s., 1H), 9.81 (s, 1H), 8.37 (s, |
| | | | 1H), 8.20 (s, 1H), 7.59 (d, J = 7.8 Hz, 1H), 7.48 (t, J = 8.6 Hz, |
| | | | 1H), 7.37 (td, J = 8.0, 5.5 Hz, 1H), 7.26 (td, J = 8.6, 5.9 Hz, 1H), |
| | | | 7.19 (td, J = 9.0, 1.2 Hz, 1H), 2.48 (d, J = 2.0 Hz, 3H), 2.41 (br. |
| | | | s., 3H), 2.13 (s, 3H) |
| 2 | A | 583.1 | ¹H NMR (DMSO-d₆) δ: 10.49 (br. s., 1H), 9.84 (s, 1H), 8.75 (s, |
| | | | 1H), 8.37 (s, 1H), 7.68 (br. s., 1H), 7.60 (d, J = 7.8 Hz, 1H), 7.47 |
| | | | (t, J = 8.8 Hz, 1H), 7.37 (td, J = 7.8, 5.9 Hz, 1H), 7.29 (td, J = |
| | | | 8.6, 5.9 Hz, 1H), 7.21 (t, J = 9.2 Hz, 1H), 7.13 (t, J = 8.0 Hz, 1H), |
| | | | 6.60 (d, J = 7.4 Hz, 1H), 5.61 (s, 2H), 2.49 (br. s., 3H) |
| 3 | A | 619.2 | ¹H NMR (DMSO-d₆) δ: 10.71 (br. s., 1H), 9.84 (s, 1H), 8.75 (s, |
| | | | 1H), 8.36 (s, 1H), 7.93 (d, J = 7.4 Hz, 1H), 7.88 (dd, J = 7.8, 1.2 |
| | | | Hz, 1H), 7.67 (br. s., 1H), 7.50 (t, J = 8.0 Hz, 1H), 7.25-7.34 |
| | | | (m, 1H), 7.16-7.25 (m, 1H), 7.13 (t, J = 8.0 Hz, 1H), 6.60 (dd, |
| | | | J = 7.8, 0.8 Hz, 1H), 5.61 (br. s., 2H) |
| 4 | A | 604.2 | ¹H NMR (DMSO-d₆) δ: 10.73 (s, 1H), 9.90 (s, 1H), 9.02 (s, 1H), |
| | | | 8.65 (br. s., 1H), 8.39 (s, 1H), 7.92 (d, J = 8.2 Hz, 1H), 7.88 (d, |
| | | | J = 7.8 Hz, 1H), 7.80-7.85 (m, 1H), 7.40-7.57 (m, 3H), 7.26- |
| | | | 7.36 (m, 1H), 7.15-7.26 (m, 1H) |
| 5 | A | 599.2 | ¹H NMR (DMSO-d₆) δ: 10.55 (s, 1H), 9.84 (s, 1H), 8.75 (s, 1H), |
| | | | 8.37 (s, 1H), 7.74 (t, J = 8.0 Hz, 2H), 7.66 (br. s., 1H), 7.35 (t, |
| | | | J = 8.0 Hz, 1H), 7.29 (td, J = 8.6, 5.9 Hz, 1H), 7.22 (t, J = 9.0 Hz, |
| | | | 1H), 7.13 (t, J = 7.8 Hz, 1H), 6.60 (d, J = 8.6 Hz, 1H), 5.61 (s, |
| | | | 2H), 2.65 (s, 3H) |
| 6 | A | 584.2 | ¹H NMR (DMSO-d₆) δ: 10.57 (s, 1H), 9.91 (s, 1H), 9.02 (s, 1H), |
| | | | 8.64 (br. s., 1H), 8.39 (s, 1H), 7.79-7.89 (m, 1H), 7.74 (t, J = |
| | | | 7.8 Hz, 2H), 7.41-7.53 (m, 2H), 7.27-7.40 (m, 2H), 7.24 (t, J = |
| | | | 9.0 Hz, 1H), 2.65 (s, 3H) |
| 7 | A | 568.4 | ¹H NMR (DMSO-d₆) δ: 10.51 (s, 1H), 9.91 (s, 1H), 9.02 (s, 1H), |
| | | | 8.65 (br. s., 1H), 8.39 (s, 1H), 7.78-7.90 (m, 1H), 7.59 (d, J = |
| | | | 7.8 Hz, 1H), 7.42-7.53 (m, 3H), 7.38 (dd, J = 7.6, 6.1 Hz, 1H), |
| | | | 7.27-7.35 (m, 1H), 7.24 (t, J = 9.0 Hz, 1H), 2.49 (br. s., 3H) |
| 8 | C | 645.4 | ¹H NMR (DMSO-d₆) δ: 10.52 (s, 1H), 9.96 (s, 1H), 9.22 (d, J = |
| | | | 2.0 Hz, 1H), 9.12 (s, 1H), 8.78 (br. s., 1H), 8.63 (dd, J = 4.7, 1.6 |
| | | | Hz, 1H), 8.42 (dt, J = 8.2, 2.0 Hz, 1H), 8.41 (s, 1H), 7.74 (d, J = |
| | | | 7.0 Hz, 1H), 7.54-7.65 (m, 3H), 7.47 (t, J = 8.6 Hz, 1H), 7.33- |
| | | | 7.42 (m, 1H), 7.30 (dd, J = 8.0, 6.1 Hz, 1H), 7.24 (t, J = 9.0 Hz, |
| | | | 1H), 2.49 (br. s., 3H) |
| 9 | C | 681.4 | ¹H NMR (DMSO-d₆) δ: 10.73 (br. s., 1H), 9.94 (s, 1H), 9.23 (br. |
| | | | s., 1H), 9.10 (s, 1H), 8.64 (br. s., 2H), 8.41 (d, J = 8.2 Hz, 1H), |
| | | | 8.39 (s, 1H), 7.82-7.97 (m, 2H), 7.72 (d, J = 7.4 Hz, 1H), 7.52- |
| | | | 7.66 (m, 2H), 7.48 (t, J = 7.4 Hz, 1H), 7.25-7.35 (m, 1H), 7.14- |
| | | | 7.25 (m, 1H) |
| 10 | C | 661.2 | ¹H NMR (DMSO-d₆) δ: 10.58 (br. s., 1H), 9.96 (s, 1H), 9.22 (d, |
| | | | J = 1.2 Hz, 1H), 9.12 (s, 1H), 8.73 (br. s., 1H), 8.63 (d, J = 3.5 Hz, |
| | | | 1H), 8.42 (dt, J = 7.8, 1.8 Hz, 1H), 8.41 (s, 1H), 7.68-7.82 (m, |
| | | | 3H), 7.59-7.66 (m, 1H), 7.57 (dd, J = 7.6, 4.5 Hz, 1H), 7.28- |
| | | | 7.42 (m, 2H), 7.24 (t, J = 9.0 Hz, 1H), 2.66 (s, 3H) |
| 11 | D | 787.2 | ¹H NMR (DMSO-d₆) δ: 10.74 (br. s., 1H), 9.93 (s, 1H), 8.84 (br. |
| | | | s., 1H), 8.69 (s, 1H), 8.56 (br. s., 2H), 8.39 (s, 1H), 8.00 (dd, J = |
| | | | 7.4, 1.2 Hz, 1H), 7.93 (d, J = 8.2 Hz, 1H), 7.88 (d, J = 7.8 Hz, |
| | | | 1H), 7.43-7.61 (m, 3H), 7.36 (d, J = 5.1 Hz, 2H), 7.26-7.34 (m, |
| | | | 1H), 7.22 (t, J = 9.0 Hz, 1H), 4.37 (s, 2H), 2.76 (s, 3H) |
| 12 | D | 765.2 | ¹H NMR (DMSO-d₆) δ: 10.72 (br. s., 1H), 9.92 (s, 1H), 8.79 (br. |
| | | | s., 1H), 8.55 (s, 1H), 8.39 (s, 1H), 7.90-7.97 (m, 2H), 7.88 (d, |
| | | | J = 8.2 Hz, 1H), 7.42-7.60 (m, 3H), 7.29 (td, J = 8.7, 5.7 Hz, 1H), |
| | | | 7.20 (td, J = 9.0, 0.8 Hz, 1H), 3.09 (br. s., 4H), 2.43 (br. s., 4H), |
| | | | 2.16 (s, 3H) |
| 13 | A | 629.4 | ¹H NMR (DMSO-d₆) δ: 10.38 (br. s., 1H), 9.83 (s, 1H), 8.75 (s, |
| | | | 1H), 8.35 (s, 1H), 7.75 (d, J = 9.0 Hz, 1H), 7.68 (br. s., 1H), 7.28 |
| | | | (td, J = 8.6, 5.9 Hz, 1H), 7.19 (t, J = 9.0 Hz, 1H), 7.03-7.16 (m, |
| | | | 2H), 6.60 (d, J = 7.4 Hz, 1H), 5.61 (s, 2H), 3.90 (s, 3H), 2.65 (s, 3H) |
| 14 | A | 614.2 | ¹H NMR (DMSO-d₆) δ: 10.40 (br. s., 1H), 9.90 (s, 1H), 9.02 (s, |
| | | | 1H), 8.67 (br. s., 1H), 8.37 (s, 1H), 7.79-7.89 (m, 1H), 7.75 (d, |
| | | | J = 9.0 Hz, 1H), 7.38-7.57 (m, 2H), 7.30 (td, J = 8.6, 5.9 Hz, |
| | | | 1H), 7.21 (t, J = 9.0 Hz, 1H), 7.10 (d, J = 8.6 Hz, 1H), 3.90 (s, |
| | | | 3H), 2.65 (s, 3H) |
| 15 | A | 613.2 | ¹H NMR (DMSO-d₆) δ: 10.34 (br. s., 1H), 9.81 (s, 1H), 8.75 (s, |
| | | | 1H), 8.36 (s, 1H), 7.69 (br. s., 1H), 7.56 (d, J = 9.0 Hz, 1H), 7.21- |
| | | | 7.33 (m, 1H), 6.99-7.21 (m, 3H), 6.60 (d, J = 7.8 Hz, 1H), 5.61 |
| | | | (s, 2H), 3.87 (s, 3H), 2.47 (d, J = 2.3 Hz, 3H) |
| 16 | A | 598.2 | ¹H NMR (DMSO-d₆) δ: 10.33 (s, 1H), 9.90 (s, 1H), 9.03 (s, 1H), |
| | | | 8.66 (br. s., 1H), 8.38 (s, 1H), 7.78-7.90 (m, 1H), 7.56 (d, J = |
| | | | 9.0 Hz, 1H), 7.40-7.52 (m, 2H), 7.30 (td, J = 9.0, 5.9 Hz, 1H), |
| | | | 7.22 (t, J = 9.0 Hz, 1H), 7.10 (t, J = 8.4 Hz, 1H), 3.87 (s, 3H), |
| | | | 2.47 (d, J = 2.3 Hz, 3H) |
| 17 | See text | 672.2 | ¹H NMR (DMSO-d₆) δ: 10.74 (br. s., 1H), 9.99 (s, 1H), 9.21 (s, |
| | | | 1H), 9.03 (br. s., 1H), 8.41 (s, 1H), 7.93 (d, J = 8.2 Hz, 1H), 7.88 |
| | | | (d, J = 7.8 Hz, 1H), 7.80 (d, J = 7.4 Hz, 1H), 7.63 (t, J = 7.4 Hz, |
| | | | 1H), 7.50 (t, J = 7.6 Hz, 1H), 7.31 (td, J = 8.5, 6.1 Hz, 1H), 7.24 |
| | | | (t, J = 9.0 Hz, 1H) |
| 18 | A | 622.2 | ¹H NMR (DMSO-d₆) δ: 10.73 (s, 1H), 9.95 (s, 1H), 9.08 (s, 1H), |
| | | | 8.50 (br. s., 1H), 8.40 (s, 1H), 7.94 (dd, J = 7.8, 1.2 Hz, 1H), 7.87 |
| | | | (d, J = 7.8 Hz, 1H), 7.40-7.57 (m, 2H), 7.28-7.36 (m, 2H), 7.25 |
| | | | (t, J = 9.0 Hz, 1H) |
| 19 | A | 640.2 | ¹H NMR (DMSO-d₆) δ: 10.74 (s, 1H), 9.98 (s, 1H), 9.15 (s, 1H), |
| | | | 8.92 (br. s., 1H), 8.38 (s, 1H), 7.98 (dd, J = 10.8, 7.6 Hz, 1H), |
| | | | 7.94 (d, J = 8.2 Hz, 1H), 7.88 (d, J = 7.8 Hz, 1H), 7.52 (t, J = 7.8 |
| | | | Hz, 1H), 7.31 (td, J = 9.0, 5.8 Hz, 1H), 7.25 (t, J = 9.0 Hz, 1H) |
| 20 | A | 632.2 | ¹H NMR (DMSO-d₆) δ: 10.73 (s, 1H), 9.81 (s, 1H), 8.87 (s, 1H), |
| | | | 8.37 (br. s, 2H), 7.94 (d, J = 8.2 Hz, 1H), 7.88 (d, J = 7.4 Hz, |
| | | | 1H), 7.60 (s, 1H), 7.51 (t, J = 8.0 Hz, 1H), 7.31 (td, J = 9.0, 5.8 |
| | | | Hz, 1H), 7.24 (t, J = 9.0 Hz, 1H), 2.44 (s, 3H), 2.37 (s, 3H) |
| 21 | A | 605.2 | ¹H NMR (DMSO-d₆) δ: 10.75 (s, 1H), 10.05 (s, 1H), 8.89 (br. s., |
| | | | 1H), 8.44 (s, 1H), 8.31 (d, J = 8.2 Hz, 1H), 7.93 (d, J = 8.6 Hz, |
| | | | 1H), 7.88 (d, J = 7.8 Hz, 1H), 7.84 (t, J = 6.7 Hz, 1H), 7.67 (t, |
| | | | J = 7.6 Hz, 1H), 7.50 (t, J = 8.0 Hz, 1H), 7.29-7.40 (m, 1H), 7.26 |
| | | | (t, J = 9.0 Hz, 1H) |
| 22 | A | 569.2 | ¹H NMR (DMSO-d₆) δ: 10.53 (s, 1H), 10.04 (s, 1H), 8.90 (br. s., |
| | | | 1H), 8.44 (s, 1H), 8.31 (d, J = 8.6 Hz, 1H), 7.84 (t, J = 7.0 Hz, |
| | | | 1H), 7.68 (ddd, J = 8.2, 7.4, 0.8 Hz, 1H), 7.60 (d, J = 7.8 Hz, |
| | | | 1H), 7.47 (t, J = 9.0 Hz, 1H), 7.35-7.42 (m, 1H), 7.29-7.35 (m, |
| | | | 1H), 7.25 (t, J = 9.0 Hz, 1H), 2.46-2.49 (m, 3H) |
| 23 | B | 716.4 | ¹H NMR (DMSO-d₆) δ: 10.50 (br. s., 1H), 9.86 (d, J = 12.9 Hz, |
| | | | 1H), 8.90 (br. s., 1H), 8.62 (d, J = 17.6 Hz, 1H), 8.54 (dd, J = |
| | | | 4.7, 1.2 Hz, 1H), 8.33 (d, J = 13.7 Hz, 1H), 8.05-8.14 (m, 1H), |
| | | | 7.83 (dd, J = 7.8, 1.6 Hz, 1H), 7.64-7.77 (m, 1H), 7.49-7.61 |
| | | | (m, 2H), 7.39-7.49 (m, 2H), 7.32-7.39 (m, 1H), 7.25-7.32 (m, |
| | | | 1H), 7.09-7.25 (m, 1H), 4.99 (s, 1H), 4.84 (s, 1H), 3.31 (s, |
| | | | 1.5H), 3.08 (s, 1.5H), 2.47 (br. s., 3H) |
| 24 | B | 702.3 | ¹H NMR (DMSO-d₆) δ: 10.52 (s, 1H), 9.91 (s, 1H), 9.47 (t, J = |
| | | | 6.1 Hz, 1H), 9.01 (br. s., 1H), 8.64 (s, 1H), 8.49 (br. s., 1H), 8.37 |
| | | | (s, 1H), 8.32 (d, J = 8.2 Hz, 1H), 7.82 (d, J = 7.8 Hz, 1H), 7.67- |
| | | | 7.77 (m, 1H), 7.59 (d, J = 8.2 Hz, 1H), 7.56 (t, J = 7.4 Hz, 1H), |
| | | | 7.47 (t, J = 9.0 Hz, 1H), 7.34-7.42 (m, 2H), 7.29-7.34 (m, 1H), |
| | | | 7.25 (t, J = 9.0 Hz, 1H), 4.60 (d, J = 6.3 Hz, 2H), 2.48 (br. s., 3H) |
| 25 | B | 730.4 | ¹H NMR (DMSO-d₆) δ: 10.50 (s, 1H), 9.86 (s, 1H), 8.88 (br. s., |
| | | | 1H), 8.49-8.57 (m, 0.4H), 8.27-8.39 (m, 1H), 8.14-8.23 (m, |
| | | | 0.6H), 7.94 (d, J = 8.2 Hz, 0.4H), 7.84 (d, J = 7.8 Hz, 0.6H), 7.64- |
| | | | 7.78 (m, 1.4H), 7.51-7.61 (m, 2H), 7.41-7.50 (m, 2H), 7.23- |
| | | | 7.41 (m, 3H), 7.22 (d, J = 7.8 Hz, 1H), 6.96-7.03 (m, 0.6H), |
| | | | 4.13 (t, J = 7.0 Hz, 1.2H), 3.93 (t, J = 7.1 Hz, 0.8H), 3.22 (s, |
| | | | 1.2H), 3.15 (t, J = 7.8 Hz, 2H), 3.12 (s, 1.8H), 2.47 (br. s., 3H) |
| 26 | B | 683.4 | ¹H NMR (DMSO-d₆) δ: 10.52 (s, 1H), 9.88 (s, 1H), 8.90 (br. s., |
| | | | 1H), 8.37 (s, 1H), 8.03 (dd, J = 7.8, 5.9 Hz, 1H), 7.66-7.80 (m, |
| | | | 1H), 7.60 (d, J = 7.8 Hz, 1H), 7.50-7.57 (m, 1H), 7.47 (t, J = 8.6 |
| | | | Hz, 1H), 7.33-7.40 (m, 1H), 7.27-7.33 (m, 1H), 7.24 (t, J = 9.0 |
| | | | Hz, 1H), 3.86 (t, J = 5.5 Hz, 1H), 3.77 (t, J = 5.5 Hz, 1H), 3.60- |
| | | | 3.68 (m, 2H), 3.34 (s, 1.5H), 3.32 (s, 1.5H), 3.19 (s, 1.5H), 3.15 |
| | | | (s, 1.5H), 2.49 (br. s., 3H) |
| 27 | B | 696.4 | ¹H NMR (DMSO-d₆) δ: 10.40 (br. s., 1H), 9.91 (s, 1H), 8.96 (br. |
| | | | s., 1H), 8.38 (s, 1H), 8.00-8.16 (m, 1H), 7.75 (br. s., 1H), 7.53- |
| | | | 7.65 (m, 2H), 7.48 (t, J = 9.0 Hz, 1H), 7.34-7.43 (m, 1H), 7.28- |
| | | | 7.33 (m, 1H), 7.25 (t, J = 9.0 Hz, 1H), 3.90-4.04 (m, 2H), 3.40 |
| | | | (s, 4H), 3.16 (s, 1H), 2.91 (s, 4H), 2.73 (br. s., 1H), 2.49 (br. s., |
| | | | 3H), 1H under water peak |
| 28 | B | 729.4 | ¹H NMR (DMSO-d₆) δ: 10.51 (br. s., 1H), 9.81 (s, 1H), 8.46 (br. |
| | | | s, 1H), 8.36 (s, 1H), 8.06 (br. s., 1H), 7.70 (br. s., 1H), 7.60 (d, |
| | | | J = 7.8 Hz, 2H), 7.40-7.50 (m, 2H), 7.32-7.40 (m, 3H), 7.25- |
| | | | 7.32 (m, 2H), 7.22 (br. s., 2H), 3.88 (t, J = 7.2 Hz, 2H), 3.10 (s, |
| | | | 5H), 2.49 (br. s., 3H) |
| 29 | B | 701.3 | ¹H NMR (DMSO-d₆) δ: 10.51 (s, 1H), 9.86 (s, 1H), 8.99 (t, J = |
| | | | 5.7 Hz, 1H), 8.84 (br. s., 1H), 8.67 (s, 1H), 8.63 (d, J = 2.0 Hz, |
| | | | 1H), 8.49 (dd, J = 4.7, 1.6 Hz, 1H), 8.36 (s, 1H), 8.24-8.31 (m, |
| | | | 1H), 7.80 (dt, J = 7.8, 2.0 Hz, 1H), 7.59 (d, J = 7.8 Hz, 1H), 7.47 |
| | | | (t, J = 8.6 Hz, 1H), 7.34-7.43 (m, 4H), 7.27-7.34 (m, 1H), 7.23 |
| | | | (t, J = 9.0 Hz, 1H), 4.56 (d, J = 5.9 Hz, 2H), 2.49 (br. s., 3H) |
| 30 | B | 715.4 | ¹H NMR (DMSO-d₆) δ: 10.51 (s, 1H), 9.83 (s, 1H), 8.84 (br. s, |
| | | | 1H), 8.64 (d, J = 3.9 Hz, 2H), 8.36 (s, 2H), 7.81 (d, J = 7.0 Hz, |
| | | | 1H), 7.59 (d, J = 8.2 Hz, 1H), 7.34-7.55 (m, 6H), 7.26-7.34 (m, |
| | | | 1H), 7.24 (t, J = 9.0 Hz, 1H), 4.85 (s, 2H), 3.18 (br. s., 3H), 2.49 |
| | | | (br. s., 3H) |
| 31 | B | 715.4 | ¹H NMR (DMSO-d₆) δ: 10.51 (br. s., 1H), 9.82 (s, 1H), 8.60 (br. |
| | | | s., 1H), 8.53 (d, J = 3.5 Hz, 1H), 8.35 (s, 2H), 7.78 (d, J = 7.4 |
| | | | Hz, 2H), 7.60 (d, J = 7.8 Hz, 1H), 7.32-7.53 (m, 6H), 7.25-7.32 |
| | | | (m, 1H), 7.21 (br. s., 1H), 4.80 (s, 2H), 3.12 (br. s., 3H), 2.49 (br. |
| | | | s., 3H) |
| 32 | B | 695.4 | ¹H NMR (DMSO-d₆) δ: 10.52 (br. s., 1H), 9.89 (s, 1H), 8.91 (br. |
| | | | s., 1H), 8.37 (s, 1H), 8.25 (dd, J = 8.2, 5.5 Hz, 1H), 7.72 (br. s., |
| | | | 1H), 7.60 (d, J = 7.8 Hz, 1H), 7.54 (t, J = 7.6 Hz, 1H), 7.46 (t, |
| | | | J = 8.4 Hz, 1H), 7.33-7.40 (m, 1H), 7.27-7.33 (m, 1H), 7.23 (t, |
| | | | J = 9.0 Hz, 1H), 4.76 (dt, J = 11.0, 5.5 Hz, 1H), 4.04-4.13 (m, |
| | | | 1H), 3.92-4.01 (m, 0.5H), 3.69-3.80 (m, 1.5H), 3.55-3.66 (m, |
| | | | 0.5H), 3.45-3.53 (m, 1.5H), 3.37-3.44 (m, 1H), 2.35-2.45 (m, |
| | | | 1H), 1.95-2.12 (m, 1H), 1.67-1.85 (m, 1H), 2.49 (br. s., 3H) |
| 33 | B | 694.4 | ¹H NMR (DMSO-d₆) δ: 10.49 (br. s., 1H), 9.79 (s, 1H), 8.76 (br. |
| | | | s., 1H), 8.34 (s, 1H), 8.27 (br. s., 1H), 7.96 (br. s., 1H), 7.59 (d, |
| | | | J = 8.2 Hz, 1H), 7.29-7.50 (m, 4H), 7.05-7.29 (m, 2H), 4.60- |
| | | | 4.78 (m, 1H), 3.68-3.88 (m, 2H), 3.58-3.68 (m, 1H), 3.38- |
| | | | 3.58 (m, 3H), 2.33-2.41 (m, 1H), 1.91-2.04 (m, 1H), 1.59- |
| | | | 1.81 (m, 1H), 2.49 (br. s., 3H) |
| 34 | B | 707.4 | ¹H NMR (DMSO-d₆) δ: 10.52 (s, 1H), 9.90 (s, 1H), 8.94 (br. s., |
| | | | 1H), 8.37 (s, 1H), 8.20 (d, J = 8.2 Hz, 1H), 7.74 (t, J = 8.2 Hz, |
| | | | 1H), 7.60 (d, J = 7.8 Hz, 1H), 7.56 (t, J = 7.8 Hz, 1H), 7.47 (t, |
| | | | J = 8.6 Hz, 1H), 7.33-7.42 (m, 1H), 7.28-7.33 (m, 1H), 7.24 (t, |
| | | | J = 9.0 Hz, 1H), 5.04 (d, J = 4.3 Hz, 1H), 4.74 (d, J = 6.3 Hz, |
| | | | 1H), 3.87 (d, J = 11.0 Hz, 1H), 3.80 (dd, J = 10.6, 1.6 Hz, 1H), |
| | | | 3.73 (s, 2H), 1.90-2.07 (m, 4H), 2.49 (br. s., 3H) |
| 35 | A | 586.2 | ¹H NMR (DMSO-d₆) δ: 10.51 (br. s., 1H), 9.93 (s, 1H), 9.08 (s, |
| | | | 1H), 8.53 (br. s., 1H), 8.40 (s, 1H), 7.60 (d, J = 7.8 Hz, 1H), 7.41- |
| | | | 7.53 (m, 2H), 7.34-7.41 (m, 1H), 7.26-7.34 (m, 2H), 7.22 (t, |
| | | | J = 9.0 Hz, 1H), 2.48 (br. s., 3H) |
| 36 | A | 602.2 | ¹H NMR (DMSO-d₆) δ: 10.57 (br. s., 1H), 9.94 (s, 1H), 9.08 (s, |
| | | | 1H), 8.50 (br. s., 1H), 8.41 (s, 1H), 7.75 (d, J = 7.8 Hz, 1H), 7.72 |
| | | | (d, J = 7.8 Hz, 1H), 7.45 (br. s., 1H), 7.27-7.40 (m, 3H), 7.23 (t, |
| | | | J = 9.0 Hz, 1H), 2.65 (s, 3H) |
| 37 | A | 570.2 | ¹H NMR (DMSO-d₆) δ: 10.53 (s, 1H), 9.91 (s, 1H), 9.03 (s, 1H), |
| | | | 8.66 (br. s., 1H), 8.39 (s, 1H), 7.89 (d, J = 7.0 Hz, 1H), 7.81- |
| | | | 7.86 (m, 1H), 7.68 (dd, J = 8.2, 1.2 Hz, 1H), 7.64 (td, J = 7.0, 1.6 |
| | | | Hz, 1H), 7.40-7.54 (m, 3H), 7.29 (td, J = 9.0, 5.9 Hz, 1H), 7.22 |
| | | | (t, J = 9.0 Hz, 1H) |
| 38 | A | 588.2 | ¹H NMR (DMSO-d₆) δ: 10.53 (s, 1H), 9.94 (s, 1H), 9.08 (s, 1H), |
| | | | 8.54 (br. s., 1H), 8.40 (s, 1H), 7.89 (d, J = 7.4 Hz, 1H), 7.68 (dd, |
| | | | J = 7.8, 1.2 Hz, 1H), 7.64 (td, J = 7.1, 1.6 Hz, 1H), 7.41-7.55 |
| | | | (m, 2H), 7.25-7.35 (m, 2H), 7.22 (t, J = 9.0 Hz, 1H) |
| 39 | B | 695.4 | ¹H NMR (DMSO-d₆) δ: 9.83 (s, 1H), 8.79 (br. s., 1H), 8.36 (s, |
| | | | 1H), 8.26 (s, 1H), 7.79 (d, J = 7.8 Hz, 1H), 7.60 (d, J = 7.8 Hz, |
| | | | 1H), 7.35-7.51 (m, 4H), 7.18-7.34 (m, 2H), 3.80 (br. s., 2H), |
| | | | 3.17 (s, 6H), 2.75 (br. s., 4H), 2.47 (br. s., 3H), 1H exch., 1H |
| | | | under water peak |
| 40 | B | 706.4 | ¹H NMR (DMSO-d₆) δ: 10.51 (s, 1H), 9.82 (s, 1H), 8.78 (br. s., |
| | | | 1H), 8.36 (s, 1H), 8.27 (s, 1H), 7.79 (dd, J = 7.0, 2.0 Hz, 1H), |
| | | | 7.60 (d, J = 7.8 Hz, 1H), 7.33-7.52 (m, 4H), 7.16-7.33 (m, 2H), |
| | | | 4.41 (br. s., 2H), 3.72 (d, J = 11.0 Hz, 2H), 3.62 (d, J = 10.2 Hz, |
| | | | 2H), 2.49 (br. s., 3H), 1.95 (br. s., 4H) |
| 41 | D | 767.2 | ¹H NMR (DMSO-d₆) δ: 10.73 (s, 1H), 9.95 (s, 1H), 9.40 (br. s., |
| | | | 1H), 8.86 (br. s., 1H), 8.69 (s, 1H), 8.40 (s, 1H), 7.97-8.03 (m, |
| | | | 1H), 7.95 (dd, J = 8.0, 1.4 Hz, 1H), 7.88 (d, J = 7.8 Hz, 1H), 7.45- |
| | | | 7.60 (m, 3H), 7.31 (td, J = 8.6, 5.9 Hz, 1H), 7.25 (td, J = 9.0, |
| | | | 1.2 Hz, 1H), 3.42 (t, J = 5.9 Hz, 2H), 3.36 (t, J = 9.0 Hz, 2H), |
| | | | 2.88 (br. s., 6H), 2.84 (s, 3H); TFA salt |
| 42 | B | 720.4 | ¹H NMR (DMSO-d₆) δ: 10.52 (br. s., 1H), 9.93 (s, 1H), 9.47 (br. |
| | | | s., 1H), 8.97 (br. s., 1H), 8.39 (s, 1H), 8.24 (d, J = 7.8 Hz, 1H), |
| | | | 7.76 (t, J = 7.6 Hz, 1H), 7.53-7.66 (m, 2H), 7.48 (t, J = 9.0 Hz, |
| | | | 1H), 7.34-7.43 (m, 1H), 7.28-7.34 (m, 1H), 7.25 (t, J = 9.0 Hz, |
| | | | 1H), 5.41 (br. s., 1H), 4.99 (br. s., 1H), 3.71 (br. s., 1H), 3.58 (br. |
| | | | s., 2H), 2.87 (br. s., 3H), 2.49 (br. s., 3H), 1.94-2.28 (m, 4H) |
| 43 | B | 682.4 | ¹H NMR (DMSO-d₆) δ: 10.51 (s, 1H), 9.82 (s, 1H), 8.77 (br. s., |
| | | | 1H), 8.35 (s, 1H), 8.29 (br. s., 1H), 7.73 (d, J = 8.2 Hz, 1H), 7.56- |
| | | | 7.66 (m, 1H), 7.45-7.52 (m, 2H), 7.35-7.41 (m, 2H), 7.27- |
| | | | 7.34 (m, 1H), 7.24 (t, J = 9.0 Hz, 1H), 3.70 (t, J = 5.1 Hz, 2H), |
| | | | 3.58 (br. s., 2H), 3.33 (br. s., 3H), 3.12 (br. s., 3H), 2.47 (br. s., 3H) |
| 44 | B | 719.4 | ¹H NMR (DMSO-d₆) δ: 10.51 (br. s., 1H), 9.86 (s, 1H), 9.34 (br. |
| | | | s., 1H), 8.78 (br. s., 1H), 8.37 (s, 1H), 8.26 (s, 1H), 7.85 (br. s., |
| | | | 1H), 7.60 (d, J = 8.2 Hz, 1H), 7.34-7.51 (m, 3H), 7.27-7.34 (m, |
| | | | 1H), 7.24 (t, J = 9.0 Hz, 1H), 4.72 (br. s., 2H), 3.49 (br. s., 2H), |
| | | | 3.37 (br. s., 2H), 2.85 (br. s., 3H), 2.49 (br. s., 3H), 2.11 (br. s., |
| | | | 2H), 1.97 (br. s., 2H) |
| 45 | B | 751.2 | ¹H NMR (DMSO-d₆) δ: 10.73 (s, 1H), 9.84 (s, 1H), 8.78 (br. s, |
| | | | 1H), 8.58 (d, J = 4.7 Hz, 2H), 8.35 (s, 1H), 7.93 (d, J = 8.2 Hz, |
| | | | 1H), 7.87 (d, J = 7.4 Hz, 1H), 7.81 (d, J = 7.0 Hz, 1H), 7.45- |
| | | | 7.54 (m, 2H), 7.33-7.45 (m, 4H), 7.27-7.33 (m, 1H), 7.24 (t, |
| | | | J = 9.0 Hz, 1H), 4.81 (s, 2H), 3.15 (br. s., 3H) |
| 46 | B | 751.4 | ¹H NMR (DMSO-d₆) δ: 10.73 (br. s., 1H), 9.82 (br. s., 1H), 8.80 |
| | | | (br. s., 1H), 8.61 (br. s., 1H), 8.53 (d, J = 4.3 Hz, 1H), 8.35 (s, |
| | | | 2H), 7.92 (br. s., 1H), 7.87 (d, J = 7.4 Hz, 1H), 7.69-7.82 (m, |
| | | | 2H), 7.36-7.54 (m, 4H), 7.16-7.35 (m, 2H), 4.80 (s, 2H), 3.12 |
| | | | (br. s., 3H) |
| 47 | See text | 672.2 | ¹H NMR (DMSO-d₆) δ: 10.73 (br. s., 1H), 9.98 (br. s., 1H), 9.18 |
| | | | (s, 1H), 8.99 (br. s., 1H), 8.39 (s, 1H), 8.18 (s, 1H), 7.89 (d, J = |
| | | | 7.4 Hz, 1H), 7.84 (br. s., 1H), 7.38-7.55 (m, 1H), 7.23 (br. s., |
| | | | 1H), 7.12 (br. s., 1H) |
| 48 | D | 754.4 | ¹H NMR (DMSO-d₆) δ: 10.73 (br. s., 1H), 9.92 (s, 1H), 8.79 (br. |
| | | | s., 1H), 8.58 (s, 1H), 8.38 (s, 1H), 7.90-7.99 (m, 2H), 7.88 (d, |
| | | | J = 7.8 Hz, 1H), 7.43-7.59 (m, 3H), 7.30 (td, J = 8.5, 5.7 Hz, 1H), |
| | | | 7.23 (t, J = 9.4 Hz, 1H), 3.50 (t, J = 5.7 Hz, 2H), 3.28 (t, J = 5.5 |
| | | | Hz, 2H), 3.23 (s, 3H), 2.84 (s, 3H) |
| 49 | See text | 640.2 | ¹H NMR (DMSO-d₆) δ: 10.74 (s, 1H), 9.98 (s, 1H), 9.17 (s, 1H), |
| | | | 8.58 (br. s., 1H), 8.40 (s, 1H), 7.93 (d, J = 7.8 Hz, 1H), 7.87 (d, |
| | | | J = 7.8 Hz, 1H), 7.42-7.61 (m, 2H), 7.28-7.38 (m, 1H), 7.24 (t, |
| | | | J = 9.0 Hz, 1H) |
| 50 | B | 765.2 | ¹H NMR (DMSO-d₆) δ: 10.73 (s, 1H), 9.82 (s, 1H), 8.77 (br. s, |
| | | | 1H), 8.46 (br. s., 1H), 8.35 (s, 1H), 8.06 (br. s, 1H), 7.93 (d, J = |
| | | | 8.2 Hz, 1H), 7.86 (d, J = 8.6 Hz, 1H), 7.70 (br. s., 1H), 7.60 (d, |
| | | | J = 7.8 Hz, 1H), 7.49 (br. s., 1H), 7.38-7.45 (m, 1H), 7.35 (t, J = |
| | | | 7.0 Hz, 1H), 7.28-7.33 (m, 1H), 7.16-7.28 (m, 3H), 3.88 (t, J = |
| | | | 7.4 Hz, 2H), 3.10 (s, 5H) |
| 51 | B | 742.2 | ¹H NMR (DMSO-d₆) δ: 10.73 (s, 1H), 9.83 (s, 1H), 8.77 (br. s., |
| | | | 1H), 8.36 (s, 1H), 8.27 (s, 1H), 7.93 (br. s., 1H), 7.88 (d, J = 7.8 |
| | | | Hz, 1H), 7.79 (d, J = 7.3 Hz, 1H), 7.45-7.56 (m, 1H), 7.37-7.45 |
| | | | (m, 2H), 7.13-7.36 (m, 2H), 4.40 (br. s., 2H), 3.72 (d, J = 11.0 |
| | | | Hz, 2H), 3.62 (d, J = 10.6 Hz, 2H), 1.95 (br. s., 4H) |
| 52 | B | 730.2 | ¹H NMR (DMSO-d₆) δ: 10.73 (s, 1H), 9.83 (br. s., 1H), 8.76 (br. |
| | | | s., 1H), 8.36 (s, 1H), 8.29 (br. s., 1H), 7.97 (br. s., 1H), 7.92 (br. |
| | | | s., 1H), 7.87 (d, J = 7.4 Hz, 1H), 7.48 (br. s., 1H), 7.34-7.45 (m, |
| | | | 2H), 7.30 (br. s., 2H), 4.72 (d, J = 14.9 Hz, 1H), 3.79 (br. s., |
| | | | 1.5H), 3.66 (br. s., 1H), 3.52 (br. s., 1H), 3.46 (br. s., 2H), 3.37 |
| | | | (br. s., 0.5H), 2.38 (dt, J = 14.0, 6.9 Hz, 1H), 1.98 (td, J = 12.3, |
| | | | 7.0 Hz, 1H), 1.71 (br. s., 1H) |
| 53 | B | 716.2 | ¹H NMR (DMSO-d₆) δ: 10.73 (s, 1H), 9.82-9.90 (m, 1H), 8.76 |
| | | | (br. s., 1H), 8.34-8.39 (m, 1H), 8.29 (s, 1H), 8.10-8.18 (m, 1H), |
| | | | 7.90-8.03 (m, 1H), 7.87 (d, J = 8.2 Hz, 1H), 7.45-7.54 (m, 1H), |
| | | | 7.37-7.45 (m, 2H), 7.16-7.35 (m, 2H), 5.03 (d, J = 9.8 Hz, |
| | | | 0.5H), 4.66 (br. s., 2.5H), 4.36 (br. s., 0.5H), 4.07-4.29 (m, |
| | | | 0.5H), 3.85-3.96 (m, 1H), 3.60-3.75 (m, 0.5H), 3.41-3.59 (m, |
| | | | 0.5H), 1.98 (s, 0.5H), 1.89 (br. s., 0.5H), 1H exch. |
| 54 | B | 711.2 | ¹H NMR (DMSO-d₆) δ: 10.73 (s, 1H), 9.85 (d, J = 7.8 Hz, 1H), |
| | | | 8.75 (br. s., 1H), 8.36 (d, J = 4.7 Hz, 1H), 8.29 (s, 1H), 8.13 (d, |
| | | | J = 7.4 Hz, 0.5H), 8.00 (d, J = 7.4 Hz, 0.5H), 7.93 (br. s., 1H), |
| | | | 7.87 (d, J = 7.8 Hz, 1H), 7.49 (br. s., 1H), 7.35-7.45 (m, 2H), |
| | | | 7.30 (br. s., 1H), 7.24 (br. s., 1H), 5.03 (d, J = 8.6 Hz, 0.5H), 4.62 |
| | | | (br. s. 2H), 4.35 (br. s., 0.5H), 3.90 (br. s., 1H), 3.61 (br. s., 1H) |
| 55 | B | 730.2 | ¹H NMR (DMSO-d₆) δ: 10.73 (s, 1H), 9.81 (s, 1H), 8.78 (br. s, |
| | | | 1H), 8.35 (s, 1H), 8.23 (s, 1H), 7.91 (br. s., 1H), 7.88 (d, J = 7.8 |
| | | | Hz, 1H), 7.68 (d, J = 6.7 Hz, 1H), 7.48 (br. s., 1H), 7.34-7.45 |
| | | | (m, 2H), 7.27 (br. s., 1H), 7.20 (br. s., 1H), 4.95 (br. s., 1H), 3.73 |
| | | | (br. s., 1H), 3.58 (br. s., 1H), 3.42 (br. s., 3H), 1.87 (br. s., 1H), |
| | | | 1.76 (br. s., 1H), 1.46 (br. s., 2H) |
| 56 | B | 755.2 | ¹H NMR (DMSO-d₆) δ: 10.71 (br. s., 1H), 9.86 (s, 1H), 8.81 (br. |
| | | | s., 1H), 8.37 (s, 1H), 8.26 (s, 1H), 7.94 (dd, J = 8.0, 1.4 Hz, 1H), |
| | | | 7.88 (dd, J = 8.2, 0.8 Hz, 1H), 7.84 (d, J = 7.8 Hz, 1H), 7.51 (t, |
| | | | J = 8.0 Hz, 1H), 7.38-7.48 (m, 2H), 7.31 (td, J = 8.5, 5.7 Hz, 1H), |
| | | | 7.24 (td, J = 9.0, 1.2 Hz, 1H), 4.68 (br. s., 2H), 3.36 (br. s., 4H), |
| | | | 2.75 (br. s., 3H), 2.07 (br. s, 2H), 1.96 (d, J = 7.8 Hz, 2H) |
| 57 | B | 736.2 | ¹H NMR (DMSO-d₆) δ: 10.73 (s, 1H), 9.86 (s, 1H), 8.75 (br. s., |
| | | | 1H), 8.36 (s, 1H), 8.24 (s, 1H), 8.16 (dd, J = 7.0, 1.6 Hz, 1H), |
| | | | 7.93 (d, J = 8.2 Hz, 1H), 7.87 (d, J = 7.4 Hz, 1H), 7.49 (t, J = 7.8 |
| | | | Hz, 1H), 7.37-7.46 (m, 2H), 7.27-7.36 (m, 1H), 7.24 (t, J = 9.0 |
| | | | Hz, 1H), 6.41 (td, J = 56.0, 4.3 Hz, 1H), 4.62 (br. s., 1H), 4.49 |
| | | | (br. s., 1H), 4.21 (br. s., 1H), 4.03 (br. s., 1H), 3.13-3.28 (m, 1H) |
| 58 | B | 756.4 | ¹H NMR (DMSO-d₆) δ: 10.73 (br. s., 1H), 9.83 (s, 1H), 8.77 (br. |
| | | | s., 1H), 8.37 (s, 1H), 8.21 (s, 1H), 8.17 (dd, J = 7.0, 1.6 Hz, 1H), |
| | | | 7.91 (br. s., 1H), 7.88 (d, J = 8.2 Hz, 1H), 7.44-7.55 (m, 1H), |
| | | | 7.35-7.44 (m, 2H), 7.27 (br. s., 1H), 7.19 (br. s., 1H), 4.27 (br. |
| | | | s., 2H), 3.85 (br. s., 2H), 3.56 (t, J = 4.9 Hz, 4H), 1.76 (t, J = 5.1 |
| | | | Hz, 4H) |
| 59 | B | 702.2 | ¹H NMR (DMSO-d₆) δ: 10.73 (s, 1H), 9.85 (s, 1H), 8.71 (br. s., |
| | | | 1H), 8.36 (s, 1H), 8.20 (s, 1H), 8.15 (dd, J = 7.0, 1.6 Hz, 1H), |
| | | | 7.93 (d, J = 8.2 Hz, 1H), 7.87 (d, J = 7.8 Hz, 1H), 7.49 (t, J = 7.6 |
| | | | Hz, 1H), 7.36-7.46 (m, 2H), 7.31 (td, J = 9.0, 6.3 Hz, 1H), 7.24 |
| | | | (t, J = 9.0 Hz, 1H), 5.80 (d, J = 6.3 Hz, 1H), 4.65 (br. s., 1H), |
| | | | 4.52-4.61 (m, 1H), 4.29 (br. s., 2H), 3.84 (br. s., 1H) |
| 60 | B | 725.2 | ¹H NMR (DMSO-d₆) δ: 10.72 (s, 1H), 9.77 (br. s., 1H), 8.80 (br. |
| | | | s., 1H), 8.37 (s, 1H), 8.20 (br. s., 1H), 8.12-8.18 (m, 1H), 7.90 |
| | | | (d, J = 8.2 Hz, 1H), 7.32-7.52 (m, 3H), 7.17 (br. s, 1H), 4.67 (br. |
| | | | s., 1H), 4.25 (br. s., 2H), 3.84 (br. s., 1H), 2.99-3.09 (m, 1H), |
| | | | 2.94 (d, J = 6.7 Hz, 2H), 2H missing |
| 61 | E | 682.2 | ¹H NMR (DMSO-d₆) δ: 10.73 (br. s., 1H), 9.90 (s, 1H), 8.77 (br. |
| | | | s., 1H), 8.30-8.42 (m, 2H), 7.97-8.04 (m, 1H), 7.81-7.97 (m, |
| | | | 2H), 7.65 (s, 2H), 7.42-7.57 (m, 3H), 7.25-7.35 (m, 1H), 7.21 |
| | | | (br. s., 1H) |
| 62 | E | 740.2 | ¹H NMR (DMSO-d₆) δ: 10.73 (s, 1H), 9.91 (s, 1H), 8.76 (br. s., |
| | | | 1H), 8.45 (s, 1H), 8.37 (s, 1H), 7.96-8.04 (m, 2H), 7.93 (d, J = |
| | | | 7.8 Hz, 1H), 7.87 (d, J = 7.8 Hz, 1H), 7.43-7.56 (m, 3H), 7.31 |
| | | | (td, J = 9.0, 6.3 Hz, 1H), 7.23 (t, J = 9.4 Hz, 1H), 3.31 (br. s., |
| | | | 2H), 3.12 (s, 3H), 3.04 (q, J = 5.9 Hz, 2H) |
| 63 | F | 695.2 | ¹H NMR (DMSO-d₆) δ: 10.73 (br. s., 1H), 9.93 (br. s., 1H), 8.82 |
| | | | (br. s., 1H), 8.63 (s, 1H), 8.39 (s, 1 H), 7.94 (d, J = 8.6 Hz, 1H), |
| | | | 7.88 (d, J = 7.4 Hz, 2H), 7.41-7.60 (m, 3H), 7.27 (br. s., 1H), |
| | | | 7.19 (br. s., 1H), 3.40-3.48 (q, J = 7.4 Hz, 2H), 1.22 (t, J = 7.2 |
| | | | Hz, 3H) |
| 64 | F | 735.2 | ¹H NMR (DMSO-d₆) δ: 10.73 (s, 1H), 9.94 (s, 1H), 8.80 (br. s., |
| | | | 1H), 8.66 (s, 1H), 8.39 (s, 1H), 7.95 (t, J = 8.0 Hz, 2H), 7.87 (d, |
| | | | J = 8.2 Hz, 1H), 7.45-7.59 (m, 3H), 7.31 (td, J = 8.6, 5.9 Hz, |
| | | | 1H), 7.24 (t, J = 9.4 Hz, 1H), 3.81-3.94 (m, 1H), 1.96-2.10 (m, |
| | | | 2H), 1.83-1.95 (m, 2H), 1.52-1.73 (m, 4H) |
| 65 | F | 765.2 | ¹H NMR (DMSO-d₆) δ: 10.73 (br. s., 1H), 9.94 (s, 1H), 8.82 (br. |
| | | | s., 1H), 8.65 (s, 1H), 8.39 (s, 1H), 7.94-8.00 (m, 1H), 7.92 (d, |
| | | | J = 7.8 Hz, 1H), 7.87 (d, J = 7.8 Hz, 1H), 7.44-7.60 (m, 3H), 7.26- |
| | | | 7.35 (m, 1H), 7.22 (t, J = 8.6 Hz, 1H), 3.78-3.82 (m, 1H), 3.74- |
| | | | 3.78 (m, 1H), 3.44 (d, J = 6.3 Hz, 2H), 3.24-3.31 (m, 2H), 2.13- |
| | | | 2.27 (m, 1H), 1.75-1.80 (m, 1H), 1.71-1.75 (m, 1H), 1.31- |
| | | | 1.45 (m, 2H) |
| 66 | E | 766.2 | ¹H NMR (DMSO-d₆) δ: 10.73 (br. s., 1H), 9.90 (s, 1H), 8.83 (br. |
| | | | s., 1H), 8.59 (s, 1H), 8.38 (s, 1H), 7.83-8.02 (m, 3H), 7.45- |
| | | | 7.61 (m, 3H), 7.25-7.35 (m, 1H), 7.20 (br. s., 1H), 4.68-4.84 |
| | | | (m, 1H), 3.75 (td, J = 8.4, 4.7 Hz, 1H), 3.45-3.56 (m, 2H), 3.39- |
| | | | 3.45 (m, 1H), 2.76 (s, 3H), 1.82-1.99 (m, 1H), 1.58 (dtd, J = |
| | | | 13.0, 8.0, 8.0, 5.1 Hz, 1H) |
| 67 | D | 778.2 | ¹H NMR (DMSO-d₆) δ: 10.73 (s, 1H), 9.91 (s, 1H), 8.83 (br. s., |
| | | | 1H), 8.63 (s, 1H), 8.38 (s, 1H), 7.91-7.96 (m, 2H), 7.88 (d, J = |
| | | | 7.4 Hz, 1H), 7.43-7.58 (m, 3H), 7.28 (br. s., 1H), 7.21 (br. s., |
| | | | 1H), 4.24 (br. s., 2H), 3.58 (s, 4H), 1.66-1.75 (m, 2H), 1.35- |
| | | | 1.45 (m, 2H) |
| 68 | D | 791.2 | ¹H NMR (DMSO-d₆) δ: 10.73 (br. s., 1H), 9.94 (s, 1H), 9.29 (br. |
| | | | s., 1H), 8.85 (br. s., 1H), 8.69 (s, 1H), 8.39 (s, 1H), 7.95 (t, J = |
| | | | 7.0 Hz, 2H), 7.87 (d, J = 7.8 Hz, 1H), 7.46-7.59 (m, 3H), 7.31 |
| | | | (td, J = 9.0, 6.3 Hz, 1H), 7.25 (t, J = 9.0 Hz, 1H), 4.59 (br. s., |
| | | | 2H), 3.50 (br. s., 2H), 3.23 (br. s., 2H), 2.77 (br. s., 3H), 1.77 (m, |
| | | | 2H), 1.50 (m, 2H), TFA salt |
| 69 | E | 766.2 | ¹H NMR (DMSO-d₆) δ: 10.72 (br. s., 1H), 9.88 (br. s., 1H), 8.83 |
| | | | (br. s., 1H), 8.58 (s, 1H), 8.38 (s, 1H), 7.88 (d, J = 7.8 Hz, 3H), |
| | | | 7.40-7.59 (m, 3H), 7.25 (br. s., 1H), 7.15 (br. s., 1H), 3.97-4.07 |
| | | | (m, 1H), 3.76 (d, J = 9.8 Hz, 1H), 3.50-3.62 (m, 2H), 3.17-3.28 |
| | | | (m, 3H), 1.14 (d, J = 6.7 Hz, 3H) |
| 70 | E | 768.2 | ¹H NMR (DMSO-d₆) δ: 10.73 (br. s., 1H), 9.91 (s, 1H), 8.79 (br. |
| | | | s., 1H), 8.59 (s, 1H), 8.38 (s, 1H), 7.92 (d, J = 7.0 Hz, 2H), 7.87 |
| | | | (d, J = 7.8 Hz, 1H), 7.44-7.56 (m, 3H), 7.26-7.34 (m, 1H), 7.22 |
| | | | (t, J = 8.6 Hz, 1H), 3.48 (t, J = 5.9 Hz, 2H), 3.39 (t, J = 5.9 Hz, |
| | | | 2H), 3.30 (br. s., 2H), 3.22 (s, 3H), 1.08 (t, J = 7.0 Hz, 3H) |
| 71 | See text | 640.2 | ¹H NMR (DMSO-d₆) δ: 10.74 (s, 1H), 10.00 (s, 1H), 9.12 (s, 1H), |
| | | | 8.57 (br. s., 1H), 8.39 (s, 1H), 7.94 (d, J = 7.8 Hz, 1H), 7.88 (d, |
| | | | J = 7.8 Hz, 1H), 7.52 (t, J = 8.0 Hz, 1H), 7.40 (td, J = 10.3, 2.2 |
| | | | Hz, 1H), 7.31 (td, J = 8.6, 5.9 Hz, 1H), 7.24 (t, J = 9.0 Hz, 1H) |
| 72 | See text | 681.2 | ¹H NMR (DMSO-d₆) δ: 10.73 (s, 1H), 9.95 (s, 1H), 8.83 (br. s., |
| | | | 1H), 8.64 (s, 1H), 8.39 (s, 1H), 7.97 (dd, J = 7.0, 1.6 Hz, 1H), |
| | | | 7.93 (d, J = 7.8 Hz, 1H), 7.87 (d, J = 7.4 Hz, 1H), 7.44-7.61 (m, |
| | | | 3H), 7.31 (td, J = 8.6, 5.9 Hz, 1H), 7.23 (t, J = 9.2 Hz, 1H), 3.39 |
| | | | (s, 3H) |
| 73 | F | 709.2 | ¹H NMR (DMSO-d₆) δ: 10.73 (s, 1H), 9.95 (s, 1H), 8.79 (br. S., |
| | | | 1H), 8.62 (s, 1H), 8.39 (s, 1H), 7.93 (dd, J = 7.0, 1.6 Hz, 2H), |
| | | | 7.88 (d, J = 8.2 Hz, 1H), 7.44-7.61 (m, 3H), 7.26-7.36 (m, 1H), |
| | | | 7.23 (t, J = 9.0 Hz, 1H), 3.50 (quin, J = 6.7 Hz, 1H), 1.28 (d, J = |
| | | | 6.7 Hz, 6H) |
| 74 | E | 752.2 | ¹H NMR (DMSO-d₆) δ: 10.73 (s, 1H), 9.93 (br. s., 1H), 8.83 (br. |
| | | | s., 1H), 8.64 (s, 1H), 8.39 (s, 1H), 7.94 (d, J = 8.2 Hz, 2H), 7.88 |
| | | | (d, J = 7.8 Hz, 1H), 7.45-7.58 (m, 3H), 7.29 (br. s., 1H), 7.22 |
| | | | (br. s., 1H), 4.60 (t, J = 5.1 Hz, 1H), 3.82 (t, J = 8.2 Hz, 2H), 3.55- |
| | | | 3.66 (m, 2H), 3.40 (br. s., 1H), 3.24 (t, J = 5.5 Hz, 2H) |
| 75 | E | 760.2 | ¹H NMR (DMSO-d₆) δ: 10.73 (br. s., 1H), 9.92 (s, 1H), 8.83 (br. |
| | | | s., 1H), 8.70 (s, 1H), 8.39 (s, 1H), 7.95 (dd, J = 7.0, 1.6 Hz, 1H), |
| | | | 7.83-7.93 (m, 2H), 7.41-7.60 (m, 3H), 7.24-7.35 (m, 1H), |
| | | | 7.20 (br. s., 1H), 6.25 (tt, J = 55.2, 3.9 Hz, 1H), 3.61 (td, J = 14.9, |
| | | | 3.9 Hz, 2H), 2.91 (s, 3H) |
| 76 | E | 772.2 | ¹H NMR (DMSO-d₆) δ: 10.74 (br. s., 1H), 9.95 (s, 1H), 8.85 (br. |
| | | | s., 1H), 8.73 (s, 1H), 8.39 (s, 1H), 7.94 (dd, J = 8.6, 7.4 Hz, 2H), |
| | | | 7.88 (d, J = 7.8 Hz, 1H), 7.42-7.63 (m, 3H), 7.26-7.36 (m, 1H), |
| | | | 7.22 (t, J = 9.0 Hz, 1H), 6.08 (td, J = 56.3, 3.5 Hz, 1H), 3.94 (t, |
| | | | J = 8.8 Hz, 2H), 3.79 (dd, J = 8.8, 6.1 Hz, 2H), 2.89-3.09 (m, 1H) |
| 77 | B | 758.2 | ¹H NMR (DMSO-d₆) δ: 10.73 (s, 1H), 9.85 (s, 1H), 8.77 (br. s., |
| | | | 1H), 8.35 (s, 2H), 7.93 (d, J = 7.4 Hz, 1H), 7.82-7.91 (m, 2H), |
| | | | 7.50 (t, J = 7.8 Hz, 1H), 7.35-7.46 (m, 2H), 7.31 (td, J = 8.7, 5.7 |
| | | | Hz, 1H), 7.24 (t, J = 9.0 Hz, 1H), 4.95 (br. s., 1H), 4.41 (t, J = 7.0 |
| | | | Hz, 1H), 3.60-3.83 (m, 2H), 1.82-2.04 (m, 3H), 1.66 (br. s., |
| | | | 1H), 1.17 (d, J = 10.6 Hz, 6H) |
| 78 | B | 744.2 | ¹H NMR (DMSO-d₆) δ: 10.73 (br. s., 1H), 9.79 (s, 1H), 8.74 (br. |
| | | | s., 1H), 8.35 (s, 1H), 8.21 (s, 1H), 7.88 (d, J = 7.8 Hz, 2H), 7.85 |
| | | | (d, J = 7.8 Hz, 1H), 7.47 (br. s., 1H), 7.32-7.44 (m, 2H), 7.27 |
| | | | (br. s., 1H), 7.17 (br. s., 1H), 4.98 (t, J = 5.7 Hz, 1H), 3.94 (dd, |
| | | | J = 10.6, 6.3 Hz, 1H), 3.57-3.77 (m, 3H), 2.19 (dt, J = 12.1, 7.6 |
| | | | Hz, 1H), 1.72-1.89 (m, 2H), 1.66 (dt, J = 12.2, 5.8 Hz, 1H), 1.47 |
| | | | (s, 3H) |
| 79 | See text | 696.2 | ¹H NMR (DMSO-d₆) δ: 10.73 (br. s., 1H), 9.92 (s, 1H), 9.04 (s, |
| | | | 1H), 8.39 (s, 1H), 8.36 (br. s., 1H), 7.91 (d, J = 9.0 Hz, 1H), 7.87 |
| | | | (d, J = 8.2 Hz, 1H), 7.40-7.56 (m, 1H), 7.25-7.39 (m, 2H), 7.05- |
| | | | 7.25 (m, 1H), 4.24-4.32 (m, 2H), 3.66-3.75 (m, 2H), 3.34 (s, 3H) |
| 80 | See text | 682.2 | ¹H NMR (DMSO-d₆) δ: 10.73 (s, 1H), 9.94 (s, 1H), 9.04 (s, 1H), |
| | | | 8.39 (s, 1H), 8.35 (br. s., 1H), 7.93 (d, J = 8.2 Hz, 1H), 7.87 (d, |
| | | | J = 8.2 Hz, 1H), 7.50 (t, J = 7.8 Hz, 1H), 7.28-7.38 (m, 2H), |
| | | | 7.25 (t, J = 9.0 Hz, 1H), 4.17 (t, J = 4.9 Hz, 2H), 3.77 (t, J = 4.7 |
| | | | Hz, 2H), 1H exchangeable, TFA salt |
| 81 | See text | 776.2 | ¹H NMR (DMSO-d₆) δ: 10.72 (s, 1H), 9.87 (s, 1H), 8.81 (br. s., |
| | | | 1H), 8.35 (s, 1H), 8.32 (s, 1H), 7.91 (d, J = 7.8 Hz, 1H), 7.85 (d, |
| | | | J = 7.8 Hz, 1H), 7.81 (d, J = 2.3 Hz, 1H), 7.48 (t, J = 7.6 Hz, 1H), |
| | | | 7.39 (d, J = 9.0 Hz, 1H), 7.30 (td, J = 8.4, 5.9 Hz, 1H), 7.23 (t, |
| | | | J = 9.0 Hz, 1H), 4.45 (br. s., 2H), 3.71 (d, J = 10.6 Hz, 2H), 3.61 |
| | | | (d, J = 10.2 Hz, 2H), 1.93 (s, 4H) |
| 82 | E | 779.2 | ¹H NMR (DMSO-d₆) δ: 10.74 (s, 1H), 9.97 (s, 1H), 9.13 (br. s., |
| | | | 1H), 8.83 (br. s., 1H), 8.66 (s, 1H), 8.40 (s, 1H), 8.03-8.21 (m, |
| | | | 1H), 7.93-8.01 (m, 1H), 7.88 (d, J = 7.8 Hz, 1H), 7.50-7.58 (m, |
| | | | 3H), 7.31 (td, J = 9.0, 6.3 Hz, 1H), 7.24 (t, J = 9.8 Hz, 1H), 3.98 |
| | | | (d, J = 10.2 Hz, 2H), 3.40 (br. s., 2H), 2.43 (t, J = 12.1 Hz, 2H), |
| | | | 1.18 (d, J = 6.3 Hz, 6H) |
| 83 | E | 809.2 | ¹H NMR (DMSO-d₆) δ: 10.74 (br. s., 1H), 9.95 (s, 1H), 9.78 (br. |
| | | | s., 1H), 8.85 (br. s., 1H), 8.67 (s, 1H), 8.39 (s, 1H), 7.99 (d, J = |
| | | | 8.2 Hz, 1H), 7.94 (d, J = 8.2 Hz, 1H), 7.88 (d, J = 8.2 Hz, 1H), |
| | | | 7.44-7.61 (m, 3H), 7.31 (td, J = 9.0, 6.3 Hz, 1H), 7.24 (t, J = 9.4 |
| | | | Hz, 1H), 4.01 (br. s., 1H), 3.74 (br. s., 1H), 3.65 (br. s., 9H), 3.17 |
| | | | (br. s., 1H), 2.86 (s, 3H) |
| 84 | B | 728.2 | ¹H NMR (DMSO-d₆) δ: 10.73 (s, 1H), 9.84 (s, 1H), 8.76 (br. s., |
| | | | 1H), 8.36 (s, 1H), 8.32 (br. s., 0.6H), 8.23 (br. s., 0.4H), 8.05 (br. |
| | | | s., 0.6H), 7.93 (d, J = 7.8 Hz, 1H), 7.87 (d, J = 7.4 Hz, 1H), 7.84 |
| | | | (br. s., 0.4H), 7.49 (t, J = 8.2 Hz, 1H), 7.35-7.46 (m, 2H), 7.27- |
| | | | 7.35 (m, 1H), 7.23 (t, J = 9.0 Hz, 1H), 4.97 (br. s., 0.6H), 4.81 |
| | | | (br. s., 0.4H), 4.69 (s, 1H), 3.78-3.98 (m, 2H), 3.75 (br. s., 0.4H), |
| | | | 3.66 (br. d, J = 8.6 Hz, 0.6H), 3.57 (br. s., 0.4H), 3.45 (br. s., |
| | | | 0.6H), 1.94 (br. d, J = 9.0 Hz, 1H), 1.86 (br. d, J = 8.6 Hz, 1H) |
| 85 | B | 744.2 | ¹H NMR (DMSO-d₆) δ: 10.73 (s, 1H), 9.81 (s, 1H), 8.78 (br. s., |
| | | | 1H), 8.35 (s, 1H), 8.21 (s, 1H), 7.90-8.00 (m, 1H), 7.87 (d, J = |
| | | | 8.6 Hz, 1H), 7.68 (d, J = 7.8 Hz, 1H), 7.49 (t, J = 7.6 Hz, 1H), |
| | | | 7.35-7.45 (m, 2H), 7.28 (br. s., 1H), 7.23 (br. s., 1H), 4.46 (s, |
| | | | 1H), 3.95 (br. s, 2H), 3.43 (br. s., 2H), 1.52 (br. s., 4H), 1.18 (s, 3H) |
| 86 | B | 756.4 | ¹H NMR (DMSO-d₆) δ: 10.73 (s, 1H), 9.83 (s, 1H), 8.71 (br. s., |
| | | | 1H), 8.35 (s, 1H), 8.24 (s, 1H), 7.93 (d, J = 7.4 Hz, 1H), 7.87 (d, |
| | | | J = 7.4 Hz, 1H), 7.75 (dd, J = 7.0, 1.6 Hz, 1H), 7.45-7.55 (m, |
| | | | 1H), 7.35-7.45 (m, 2H), 7.29 (br. s., 1H), 7.23 (br. s., 1H), 4.64 |
| | | | (d, J = 5.5 Hz, 1H), 4.61 (br. s., 1H), 4.34 (br. s., 1H), 4.00 (tq, |
| | | | J = 10.7, 5.6 Hz, 1H), 2.00 (br. s., 2H), 1.89 (br. s., 2H), 1.73 (d, |
| | | | J = 7.8 Hz, 2H), 1.63 (br. s., 1H), 1.41 (br. s., 1H) |
| 87 | B | 744.2 | ¹H NMR (DMSO-d₆) δ: 10.73 (s, 1H), 9.86 (s, 1H), 8.74 (br. s., |
| | | | 1H), 8.36 (s, 1H), 8.19 (s, 1H), 8.16 (dd, J = 7.0, 1.6 Hz, 1H), |
| | | | 7.93 (d, J = 7.8 Hz, 1H), 7.87 (d, J = 8.2 Hz, 1H), 7.49 (t, J = 8.0 |
| | | | Hz, 1H), 7.36-7.46 (m, 2H), 7.27-7.35 (m, 1H), 7.23 (t, J = 9.0 |
| | | | Hz, 1H), 4.57 (s, 1H), 4.44 (t, J = 8.0 Hz, 1H), 4.35 (t, J = 7.0 Hz, |
| | | | 1H), 3.88-4.10 (m, 2H), 2.60-2.75 (m, 1H), 1.09 (s, 6H) |
| 88 | A | 645.2 | ¹H NMR (DMSO-d₆) δ: 10.73 (s, 1H), 9.91 (s, 1H), 8.87 (s, 1H), |
| | | | 8.81 (br. s., 1H), 8.38 (s, 1H), 8.25-8.34 (m, 1H), 7.93 (d, J = |
| | | | 8.2 Hz, 1H), 7.87 (d, J = 7.8 Hz, 1H), 7.49 (t, J = 8.2 Hz, 1H), |
| | | | 7.37-7.47 (m, 2H), 7.32 (td, J = 8.5, 6.1 Hz, 1H), 7.24 (t, J = 9.2 |
| | | | Hz, 1H), 2.63 (s, 3H) |
| 89 | B | 784.4 | ¹H NMR (DMSO-d₆) δ: 10.73 (br. s., 1H), 9.82 (s, 1H), 8.75 (br. |
| | | | s., 1H), 8.35 (s, 1H), 8.19 (s, 1H), 7.93 (d, J = 8.2 Hz, 1H), 7.87 |
| | | | (d, J = 7.8 Hz, 1H), 7.66 (d, J = 7.4 Hz, 1H), 7.45-7.57 (m, 1H), |
| | | | 7.35-7.45 (m, 2H), 7.27-7.35 (m, 1H), 7.23 (t, J = 9.0 Hz, 1H), |
| | | | 4.44 (s, 1H), 4.02 (br. s., 1H), 3.75 (br. s., 1H), 3.51 (br. s., 2H), |
| | | | 1.94 (br. s., 1H), 1.60 (br. d, J = 16.8 Hz, 4H), 1.35 (d, J = 9.0 |
| | | | Hz, 3H), 1.21 (br. s., 3H) |
| 90 | B | 758.4 | ¹H NMR (DMSO-d₆) δ: 10.73 (s, 1H), 9.84 (s, 1H), 8.74 (br. s, |
| | | | 1H), 8.35 (s, 1H), 8.27 (s, 1H), 7.90-8.01 (m, 2H), 7.87 (d, J = |
| | | | 7.0 Hz, 1H), 7.49 (t, J = 7.4 Hz, 1H), 7.34-7.45 (m, 2H), 7.27- |
| | | | 7.34 (m, 1H), 7.23 (t, J = 9.0 Hz, 1H), 4.40 (d, J = 13.7 Hz, 1H), |
| | | | 3.59-3.87 (m, 3H), 3.40-3.53 (m, 1H), 2.18-2.31 (m, 1H), |
| | | | 1.73-1.98 (m, 2H), 1.16 (d, J = 4.3 Hz, 3H), 1.10 (d, J = 3.5 Hz, 3H) |
| 91 | See text | 659.2 | ¹H NMR (DMSO-d₆) δ: 10.74 (s, 1H), 9.90 (s, 1H), 8.86 (s, 1H), |
| | | | 8.80 (br. s., 1H), 8.27-8.43 (m, 2H), 7.93 (d, J = 8.2 Hz, 1H), |
| | | | 7.87 (d, J = 7.8 Hz, 1H), 7.49 (t, J = 7.8 Hz, 1H), 7.39-7.47 (m, |
| | | | 2H), 7.32 (td, J = 8.2, 6.3 Hz, 1H), 7.24 (t, J = 8.6 Hz, 1H), 3.09 |
| | | | (q, J = 7.4 Hz, 2H), 1.15 (t, J = 7.2 Hz, 3H) |
| 92 | See text | 673.2 | ¹H NMR (DMSO-d₆) δ: 10.74 (br. s., 1H), 9.89 (s, 1H), 8.91 (s, |
| | | | 1H), 8.84 (br. s., 1H), 8.29-8.42 (m, 2H), 7.92 (d, J = 7.4 Hz, |
| | | | 1H), 7.87 (d, J = 7.4 Hz, 1H), 7.46-7.54 (m, 1H), 7.44 (s, 2H), |
| | | | 7.30 (s, 1H), 7.22 (t, J = 7.8 Hz, 1H), 3.69 (spt, J = 6.3 Hz, 1H), |
| | | | 1.19 (d, J = 6.7 Hz, 6H) |
| 93 | B | 760.2 | ¹H NMR (DMSO-d₆) δ: 10.73 (s, 1H), 9.85 (s, 1H), 8.64 (br. s., |
| | | | 1H), 8.36 (s, 1H), 8.30 (s, 1H), 7.93 (d, J = 7.8 Hz, 1H), 7.87 (d, |
| | | | J = 8.2 Hz, 1H), 7.50 (t, J = 8.0 Hz, 1H), 7.35-7.46 (m, 1H), |
| | | | 7.26-7.35 (m, 1H), 7.14-7.26 (m, 2H), 4.59 (br. s., 1H), 3.96 |
| | | | (br. s., 1H), 3.69 (br. s., 2H), 3.59 (br. s., 1H), 3.50 (br. s., 1H), |
| | | | 1.95 (br. s., 4H) |
| 94 | B | 716.4 | ¹H NMR (DMSO-d₆) δ: 10.73 (s, 1H), 9.85 (s, 1H), 8.73 (br. s., |
| | | | 1H), 8.36 (s, 1H), 8.19 (s, 1H), 8.17 (dd, J = 7.4, 1.6 Hz, 1H), |
| | | | 7.93 (d, J = 8.2 Hz, 1H), 7.87 (d, J = 7.4 Hz, 1H), 7.49 (t, J = 7.6 |
| | | | Hz, 1H), 7.36-7.46 (m, 2H), 7.27-7.35 (m, 1H), 7.23 (t, J = 9.0 |
| | | | Hz, 1H), 5.72 (s, 1H), 4.39 (br. s., 1H), 4.31 (br. s., 1H), 3.95 (br. |
| | | | s., 2H), 1.45 (s, 3H) |
| 95 | B | 730.2 | ¹H NMR (DMSO-d₆) δ: 10.73 (s, 1H), 9.85 (s, 1H), 8.72 (br. s., |
| | | | 1H), 8.36 (s, 1H), 8.20 (s, 1H), 8.17 (dd, J = 7.0, 1.6 Hz, 1H), |
| | | | 7.93 (d, J = 7.8 Hz, 1H), 7.87 (d, J = 7.4 Hz, 1H), 7.49 (t, J = 8.2 |
| | | | Hz, 1H), 7.36-7.46 (m, 2H), 7.31 (td, J = 8.5, 5.7 Hz, 1H), 7.24 |
| | | | (t, J = 9.2 Hz, 1H), 5.62 (s, 1H), 4.39 (br. s., 1H), 4.26 (br. s., |
| | | | 1H), 3.98 (br. s., 1H), 3.89 (br. s., 1H), 1.72 (q, J = 7.2 Hz, 2H), |
| | | | 0.93 (t, J = 7.4 Hz, 3H) |
| 96 | B | 770.2 | ¹H NMR (DMSO-d₆) δ: 10.73 (s, 1H), 9.86 (s, 1H), 8.78 (br. s., |
| | | | 1H), 8.37 (s, 1H), 8.32 (s, 1H), 8.16 (dd, J = 7.0, 2.0 Hz, 1H), |
| | | | 7.94 (d, J = 8.6 Hz, 1H), 7.87 (d, J = 7.8 Hz, 1H), 7.47-7.54 (m, |
| | | | 2H), 7.38-7.47 (m, 2H), 7.31 (td, J = 8.4, 5.9 Hz, 1H), 7.24 (t, |
| | | | J = 8.8 Hz, 1H), 4.89 (br. s., 1H), 4.50 (br. s., 1H), 4.34 (br. s., 1H), |
| | | | 4.13 (br. s., 1H) |
| 97 | B | 798.2 | ¹H NMR (DMSO-d₆) δ: 10.73 (s, 1H), 9.83 (s, 1H), 8.79 (br. s., |
| | | | 1H), 8.35 (s, 1H), 8.29 (s, 1H), 7.93 (d, J = 7.8 Hz, 1H), 7.87 (d, |
| | | | J = 7.8 Hz, 1H), 7.70 (d, J = 8.2 Hz, 1H), 7.50 (t, J = 7.8 Hz, 1H), |
| | | | 7.35-7.46 (m, 2H), 7.27-7.35 (m, 1H), 7.24 (t, J = 9.0 Hz, 1H), |
| | | | 6.17 (s, 1H), 4.23 (br. s., 2H), 3.22 (br. s., 2H), 1.74 (br. s., 4H) |
| 98 | See text | 785.4 | ¹H NMR (DMSO-d₆) δ: 10.66 (br. s., 1H), 9.84 (s, 1H), 8.74 (br. |
| | | | s., 1H), 8.36 (s, 1H), 8.24 (s, 1H), 7.93 (d, J = 7.8 Hz, 1H), 7.87 |
| | | | (d, J = 6.7 Hz, 1H), 7.76 (d, J = 8.2 Hz, 1H), 7.50 (t, J = 7.4 Hz, |
| | | | 1H), 7.36-7.46 (m, 2H), 7.27-7.35 (m, 1H), 7.23 (t, J = 9.0 Hz, |
| | | | 1H), 4.20-4.72 (m, 3H), 3.51 (br. s., 2H), 3.38 (br. s., 2H), 2.81 |
| | | | (br. s., 2H), 2.41 (br. s., 2H), 1.90 (br. s., 4H) |
| 99 | See text | 634.0 | ¹H NMR (DMSO-d₆) δ: 10.73 (br. s., 1H), 9.90 (s, 1H), 8.89 (s, |
| | | | 1H), 8.38 (s, 1H), 8.20 (br. s., 1H), 7.93 (d, J = 7.4 Hz, 1H), 7.87 |
| | | | (d, J = 7.4 Hz, 1H), 7.50 (t, J = 8.0 Hz, 1H), 7.38 (t, J = 8.0 Hz, |
| | | | 1H), 7.26-7.34 (m, 1H), 7.22 (t, J = 9.0 Hz, 1H), 6.99 (d, J = 8.2 |
| | | | Hz, 1H), 3.99 (s, 3H) |
| 100 | See text | 682.0 | ¹H NMR (DMSO-d₆) δ: 10.70 (s, 1H), 10.03 (s, 1H), 8.90 (s, 1H), |
| | | | 8.38 (s, 1H), 7.94 (d, J = 7.8 Hz, 1H), 7.92 (br. s., 1H), 7.88 (d, |
| | | | J = 7.8 Hz, 1H), 7.51 (t, J = 8.0 Hz, 1H), 7.30 (td, J = 8.6, 5.9 Hz, |
| | | | 1H), 7.23 (t, J = 9.2 Hz, 1H), 7.04 (dd, J = 12.3, 1.8 Hz, 1H), 4.92 |
| | | | (t, J = 5.3 Hz, 1H), 4.16 (t, J = 4.7 Hz, 2H), 3.77 (q, J = 5.1 Hz, 2H) |
| 101 | B | 776.2 | ¹H NMR (DMSO-d₆) δ: 10.73 (s, 1H), 9.88 (s, 1H), 8.88 (br. s, |
| | | | 1H), 8.43 (s, 1H), 8.36 (s, 1H), 7.93 (d, J = 8.2 Hz, 1H), 7.87 (d, |
| | | | J = 8.2 Hz, 1H), 7.63 (d, J = 9.0 Hz, 1H), 7.49 (t, J = 7.6 Hz, 1H), |
| | | | 7.28-7.37 (m, 1H), 7.24 (t, J = 8.8 Hz, 2H), 4.88 (br. s., 1H), |
| | | | 4.41 (t, J = 5.5 Hz, 1H), 3.61-3.87 (m, 2H), 1.81-2.05 (m, 3H), |
| | | | 1.67 (br. s., 1H), 1.18 (s, 3H), 1.15 (s, 3H) |
| 102 | B | 760.2 | ¹H NMR (DMSO-d₆) δ: 10.73 (s, 1H), 9.87 (s, 1H), 8.83 (br. s., |
| | | | 1H), 8.36 (s, 1H), 8.34 (s, 1H), 7.93 (d, J = 7.8 Hz, 1H), 7.87 (d, |
| | | | J = 7.8 Hz, 1H), 7.55 (dd, J = 9.2, 2.5 Hz, 1H), 7.49 (t, J = 8.0 |
| | | | Hz, 1H), 7.28-7.37 (m, 1H), 7.14-7.28 (m, 2H), 4.45 (br. s., |
| | | | 2H), 3.73 (d, J = 10.6 Hz, 2H), 3.62 (d, J = 11.0 Hz, 2H), 1.95 |
| | | | (br. s., 4H) |
| 103 | A | 582.2 | ¹H NMR (DMSO-d₆) δ: 10.69 (br. s., 1H), 9.81 (s, 1H), 8.37 (s, |
| | | | 1H), 8.20 (s, 1H), 7.93 (d, J = 7.8 Hz, 1H), 7.88 (d, J = 7.8 Hz, |
| | | | 1H), 7.50 (t, J = 8.0 Hz, 1H), 7.26 (td, J = 9.0, 5.5 Hz, 1H), 7.18 |
| | | | (t, J = 9.0 Hz, 1H), 2.40 (br. s., 3H), 2.13 (s, 3H) |
| 104 | See text | 671.2 | ¹H NMR (DMSO-d₆) δ: 10.74 (br. s., 1H), 9.92 (br. s., 1H), 9.10 |
| | | | (s, 1H), 8.74 (br. s., 1H), 8.39 (s, 1H), 7.87-7.90 (m, 2H), 7.41- |
| | | | 7.61 (m, 3H), 7.27 (br. s., 1H), 7.19 (br. s., 1H), 1.95-2.01 (m, 6H) |
| 105 | See text | 635.2 | ¹H NMR (DMSO-d₆) δ: 10.73 (br. s., 1H), 9.96 (s, 1H), 9.01 (s, |
| | | | 1H), 8.40 (s, 1H), 8.29 (br. s., 1H), 8.13 (d, J = 5.1 Hz, 1H), 7.92 |
| | | | (d, J = 7.0 Hz, 1H), 7.88 (d, J = 7.8 Hz, 1H), 7.50 (t, J = 8.0 Hz, |
| | | | 1H), 7.26-7.37 (m, 1H), 7.10-7.26 (m, 1H), 4.07 (s, 3H) |
| 106 | See text | 689.2 | ¹H NMR (DMSO-d₆) δ: 10.73 (s, 1H), 9.91 (s, 1H), 8.93 (s, 1H), |
| | | | 8.83 (br. s., 1H), 8.38 (s, 1H), 8.24-8.35 (m, 1H), 7.93 (d, J = |
| | | | 7.8 Hz, 1H), 7.87 (d, J = 7.4 Hz, 1H), 7.37-7.58 (m, 3H), 7.32 |
| | | | (td, J = 9.0, 5.9 Hz, 1H), 7.25 (t, J = 9.0 Hz, 1H), 3.76 (t, J = 6.1 |
| | | | Hz, 2H), 3.30 (d, J = 6.3 Hz, 2H), 3.27 (s, 3H) |
| 107 | See text | 713.0 | ¹H NMR (DMSO-d₆) δ: 10.73 (br. s., 1H), 9.92 (s, 1H), 8.79 (br. |
| | | | s., 1H), 8.68 (s, 1H), 8.39 (s, 1H), 8.18-8.27 (m, 1H), 8.15 (d, |
| | | | J = 3.9 Hz, 1H), 8.11 (d, J = 5.1 Hz, 1H), 7.92 (d, J = 7.0 Hz, 1H), |
| | | | 7.87 (d, J = 7.8 Hz, 1H), 7.41-7.56 (m, 3H), 7.34-7.39 (m, 1H), |
| | | | 7.25-7.34 (m, 1H), 7.21 (br. s., 1H) |
| 108 | See text | 648.2 | ¹H NMR (DMSO-d₆) δ: 10.73 (s, 1H), 9.90 (s, 1H), 8.88 (s, 1H), |
| | | | 8.38 (s, 1H), 8.15 (br. s., 1H), 7.93 (d, J = 8.2 Hz, 1H), 7.87 (d, |
| | | | J = 7.8 Hz, 1H), 7.50 (t, J = 7.8 Hz, 1H), 7.36 (t, J = 7.8 Hz, 1H), |
| | | | 7.31 (td, J = 8.6, 5.9 Hz, 1H), 7.23 (t, J = 8.6 Hz, 1H), 6.97 (d, |
| | | | J = 7.8 Hz, 1H), 4.31 (q, J = 6.9 Hz, 2H), 1.43 (t, J = 7.0 Hz, 3H) |
| 109 | A | 630.2 | ¹H NMR (DMSO-d₆) δ: 10.69 (s, 1H), 9.92 (s, 1H), 8.56 (s, 1H), |
| | | | 8.42 (s, 1H), 8.37 (s, 1H), 7.95 (dd, J = 7.8, 1.2 Hz, 1H), 7.85- |
| | | | 7.93 (m, 3H), 7.52 (t, J = 8.0 Hz, 1H), 7.45 (t, J = 7.6 Hz, 2H), |
| | | | 7.32 (tt, J = 7.4, 1.2 Hz, 1H), 7.28 (td, J = 8.6, 5.9 Hz, 1H), 7.20 |
| | | | (t, J = 8.6 Hz, 1H) |
| 110 | See text | 686.2 | ¹H NMR (DMSO-d₆) δ: 10.72 (br. s., 1H), 9.76 (br. s., 1H), 8.79 |
| | | | (br. s., 1H), 8.33 (s, 1H), 8.14 (s, 1H), 7.87 (d, J = 7.8 Hz, 2H), |
| | | | 7.83 (d, J = 7.8 Hz, 1H), 7.46 (t, J = 7.4 Hz, 1H), 7.40 (t, J = 7.8 |
| | | | Hz, 1H), 7.32 (t, J = 7.4 Hz, 1H), 7.26 (br. s., 1H), 7.18 (br. s., |
| | | | 1H), 4.06 (t, J = 7.0 Hz, 2H), 2.52-2.56 (m, 2H), 2.20 (quin, J = |
| | | | 7.5 Hz, 2H) |
| 111 | See text | 664.2 | ¹H NMR (DMSO-d₆) δ: 10.73 (s, 1H), 9.90 (s, 1H), 8.89 (s, 1H), |
| | | | 8.38 (s, 1H), 8.19 (br. s., 1H), 7.93 (d, J = 7.8 Hz, 1H), 7.87 (d, |
| | | | J = 7.8 Hz, 1H), 7.50 (t, J = 8.0 Hz, 1H), 7.36 (t, J = 7.8 Hz, 1H), |
| | | | 7.30 (td, J = 8.6, 6.3 Hz, 1H), 7.23 (t, J = 9.0 Hz, 1H), 6.99 (d, |
| | | | J = 7.8 Hz, 1H), 4.94 (t, J = 5.5 Hz, 1H), 4.28 (t, J = 5.1 Hz, 2H), |
| | | | 3.82 (q, J = 5.1 Hz, 2H) |
| 112 | See text | 635.4 | ¹H NMR (DMSO-d₆) δ: 10.51 (br. s., 1H), 9.94 (s, 1H), 9.10 (s, |
| | | | 1H), 8.73 (br. s., 1H), 8.40 (s, 1H), 7.60 (d, J = 7.8 Hz, 1H), 7.42- |
| | | | 7.56 (m, 3H), 7.34-7.42 (m, 1H), 7.26-7.34 (m, 1H), 7.23 (t, |
| | | | J = 8.6 Hz, 1H), 2.48 (br. s., 3H), 1.98 (s, 6H) |
| 113 | G | 717.2 | ¹H NMR (DMSO-d₆) δ: 10.72 (s, 1H), 9.89 (s, 1H), 8.81 (s, 1H), |
| | | | 8.38 (s, 1H), 8.04 (br. s., 1H), 7.94 (d, J = 8.2 Hz, 1H), 7.87 (d, |
| | | | J = 7.8 Hz, 1H), 7.50 (t, J = 8.2 Hz, 1H), 7.25-7.35 (m, 2H), |
| | | | 7.23 (t, J = 9.4 Hz, 1H), 6.81 (d, J = 8.2 Hz, 1H), 4.31 (s, 1H), |
| | | | 3.78 (dt, J = 11.9, 4.2 Hz, 2H), 3.34-3.43 (m, 2H), 1.56-1.75 |
| | | | (m, 4H), 1.20 (s, 3H) |
| 114 | A | 603.2 | ¹H NMR (DMSO-d₆) δ: 10.73 (s, 1H), 9.77 (s, 1H), 8.73 (br. s., |
| | | | 1H), 8.33 (s, 1H), 7.96 (d, J = 3.5 Hz, 1H), 7.93 (dd, J = 8.2, 1.2 |
| | | | Hz, 1H), 7.87 (d, J = 7.8 Hz, 1H), 7.70 (d, J = 7.4 Hz, 1H), 7.49 |
| | | | (t, J = 8.0 Hz, 1H), 7.37 (t, J = 7.2 Hz, 1H), 7.27-7.34 (m, 2H), |
| | | | 7.23 (t, J = 9.2 Hz, 1H), 6.93 (d, J = 3.5 Hz, 1H) |
| 115 | A | 604.2 | ¹H NMR (DMSO-d₆) δ: 10.73 (s, 1H), 9.82 (s, 1H), 9.11 (br. s., |
| | | | 1H), 8.57 (d, J = 4.3 Hz, 1H), 8.34 (s, 1H), 8.32 (d, J = 3.5 Hz, |
| | | | 1H), 7.93 (d, J = 7.8 Hz, 1H), 7.87 (d, J = 7.8 Hz, 1H), 7.50 (t, |
| | | | J = 7.8 Hz, 1H), 7.34-7.43 (m, 1H), 7.31 (td, J = 8.7, 6.1 Hz, 1H), |
| | | | 7.23 (t, J = 9.0 Hz, 1H), 7.04 (d, J = 3.5 Hz, 1H) |
| 116 | See text | 651.2 | ¹H NMR (DMSO-d₆) δ: 10.57 (br. s., 1H), 9.94 (s, 1H), 9.10 (s, |
| | | | 1H), 8.71 (br. s., 1H), 8.40 (s, 1H), 7.76 (d, J = 8.2 Hz, 1H), 7.73 |
| | | | (d, J = 8.6 Hz, 1H), 7.51 (d, J = 2.3 Hz, 2H), 7.35 (t, J = 7.8 Hz, |
| | | | 1H), 7.26-7.32 (m, 1H), 7.23 (t, J = 9.0 Hz, 1H), 2.65 (s, 3H), |
| | | | 1.98 (s, 6H) |
| 117 | See text | 681.4 | ¹H NMR (DMSO-d₆) δ: 10.40 (br. s., 1H), 9.94 (s, 1H), 9.10 (s, |
| | | | 1H), 8.72 (br. s., 1H), 8.38 (s, 1H), 7.75 (d, J = 8.6 Hz, 1H), 7.51 |
| | | | (br. s., 2H), 7.30 (td, J = 8.4, 5.9 Hz, 1H), 7.22 (t, J = 8.6 Hz, |
| | | | 1H), 7.10 (d, J = 9.0 Hz, 1H), 3.90 (s, 3H), 2.64 (s, 3H), 1.98 (s, 6H) |
| 118 | See text | 665.2 | ¹H NMR (DMSO-d₆) δ: 10.34 (br. s., 1H), 9.94 (s, 1H), 9.10 (s, |
| | | | 1H), 8.73 (br. s., 1H), 8.39 (s, 1H), 7.56 (d, J = 9.0 Hz, 1H), 7.51 |
| | | | (br. s., 2H), 7.30 (td, J = 9.0, 6.3 Hz, 1H), 7.22 (t, J = 9.0 Hz, |
| | | | 1H), 7.10 (t, J = 8.4 Hz, 1H), 3.87 (s, 3H), 2.47 (d, J = 2.0 Hz, |
| | | | 3H), 1.98 (s, 6H) |
| 119 | See text | 678.2 | ¹H NMR (DMSO-d₆) δ: 10.72 (s, 1H), 9.90 (s, 1H), 8.89 (s, 1H), |
| | | | 8.38 (s, 1H), 8.19 (br. s., 1H), 7.93 (d, J = 7.8 Hz, 1H), 7.87 (d, |
| | | | J = 7.0 Hz, 1H), 7.50 (t, J = 8.2 Hz, 1H), 7.36 (t, J = 8.1 Hz, 1H), |
| | | | 7.30 (td, J = 8.2, 5.9 Hz, 1H), 7.23 (t, J = 9.0 Hz, 1H), 6.99 (d, |
| | | | J = 7.8 Hz, 1H), 4.33-4.44 (m, 2H), 3.74-3.81 (m, 2H), 3.36 (s, 3H) |
| 120 | See text | 629.0 | ¹H NMR (DMSO-d₆; 600 MHz) δ: 10.74 (br. s., 1H), 9.99 (s, 1H), |
| | | | 9.29 (s, 1H), 9.11 (br. s., 1H), 8.41 (s, 1H), 7.97 (d, J = 7.3 Hz, |
| | | | 1H), 7.93 (d, J = 7.3 Hz, 1H), 7.87 (d, J = 7.0 Hz, 1H), 7.63 (br. |
| | | | s., 1H), 7.50 (br. s., 1H), 7.28-7.35 (m, 1H), 7.24 (t, J = 8.2 Hz, 1H) |
| 121 | A | 598.2 | ¹H NMR (DMSO-d₆; 600 MHz) δ: 10.68 (br. s., 1H), 9.86 (s, 1H), |
| | | | 8.40 (br. s., 1H), 8.34 (s, 1H), 7.94 (d, J = 7.9 Hz, 1H), 7.88 (dd, |
| | | | J = 7.9, 1.5 Hz, 1H), 7.67 (br. s., 1H), 7.51 (t, J = 8.0 Hz, 1H), |
| | | | 7.26 (dd, J = 8.8, 5.6 Hz, 1H), 7.18 (t, J = 8.8 Hz, 1H), 4.68 (t, |
| | | | J = 5.3 Hz, 1H), 3.68 (q, J = 6.7 Hz, 2H), 2.71 (t, J = 6.9 Hz, 2H) |
| 122 | See text | 669.2 | ¹H NMR (DMSO-d₆; 600 MHz) δ: 10.73 (br. s., 1H), 9.94 (s, 1H), |
| | | | 9.07 (s, 1H), 8.72 (br. s., 1H), 8.39 (s, 1H), 7.92 (d, J = 5.3 Hz, |
| | | | 1H), 7.88 (d, J = 7.6 Hz, 1H), 7.40-7.59 (m, 3H), 7.27-7.34 (m, |
| | | | 1H), 7.22 (br. s., 1H), 1.85-1.90 (m, 2H), 1.81-1.85 (m, 2H) |
| 123 | See text | 647.2 | ¹H NMR (DMSO-d₆; 600 MHz) δ: 10.74 (br. s., 1H), 9.98 (s, 1H), |
| | | | 9.27 (s, 1H), 8.95 (br. s., 1H), 8.88 (d, J = 2.0 Hz, 1H), 8.41 (s, |
| | | | 1H), 8.08 (d, J = 7.6 Hz, 1H), 7.90-7.99 (m, 2H), 7.88 (d, J = |
| | | | 7.6 Hz, 1H), 7.60 (br. s., 1H), 7.50 (br. s., 1H), 7.27-7.36 (m, |
| | | | 1H), 7.23 (br. s., 1H) |
| 124 | A | 593.2 | ¹H NMR (DMSO-d₆; 600 MHz) δ: 10.68 (s, 1H), 9.91 (s, 1H), |
| | | | 8.53 (br. s., 1H), 8.36 (s, 1H), 7.94 (d, J = 7.9 Hz, 1H), 7.88 (dd, |
| | | | J = 7.9, 1.5 Hz, 2H), 7.52 (t, J = 8.0 Hz, 1H), 7.23-7.32 (m, 1H), |
| | | | 7.19 (t, J = 8.5 Hz, 1H), 4.03 (s, 2H) |
| 125 | A | 568.2 | ¹H NMR (DMSO-d₆; 600 MHz) δ: 10.68 (br. s., 1H), 9.84 (s, 1H), |
| | | | 8.38 (br. s., 1H), 8.34 (s, 1H), 7.94 (d, J = 7.9 Hz, 1H), 7.88 (dd, |
| | | | J = 7.9, 1.2 Hz, 1H), 7.61 (br. s., 1H), 7.51 (t, J = 8.0 Hz, 1H), |
| | | | 7.22-7.30 (m, 1H), 7.18 (t, J = 9.1 Hz, 1H), 2.20 (s, 3H) |
| 126 | See text | 648.2 | ¹H NMR (DMSO-d₆) δ: 10.73 (s, 1H), 9.90 (s, 1H), 8.97 (s, 1H), |
| | | | 8.47 (br. s., 1H), 8.38 (s, 1H), 7.92 (d, J = 7.4 Hz, 1H), 7.87 (d, |
| | | | J = 7.8 Hz, 1H), 7.49 (t, J = 8.2 Hz, 1H), 7.37 (t, J = 7.6 Hz, 1H), |
| | | | 7.26-7.34 (m, 2H), 7.22 (t, J = 9.0 Hz, 1H), 4.75 (t, J = 5.3 Hz, |
| | | | 1H), 3.77 (q, J = 7.0 Hz, 2H), 3.17 (t, J = 7.2 Hz, 2H) |
| 127 | G | 701.2 | ¹H NMR (DMSO-d₆) δ: 10.72 (s, 1H), 9.87 (s, 1H), 8.76 (s, 1H), |
| | | | 8.37 (s, 1H), 7.92 (d, J = 7.0 Hz, 1H), 7.88 (d, J = 7.8 Hz, 1H), |
| | | | 7.49 (t, J = 7.4 Hz, 1H), 7.19-7.32 (m, 3H), 6.51 (d, J = 7.8 Hz, |
| | | | 1H), 5.54 (s, 1H), 4.67 (s, 1H), 3.83 (d, J = 6.7 Hz, 1H), 3.74- |
| | | | 3.79 (m, 2H), 2.05-2.11 (m, 2H), 1.97-2.01 (m, 1H), 1.87- |
| | | | 1.92 (m, 1H) |
| 128 | G | 705.2 | ¹H NMR (DMSO-d₆) δ: 10.72 (s, 1H), 9.87 (s, 1H), 8.79 (s, 1H), |
| | | | 8.37 (s, 1H), 7.93 (d, J = 8.2 Hz, 1H), 7.89 (br. s., 1H), 7.87 (d, |
| | | | J = 7.0 Hz, 1H), 7.49 (t, J = 8.0 Hz, 1H), 7.12-7.38 (m, 3H), |
| | | | 6.61 (d, J = 8.2 Hz, 1H), 3.91 (t, J = 7.0 Hz, 2H), 3.37 (t, J = 6.3 |
| | | | Hz, 2H), 3.20 (s, 3H), 3.11 (s, 3H), 1.82 (quin, J = 7.0 Hz, 2H) |
| 129 | G | 729.2 | ¹H NMR (DMSO-d₆) δ: 10.72 (s, 1H), 9.87 (s, 1H), 8.78 (s, 1H), |
| | | | 8.38 (s, 1H), 7.92 (d, J = 7.8 Hz, 1H), 7.88 (d, J = 7.0 Hz, 2H), |
| | | | 7.49 (t, J = 7.8 Hz, 1H), 7.16-7.35 (m, 3H), 6.76 (d, J = 8.2 Hz, |
| | | | 1H), 5.07 (br. s., 2H), 4.36 (d, J = 5.9 Hz, 1H), 3.93-4.04 (m, |
| | | | 1H), 1.95-2.02 (m, 2H), 1.72-1.79 (m, 4H), 1.53 (t, J = 10.8 |
| | | | Hz, 2H) |
| 130 | G | 730.2 | ¹H NMR (DMSO-d₆) δ: 10.72 (br. s., 1H), 9.88 (s, 1H), 8.88 (s, |
| | | | 1H), 8.38 (s, 1H), 8.17 (br. s., 1H), 7.88 (d, J = 7.8 Hz, 2H), 7.47 |
| | | | (t, J = 7.4 Hz, 1H), 7.32 (t, J = 7.8 Hz, 1H), 7.26 (br. s., 1H), 7.16 |
| | | | (br. s., 1H), 6.82 (d, J = 7.8 Hz, 1H), 3.62-3.76 (m, 4H), 3.51- |
| | | | 3.62 (m, 2H), 3.41-3.51 (m, 2H), 2.07 (s, 3H) |
| 131 | See text | 722.2 | ¹H NMR (DMSO-d₆) δ: 10.73 (s, 1H), 9.91 (s, 1H), 8.89 (s, 1H), |
| | | | 8.38 (s, 1H), 8.20 (br. s., 1H), 7.94 (d, J = 8.2 Hz, 1H), 7.87 (d, |
| | | | J = 7.8 Hz, 1H), 7.50 (t, J = 7.6 Hz, 1H), 7.36 (t, J = 9.0 Hz, 1H), |
| | | | 7.26-7.34 (m, 1H), 7.24 (t, J = 9.0 Hz, 1H), 7.00 (d, J = 7.8 Hz, |
| | | | 1H), 4.39 (dd, J = 5.5, 3.9 Hz, 2H), 3.80-3.89 (m, 2H), 3.60- |
| | | | 3.69 (m, 2H), 3.49 (dd, J = 5.7, 3.7 Hz, 2H), 3.26 (s, 3H) |
| 132 | G | 717.2 | ¹H NMR (DMSO-d₆) δ: 10.72 (s, 1H), 9.86 (s, 1H), 8.76 (s, 1H), |
| | | | 8.37 (s, 1H), 7.92 (d, J = 7.8 Hz, 1H), 7.88 (d, J = 8.2 Hz, 1H), |
| | | | 7.82 (br. s., 1H), 7.49 (t, J = 7.8 Hz, 1H), 7.12-7.34 (m, 3H), |
| | | | 6.50 (d, J = 8.2 Hz, 1H), 4.74-4.95 (m, 1H), 3.70-3.79 (m, 1H), |
| | | | 3.57 (dd, J = 9.2, 3.3 Hz, 1H), 3.36-3.46 (m, 1H), 3.29-3.30 |
| | | | (m, 1H), 3.28 (s, 3H), 1.89-2.08 (m, 4H) |
| 133 | See text | 696.2 | ¹H NMR (DMSO-d₆) δ: 9.77 (s, 1H), 9.04 (s, 1H), 8.87 (br. s., |
| | | | 1H), 8.41 (s, 1H), 8.18 (s, 1H), 8.14 (br. s., 1H), 8.07 (br. s., 1H), |
| | | | 7.94 (dd, J = 7.8, 1.5 Hz, 1H), 7.67-7.75 (br. s., 1H), 7.64 (d, |
| | | | J = 7.8 Hz, 1H), 7.54 (br. s., 1H), 7.38-7.45 (m, 1H), 7.35 (t, J = |
| | | | 8.0 Hz, 1H), 7.24-7.32 (m, 1H), 7.16 (br. s., 1H), 7.01-7.12 (m, |
| | | | 1H), 6.80 (t, J = 9.2 Hz, 1H), 6.74 (br. s., 1H), 6.58 (br. s., 1H), |
| | | | 5.90 (br. s., 2H) (4H extra) |
| 134 | See text | 696.2 | ¹H NMR (DMSO-d₆) δ: 12.72 (br. s., 1H), 10.73 (br. s, 1H), 9.86 |
| | | | (br. s., 1H), 9.05 (s, 1H), 8.78 (br. s., 1H), 8.40 (s, 1H), 8.14 (s, |
| | | | 1H), 8.05 (d, J = 4.0 Hz, 1H), 7.91 (d, J = 8.0 Hz, 1H), 7.78 (br. |
| | | | s., 1H), 7.56 (br. s., 1H), 7.53 (dd, J = 7.3, 1.6 Hz, 1H), 7.42 (d, |
| | | | J = 6.5 Hz, 2H), 7.18 (br. s., 1H), 6.99 (br. s., 1H), 6.73 (dd, J = |
| | | | 7.3, 4.9 Hz, 1H), 5.60 (s, 2H), formic acid salt |
| 135 | G | 731.2 | ¹H NMR (DMSO-d₆) δ: 10.72 (br. s., 1H), 9.87 (s, 1H), 8.82 (s, |
| | | | 1H), 8.38 (s, 1H), 8.07 (br. s., 1H), 7.84-7.94 (m, 2H), 7.48 (t, |
| | | | J = 7.8 Hz, 1H), 7.22-7.34 (m, 2H), 7.17 (br. s., 1H), 6.81 (d, |
| | | | J = 8.0 Hz, 1H), 3.78-3.84 (m, 2H), 3.21-3.29 (m, 2H), 3.16 (s, |
| | | | 3H), 1.84 (s, 2H), 1.64-1.73 (m, 2H), 1.18 (s, 3H) |
| 136 | G | 743.2 | ¹H NMR (DMSO-d₆) δ: 10.66 (br. s., 1H), 9.82 (s, 1H), 8.76 (s, |
| | | | 1H), 8.32 (s, 1H), 8.01 (br. s., 1H), 7.86 (br. s., 1H), 7.82 (d, J = |
| | | | 8.4 Hz, 1H), 7.42 (t, J = 7.9 Hz, 1H), 7.18-7.26 (m, 2H), 7.12 (t, |
| | | | J = 8.3 Hz, 1H), 6.75 (d, J = 8.0 Hz, 1H), 3.71 (t, J = 6.7 Hz, 2H), |
| | | | 3.55-3.62 (m, 2H), 3.46 (ddd, J = 11.9, 7.6, 4.5 Hz, 2H), 1.85 |
| | | | (quin, J = 7.0 Hz, 2H), 1.63-1.71 (m, 6H) |
| 137 | G | 771.2 | ¹H NMR (DMSO-d₆) δ: 10.72 (br. s., 1H), 9.88 (s, 1H), 8.85 (s, |
| | | | 1H), 8.38 (s, 1H), 8.11 (br. s., 1H), 7.86-7.92 (m, 2H), 7.46- |
| | | | 7.54 (m, 1H), 7.24-7.33 (m, 2H), 7.18 (br. s., 1H), 6.87 (d, J = |
| | | | 7.9 Hz, 1H), 6.01 (s, 1H), 4.23 (d, J = 11.8 Hz, 2H), 3.11 (t, J = |
| | | | 11.6 Hz, 2H), 1.92 (td, J = 12.5, 4.2 Hz, 2H), 1.80 (d, J = 12.6 |
| | | | Hz, 2H) |
| 138 | G | 731.2 | ¹H NMR (DMSO-d₆) δ: 10.72 (br. s., 1H), 9.87 (s, 1H), 8.82 (s, |
| | | | 1H), 8.38 (s, 1H), 8.06 (br. s., 1H), 7.85-7.95 (m, 2H), 7.48 (t, |
| | | | J = 7.8 Hz, 1H), 7.22-7.32 (m, 2H), 7.18 (br. s., 1H), 6.81 (d, |
| | | | J = 8.0 Hz, 1H), 4.55 (t, J = 5.4 Hz, 1H), 3.75-3.83 (m, 2H), 3.22- |
| | | | 3.28 (m, 4H), 1.65-1.74 (m, 2H), 1.36-1.43 (m, 2H), 0.96 (s, 3H) |
| 139 | G | 717.2 | ¹H NMR (DMSO-d₆) δ: 10.72 (br. s., 1H), 9.88 (s, 1H), 8.83 (s, |
| | | | 1H), 8.38 (s, 1H), 8.07 (br. s., 1H), 7.91 (d, J = 8.1 Hz, 1H), 7.88 |
| | | | (d, J = 7.9 Hz, 1H), 7.49 (t, J = 7.9 Hz, 1H), 7.24-7.35 (m, 2H), |
| | | | 7.19 (t, J = 8.8 Hz, 1H), 6.81 (d, J = 8.0 Hz, 1H), 3.87-4.04 (m, |
| | | | 2H), 3.40 (tt, J = 8.4, 4.0 Hz, 1H), 3.30 (s, 3H), 3.07-3.23 (m, |
| | | | 2H), 1.95-2.10 (m, 2H), 1.54-1.70 (m, 2H) |
| 140 | G | 712.2 | ¹H NMR (DMSO-d₆) δ: 10.72 (br. s., 1H), 9.89 (s, 1H), 8.86 (s, |
| | | | 1H), 8.38 (s, 1H), 8.11 (br. s., 1H), 7.91 (d, J = 7.9 Hz, 1H), 7.88 |
| | | | (d, J = 8.0 Hz, 1H), 7.49 (t, J = 7.8 Hz, 1H), 7.24-7.35 (m, 2H), |
| | | | 7.15-7.24 (m, 1H), 6.84 (d, J = 8.0 Hz, 1H), 3.73-3.82 (m, 2H), |
| | | | 3.35-3.43 (m, 2H), 3.08-3.17 (m, 1H), 2.03-2.12 (m, 2H), |
| | | | 1.87-1.98 (m, 2H) |
| 141 | G | 719.2 | ¹H NMR (DMSO-d₆) δ: 10.72 (br. s., 1H), 9.89 (s, 1H), 8.86 (s, |
| | | | 1H), 8.38 (s, 1H), 8.12 (br. s., 1H), 7.85-7.95 (m, 2H), 7.48 (t, |
| | | | J = 7.8 Hz, 1H), 7.23-7.36 (m, 2H), 7.19 (br. s., 1H), 6.80 (d, |
| | | | J = 7.9 Hz, 1H), 4.79 (t, J = 5.8 Hz, 1H), 4.21 (d, J = 11.9 Hz, 1H), |
| | | | 4.15 (d, J = 11.5 Hz, 1H), 3.94-4.02 (m, 1H), 3.78 (td, J = 11.2, |
| | | | 1.8 Hz, 1H), 3.66-3.74 (m, 1H), 3.54 (dt, J = 11.1, 5.4 Hz, 1H), |
| | | | 3.44 (dt, J = 11.4, 5.8 Hz, 1H), 2.88 (td, J = 11.7, 3.2 Hz, 1H), |
| | | | 2.57-2.69 (m, 1H) |
| 142 | G | 735.2 | ¹H NMR (DMSO-d₆) δ: 10.72 (br. s., 1H), 9.89 (s, 1H), 8.85 (s, |
| | | | 1H), 8.38 (s, 1H), 8.09 (br. s., 1H), 7.92 (d, J = 8.0 Hz, 1H), 7.88 |
| | | | (d, J = 8.0 Hz, 1H), 7.49 (t, J = 7.8 Hz, 1H), 7.25-7.33 (m, 2H), |
| | | | 7.16-7.24 (m, 1H), 6.85 (d, J = 8.1 Hz, 1H), 5.01 (t, J = 5.9 Hz, |
| | | | 1H), 4.07-4.12 (m, 1H), 4.05 (br. s., 1H), 3.52 (d, J = 5.9 Hz, |
| | | | 1H), 3.47 (d, J = 6.0 Hz, 1H), 3.11-3.22 (m, 2H), 1.80-1.99 (m, 4H) |
| 143 | See text | 674.2 | ¹H NMR (DMSO-d₆) δ: 10.73 (br. s., 1H), 9.90 (s, 1H), 8.96 (s, |
| | | | 1H), 8.48 (br. s., 1H), 8.38 (s, 1H), 7.92 (d, J = 7.5 Hz, 1H), 7.87 |
| | | | (d, J = 7.9 Hz, 1H), 7.49 (t, J = 8.1 Hz, 1H), 7.32-7.42 (m, 2H), |
| | | | 7.25-7.32 (m, 1H), 7.21 (t, J = 9.0 Hz, 1H), 4.69 (t, J = 5.8 Hz, |
| | | | 1H), 3.74 (d, J = 5.8 Hz, 2H), 0.95 (d, J = 2.9 Hz, 4H) |
| 144 | G | 703.2 | ¹H NMR (DMSO-d₆) δ: 10.72 (br. s., 1H), 9.87 (br. s., 1H), 8.82 |
| | | | (s, 1H), 8.38 (s, 1H), 8.08 (br. s., 1H), 7.88 (d, J = 7.0 Hz, 2H), |
| | | | 7.48 (t, J = 7.8 Hz, 1H), 7.21-7.34 (m, 2H), 7.16 (br. s., 1H), |
| | | | 6.80 (d, J = 8.0 Hz, 1H), 4.71 (br. s., 1H), 4.02 (d, J = 11.6 Hz, |
| | | | 2H), 3.68 (br. s., 1H), 3.08 (t, J = 10.8 Hz, 2H), 1.90 (d, J = 11.1 |
| | | | Hz, 2H), 1.52-1.66 (m, 2H) |
| 145 | G | 726.2 | ¹H NMR (DMSO-d₆) δ: 10.72 (br. s., 1H), 9.89 (s, 1H), 8.86 (s, |
| | | | 1H), 8.38 (s, 1H), 8.13 (br. s., 1H), 7.91 (d, J = 7.9 Hz, 1H), 7.88 |
| | | | (d, J = 8.0 Hz, 1H), 7.49 (t, J = 7.5 Hz, 1H), 7.24-7.36 (m, 2H), |
| | | | 7.15-7.24 (m, 1H), 6.88 (d, J = 7.8 Hz, 1H), 4.25 (d, J = 12.4 |
| | | | Hz, 2H), 3.01 (t, J = 12.2 Hz, 2H), 2.04 (d, J = 13.1 Hz, 2H), 1.80 |
| | | | (t, J = 12.3 Hz, 2H), 1.44 (s, 3H) |
| 146 | G | 743.2 | ¹H NMR (DMSO-d₆) δ: 10.72 (s, 1H), 9.89 (s, 1H), 8.83 (s, 1H), |
| | | | 8.38 (s, 1H), 8.08 (br. s., 1H), 7.92 (d, J = 7.6 Hz, 1H), 7.88 (d, |
| | | | J = 7.8 Hz, 1H), 7.49 (t, J = 7.4 Hz, 1H), 7.29 (br. s., 2H), 7.17- |
| | | | 7.24 (m, 1H), 6.81 (d, J = 7.9 Hz, 1H), 3.78 (t, J = 6.8 Hz, 2H), |
| | | | 3.54-3.64 (m, 2H), 3.53 (s, 2H), 3.44-3.51 (m, 2H), 1.78 (t, |
| | | | J = 6.8 Hz, 2H), 1.72 (s, 4H) |
| 147 | G | 703.2 | ¹H NMR (DMSO-d₆) δ: 10.72 (s, 1H), 9.87 (s, 1H), 8.83 (s, 1H), |
| | | | 8.38 (s, 1H), 8.07 (br. s., 1H), 7.88 (d, J = 7.9 Hz, 2H), 7.42- |
| | | | 7.53 (m, 1H), 7.22-7.34 (m, 2H), 7.18 (br. s., 1H), 6.79 (d, J = |
| | | | 7.8 Hz, 1H), 4.87 (d, J = 3.9 Hz, 1H), 4.21 (d, J = 10.5 Hz, 1H), |
| | | | 4.05 (d, J = 12.5 Hz, 1H), 3.70 (s, 1H), 2.81 (t, J = 11.4 Hz, 1H), |
| | | | 2.56-2.69 (m, 1H), 1.97 (d, J = 10.4 Hz, 1H), 1.81 (d, J = 11.5 |
| | | | Hz, 1H), 1.59-1.72 (m, 1H), 1.28-1.37 (m, 1H) |
| 148 | G | 703.2 | ¹H NMR (DMSO-d₆) δ: 10.72 (s, 1H), 9.88 (s, 1H), 8.83 (s, 1H), |
| | | | 8.32-8.42 (m, 1H), 8.08 (br. s., 1H), 7.84-7.95 (m, 2H), 7.48 |
| | | | (t, J = 7.5 Hz, 1H), 7.27 (d, J = 7.4 Hz, 2H), 7.18 (br. s., 1H), |
| | | | 6.79 (d, J = 7.9 Hz, 1H), 4.87 (d, J = 3.4 Hz, 1H), 4.21 (d, J = |
| | | | 10.4 Hz, 1H), 4.05 (d, J = 11.5 Hz, 1H), 3.66-3.76 (m, 1H), 2.81 |
| | | | (t, J = 11.5 Hz, 1H), 2.61 (t, J = 10.3 Hz, 1H), 1.97 (d, J = 9.8 |
| | | | Hz, 1H), 1.81 (d, J = 13.1 Hz, 1H), 1.58-1.72 (m, 1H), 1.24- |
| | | | 1.37 (m, 1H) |
| 149 | G | 731.2 | ¹H NMR (DMSO-d₆) δ: 10.72 (br. s., 1H), 9.86 (s, 1H), 8.81 (s, |
| | | | 1H), 8.38 (s, 1H), 8.07 (br. s., 1H), 7.88 (d, J = 7.8 Hz, 2H), 7.47 |
| | | | (t, J = 7.0 Hz, 1H), 7.27 (s, 2H), 7.15 (br. s., 1H), 6.81 (d, J = 8.0 |
| | | | Hz, 1H), 4.09 (s, 1H), 3.91 (d, J = 11.3 Hz, 2H), 3.21-3.29 (m, |
| | | | 2H), 1.57-1.70 (m, 4H), 1.40-1.50 (m, 2H), 0.88 (t, J = 7.1 Hz, 3H) |
| 150 | G | 731.2 | ¹H NMR (DMSO-d₆) δ: 10.71 (s, 1H), 9.85 (s, 1H), 8.73 (s, 1H), |
| | | | 8.37 (s, 1H), 7.75-7.98 (m, 3H), 7.48 (t, J = 6.8 Hz, 1H), 7.27 |
| | | | (s, 1H), 7.21 (t, J = 7.4 Hz, 2H), 6.57 (d, J = 8.0 Hz, 1H), 4.25 (s, |
| | | | 1H), 4.06 (dd, J = 11.9, 3.2 Hz, 1H), 3.61-3.79 (m, 3H), 2.03- |
| | | | 2.16 (m, 1H), 1.83-1.93 (m, 1H), 1.70-1.83 (m, 2H), 1.59- |
| | | | 1.69 (m, 1H), 1.43-1.52 (m, 1H), 1.15 (s, 3H) |
| 151 | G | 755.2 | ¹H NMR (DMSO-d₆) δ: 10.72 (s, 1H), 9.89 (s, 1H), 8.83 (s, 1H), |
| | | | 8.38 (s, 1H), 8.01-8.19 (m, 1H), 7.92 (d, J = 7.5 Hz, 1H), 7.88 |
| | | | (d, J = 7.9 Hz, 1H), 7.49 (t, J = 7.4 Hz, 1H), 7.24-7.33 (m, 2H), |
| | | | 7.20 (s, 1H), 6.82 (d, J = 7.8 Hz, 1H), 4.08 (d, J = 11.9 Hz, 2H), |
| | | | 3.09 (t, J = 11.8 Hz, 2H), 2.29 (t, J = 7.4 Hz, 2H), 1.94-2.01 (m, |
| | | | 2H), 1.89 (q, J = 7.0 Hz, 2H), 1.76 (t, J = 11.3 Hz, 2H), 1.51 (d, |
| | | | J = 12.5 Hz, 2H) |
| 152 | G | 732.2 | ¹H NMR (DMSO-d₆) δ: 10.54 (br. s., 1H), 9.85 (s, 1H), 8.85 (s, |
| | | | 1H), 8.38 (s, 1H), 8.15 (br. s., 1H), 7.89 (d, J = 8.0 Hz, 1H), 7.84 |
| | | | (d, J = 7.4 Hz, 1H), 7.45 (t, J = 6.9 Hz, 1H), 7.26-7.35 (m, 1H), |
| | | | 7.17-7.26 (m, 1H), 7.03-7.14 (m, 1H), 6.80 (d, J = 7.5 Hz, 1H), |
| | | | 4.60 (s, 1H), 3.59 (s, 6H), 2.80 (s, 4H), 2.62 (s, 2H) |
| 153 | G | 765.2 | ¹H NMR (DMSO-d₆) δ: 10.72 (br. s., 1H), 9.90 (s, 1H), 8.87 (s, |
| | | | 1H), 8.38 (s, 1H), 8.11 (br. s., 1H), 7.92 (d, J = 7.6 Hz, 1H), 7.88 |
| | | | (d, J = 7.9 Hz, 1H), 7.49 (t, J = 7.9 Hz, 1H), 7.25-7.38 (m, 2H), |
| | | | 7.21 (t, J = 9.0 Hz, 1H), 6.85 (d, J = 7.9 Hz, 1H), 4.45 (d, J = |
| | | | 11.6 Hz, 2H), 3.34-3.41 (m, 1H), 2.98 (s, 3H), 2.90 (t, J = 12.1 |
| | | | Hz, 2H), 2.15 (d, J = 12.0 Hz, 2H), 1.83 (q, J = 12.1 Hz, 2H) |
| 154 | See text | 535.2 | ¹H NMR (DMSO-d₆) δ: 10.46 (br. s., 1H), 9.96 (s, 1H), 9.36 (s, |
| | | | 1H), 8.71 (s, 1H), 8.36 (s, 1H), 7.60 (d, J = 7.9 Hz, 1H), 7.47 (t, |
| | | | J = 8.8 Hz, 1H), 7.38 (td, J = 7.9, 5.6 Hz, 1H), 7.26 (td, J = 8.7, |
| | | | 5.8 Hz, 1H), 7.17 (t, J = 9.1 Hz, 1H), 2.48 (d, J = 2.1 Hz, 3H) |
| 155 | G | 766.2 | ¹H NMR (DMSO-d₆) δ: 10.72 (s, 1H), 9.88 (s, 1H), 8.89 (s, 1H), |
| | | | 8.38 (s, 1H), 8.19 (br. s., 1H), 7.88 (d, J = 7.8 Hz, 2H), 7.47 (t, |
| | | | J = 7.6 Hz, 1H), 7.33 (t, J = 7.4 Hz, 1H), 7.25 (br. s., 1H), 7.15 (br. |
| | | | s., 1H), 6.85 (d, J = 8.0 Hz, 1H), 3.59-3.70 (m, 4H), 3.34-3.37 |
| | | | (m, 4H), 2.96 (s, 3H) |
| 156 | See text | 688.2 | ¹H NMR (DMSO-d₆) δ: 10.66 (br. s., 1H), 9.90 (s, 1H), 8.91 (s, |
| | | | 2H), 8.39 (s, 1H), 8.23 (br. s., 1H), 7.84-7.94 (m, 2H), 7.48 (t, |
| | | | J = 7.8 Hz, 1H), 7.35 (t, J = 7.8 Hz, 1H), 7.23-7.31 (m, 1H), |
| | | | 7.13-7.23 (m, 1H), 6.88 (d, J = 8.0 Hz, 1H), 3.67-3.84 (m, 4H), |
| | | | 3.34-3.44 (m, 4H) |
| 157 | G | 716.2 | ¹H NMR (DMSO-d₆) δ: 9.71 (br. s., 1H), 8.85 (s, 1H), 8.40 (s, |
| | | | 1H), 8.23 (br. s., 1H), 8.15 (s, 1H), 7.92 (d, J = 7.8 Hz, 1H), 7.65 |
| | | | (d, J = 7.6 Hz, 1H), 7.32-7.41 (m, 1H), 7.29 (br. s., 1H), 6.98- |
| | | | 7.13 (m, 1H), 6.67-6.90 (m, 2H), 3.87-4.14 (m, 2H), 3.25- |
| | | | 3.23-3.29 (m, 2H), 1.87-2.03 (m, 2H), 1.81 (d, J = 13.0 Hz, |
| | | | 2H), 1.38 (s, 3H), 2H exch. |
| 158 | See text | 737.2 | ¹H NMR (DMSO-d₆) δ: 10.72 (s, 1H), 9.90 (s, 1H), 8.91 (s, 1H), |
| | | | 8.38 (s, 1H), 8.17 (br. s., 1H), 7.92 (d, J = 5.8 Hz, 1H), 7.88 (d, |
| | | | J = 7.8 Hz, 1H), 7.44-7.55 (m, 1H), 7.24-7.38 (m, 2H), 7.21 |
| | | | (br. s., 1H), 6.94 (d, J = 8.0 Hz, 1H), 4.16 (br. s., 4H), 3.26-3.29 |
| | | | (m, 4H) |

TABLE 4

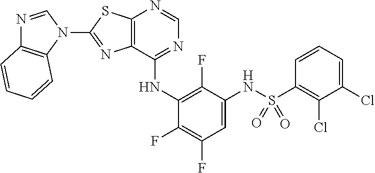 [SMILES: O=S(=O)(Nc1cc(F)c(F)c(Nc2ncnc3sc(-n4cnc5ccccc54)nc23)c1F)c1cccc(Cl)c1Cl]

| | HCT116 | Kinase IC₅₀ | Kinase IC₅₀ |
|---|---|---|---|
| | pERK IC₅₀ | BRAF | CRAF |
| Example | (μM) (Yₘᵢₙ %) | (nM) | (nM) |
| 159 | +++ (−6) | ** | §§§ |

For pERK assays, + denotes a 1-10 μM IC₅₀ range, ++ denotes a 0.5-1 μM IC₅₀ range and +++ denotes an IC₅₀ <0.5 μM. The % Yₘᵢₙ value indicates the lowest value of each IC₅₀ curve. Compounds that exhibit IC₅₀ curves with Yₘᵢₙ values above −20% are considered to display minimal or no induction and do not cause detectable paradoxical activation of the pathway. For the BRAF biochemical kinase assay, * denotes an IC₅₀ >10 nM, ** denotes a 1-10 nM IC₅₀ range and *** denotes an IC₅₀ < 1 nM. For the CRAF biochemical
kinase assay, § denotes an IC₅₀ >50 nM, §§ denotes a 10-50 nM IC₅₀ range, and §§§ denotes an IC₅₀ < 10 nM.

Characterization of Example 159 from Table 4

| | | MS | |
|---|---|---|---|
| | Synthesis | m/z | |
| Ex. | Method | (MH⁺) | ¹H NMR (400 MHz) |
| 159 | See text | 622.2 | ¹H NMR (DMSO-d₆) δ: 11.03 (br. s., 1H), |
| | | | 10.07 (s, 1H), 9.03 (s, 1H), 8.62 (br. s., 1H), |
| | | | 8.43 (s, 1H), 7.88-7.98 (m, 2H), 7.80-7.87 |
| | | | (m, 1H), 7.39-7.56 (m, 4H) |

Numerous modifications could be made to any of the embodiments described above without departing from the scope of the present invention. Any references, patents or scientific literature documents referred to in the present document are incorporated herein by reference in their entirety for all purposes.

1. A compound of Formula I: 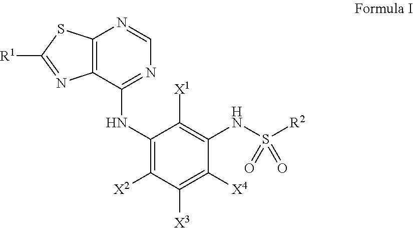
wherein:
R¹ is selected from substituted or unsubstituted OR³, SR³, NH₂, NHR³, N(R³)₂, C₃₋₈cycloalkyl, C₄₋₈heterocycloalkyl, C₆₋₁₀aryl and C₅₋₁₀heteroaryl;
R² is selected from substituted C₆aryl or C₅₋₁₀heteroaryl, substituted or unsubstituted C₄₋₈heterocycloalkyl, and N(R³)₂;
R³ is independently in each occurrence selected from substituted or unsubstituted C₁₋₈alkyl, C₃₋₈cycloalkyl, C₄₋₈heterocycloalkyl, C₅₋₁₀aryl and C₅₋₁₀heteroaryl;
X¹ is halo or an electron-withdrawing group;
X² is selected from H, halo, and an electron-withdrawing group;
X³ and X⁴ are each selected from H, halo, an electron-withdrawing group, C₁₋₃alkyl, C₃₋₄cycloalkyl, and OC₁₋₃alkyl;
or a pharmaceutically acceptable salt or solvate thereof.

2. The compound of claim 1, wherein R² is a substituted C₆aryl or C₅₋₁₀heteroaryl.

3. The compound of claim 2, wherein R² is a C₆aryl substituted with at least one group selected from F, Cl, Br, CN, NO₂, and a substituted or unsubstituted C₁₋₃alkyl, C₃₋₄cycloalkyl or OC₁₋₃alkyl.

4. The compound of claim 2, wherein R² is a group of the formula: 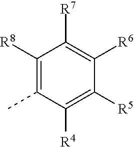
wherein:
R⁴ is selected from H, F, Cl, Br, CN, and a substituted or unsubstituted C₁₋₃alkyl, C₃₋₄cycloalkyl or OC₁₋₃alkyl;
R⁵ is selected from H, F, Cl, CN, and a substituted or unsubstituted C₁₋₃alkyl, C₃₋₄cycloalkyl or OC₁₋₃alkyl;
R⁶ is selected from H, F, Cl, Br, NO₂, NH₂, and a substituted or unsubstituted C₁₋₃alkyl, C₃₋₄cycloalkyl or OC₁₋₃alkyl;
R⁷ is selected from H, F, Cl, and a substituted or unsubstituted C₁₋₃alkyl;
R³ is selected from H, F, and a substituted or unsubstituted C₁₋₃alkyl;
or R⁴ and R⁵ or R⁵ and R⁶ are taken together with their adjacent carbon atoms to form a substituted or unsubstituted carbocycle or heterocycle provided that the heterocycle is not a benzoxazolinone; and
( - - - ) represents a bond serving as a point of attachment between R² and the rest of the molecule;
wherein when R⁴ is H or F, then at least one of R⁵, R⁶, R⁷ or R³ is other than H or F; and
wherein when R⁵ is CN, then at least one of R⁴, R⁶, R⁷ or R³ is other than H.

5. The compound of claim 4, wherein R⁴ is selected from H, F, Cl, Br, Me, Et, CN, CHF₂, and CF₃.

6. The compound of claim 4 or 5, wherein R⁵ is selected from H, F, Me, CF₃, CN, and CI.

7. The compound of any one of claims 4 to 6, wherein R⁶ is selected from H, F, Cl, Br, and a substituted or unsubstituted C₁₋₃alkyl, C₃₋₄cycloalkyl or OC₁₋₃alkyl.

8. The compound of any one of claims 4 to 7, wherein R⁶ is selected from H, F, Cl, Me, Et, and OMe.

9. The compound of any one of claims 4 to 8, wherein R⁷ is selected from H, Me, F, and Cl.

10. The compound of any one of claims 4 to 9, wherein R³ is selected from H, Me and F.

11. The compound of claim 4, wherein:
R⁴ is selected from Cl and a substituted or unsubstituted C₁₋₃alkyl;
R⁵ is selected from H, F, Cl, and a substituted or unsubstituted C₁₋₃alkyl;
R⁶ is selected from H, F, Cl, a substituted or unsubstituted C₁₋₃alkyl, and a substituted or unsubstituted OC₁₋₃alkyl; and
R⁷ and R³ are each H.

12. The compound of claim 11, wherein R⁴ is selected from Cl and CH₃.

13. The compound of claim 11 or 12, wherein R⁵ is selected from F, Cl and CH₃.

14. The compound of any one of claims 11 to 13, wherein R⁶ is H or F.

15. The compound of any one of claims 11 to 13, wherein R⁶ is Cl, a substituted or unsubstituted C₁₋₃alkyl, or a substituted or unsubstituted OC₁₋₃alkyl.

16. The compound of claim 15, wherein R⁶ is CH₃ or OCH₃.

17. The compound of claim 1, wherein R² is a group of the formula: 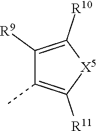
wherein:
X⁵ is selected from NH, NC₁₋₃alkyl, NC₃₋₄cycloalkyl, O and S;
R⁹, R¹⁰, R¹¹ are each independently selected from H, F, Cl, CN, and a substituted or unsubstituted C₁₋₃alkyl, C₃₋₄cycloalkyl, C(O)OC₁₋₃alkyl or OC₁₋₃alkyl, provided that one of R⁹ and R¹¹ is H and the other is different from H; and
( - - - ) represents a bond serving as a point of attachment between R² and the rest of the molecule.

18. The compound of claim 1, wherein R² is a group of the formula: 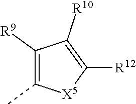
wherein:
X⁵ is selected from NH, NC₁₋₃alkyl, NC₃₋₄cycloalkyl, O and S;
R⁹ is selected from F, Cl, CN, and a substituted or unsubstituted C₁₋₃alkyl, C₃₋₄cycloalkyl, C(O)OC₁₋₃alkyl or OC₁₋₃alkyl;
R¹⁰ and R¹² are each independently selected from H, F, Cl, CN, and a substituted or unsubstituted C₁₋₃alkyl, C₃₋₄cycloalkyl, C(O)OC₁₋₃alkyl or OC₁₋₃alkyl; and
( - - - ) represents a bond serving as a point of attachment between R² and the rest of the molecule.

19. The compound of claim 17 or 18, wherein R⁹ and R¹⁰ are each independently selected from F, Cl, CN, and a substituted or unsubstituted C₁₋₃alkyl, C₃₋₄cycloalkyl, C(O)OC₁₋₃alkyl or OC₁₋₃alkyl.

20. The compound of claim 19, wherein R⁹ and R¹⁰ are each independently selected from Cl and a substituted or unsubstituted C₁₋₃alkyl.

21. The compound of claim 19, wherein R⁹ and R¹⁰ are both Cl.

22. The compound of any one of claims 17 to 21, wherein X⁵ is O or S, preferably S.

23. The compound of claim 2, wherein R² is a group of the formula: 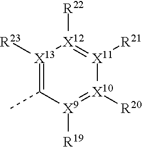
wherein:
X⁹, X¹⁰, X¹¹, X¹², and X¹³ are independently selected from N and C, wherein at least one and at most two of X⁹, X¹⁰, X¹¹, X¹², and X¹³ are N; and
R¹⁹, R²⁰, R²¹, R²² and R²³ are selected from H, F, Cl, Br, CN, NO₂, NH₂, and a substituted or unsubstituted C₁₋₃alkyl, C₃₋₄cycloalkyl or OC₁₋₃alkyl, or are absent when their attached X⁹, X¹⁰, X¹¹, X¹², or X¹³ is N;
wherein at least one of X⁹ and X¹³ is not N;
wherein when one of X⁹ and X¹³ is N, then the other is not N or CH; and
( - - - ) represents a bond serving as a point of attachment between R² and the rest of the molecule.

24. The compound of claim 1, wherein R² is a group of the formula: 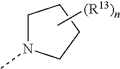
wherein:
R¹³ is independently in each occurrence selected from F, Cl, and a substituted or unsubstituted C₁₋₃alkyl, C₃₋₄cycloalkyl, or C₁₋₃alkoxy;
n is an integer selected from 0 to 8; or
is between 2 and 8 and two R¹³ are taken together with their adjacent carbon atoms to form a C₃₋₄cycloalkyl; and
( - - - ) represents a bond serving as a point of attachment between R² and the rest of the molecule.

25. The compound of claim 24, wherein R¹³ is F, Me, OMe, and CH₂OMe, and n is 1 or 2.

26. The compound of claim 24 or 25, wherein R¹³ is in the 3-position.

27. The compound of claim 1, wherein R² is N(R³)₂.

28. The compound of claim 27, wherein R³ is selected from substituted or unsubstituted C₁₋₈alkyl or C₃₋₈cycloalkyl.

29. The compound of claim 1, wherein R² is selected from groups B1 to B77.

30. The compound of claim 29, wherein R² is selected from groups B1 to B6.

31. The compound of any one of claims 1 to 30, wherein R¹ is OR³ or SR³.

32. The compound of claim 31, wherein R¹ is SR³.

33. The compound of any one of claims 1 to 32, wherein R³ is a substituted or unsubstituted C₁₋₈ alkyl (e.g. C₁₋₃alkyl).

34. The compound of any one of claims 1 to 30, wherein R¹ is a substituted or unsubstituted C₅₋₆ heteroaryl group.

35. The compound of any one of claims 1 to 30, wherein R¹ is a substituted or unsubstituted C₉heteroaryl group.

36. The compound of any one of claims 1 to 30, wherein R¹ is a substituted or unsubstituted group selected from imidazolyl, pyrazolyl, triazolyl, indolyl, indazolyl, benzimidazolyl, benzotriazolyl, pyrrolopyridinyl (e.g. pyrrolo[3,2-b]pyridinyl or pyrrolo[3,2-c]pyridinyl), pyrazolopyridinyl (e.g. pyrazolo[1,5-a]pyridinyl), purinyl, and imidazopyrazinyl (e.g. imidazo[4,5-b]pyrazinyl), preferably attached to the thiazolopyrimidine core through a nitrogen atom.

37. The compound of any one of claims 1 to 30, wherein R¹ is a substituted or unsubstituted C₄₋₆ heterocycloalkyl group.

38. The compound of any one of claims 1 to 30, wherein R¹ is a substituted or unsubstituted group selected from: 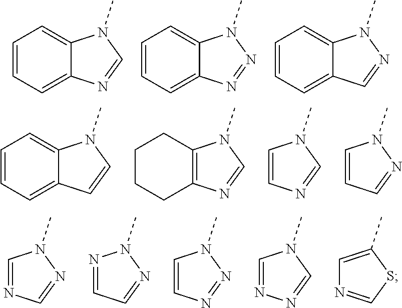
wherein ( - - - ) represents a bond serving as a point of attachment between R¹ and the rest of the molecule.

39. The compound of claim 38, wherein R¹ is a substituted or unsubstituted group selected from: 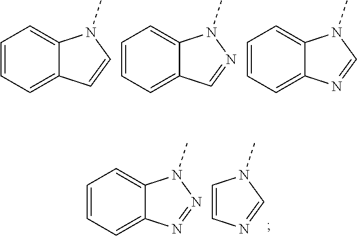
wherein ( - - - ) represents a bond serving as a point of attachment between R¹ and the rest of the molecule.

40. The compound of any one of claims 1 to 39, wherein R¹ is substituted with at least one substituent selected from OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein:
R¹⁴ is independently in each occurrence selected from H, C₁₋₆alkyl, C₂₋₆alkenyl, C₂₋₆ alkynyl, C₃₋₁₀cycloalkyl, C₄₋₁₀heterocycloalkyl, C₆aryl, and C₅₋₁₀heteroaryl, or two R¹⁴ are taken together with their adjacent nitrogen atom to form a C₄₋₁₀heterocycloalkyl group;
R¹⁵ is independently in each occurrence selected from C₁₋₆alkyl, C₂₋₆alkenyl, C₂₋₆alkynyl, C₃₋₁₀cycloalkyl, C₆aryl, and C₅₋₁₀heteroaryl; and
R¹⁶ is independently in each occurrence selected from H, C₁₋₆alkyl, C₂₋₆alkenyl, C₂₋₆alkynyl, C₃₋₁₀cycloalkyl, C₆aryl, and C₅₋₁₀heteroaryl;
wherein said alkyl, alkenyl, alkynyl, cycloalkyl, heterocycloalkyl, aryl or heteroaryl group is optionally further substituted.

41. The compound of any one of claims 1 to 30, wherein R¹ is a group of the formula: 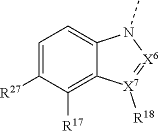
wherein:
R¹⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
R²⁷ is selected from H, OH, halo, CN, NO₂, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, preferably H, halo, optionally substituted C₁₋₆alkyl, or optionally substituted OC₁₋₆alkyl;
X⁶ is N or CH; and
X⁷ is N and R¹⁸ is absent; or
X⁷ is C and R¹⁸ is selected from C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂;
wherein R¹⁴, R¹⁵, and R¹⁶ are as defined in claim 40;
wherein said alkyl, alkenyl, alkynyl, cycloalkyl, heterocycloalkyl, or heteroaryl is optionally further substituted; and
wherein ( - - - ) represents a bond serving as a point of attachment between R¹ and the rest of the molecule.

42. The compound of claim 41, wherein X⁶ is N.

43. The compound of claim 41, wherein X⁶ is CH.

44. The compound of any one of claims 41 to 43, wherein X⁷ is N, R¹⁷ is selected from H, halo, OH, CN, C₁₋₆alkyl, C₂₋₆alkenyl, C₂₋₆alkynyl, OC₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, and R¹⁸ is absent, wherein said alkyl, alkenyl, alkynyl, cycloalkyl, heterocycloalkyl, or heteroaryl is optionally further substituted.

45. The compound of claim 44, wherein R¹⁷ is selected from C₁₋₆alkyl, C₅₋₁₀heteroaryl, C₄₋₁₀ heterocycloalkyl, N(R¹⁴)₂, N(R¹⁶)C(O)R¹⁵, N(R¹⁶)SO₂R¹⁵, C(O)N(R¹⁴)₂, and SO₂N(R¹⁴)₂, wherein said alkyl, alkenyl, alkynyl, cycloalkyl, heterocycloalkyl, or heteroaryl is optionally further substituted.

46. The compound of claim 44, wherein R¹⁷ is selected from H, F, NH₂, and an optionally substituted C₅₋₁₀heteroaryl or C₄₋₁₀heterocycloalkyl, preferably an optionally substituted C₅₋₁₀ heteroaryl or C₄₋₁₀heterocycloalkyl.

47. The compound of any one of claims 41 to 46, wherein R¹⁷ is an optionally substituted C₄₋₁₀heterocycloalkyl, wherein said heterocycloalkyl is mono or bicyclic and include from 1 to 3 heteroatoms, preferably wherein X⁷ is N.

48. The compound of claim 47, wherein the heterocycloalkyl is substituted with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.).

49. The compound of any one of claims 47 or 48, wherein the heterocycloalkyl is selected from piperidine, piperazine, thiomorpholine, and morpholine groups, or a bicyclic structure (bridged or spiro) containing a piperidine, piperazine, thiomorpholine, or morpholine ring.

50. The compound of any one of claims 41 to 43, wherein X⁷ is C.

51. The compound of claim 50, wherein R¹⁸ is selected from C₁₋₆alkyl, C₅₋₁₀heteroaryl, C₃₋₁₀cycloalkyl, C₄₋₁₀heterocycloalkyl, C(O)R¹⁵, C(O)N(R¹⁴)₂, SO₂R¹⁵, SO₂N(R¹⁴)₂, N(R¹⁶)C(O)R¹⁵, N(R¹⁶)SO₂R¹⁵, N(R¹⁶)C(O)N(R¹⁴)₂, N(R¹⁶)SO₂N(R¹⁴)₂, N(R¹⁴)₂, P(O)(R¹⁵)₂, CH₂C(O)R¹⁵, CH₂C(O)N(R¹⁴)₂, CH₂SO₂R¹⁵, CH₂SO₂N(R¹⁴)₂, CH₂N(R¹⁶)C(O)R¹⁵, CH₂N(R¹⁶)SO₂R¹⁵, CH₂N(R¹⁶)C(O)N(R¹⁴)₂, CH₂N(R¹⁶)SO₂N(R¹⁴)₂, and CH₂N(R¹⁴)₂, wherein said alkyl, alkenyl, alkynyl, cycloalkyl, heterocycloalkyl, aryl or heteroaryl is optionally further substituted.

52. The compound of claim 51, wherein R¹⁸ is selected from C(O)N(R¹⁴)₂, SO₂R¹⁵, and SO₂N(R¹⁴)₂.

53. The compound of any one of claims 50 to 52, wherein R¹⁷ is selected from H, OH, halo, C₁₋₆ alkyl, N(R¹⁴)₂, and an optionally substituted C₅₋₁₀heteroaryl.

54. The compound of claim 53, wherein R¹⁷ is selected from H, F, NH₂, and an optionally substituted C₅₋₁₀heteroaryl, preferably H, F, or NH₂.

55. The compound of any one of claims 40 to 54, wherein R¹⁴ is independently in each occurrence selected from H, optionally substituted C₁₋₆alkyl, optionally substituted C₃₋₁₀cycloalkyl, optionally substituted C₄₋₁₀heterocycloalkyl, and optionally substituted C₅₋₆ heteroaryl, or two R¹⁴ are taken together with their adjacent nitrogen atom to form an optionally substituted C₄₋₁₀heterocycloalkyl group.

56. The compound of claim 55, wherein two R¹⁴ are taken together with their adjacent nitrogen atom to form an optionally substituted C₄₋₁₀heterocycloalkyl group, wherein said heterocycloalkyl is a mono or bicyclic and include from 1 to 3 heteroatoms.

57. The compound of claim 56, wherein the heterocycloalkyl is substituted with at least one group selected from F, OH, oxo, CN, C₁₋₄alkyl and OC₁₋₄alkyl, wherein said C₁₋₄alkyl is optionally further substituted (e.g. with F, OH, OC₁₋₃alkyl, etc.).

58. The compound of any one of claims 55 to 57, wherein the heterocycloalkyl is selected from piperidine, piperazine, thiomorpholine, and morpholine groups, or a bicyclic structure (bridged or spiro) containing a piperidine, piperazine, thiomorpholine, or morpholine ring.

59. The compound of any one of claims 1 to 30, wherein R¹ is selected from: 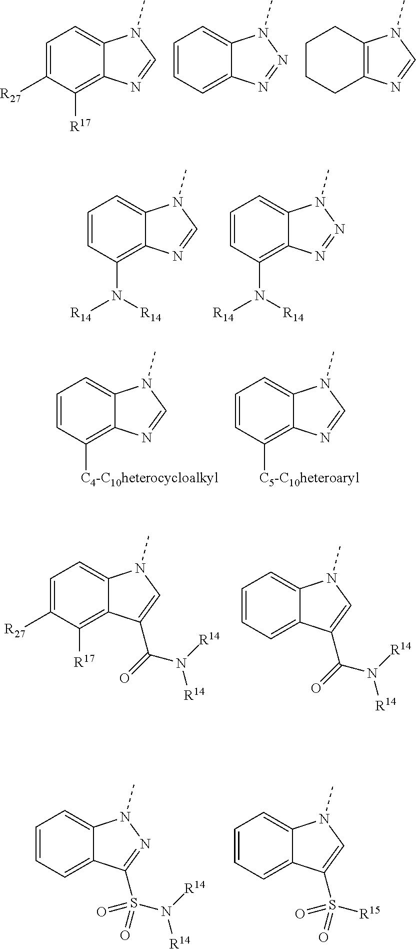
wherein R¹⁴, R¹⁷, and R²⁷ are as defined herein and ( - - - ) represents a bond serving as a point of attachment between R¹ and the rest of the molecule.

60. The compound of claim 59, wherein R¹ is selected from: 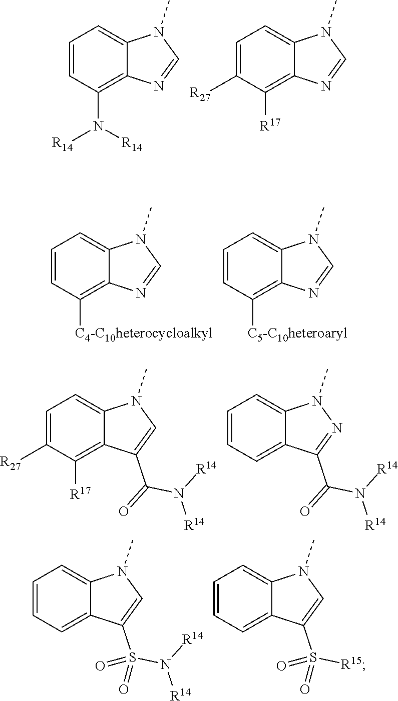
wherein R¹⁴, R¹⁷, and R²⁷ are as defined herein and ( - - - ) represents a bond serving as a point of attachment between R¹ and the rest of the molecule.

61. The compound of any one of claims 1 to 30, wherein R¹ is a group of the formula: 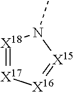
wherein:
X¹⁵, X¹⁶, X¹⁷, and X¹⁸ are independently selected from O, N, S, and CR¹⁷, wherein R¹⁷ is as previously defined;
wherein at most two of X¹⁵, X¹⁶, X¹⁷, and X¹⁸ are O, N, or S; and
wherein ( - - - ) represents a bond serving as a point of attachment between R¹ and the rest of the molecule.

62. The compound of any one of claims 1 to 30, wherein R¹ is selected from groups A1 to A550.

63. The compound of claim 62, wherein R¹ is selected from groups A1 to A3, A8, A19, A20, A22, A23, A25, A28, A29, A32 to A39, A60, A63 to A66, A69, A72 to A78, A81 to A83, A86, A89, A93, A96, A100, A101, A104, A105, A109 to A111, A113, A115, A118, A121 to A123, A127 and A132.

64. The compound of claim 62, wherein R¹ is selected from groups A1 to A3, A8, A19, A22, A25, A28, A29, A32, A36, A37, A64, A67, A74, A77, A78, A82, A83, A89, A96, A109, A110 and A111.

65. The compound of any one of claims 1 to 64, wherein X¹ is Cl and X² is F.

66. The compound of any one of claims 1 to 64, wherein X¹ is F and X² is H.

67. The compound of any one of claims 1 to 64, wherein both X¹ and X² are F.

68. The compound of any one of claims 1 to 67, wherein X³ and X⁴ are each H.

69. The compound of any one of claims 1 to 67, wherein X³ is F and X⁴ is H.

70. The compound of claim 67, wherein said compound is of Formula II: 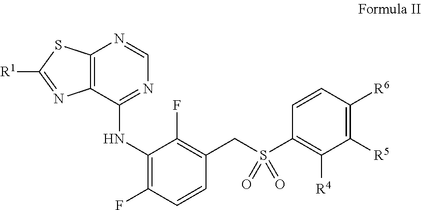
wherein R¹, R⁴, R⁵, and R⁶ are each independently as defined herein, preferably R⁴ is selected from Cl, Br and methyl; R⁵ is selected from H, F, Cl and methyl; R⁶ is selected from H, F, Cl, Me and OMe.

71. The compound of claim 70, wherein said compound is of Formula IV: 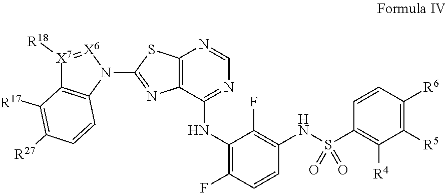
wherein X⁶, X⁷, R⁴, R⁵, R⁶, R¹⁷, R²⁷, and R¹⁸ are each independently as previously defined.

72. The compound of claim 70, wherein said compound is a compound of Formula V: 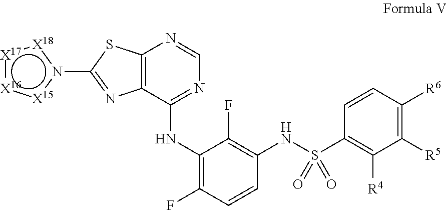
wherein R⁴, R⁵, R⁶, X¹⁵, X¹⁶, X¹⁷, and X¹³ are each independently as previously defined.

73. The compound of claim 67, wherein said compound is of Formula III: 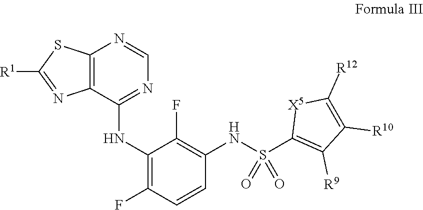
wherein R¹, R⁹, R¹⁰, R¹², and X⁵ are each independently as previously defined.

74. The compound of claim 73, wherein said compound is of Formula VI: 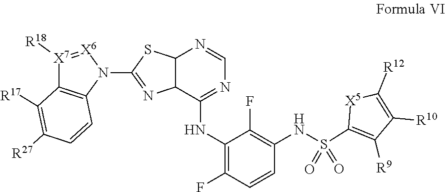
wherein R⁹, R¹⁰, R¹², R¹⁷, R¹⁸, R²⁷, X⁵, X⁶, and X⁷, are each independently as previously defined.

75. The compound of claim 73, wherein said compound is of Formula VII: 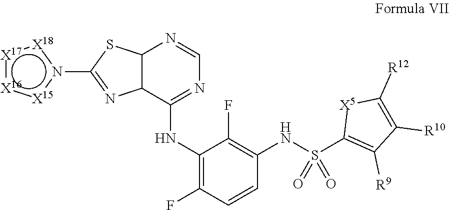
wherein R⁹, R¹⁰, R¹², X⁵, X¹⁵, X¹⁶, X¹⁷ and X¹⁸ are each independently as previously defined.

76. The compound of claim 1, wherein said compound is selected from Examples 1 to 159 as defined herein, or a salt and/or solvate thereof.

77. The compound of claim 76, wherein said compound is selected from Examples 2, 4, 6, 7, 14, 16, 18, 30, 31, 33 to 37, 40, 43 to 46, 49, 51 to 60, 81, 84 to 88, 90, 93 to 99, 102 to 105, 108, 111, 112, 116 to 119, 122, 126, 127, 130, 131, 135 to 137, 139, 141, 144, 147, 148, 149, 153, and 158, or a salt and/or solvate thereof.

78. The compound of claim 76, wherein said compound is selected from Examples 4, 6, 7, 14, 16, 18, 30, 33, 35, 36, 37, 40, 43 to 45, 49, 51, 56 to 58, 85, 88, 95, 98, 99, 103, 104, 105, 111, 112, 116, 122, 135, and 136, or a salt and/or solvate thereof.

79. A pharmaceutical composition comprising a compound as defined in any one of claims 1 to 78, together with a pharmaceutically acceptable carrier, diluent or excipient.

80. Use of a compound as defined in any one of claims 1 to 78 for the treatment of a disease or disorder selected from a proliferative disease or disorder, a developmental anomaly caused by dysregulation of the RAS-ERK signaling cascade (RASopathies), or an inflammatory disease or an immune system disorder.

81. The use of claim 80, wherein the disease or disorder is selected from a neoplasm and a developmental anomaly.

82. The use of claim 80 or 81, wherein said disease or disorder is associated with a RAF gene mutation (e.g. ARAF, BRAF or CRAF).

83. The use of any one of claims 80 to 82, wherein said disease or disorder is associated with a RAS gene mutation (e.g. KRAS).

84. The use of any one of claims 80 to 83, wherein said disease or disorder is associated with a receptor tyrosine kinase mutation or amplification (e.g. EGFR, HER2) or a mutation or amplification in a regulator of RAS downstream of the receptor (e.g. SOS1 gain of function, NF1 loss of function).

85. The use of any one of claims 80 to 84, wherein said disease or disorder is a neoplasm.

86. The use of claim 85, wherein said neoplasm is selected from melanoma, thyroid carcinoma (e.g. papillary thyroid carcinoma), colorectal, ovarian, breast cancer, endometrial cancer, liver cancer, sarcoma, stomach cancer, pancreatic carcinoma, Barret's adenocarcinoma, glioma (e.g. ependymoma), lung cancer (e.g. non-small cell lung cancer), head and neck cancer, acute lymphoblastic leukemia, acute myelogenous leukemia, non-Hodgkin's lymphoma, and hairy-cell leukemia.

87. The use of claim 85, wherein said neoplasm is selected from colon or colorectal cancer, lung cancer, pancreatic cancer, thyroid cancer, breast cancer and melanoma.

88. The use of any one of claims 80 to 87, wherein said treatment comprises inhibiting the RAS-ERK signaling pathway without substantial induction of a paradoxical pathway.

89. A method for the treatment of a disease or disorder selected from a proliferative disease or disorder, a developmental anomaly caused by dysregulation of the RAS-ERK signaling cascade (RASopathies), or an inflammatory disease or an immune system disorder, comprising administering a compound as defined in any one of claims 1 to 78 to a subject in need thereof.

90. The method of claim 89, wherein the disease or disorder is selected from a neoplasm and a developmental anomaly.

91. The method of claim 89 or 90, wherein said disease or disorder is associated with a RAF gene mutation (e.g. ARAF, BRAF or CRAF).

92. The method of any one of claims 89 to 91, wherein said disease or disorder is associated with a RAS mutation (e.g. KRAS).

93. The method of any one of claims 89 to 92, wherein said disease or disorder is associated with a receptor tyrosine kinase mutation or amplification (e.g. EGFR, HER2) or a mutation or amplification in a regulator of RAS downstream of the receptor (e.g. SOS1 gain of function, NF1 loss of function).

94. The method of any one of claims 89 to 93, wherein said disease or disorder is a neoplasm.

95. The method of claim 94, wherein said neoplasm is selected from melanoma, thyroid carcinoma (e.g. papillary thyroid carcinoma), colorectal, ovarian, breast cancer, endometrial cancer, liver cancer, sarcoma, stomach cancer, pancreatic carcinoma, Barret's adenocarcinoma, glioma (e.g. ependymoma), lung cancer (e.g. non-small cell lung cancer), head and neck cancer, acute lymphoblastic leukemia, acute myelogenous leukemia, non-Hodgkin's lymphoma, and hairy-cell leukemia.

96. The method of claim 94, wherein said neoplasm is selected from colon or colorectal cancer, lung cancer, pancreatic cancer, thyroid cancer, breast cancer and melanoma.

97. The method of any one of claims 89 to 96, wherein said method comprises inhibiting the RAS-ERK signaling pathway without substantial induction of a paradoxical pathway.

98. A method for inhibiting abnormal proliferation of cells, comprising contacting the cells with a compound as defined in any one of claims 1 to 78.

99. The method of claim 98, wherein said cells comprise a mutated RAF protein kinase (e.g. a mutated ARAF, BRAF or CRAF).

100. The method of claim 98 or 99, wherein said cells comprise a mutated RAS gene (e.g. mutated KRAS).

101. The method of any one of claims 98 to 100, wherein said abnormal proliferation is associated with a receptor tyrosine kinase mutation or amplification (e.g. EGFR, HER2) or a mutation or amplification in a regulator of RAS downstream of the receptor (e.g. SOS1 gain of function, NF1 loss of function).

102. The method of any one of claims 98 to 101, wherein said cells are selected from melanoma cells, thyroid carcinoma cells (e.g. papillary thyroid carcinoma cells), colorectal, ovarian, breast cancer cells, endometrial cancer cells, liver cancer cells, sarcoma cells, stomach cancer cells, pancreatic carcinoma cells, Barret's adenocarcinoma cells, glioma cells (e.g. ependymoma cells), lung cancer cells (e.g. non-small cell lung cancer cells), head and neck cancer cells, acute lymphoblastic leukemia cells, acute myelogenous leukemia cells, non-Hodgkin's lymphoma cells, and hairy-cell leukemia cells.

103. The method of any one of claims 98 to 102, wherein said cells are selected from colon or colorectal cancer cells, lung cancer cells, pancreatic cancer cells, thyroid cancer cells, breast cancer cells and melanoma cells.

104. The method of any one of claims 98 to 103, wherein said method comprises inhibiting the RAS-ERK signaling pathway without substantially inducing a paradoxical pathway.

105. The method of any one of claims 98 to 104, wherein said contacting is done in vivo.

106. The method of any one of claims 98 to 104, wherein said contacting is done ex vivo.
